(19) United States
(12) Patent Application Publication
(10) Pub. No.: US 2026/0022401 A1
(43) Pub. Date: Jan. 22, 2026

(54) Novel AAV rep ORFs and Rep polypeptides

(71) Applicant: Hoffmann-La Roche Inc., Little Falls, NJ (US)

(72) Inventors: Julia FAKHIRI, Penzberg (DE); Veronika OETTL, Penzberg (DE); Stefan SEEBER, Sindelsdorf (DE); Thomas STEININGER, Penzberg (DE)

(73) Assignee: Hoffmann-La Roche Inc., Little Falls, NJ (US)

(21) Appl. No.: 19/274,959

(22) Filed: Jul. 21, 2025

(30) Foreign Application Priority Data
Jul. 22, 2024 (EP) 24190010.9
Jan. 14, 2025 (EP) 25151734.8

(51) Int. Cl. C12N 15/86

(52) U.S. Cl. CPC C12N 15/86; C12N 2750/14122; C12N 2750/14123; C12N 2750/14143; C12N 2750/14152

(57) ABSTRACT

Herein is reported a nucleic acid encoding a functional adeno-associated virus Rep protein, characterized in that the nucleic acid comprises in 5′- to 3′-direction a first 5′-terminal part, a second 5′-terminal part, a central part, a second 3′-terminal part and a first 3′-terminal part, wherein the first 5′-terminal part is similar to a part of the rep gene of the AAV2 or AAV6 serotype and a second 5′-terminal part is similar to a part of the rep gene of an AAV1 serotype and the second 3′-terminal part is similar to a part of the rep gene of the AAV10 or AAV11 serotype and a first 3′-terminal part is similar to a part of the rep gene of an AAV13 serotype.

The current invention is in the field of recombinant adeno-associated virus particle (rAAVp) production. In more detail, herein are reported novel AAV rep ORFs which are chimeras of rep sequences derived from different AAV serotypes.

### CROSS REFERENCE TO RELATED APPLICATIONS

This application claims priority to European Patent Application Number 24190010.9, filed Jul. 22, 2024, and European Patent Application Number 25151734.8, filed Jan. 14, 2025, each of which are incorporated herein by reference in its entirety.

### SEQUENCE LISTING

This application contains a Sequence Listing which has been submitted electronically in XML format and is hereby incorporated by reference in its entirety. Said XML copy, created on Jul. 18, 2025, is named “P39442-US-1-Sequence Listing.xml” and is 114,073 bytes in size.

### BACKGROUND OF THE INVENTION

Adeno-Associated Viral (AAV) vectors hold great promise for delivering therapeutic genes. However, the high production costs of AAV gene therapy products represent a significant challenge within the field [1,2]. This is mainly attributed to the complexity of the manufacturing process, which involves intricate production steps, including the generation of high-quality viral vectors and subsequent purification. Empty AAV capsids, which lack the therapeutic gene and can impact the overall efficacy and safety of AAV vectors, are one of several potential contaminants of viral vector preparations. Purification strategies have evolved to separate full from empty capsids, but recent research revealed that empty capsids may not be truly empty [3,4], thus complicating their separation from the full-length genome-containing viral capsids. This emphasizes the need for a better understanding of genome replication and packaging mechanisms to reduce these unwanted byproducts.

To date, four nascent and overlapping Open Reading Frames (ORFs) have been identified in wild-type AAVs: Rep [5], Cap [5], AAP [6], and MAAP [7]. Within the intricate machinery of AAV vectors, the non-structural proteins (Rep) emerge as multifaceted entities crucial for the viral life cycle. Four overlapping Rep proteins (Rep78, Rep68, Rep52, and Rep40) are expressed from two promoters at map units 5 and 19 [8]. The large Rep proteins, Rep78 and Rep68, govern processes such as replication [9], transcription regulation [10], and site-specific integration [11]. They orchestrate AAV replication by utilizing endonuclease and helicase activities, ensuring the effective amplification of the viral genome within the host cell [12]. The smaller Rep proteins, Rep52 and Rep40, are believed to contribute to propelling the viral genome into pre-formed capsids [13, 14].

By regulating AAV transcription, Rep proteins impact the delicate balance between productive replication and maintenance of the latent state, both in the presence and absence of a helper virus infection [10]. Understanding these molecular interactions is crucial for manipulating recombinant AAV particles (rAAVp) for therapeutic purposes. Researchers actively explored strategies to modify Rep proteins, modulating their expression and relative levels to enhance particle production. Notably, the fine-tuning of enzymatic activities was achieved by site-directed mutagenesis of protein domains [20], modifying interactions with host factors [21], and optimizing expression profiles [22, 23, 24].

The exploration was spurred by pseudotyping, i.e., the ability to cross-package AAV2-based genomes into capsids other than AAV2 [25-27]. While Rep2 excels in its AAV2-based system, cross-complementation with Rep proteins from other AAV serotypes [26, 28-30] has been demonstrated. Some AAV vector preparation titers could even be increased using the respective ITRs belonging to the utilized Rep protein [26, 20, 31].

The widespread availability of gene therapies is hindered by the persistently high costs of producing rAAVp [1,2,45], whereby certain aspects of the production process itself continue to pose challenges. Essentially, three key components are needed for rAAVp production:
(i) the cargo, containing a transgene cassette flanked by ITRs;
(ii) AAV Rep and Cap proteins supplied in trans;
(iii) genes from a helper virus, usually Adenovirus.

Strategies to enhance this system include plasmid engineering (e.g., minicircles/nanoplasmids [46,47], Doggybone™ [48,49], process optimization [50,51], and creating stable cell lines [52,53] or Adenovirus-/Herpesvirus- and Baculovirus-based systems [54-57] that overcome plasmid dependency. Importantly, all of these methods maintain the integrity of the protein components with modification to the expression cassettes themselves to fit into the respective systems.

A major focus in both academic and industrial laboratories has been the engineering of Cap proteins [58]. The main goal is to guide the vector toward specific tissues/cells, enhancing specificity and ultimately reducing production costs. Some engineered capsids naturally show higher particle titers than their parental counterparts, especially those originating from directed evolution approaches [59].

Ward, P. and Walsh, C. E. reported the making of AAV cap chimeras using PCR shuffling and a staggered extension PCR procedure (Mol. Ther. 15 (2007) supplement 1, S32).

A major challenge in recombinant adeno-associated virus particle (rAAVp) production is the efficient packaging of the genome into the viral capsid, with empty or partially filled capsids often representing over 90% of the produced material.

So far, the Rep2 variant from the AAV2 serotype is commonly used in rAAVp production systems, partly due to historical reasons, but also the reported superiority of Rep2 [26, 28].

Mietzsch and colleagues suggested correcting the AAV8 rep nucleotide sequence in the VR-A and VR-B region. Low VP expression of AAV6 and AAV8 capsid proteins when combined to their respective Rep proteins, was linked to the DNA sequence of the region encoding the zinc finger domain. Swapping that region with the same region of rep2 rescued VP protein expression.

Rabinowitz et al. performed a study, wherein the rep2 gene was kept constant and combined with AAV capsids of different serotypes [25]. This resulted in low capsid expression, which was then reversed by appending stretches from the respective rep sequences at the 5′-end of the cap genes. The region encoding the zinc finger domain in rep2 was replaced with sequences derived from rep3, rep4, and rep5 to produce the respective AAV serotypes. This increased particle production up to 1000-fold. A small serotype-specific stretch between rep and cap was included.

Tejero et al. showed the impact of the 5′-DNA region, involving the DNA- or origin-binding domain in increasing DNA packaging ability [20]. Here, inverting four amino acid residues at the N-terminus of Rep6 to their counterpart in Rep2, restored Rep6 expression and ability to package AAV2 ITR-based genomes to packaging rates similar to Rep2 but with lower genomic titers.

WO 2011/112090 reported a method for identifying variant Rep protein encoding nucleic acids. In the first step of the method molecular diversity is created by introducing random point mutations, via an error prone PCR.

WO 2019/173538 reported compositions and methods for packaging a recombinant adeno-associated virus particle comprising inverted terminal repeats (ITRs) and rep genes of different serotypes and/or using chimeric rep genes.

Thus, there is still a need to improve packaging efficiency in rAAVp production.

### SUMMARY OF THE INVENTION

Herein is reported a nucleic acid encoding a functional adeno-associated virus Rep protein, characterized in that the nucleic acid comprises in 5′- to 3′-direction a first 5′-terminal part, a second 5′-terminal part, a central part, a second 3′-terminal part and a first 3′-terminal part, wherein the first 5′-terminal part is similar to a part of the rep gene of the AAV2 or AAV6 serotype and a second 5′-terminal part is similar to a part of the rep gene of an AAV1 serotype and the second 3′-terminal part is similar to a part of the rep gene of the AAV10 or AAV11 serotype and a first 3′-terminal part is similar to a part of the rep gene of an AAV13 serotype.

The current invention is based, at least in part, on the finding that the rAA Vp production, especially the packaging efficiency, can be improved by genetic engineering of the replication and packaging protein of AAV (Rep), i.e. by combining elements of the Reps of different serotypes.

The current invention comprises at least the following independent and dependent embodiments:
1. A nucleic acid encoding a functional adeno-associated virus Rep protein, characterized in that the nucleic acid comprises at least 14 different fragments derived from naturally occurring rep genes of at least 8 different serotypes.
2. The nucleic acid of embodiment 1, characterized in comprising at least 17 different part fragments derived from naturally occurring rep genes of at least 8 different serotypes.
3. A nucleic acid encoding a functional adeno-associated virus Rep protein, characterized in that the nucleic acid comprises a 5′-terminal part, a central part and a 3′-terminal part, wherein the 5′-terminal part, the central part and the 3′-terminal part are independently of each other derived from naturally occurring rep genes of different serotypes.
4. The nucleic acid of embodiment 3, wherein
   a) the 5′-terminal part is similar to a part of the rep gene of the AAV3 serotype and the 3′-terminal part is similar to a part of the rep gene of the AAV10 serotype;
   b) the 5′-terminal part is similar to a part of the rep gene of the AAV3 serotype and the 3′-terminal part is similar to a part of the rep gene of the AAV13 serotype;
   c) the 5′-terminal part is similar to a part of the rep gene of the AAV1 serotype and the 3′-terminal part is similar to a part of the rep gene of the AAV13 serotype;
   d) the 5′-terminal part is similar to a part of the rep gene of the AAV1 serotype and the 3′-terminal part is similar to a part of the rep gene of the AAV2 serotype;
   e) the 5′-terminal part is similar to a part of the rep gene of the AAV3 serotype and the 3′-terminal part is similar to a part of the rep gene of the AAV4 serotype;
   f) the 5′-terminal part is similar to a part of the rep gene of the AAV6 serotype and the 3′-terminal part is similar to a part of the rep gene of the AAV4 serotype;
   g) the 5′-terminal part is similar to a part of the rep gene of any AAV serotype and the 3′-terminal part is similar to a part of the rep gene of the AAV2 serotype; or
   h) the 5′-terminal part is similar to a part of the rep gene of any AAV serotype and the 3′-terminal part is similar to a part of the rep gene of the AAV13 serotype; or
   i) the 5′-terminal part is similar to a part of the rep gene of the AAV3 serotype and the 3′-terminal part is similar to a part of the rep gene of the AAV6 serotype; or
   j) the 5′-terminal part is similar to a part of the rep gene of the AAV1 serotype and the 3′-terminal part is similar to a part of the rep gene of the AAV6 serotype.
5. The nucleic acid of embodiment 3 or 4, wherein the 5′-terminal part is similar to a part of the rep gene of the AAV2 or AAV6 serotype and the 3′-terminal part is similar to a part of the rep gene of the AAV13 serotype.
6. The nucleic acid of any one of embodiments 3 to 5, characterized in that the 5′-terminal part comprises in 5′- to 3′-direction a first 5′-terminal part and a second 5′-terminal part and the 3′-terminal part comprises in 5′- to 3′-direction a second 3′-terminal part and a first 3′-terminal part.
7. The nucleic acid of any one of embodiments 3 to 6, wherein
   a) the first 5′-terminal part is similar to a part of the rep gene of the AAV3 serotype and the second 5′-terminal part is similar to a part of the rep gene of the AAV6 serotype and the second 3′-terminal part is similar to a part of the rep gene of the AAV11 serotype and the first 3′-terminal part is similar to a part of the rep gene of the AAV10 serotype;
   b) the first 5′-terminal part is similar to a part of the rep gene of the AAV3 serotype and the second 5′-terminal part is similar to a part of the rep gene of the AAV4 serotype and the second 3′-terminal part is similar to a part of the rep gene of the AAV4 serotype and the first 3′-terminal part is similar to a part of the rep gene of an AAV13 serotype;
   c) the first 5′-terminal part is similar to a part of the rep gene of the AAV3 serotype and the second 5′-terminal part is similar to a part of the rep gene of the AAV10 serotype and the second 3′-terminal part is similar to a part of the rep gene of the AAV11 serotype and the first 3′-terminal part is similar to a part of the rep gene of the AAV4 serotype;
   d) the first 5′-terminal part is similar to a part of the rep gene of the AAV6 serotype and the second 5′-terminal part is similar to a part of the rep gene of the AAV10 serotype and the second 3′-terminal part is similar to a part of the rep gene of the AAV11 serotype and the first 3′-terminal part is similar to a part of the rep gene of an AAV4 serotype;
   e) the first 5′-terminal part is similar to a part of the rep gene of any AAV serotype and the second 5′-terminal part is similar to a part of the rep gene of the AAV1 serotype and the second 3′-terminal part is similar to a part of the rep gene of the AAV10 serotype and the first 3′-terminal part is similar to a part of the rep gene of the AAV2 serotype; or
   f) the first 5′-terminal part is similar to a part of the rep gene of any AAV serotype and the second 5′-terminal part is similar to a part of the rep gene of the AAV1 serotype and the second 3′-terminal part is similar to a part of the rep gene of any AAV serotype and the first 3′-terminal part is similar to a part of the rep gene of an AAV13 serotype; or
   g) the first 5′-terminal part is similar to a part of the rep gene of the AAV3 serotype and the second 5′-terminal part is similar to a part of the rep gene of the AAV1 serotype and the second 3′-terminal part is similar to a part of the rep gene of any AAV serotype and the first 3′-terminal part is similar to a part of the rep gene of an AAV13 serotype; or
   h) the first 5′-terminal part is similar to a part of the rep gene of the AAV3 serotype and the second 5′-terminal part is similar to a part of the rep gene of the AAV1 serotype and the second 3′-terminal part is similar to a part of the rep gene of any AAV serotype and the first 3′-terminal part is similar to a part of the rep gene of an AAV6 serotype; or.
8. The nucleic acid any one of embodiments 3 to 7, wherein the first 5′-terminal part is similar to a part of the rep gene of the AAV2 or AAV6 serotype and a second 5′-terminal part is similar to a part of the rep gene of an AAV1 serotype and the second 3′-terminal part is similar to a part of the rep gene of the AAV10 or AAV11 serotype and a first 3′-terminal part is similar to a part of the rep gene of an AAV13 serotype.
8a. The nucleic acid any one of embodiments 3 to 8, wherein the central part comprises in 5′ to 3′ direction a first part that is similar to a part of the rep gene of the AAV10 serotype, a second part that is similar to a part of the rep gene of the AAV3 serotype, a third part that is similar to a part of the rep gene of the AAV13 serotype. a fourth part that is similar to a part of the rep gene of the AAV9 serotype, a fifth part that is similar to a part of the rep gene of the AAV13 serotype and a sixth part that is similar to a part of the rep gene of the AAV11 serotype.
8b. The nucleic acid according to embodiment 8a, wherein the 5′-terminal part is similar to a part of the rep gene of the AAV1 or AAV6 serotype and the 3′-terminal part is similar to a part of the rep gene of the AAV4 serotype.
9. A nucleic acid encoding a functional adeno-associated virus Rep protein comprising in 5′- to 3′-direction nucleic acid fragments derived from naturally occurring rep genes of

AAV3-AAVx-AAV6-AAVx-AAV11-AAV8-AAV11-AAV13-AAVX-
AAV6-AAV12-AAV6-AAV11-AAV10

with AAVx denoting any AAV serotype.
10. A nucleic acid encoding a functional adeno-associated virus Rep protein of clone 0.15.
11. A nucleic acid encoding a functional adeno-associated virus Rep protein that has the nucleic acid sequence of

(SEQ ID NO: 37)
ATGCCGGGGTTCTACGAGATTGTCCTGAAGGTCCCGAGTGACCTGGACGA
GCACCTGCCGGGCATTTCTAACTCGTTTGTTAACTGGGTGGCCGAGAAGG
AATGGGAGCTGCCCCCGGATTCTGACATGGATCGGAATCTGATCGAGCAG
GCACCCCTGACCGTGGCCGAGAAGCTGCAGCGCGACTTCCTGGTCCAGTG
GCGCCGCGTGAGTAAGGCCCCGGAGGCCCTCTTCTTTGTTCAGTTCGAGA
AGGGCGAGAGCTACTTTCACCTGCACGTTCTGGTCGAGACCACGGGGGTC
AAGTCCATGGTCCTGGGCCGCTTCCTGAGTCAGATCAGAGACAGGCTGGT
GCAGACCATCTACCGCGGGGTCGAGCCCACGCTGCCCAACTGGTTCGCGG
TGACCAAAGACGCGGTAATGGCGCCGGCGGGGGGGAACAAGGTGGTGGAC
GAGTGCTACATCCCCAACTACCTCCTGCCCAAGACCCAGCCCGAGCTGCA
GTGGGCGTGGACTAACATGGAGGAGTATATAAGCGCGTGTCTAAACCTCG
CGGAGCGTAAACGGCTCGTGGCGCAGCACCTGACCCACGTCAGCCAGACG
CAGGAGCAGAACAAGGAGAATCTGAACCCGAATTCTGACGCGCCCGTGAT
CAGGTCAAAAACCTCCGCGCGCTACATGGAGCTGGTCGGGTGGCTGGTGG
ACCGGGGCATCACCTCCGAGAAGCAGTGGATCCAGGAGGACCAGGCCTCG
TACATCTCCTTCAACGCCGCCTCCAACTCGCGGTCACAAATCAAGGCCGC
ACTGGACAATGCCGGCAAGATCATGGCGCTGACCAAATCCGCGCCCGACT
ACCTGGTAGGCCCGTCCTTACCCGCGGACATTAAGGCCAACCGCATCTAC
CGCATCCTGGAGCTCAACGGCTACGACCCCGCCTACGCGGCCTCCGTCTT
CCTGGGCTGGGCGCAAAAGAAGTTCGGGAAGAGGAACACCATCTGGCTCT
TTGGGCCGGCCACGACGGGTAAAACCAACATCGCGGAAGCCATCGCCCAC
GCCGTGCCCTTCTACGGCTGCGTCAACTGGACCAATGAGAACTTTCCGTT
CAACGACTGTGTCGACAAGATGGTGATCTGGTGGGAGGAGGGCAAGATGA
CGGCCAAGGTCGTGGAGTCCGCCAAGGCCATTCTCGGCGGCAGCAAGGTG
CGCGTGGACCAAAAGTGCAAGTCGTCCGCCCAGATCGATCCCACCCCCGT
GATCGTCACCTCCAACACCAACATGTGCGCCGTGATTGACGGGAACAGCA
CCACCTTCGAGCACCAGCAGCCCCTGCAGGACCGGATGTTCAAGTTTGAA
CTCACCCGCCGCCTCGACCACGACTTTGGCAAGGTCACCAAGCAGGAAGT
CAAGGACTTTTTCCGGTGGGCGCAGGATCACGTGACCGAGGTGGCGCATG
AGTTCTACGTCAGAAAGGGTGGAGCCAACAAGAGACCCGCCCCCAGTGAC
GCGGATATAAGCGAGCCCAAGCGGGCCTGCCCCTCAGTTCCGGAGCCATC
GACGTCAGACGCGGAAGCACCGGTGGACTTTGCGGACAGGTACCAAAACA
AATGTTCTCGTCACGCGGGCATGCTTCAGATGCTGTTTCCCTGCAAGACA
TGCGAGAGAATGAATCAGAATTTCAACGTCTGCTTCACGCACGGGGTCAG
AGACTGCTCAGAGTGCTTCCCCGGCGCGTCAGAATCTCAACCTGTCGTCA
GAAAAAAGACGTATCAGAAACTGTGCGCGATTCATCATCTGCTGGGGGGG
GCACCCGAGATTGCGTGTTCGGCCTGCGATCTCGTCAACGTGGACTTGGA
TGACTGKGTTTCTGAACAATAA.

12. An adeno-associated virus Rep protein with the amino acid sequence of

(SEQ ID NO: 49)

MPGFYEIVLK VPSDLDEHLP GISNSFVNWV AEKEWELPPD
SDMDRNLIEQ APLTVAEKLQ RDFLVQWRRV SKAPEALFFV
QFEKGESYFH LHVLVETTGV KSMVLGRFLS QIRDRLVQTI
YRGVEPTLPN WFAVTKDAVM APAGGNKVVD ECYIPNYLLP
KTQPELQWAW INMEEYISAC LNLAERKRLV AQHLTHVSQT
QEQNKENLNP NSDAPVIRSK TSARYMELVG WLVDRGITSE
KQWIQEDQAS YISFNAASNS RSQIKAALDN AGKIMALTKS
APDYLVGPSL PADIKANRIY RILELNGYDP AYAASVELGW
AQKKEGKRNT IWLFGPATTG KTNIAEAIAH AVPFYGCVNW
TNENFPENDC VDKMVIWWEE GKMTAKVVES AKAILGGSKV
RVDQKCKSSA QIDPTPVIVT SNTNMCAVID GNSTTFEHQQ
PLQDRMFKFE LTRRLDHDFG KVTKQEVKDF FRWAQDHVTE
VAHEFYVRKG GANKRPAPSD ADISEPKRAC PSVPEPSTSD
AEAPVDFADR YQNKCSRHAG MLQMLFPCKT CERMNQNFNV
CFTHGVRDCS ECFPGASESQ PVVRKKTYQK LCAIHHLLGR
APEIACSACD LVNVDLDDXV SEQ.

13. A nucleic acid encoding the Rep protein of embodiment 12.
14. A nucleic acid encoding a functional adeno-associated virus Rep protein comprising in 5′- to 3′-direction nucleic acid fragments derived from naturally occurring rep genes of

AAV7-AAV3-AAV4-AAV11-AAVx-AAV9-AAV12-AAV2-AAV1-
AAV10-AAV4-AAVx-AAV4-AAV12-AAV1-AAV7-AAV10-AAV4-
AAV9-AAV12-AAV3-AAV4-AAVx-AAV4-AAV13-AAV7

with AAVx denoting any AAV serotype.
15. A nucleic acid encoding a functional adeno-associated virus Rep protein of clone 1.01.
16. A nucleic acid encoding a functional adeno-associated virus Rep protein that has the nucleic acid sequence of

(SEQ ID NO: 38)
ATGCCGGGCTTCTACGAGATTGTCCTGAAGGTCCCGAGTGACCTGGACGA
GCACCTGCCGGGCATTTCTAACTCGTTTGTTAACTGGGTGGCCGAGAAGG
AATGGGAGCTGCCGCCGGATTCTGACATGGACTTGAATCTGATTGAGCAG
GCACCCCTGACCGTGGCCGAAAAGCTGCAGCGCGACTTCCTGGTCCACTG
GCGCCGCGTGAGTAAGGCCCCGGAGGCCCTCTTCTTTGTTCAGTTCGAGA
AGGGCGAGTCCTACTTCCACCTCCATATTCTGGTGGAGACCACGGGGGTC
AAATCCATGGTGCTGGGCCGCTTCCTGAGTCAGATTAGGGACAAGCTGGT
GCAGACCATCTACCGCGGGATCGAGCCGACCCTGCCCAACTGGTTCGCGG
TGACCAAGACGCGTAATGGCGCCGGCGGGGGGAACAAGGTGGTGGACGAG
TGCTACATCCCCAACTACCTGCTCCCCAAGACCCAGCCCGAGCTGCAGTG
GGCGTGGACTAACATGGAGGAGTATATAAGCGCCTGTTTGAACCTCGCGG
AGCGTAAACGGCTCGTGGCGCAGCATCTGACGCACGTGTCGCAGACGCAG
GAGCAGAACAAGGAGAATCTGAACCCGAATTCTGACGCGCCCGTGATCAG
GTCAAAAACCTCCGCGCGCTACATGGAGCTGGTCGGGTGGCTGGTGGACC
GCGGGATCACGTCAGAAAAGCAATGGATCCAGGAGGACCAGGCGTCCTAC
ATCTCCTTCAACGCCGCCTCCAACTCGCGGTCACAAATCAAGGCCGCGCT
GGACAATGCCTCCAAAATCATGAGCCTCACCAAAACGGCTCCGGACTATC
TCATCGGGCAGCAGCCCGTGGGGGACATTACCACCAACCGGATCTACAAA
ATCCTGGAACTGAACGGGTACGACCCCCAGTACGCCGCCTCCGTCTTTCT
CGGCTGGGCCCAGAAAAGGTTCGGGAAGCGCAACACCATCTGGCTGTTTG
GGCCGGCCACCACCGGCAAGACCAACATTGCGGAAGCCATCGCCCACGCC
GTGCCCTTCTACGGCTGCGTCAACTGGACCAATGAGAACTTTCCCTTCAA
CGATTGCGTCGACAAGATGGTGATCTGGTGGGAGGAGGGCAAGATGACCG
CCAAGGTCGTAGAGAGCGCCAAGGCCATCCTGGGCGGAAGCAAGGTGCGC
GTGGACCAAAAGTGCAAGTCGTCCGCCCAGATCGACCCCACTCCCGTGAT
CGTCACCTCCAACACCAACATGTGCGCCGTGATTGACGGGAACAGCACCA
CCTTCGAGCACCAGCAGCCCCTGCAGGACCGGATGTTCAAATTTGAACTT
ACCCGCCGTTTGGACCATGACTTTGGCAAGGTCACCAAGCAGGAAGTCAA
AGACTTTTTCCGGTGGGCGTCAGATCACGTGACCGAGGTGACTCACGAGT
TTTACGTCAGAAAGGGCGGAGCCAGCAAAAGACCCGCCCCCGATGACGCG
GATAAAAGCGAGCCCAAGCGGGCCTGTCCGTCAGTTGCGCAGCCATCGAC
GTCAGACGCGGAAGCTCCGGTGGACTACGCGGACAGGTACCAAAACAAAT
GTTCTCGTCACGTGGGTATGAATCTGATGCTTTTTCCCTGCCGGCAATGC
GAGAGAATGAATCAGAATGTGGACATTTGCTTCACGCACGGGGTCATGGA
CTGTGCCGAGTGCTTCCCCGTGTCAGAATCTCAACCCGTGTCTGTCGTCA
GAAAGCGGACATATCAGAAACTGTGTTTGATTCATCACATCATGGGGAGG
GCGCCCGAGGTGGCTTGTTCGGCCTGCGAACTGGCCAATGTGGACTTGGA
TGACTGTGACATGGAACAATAA.

17. An adeno-associated virus Rep protein with the amino acid sequence of

(SEQ ID NO: 50)

MPGFYEIVLK VPSDLDEHLP GISNSFVNWV AEKEWELPPD
SDMDLNLIEQ APLTVAEKLQ RDFLVHWRRV SKAPEALFFV
QFEKGESYFH LHILVETTGV KSMVLGRFLS QIRDKLVQTI
YRGIEPTLPN WFAVTKTRNG AGGGNKVVDE CYIPNYLLPK
TQPELQWAWT NMEEYISACL NLAERKRLVA QHLTHVSQTQ
EQNKENLNPN SDAPVIRSKT SARYMELVGW LVDRGITSEK
QWIQEDQASY ISFNAASNSR SQIKAALDNA SKIMSLIKTA
PDYLIGQQPV GDITINRIYK ILELNGYDPQ YAASVELGWA
QKREGKRNTI WLFGPATTGK TNIAEAIAHA VPFYGCVNWT
NENFPFNDCV DKMVIWWEEG KMTAKVVESA KAILGGSKVR
VDQKCKSSAQ IDPTPVIVIS NINMCAVIDG NSTTFEHQQP
LQDRMFKFEL TRRLDHDFGK VTKQEVKDFF RWASDHVTEV
THEFYVRKGG ASKRPAPDDA DKSEPKRACP SVAQPSTSDA
EAPVDYADRY QNKCSRHVGM NLMLFPCRQC ERMNQNVDIC
FTHGVMDCAE CFPVSESQPV SVVRKRTYQK LCLIHHIMGR
APEVACSACE LANVDLDDCD MEQ.

18. A nucleic acid encoding the Rep protein of embodiment 17.
19. A nucleic acid encoding a functional adeno-associated virus Rep protein comprising in 5′- to 3′-direction nucleic acid fragments derived from naturally occurring rep genes of

AAVx-AAV1-AAV7-AAV9-AAV6-AAV9-AAV1-AAVx-AAV6-AAV3-
AAV6-AAV2-AAV11-AAVx-AAV11-AAVx-AAV13

with AAVx denoting any AAV serotype.
20. A nucleic acid encoding a functional adeno-associated virus Rep protein of clone 1.03.
21. A nucleic acid encoding a functional adeno-associated virus Rep protein that has the nucleic acid sequence of

(SEQ ID NO: 39)
ATGCCGGGGTTTTACGAGATTGTGATTAAGGTCCCCAGCGACCTTGACGAGCATCTGCC
CGGCATTTCTGACAGCTTTGTGAACTGGGTGGCCGAGAAGGAATGGGAGCTGCCCCCGG
ATTCTGACATGGATCTGAATCTGATTGAGCAGGCACCCCTGACCGTGGCCGAGAAGCTG
CAGCGCGACTTCCTGGTCCAATGGCGCCGCGTGAGTAAGGCCCCGGAGGCCCTCTTCTT
TGTTCAGTTCGAGAAGGGCGAGAGCTACTTCCACCTTCACGTTCTGGTGGAGACCACGG
GGGTCAAGTCCATGGTGCTAGGCCGCTTCCTGAGTCAGATTCGGGAGAAGCTGGTCCAG
ACCATCTACCGCGGGATCGAGCCGACCCTGCCCAACTGGTTCGCGGTGACCAAGACGCG
TAATGGCGCCGGAGGGGGGAACAAGGTGGTGGACGAGTGCTACATCCCCAACTACCTCC
TGCCCAAGACTCAGCCCGAGCTGCAGTGGGCGTGGACTAACATGGAGGAGTATATAAGC
GCGTGCTTGAACCTGGCCGAGCGCAAACGGCTCGTGGCGCAGCACCTGACCCACGTCAG
CCAGACCCAGGAGCAGAACAAGGAGAATCTGAACCCCAATTCTGACGCGCCCGTGATCA
GGTCAAAAACCTCCGCACGCTACATGGAGCTGGTCGGGTGGCTGGTGGACCGGGGCATC
ACCTCCGAGAAGCAGTGGATCCAGGAGGACCAGGCCTCGTACATCTCCTTCAACGCCGC
CTCCAACTCGCGGTCCCAGATCAAGGCCGCGCTGGACAATGCCTCCAAGATCATGAGCC
TGACAAAGACGGCTCCGGACTACCTGGTGGGCAGCAACCCGCCGGAGGACATTACCAAA
AATCGGATCTACCAAATCCTGGAGCTGAACGGGTACGATCCGCAGTACGCGGCCTCCGT
CTTCCTGGGCTGGGCGCAAAAGAAGTTCGGGAAGAGGAACACCATCTGGCTCTTTGGGC
CGGCCACGACGGGTAAAACCAACATCGCGGAAGCCATCGCCCACGCCGTGCCCTTCTAC
GGCTGCGTCAACTGGACCAATGAGAACTTTCCCTTCAACGATTGCGTCGACAAGATGGT
GATCTGGTGGGAGGAGGGCAAGATGACGGCCAAGGTCGTGGAGTCGGCCAAAGCCATTC
TCGGAGGAAGCAAGGTGCGCGTGGACCAAAAGTGCAAGTCCTCGGCCCAGATCGACCCC
ACGCCCGTGATCGTCACCTCCAACACCAACATGTGCGCCGTGATCGACGGGAACAGCAC
CACCTTCGAGCACCAGCAGCCGCTGCAGGACCGGATGTTCAAATTTGAACTCACCCGCC
GTCTGGAGCATGACTTTGGCAAGGTGACAAAGCAGGAAGTCAAAGAGTTCTTCCGCTGG
GCGCAGGATCACGTGACCGAGGTGGCGCATGAGTTCTACGTCAGAAAGGGCGGAGCCAC
CAAAAGACCCGCCCCCAGTGACGCGGATATAAGCGAGCCCAAGCGGGCCTGCCCCTCAG
TTCCGGAGCCATCGACGTCAGACGCGGAAGCGCCGGTGGACTTTGCGGACAGGTACCAA
AACAAATGTTCTCGTCACGCGGGCATGCTTCAGATGCTGTTTCCCTGCAAGACATGCGA
GAGAATGAATCAGAATTTCAACGTCTGCTTCACGCACGGGGTCAGAGACTGCTCAGAGT
GCTTCCCCGGCGTGTCAGAATCTCAACCCGTGTCTGTCGTCAGAAAGCGGACATATCAG
AAACTGTGTCCGATTCATCACATCATGGGGAGGGCGCCCGAGATTGCTTGCTCGGCCTG
CGATCTGGTCAACGTGGACCTGGATGACTGTGTTTCTGAGCAATAA.

22. An adeno-associated virus Rep protein with the amino acid sequence of

(SEQ ID NO: 51)
MPGFYEIVIK VPSDLDEHLP GISDSFVNWV AEKEWELPPD
SDMDLNLIEQ APLTVAEKLQ RDFLVQWRRV SKAPEALFFV
QFEKGESYFH LHVLVETTGV KSMVLGRFLS QIREKLVQTI
YRGIEPTLPN WFAVTKTRNG AGGGNKVVDE CYIPNYLLPK
TQPELQWAWT NMEEYISACL NLAERKRLVA QHLTHVSQTQ
EQNKENLNPN SDAPVIRSKT SARYMELVGW LVDRGITSEK
QWIQEDQASY ISFNAASNSR SQIKAALDNA SKIMSLIKTA
PDYLVGSNPP EDITKNRIYQ ILELNGYDPQ YAASVFLGWA
QKKFGKRNTI WLFGPATTGK TNIAEAIAHA VPFYGCVNWT
NENFPFNDCV DKMVIWWEEG KMTAKVVESA KAILGGSKVR
VDQKCKSSAQ IDPTPVIVTS NTNMCAVIDG NSTTFEHQQP
LQDRMFKFEL TRRLEHDFGK VTKQEVKEFF RWAQDHVTEV
AHEFYVRKGG ATKRPAPSDA DISEPKRACP SVPEPSTSDA
EAPVDFADRY QNKCSRHAGM LQMLFPCKTC ERMNQNFNVC
FTHGVRDCSE CFPGVSESQP VSVVRKRTYQ KLCPIHHIMG
RAPEIACSAC DLVNVDLDDC VSEQ.

23. A nucleic acid encoding the Rep protein of embodiment 22.
24. A nucleic acid encoding a functional adeno-associated virus Rep protein comprising in 5′- to 3′-direction nucleic acid fragments derived from naturally occurring rep genes of

AAVx-AAV1-AAV3-AAVx-AAV10-AAVx-AAV2-AAV4-AAV13-
AAV11-AAVx-AAV12-AAVx-AAV12-AAV10-AAV7-AAV13-AAV1-
AAV10-AAV2

with AAVx denoting any AAV serotype.
25. A nucleic acid encoding a functional adeno-associated virus Rep protein of clone 1.10.
26. A nucleic acid encoding a functional adeno-associated virus Rep protein that has the nucleic acid sequence of

(SEQ ID NO: 41)
ATGCCGGGGTTTTACGAGATTGTGATTAAGGTCCCCAGCGACCTTGACGAGCATCTGCC
CGGCATTTCTGACAGCTTTGTGAACTGGGTGGCCGAGAAGGAATGGGAGCTGCCCCCGG
ATTCTGACATGGATCTGAATCTGATTGAGCAGGCACCCCTGACCGTGGCCGAGAAGCTG
CAGCGCGAGTTCCTGGTGGAGTGGCGCCGCGTGAGTAAGGCCCCGGAGGCCCTCTTTTT
TGTCCAGTTCGAAAAGGGGGAGACCTACTTCCACCTGCACGTGCTGATTGAGACCATCG
GGGTCAAATCCATGGTGGTCGGCCGCTACGTGAGCCAGATTAAAGAGAAGCTGGTGACC
CGCATCTACCGCGGGGTCGAGCCGCAGCTTCCGAACTGGTTCGCGGTGACCAAGACGCG
TAATGGCGCCGGAGGCGGGAACAAGGTGGTGGACGACTGCTACATCCCCAACTACCTGC
TCCCCAAGACCCAGCCCGAGCTGCAGTGGGCGTGGACTAACATGGAGGAGTATATAAGC
GCGTGTCTGAACCTCGCGGAGCGTAAACGGCTCGTGGCGCAGCACCTGACCCACGTCAG
CCAGACGCAGGAGCAGAACAAGGAGAATCTGAACCCCAATTCTGACGCGCCCGTGATCA
GGTCAAAAACCTCCGCGCGCTACATGGAGCTGGTCGGGTGGCTCGTGGACAAGGGGATT
ACCTCGGAGAAGCAGTGGATCCAGGAGGACCAGGCCTCGTACATCTCCTTCAACGCCGC
CTCCAACTCGCGGTCACAAATCAAGGCCGCGCTGGACAATGCCTCCAAAATCATGAGCC
TGACAAAGACGGCTCCGGACTACCTGGTGGGCCAGAACCCGCCGGAGGACATTACCAGC
AACCGGATCTACAAAATCCTCGAGATGAACGGGTACGATCCGCAGTACGCGGCCTCCGT
CTTCCTGGGCTGGGCGCAAAAGAAGTTCGGTAAACGCAACACCATCTGGCTGTTTGGGC
CTGCAACTACCGGCAAGACCAACATCGCGGAAGCCATCGCCCACGCGGTCCCCTTCTAC
GGCTGCGTCAACTGGACCAATGAGAACTTTCCCTTCAATGATTGCGTCGACAAGATGGT
GATCTGGTGGGAGGAGGGCAAGATGACGGCCAAGGTCGTGGAGTCCGCCAAGGCCATTC
TCGGCGGCAGCAAGGTGCGCGTGGACCAAAAATGCAAGGCCTCTGCGCAGATCGACCCC
ACCCCCGTGATCGTCACCTCCAACACCAACATGTGCGCCGTGATCGACGGGAACAGCAC
CACCTTCGAGCACCAGCAGCCCCTGCAGGACCGCATGTTCAAATTTGAACTCACCCGCC
GTCTGGAGCACGACTTTGGCAAGGTGACGAAGCAGGAAGTCAAAGAGTTCTTCCGCTGG
GCCAGTGATCACGTGACTGAGGTGTCTCACGAGTITTACGTCAGAAAGGGTGGAGCCAA
CAAAAGACCCGCCCCCGATGACGCGGATAAAAGCGAGCCCAAGCGGGCCTGCCCCTCAG
TTGCGGAGCCATCGACGTCAGACGCGGAAGCACCGGTGGACTTTGCGGACAGGTACCAA
AACAAATGTTCTCGTCACGCGGGCATGCTTCAGATGCTGTTTCCCTGCAGACAATGCGA
GAGAATGAATCAGAATTCAAATATCTGCTTCACTCACGGACAGAAAGACTGTTTAGAGT
GCTTTCCCGTGTCAGAATCTCAACCCGTTTCTGTCGTCAAAAAGGCGTATCAGAAACTG
TGCTACATTCATCATATCATGGGAAAGGTGCCAGACGCTTGCACTGCCTGCGATCTGGT
CAATGTGGATTTGGATGACTGCATCTTTGAACAATAA.

27. An adeno-associated virus Rep protein with the amino acid sequence of

(SEQ ID NO: 52)
MPGFYEIVIK VPSDLDEHLP GISDSFVNWV AEKEWELPPD
SDMDLNLIEQ APLTVAEKLQ REFLVEWRRV SKAPEALFFV
QFEKGETYFH LHVLIETIGV KSMVVGRYVS QIKEKLVTRI
YRGVEPQLPN WFAVTKTRNG AGGGNKVVDD CYIPNYLLPK
TQPELQWAWT NMEEYISACL NLAERKRLVA QHLTHVSQTQ
EQNKENLNPN SDAPVIRSKT SARYMELVGW LVDKGITSEK
QWIQEDQASY ISFNAASNSR SQIKAALDNA SKIMSLIKTA
PDYLVGQNPP EDITSNRIYK ILEMNGYDPQ YAASVFLGWA
QKKFGKRNTI WLFGPATTGK TNIAEAIAHA VPFYGCVNWT
NENFPFNDCV DKMVIWWEEG KMTAKVVESA KAILGGSKVR
VDQKCKASAQ IDPTPVIVTS NTNMCAVIDG NSTTFEHQQP
LQDRMFKFEL TRRLEHDFGK VTKQEVKEFF RWASDHVTEV
SHEFYVRKGG ANKRPAPDDA DKSEPKRACP SVAEPSTSDA
EAPVDFADRY QNKCSRHAGM LQMLFPCRQC ERMNQNSNIC
FTHGQKDCLE CFPVSESQPV SVVKKAYQKL CYIHHIMGKV
PDACTACDLV NVDLDDCIFE Q.

28. A nucleic acid encoding the Rep protein of embodiment 27.
29. The nucleic acid of any one of embodiment 1 to embodiment 11, embodiment 13 to embodiment 16, embodiment 18 to embodiment 21, embodiment 23 to embodiment 26 and embodiment 28, wherein the nucleic acid has a start codon of sequence ATG.
30. A plasmid comprising the nucleic acid of any one of embodiment 1 to embodiment 11, embodiment 13 to embodiment 16, embodiment 18 to embodiment 21, embodiment 23 to embodiment 26 and embodiment 28 to embodiment 29.
31. A composition comprising a first nucleic acid and a second nucleic acid, wherein the first nucleic acid comprises the nucleic acid according to any one of embodiment 1 to embodiment 11, embodiment 13 to embodiment 16, embodiment 18 to embodiment 21, embodiment 23 to embodiment 26 and embodiment 28 to embodiment 29 and a second nucleic acid comprises a cap gene.
32. The composition according to embodiment 31, wherein the cap gene is derived from or is of the naturally occurring serotype AAV2, AAV5, AAV8, AAV9 or AAVrh.74.
33. The composition according to any one of embodiment 31 to embodiment 32, wherein the first nucleic acid and the second nucleic acid are in the same plasmid or in different plasmids.
34. A cell comprising a nucleic acid according to any one of embodiment 1 to embodiment 11, embodiment 13 to embodiment 16, embodiment 18 to embodiment 21, embodiment 23 to embodiment 26 and embodiment 28 to embodiment 29.
35. The cell according to embodiment 34, wherein the cell expresses the Rep protein encoded by the nucleic acid.
36. A method for packaging a gene of interest in a recombinant adeno-associated virus particle, characterized in that the method comprises the following steps:
   contacting a cell that expresses
      i) a Rep protein according to any one of embodiment 12, embodiment 17, embodiment 22 or embodiment 27, and
      ii) adeno-associated virus Cap proteins with a nucleic acid that comprises the gene of interest to be packaged interspaced between a pair of inverted terminal repeats (ITRs),
   cultivating the cell under conditions suitable for the packaging of the gene of interest in a rAAVp and thereby packaging a gene of interest in a recombinant adeno-associated virus particle.
37. The method according to embodiment 36, wherein the Rep protein is expressed by transfecting or transducing the cell with a nucleic acid comprising a nucleic acid encoding the Rep protein.
38. The method according to any one of embodiment 36 to embodiment 37, wherein the Cap proteins are expressed by transfecting or transducing the cell with a nucleic acid comprising one or more nucleic acids encoding the Cap proteins.
39. A method for packaging a gene of interest in a recombinant adeno-associated virus particle, characterized in that the method comprises the step of:
   contacting a cell according to any one of embodiment 34 to embodiment 35 with
      i) a nucleic acid encoding adeno-associated virus Cap proteins, and
      ii) a nucleic acid comprising the gene of interest to be packaged interspaced between a pair of inverted terminal repeats (ITRs),
   cultivating the cell under conditions suitable for the packaging of the gene of interest in a rAAVp and thereby packaging a gene of interest in a recombinant adeno-associated virus particle.
40. A method for packaging a gene of interest in a recombinant adeno-associated virus particle, characterized in that the method comprises the step of:
   contacting a cell according to any one of embodiment 34 to embodiment 35 that also expresses adeno-associated virus Cap proteins, with a nucleic acid comprising the gene of interest to be packaged interspaced between a pair of inverted terminal repeats (ITRs),
   cultivating the cell under conditions suitable for the packaging of the gene of interest in a rAAVp and thereby packaging a gene of interest in a recombinant adeno-associated virus particle.
41. The method according to any one of embodiment 36 to embodiment 40, wherein the contacting is a transfecting, electroporating, nucleofecting, or microinjecting for nucleic acid transfer/transfection, preferably a transfecting.
42. The method according to any one of embodiment 36 to embodiment 41, wherein an inorganic substance, a cationic polymer or a cationic lipid is used for nucleic acid transfer/transfection.
43. The method according to any one of embodiment 36 to embodiment 42, wherein calcium phosphate, polyethylenimine or DEAE-dextran is used for nucleic acid transfer/transfection.
44. The method according to any one of embodiment 36 to embodiment 43, wherein polyethylenimine is used for nucleic acid transfer/transfection.
45. A method for producing a recombinant adeno-associated viral particle (rAAVp) comprising the steps:
   cultivating a mammalian cell comprising
      (a) a nucleic acid encoding a non-adeno-associated-virus RNA or protein, which is operably linked to two AAV inverted terminal repeats (ITRs) (i.e. the non-adeno-associated virus RNA or protein encoding nucleic acid is interspaced between the two AAV ITRs),
      (b) a nucleic acid encoding the AAV Cap protein VP1,
      (c) a nucleic acid according to any one of embodiment 1 to embodiment 11, embodiment 13 to embodiment 16, embodiment 18 to embodiment 21, embodiment 23 to embodiment 26 and embodiment 28 to embodiment 29,
      (d) a nucleic acid encoding the adenoviral helper function E4orf6, and
      (e) a nucleic acid encoding the adenoviral helper function E2A,
      (f) a nucleic acid encoding the adenoviral helper function E1A and E1B,
   recovering the rAAVp from the cell and/or the cultivation medium,
   optionally purifying the rAAVp,
   and thereby producing the rAAVp.
46. The method according to embodiment 45, wherein the method is for producing a rAAVp preparation.
47. The method according to any one of embodiment 45 to embodiment 46, wherein each nucleic acid is in an expression cassette comprising 5′ to the nucleic acid a promoter functional in the cell and 3′ to the nucleic acid a polyadenylation signal sequence functional in the cell, whereby the promoter, the nucleic acid and the polyadenylation signal sequence are operably linked.
48. The method according to any one of embodiment 45 to embodiment 47, wherein the cell comprises at least one gene encoding the AAV Cap protein VP1, VP2 and VP3.
49. The method according to any one of embodiment 45 to embodiment 48, wherein the cultivating of the mammalian cell is under conditions suitable for producing the rAAVp.
50. The method according to any one of embodiment 45 to embodiment 49, wherein the conditions suitable for producing the rAAVp are the generally established conditions as for the cultivation of eukaryotic cells.
51. The method according to any one of embodiment 45 to embodiment 50, wherein the conditions suitable for producing the rAAVp are about 37° C., 95% humidity and 8 vol.-% CO2.
52. The method according to any one of embodiment 45 to embodiment 51, wherein the cultivating is performed in suspension culture in serum free medium.
53. The method according to any one of embodiment 45 to embodiment 52, wherein
   one or both of the nucleic acids encoding the adenoviral helper functions is/are gene under control of a derepressible promoter, or/and
   the nucleic acid encoding the AAV Cap protein or/and the nucleic acid according to any one of embodiment 1 to embodiment 11, embodiment 13 to embodiment 16, embodiment 18 to embodiment 21, embodiment 23 to embodiment 26 and embodiment 28 to embodiment 29 is/are under control of a derepressible promoter, and
   the cell further comprises a nucleic acid molecule encoding a repressor element of the first and the second derepressible promoter.
54. The method according to any one of embodiment 45 to embodiment 52, wherein
   one or both of the nucleic acids encoding the adenoviral helper functions is/are under control of an inducible promoter, or/and
   the nucleic acids encoding the AAV Cap protein or/and the nucleic acid according to any one of embodiment 1 to embodiment 11, embodiment 13 to embodiment 16, embodiment 18 to embodiment 21, embodiment 23 to embodiment 26 and embodiment 28 to embodiment 29 is/are under control of an inducible promoter.
55. The method according to embodiment 54, wherein the inducible promoter is a doxycycline-inducible promoter.
56. The method according to any one of embodiment 36 to embodiment 55, wherein the cultivating is for a total of 7 to 14 days.
57. The method according to any one of embodiment 36 to embodiment 56, wherein the cultivating is at a temperature of about 36° C. to 42° C.
58. The method according to any one of embodiment 36 to embodiment 57, wherein the cultivating is in a first temperature phase at a temperature of about 36 to 38° C. and in a second temperature phase at a temperature of about 38° C. to about 42° C.
59. The method according to any one of embodiment 36 to embodiment 58, wherein the mammalian cell is a HEK cell or a CHO cell.
60. The method according to any one of embodiment 36 to embodiment 59, wherein the cultivating is at a pH value in the range of and including pH 7.4 to pH 7.6.
61. The method according to any one of embodiment 36 to embodiment 60, wherein the rAAVp is a therapeutic rAAVp.
62. The method according to any one of embodiment 36 to embodiment 61, wherein the gene of interest encodes a functional protein or a functional transcript that has a therapeutic effect.
63. The method according to any one of embodiment 36 to embodiment 62, wherein the rAAVp is for transfer of a nucleic acid that is transcribed into a polypeptide with therapeutic effect into target cells.
64. The method according to any one of embodiment 36 to embodiment 63, wherein the rAAVp is for transfer of a nucleic acid that has therapeutic effect into target cells.
65. The method according to any one of embodiment 36 to embodiment 64, wherein the rAAVp comprises at least one coding nucleic acid sequence interspaced between two adeno-associated viral inverted terminal repeats.
66. The method according to any one of embodiment 36 to embodiment 65, wherein the capsid polypeptides of the rAAVp are derived from a naturally occurring AAV serotype selected from the group consisting of AAV1, AAV2, AAV3, AAV4, AAV5, AAV6, AAV7, AAV8, AAV9, AAV10, AAV11, AAV12, AAV 218, AAV rh.74, AAV rh. 10 and AAV 7m8, as well as variants thereof.
67. The method according to any one of embodiment 36 to embodiment 66, wherein the capsid polypeptide of the rAAVp has an amino acid sequence having 70% or more sequence identity to a naturally occurring AAV serotype selected from the group consisting of AAV1, AAV2, AAV3, AAV4, AAV5, AAV6, AAV7, AAV8, AAV9, AAV10, AAV11, AAV12, AAV-218, AAV-rh.10, AAV-rh.74 and AAV-7m8.
68. The method according to any one of embodiment 36 to embodiment 67, wherein the capsid polypeptides of the rAAVp are derived from a naturally occurring AAV serotype selected from AAV2, AAV5, AAV8, AAV9 or AAV rh.74 or a variant thereof.
69. The method according to any one of embodiment 36 to embodiment 68, wherein the ITRs are naturally occurring ITRs of a serotype selected from the group consisting of AAV1, AAV2, AAV3, AAV4, AAV5, AAV6, AAV7, AAV8, AAV9, AAV10, AAV11, AAV12 and AAV13 or are variants thereof.
70. The method according to any one of embodiment 36 to embodiment 69, wherein the ITRs have a sequence that has 70% or more sequence identity to a naturally occurring ITR of a serotype selected from the group consisting of AAV1, AAV2, AAV3, AAV4, AAV5, AAV6, AAV7, AAV8, AAV9, AAV10, AAV11, AAV12 and AAV13.
71. The method according to any one of embodiment 36 to embodiment 70, wherein the ITRs have a sequence that has 95% or more sequence identity to a naturally occurring ITR of a serotype selected from the group consisting of AAV2, AAV6, AAV8 and AAV9.
72. The method according to any one of embodiment 36 to embodiment 71, wherein the ITRs have a sequence that is 70% or more identical to the sequence of a naturally occurring ITR sequence of the serotype AAV2.
73. The method according to any one of embodiment 36 to embodiment 72, wherein the rAAVp comprises capsid polypeptides of a first AAV serotype and ITRs of a second AAV serotype, whereby the first and the second AAV serotype are different.
74. The method according to any one of embodiment 36 to embodiment 73, wherein the cultivating encompasses the inoculation of the bioreactor and the harvest of the rAAVp.
75. The method according to any one of embodiment 36 to embodiment 74, wherein the cultivating starts with the inoculation of the bioreactor.
76. The method according to any one of embodiment 36 to embodiment 75, wherein one or more or all of the expression cassettes for the non-adeno-associated viral nucleic acid, which is interspaced between two AAV ITRs, for the adeno-associated virus Rep protein encoding nucleic acid, for the adeno-associated virus Cap protein encoding nucleic acid, for the adeno-associated virus E2A encoding nucleic acid, for the adeno-associated virus E4orf6 encoding nucleic acid and for the adeno-associated virus VA RNA encoding nucleic acid are introduced into the mammalian cell after the inoculation of the bioreactor.
77. The method according to any one of embodiment 36 to embodiment 76, wherein one or more or all of the expression cassettes for the non-adeno-associated viral encoding nucleic acid, which is interspaced between two AAV ITRs, for the adeno-associated virus Rep protein encoding nucleic acid, for the adeno-associated virus Cap protein encoding nucleic acid, for the adeno-associated virus E2A encoding nucleic acid, for the adeno-associated virus E4orf6 and optionally for the adeno-associated virus VA RNA encoding nucleic acid are introduced into the mammalian cell after the inoculation of the bioreactor, whereby up to three plasmids are co-transfected into the mammalian cell, whereby one of the plasmids comprises the expression cassette for the non-adeno-associated viral encoding nucleic acid, which is interspaced between two AAV ITRs, one of the plasmids comprises the expression cassettes for the Rep and Cap protein encoding nucleic acid and one of the plasmids comprises the expression cassettes for the adenoviral E2A, E4orf6 and VA RNA encoding nucleic acids.
78. The method according to any one of embodiment 36 to embodiment 77, wherein expression of one or more or all of the non-adeno-associated viral nucleic acid, which is interspaced between two AAV ITRs, the adeno-associated virus Rep protein encoding nucleic acid, the adeno-associated virus Cap protein encoding nucleic acid, the adeno-associated virus E2A encoding nucleic acid, the adeno-associated virus E4orf6 and optionally the adeno-associated virus VA RNA encoding nucleic acid is induced after the inoculation of the bioreactor.
79. The method according to any one of embodiment 76 to embodiment 78, wherein the introduction is about 16 to 32 hours after the inoculation of the bioreactor.
80. The method according to any one of embodiment 54 to embodiment 79, wherein the induction is about 16 to 32 hours after the inoculation of the bioreactor.
81. The method according to any one of embodiment 54 to embodiment 80, wherein the introduction or induction is about 24 hours after the inoculation of the bioreactor.
82. The method according to any one of embodiment 45 to embodiment 81, wherein the recovering is a harvesting of the cells and the supernatant.
83. The method according to any one of embodiment 45 to embodiment 82, wherein the recovering comprises i) harvesting of the cells and the supernatant, ii) lysing the cells and iii) removing cell debris.
84. The method according to embodiment 83, wherein the lysing is by chemical lysis or physical lysis.
85. The method according to any one of embodiment 83 to embodiment 84, wherein the lysing is by chemical lysis with a detergent or/and alkaline solution.
86. The method according to any one of embodiment 83 to embodiment 85, wherein during the lysing or after the lysing a nuclease is added.
87. The method according to embodiment 86, wherein the nuclease is benzonase.
88. The method according to any one of embodiment 83 to embodiment 87, wherein the removing of cell debris is by filtering or centrifuging.
89. The method according to embodiment 88, wherein the filtering is with a micron diameter pore size filter.
90. The method according to any one of embodiment 88 to embodiment 89, wherein the filtering is with a filter with a pore size in the range and including 0.1 μm to 10 μm.
91. The method according to any one of embodiment 88 to embodiment 90, wherein the filtering is with a filter with a pore size of 0.2 μm or 0.45 μm.
92. The method according to any one of embodiment 45 to embodiment 81, wherein the purifying is by one or more column chromatography steps and/or a CsCl or iodixanol gradient centrifugation step.
93. The method according to any one of embodiment 45 to embodiment 92, wherein the purifying is by an anion exchange chromatography, an affinity chromatography and/or a cation exchange chromatography
94. The method according to any one of embodiment 92 to embodiment 93, wherein the first chromatography step is an affinity chromatography step.
95. The method according to of embodiment 45 to embodiment 94, wherein the purifying is by a sequence of chromatography steps wherein the first is an affinity chromatography, followed by an anion exchange chromatography or a cation exchange chromatography, and an optional a size exclusion chromatography.
96. A rAAVp produced with a method according to any one of embodiment 36 to embodiment 95.
97. A pharmaceutical composition comprising the rAAVp obtained with a method according to any one of embodiment 36 to embodiment 95.
98. The pharmaceutical composition according to embodiment 97 comprising the rAAVp obtained with a method according to any one of embodiment 36 to embodiment 95 and a pharmaceutically acceptable excipient.
99. Use of the nucleic acid of any one of embodiment 1 to embodiment 11, embodiment 13 to embodiment 16, embodiment 18 to embodiment 21, embodiment 23 to embodiment 26 and embodiment 28 to embodiment 29 for increasing the yield of a recombinantly produced rAAVp.
100. Use of the nucleic acid of any one of embodiment 1 to embodiment 11, embodiment 13 to embodiment 16, embodiment 18 to embodiment 21, embodiment 23 to embodiment 26 and embodiment 28 to embodiment 29 for increasing the percentage of full rAAVp.
101. Use of the nucleic acid of any one of embodiment 1 to embodiment 11, embodiment 13 to embodiment 16, embodiment 18 to embodiment 21, embodiment 23 to embodiment 26 and embodiment 28 to embodiment 29 for decreasing Rep68 and/or Rep78 expression.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following figures form part of the present specification and are included to further demonstrate certain aspects of the present invention, which can be better understood by reference to one or more of these figures in combination with the detailed description of specific embodiments presented herein. It is to be understood that the data illustrated in the Figures does in no way limit the general applicability of the invention but represents preferred embodiments.

FIGS. 1A, 1B, 1C & 1D illustrates evaluation of different AAV Rep proteins in rAAV2p production.

FIG. 1A illustrates a schematic representation of the employed cloning strategy for natural rep ORFs of serotypes 1 to 13 or chimeric rep ORFs. The cloning procedure included a unique stretch between rep and cap in each AAV genome joining these two genes

FIG. 1B illustrates fold change in viral genomes per mL cell culture lysate (vg/mL). Adherent HEK293T cells were transfected with a rAAVp harboring a GFP encoding reporter transgene, an Adenohelper plasmid and one plasmid of the indicated Rep variant. AAV2 Cap was co-expressed from the RepX plasmids as shown in panel A. GFP titers acquired with Rep2 were averaged and set to 1, n=3.

FIG. 1C illustrates fold change in total capsids per mL cell culture lysate (vp/mL). HEK293T Cells were transfected as described in panel B. vp/mL titers acquired with Rep2 were averaged and set to 1, n=2.

FIG. 1D illustrates fold change in packaging rate calculated from panels B and C. Rates acquired with Rep2 were averaged and set to 1, n=2.

FIGS. 2A & 2B illustrates generation of a chimeric rep library using DNA family shuffling.

FIG. 2A illustrates steps of the DNA family shuffling procedure. A pool of PCR-amplified rep ORFs was digested with DNAseI under conditions indicated in respective agarose gel electrophoresis images. This involved varying times of incubation (30, 60 or 90 seconds) and DNAseI concentration (Stock=1 U/μl or 1:10 dilution). Fragments in the indicated range (100-1000 bp) were extracted from the gel and served as input for a primerless, homology-based PCR. Finally, complete ORFs were amplified with a rescue PCR using primers binding outside of the shuffled region.

FIG. 2B illustrates hybrid rep library; also denoted as R0. Each line represents one clone from the 5′- to the 3′-end. Color blocks indicate the underlying parental rep sequences as shown by the color code at the bottom. “Maximum traces” were generated as described in FIG. 7 and were used to build a graphical composition summary of each chimera. White annotated regions could not be exclusively assigned to one rep reference sequence.

FIGS. 3A & 3B illustrates directed evolution of Rep proteins for rAAVp production.

FIG. 3A illustrates schematic representation of the directed evolution approach. For wild-type rAAV2p production suspension HEK293 cells were transfected with the chimeric rep library and an Adenohelper plasmid. Cells were lysed to recover rAAV2p produced with the chimeric rep ORFs. Primers binding outside of the shuffled region were used for PCR amplification of the hybrid regions. Finally, chimeric rep ORFs were cloned into an acceptor plasmid with AAV2 cap for clonal assessment and initiation of 2nd selection round.

FIG. 3B illustrates clonal composition of rep hybrids after each selection round. Each line corresponds to one clone from the 5′- to 3′-end. Color blocks represent underlying rep reference sequences, as indicated with the color code at the bottom. R0=primary library. R1/R2=selection rounds 1 and 2.

FIGS. 4A, 4B, 4C, 4D, 4E, 4F & 4G illustrates functional validation of single rep hybrids.

FIG. 4A illustrates high-throughput screening of Rep variants in a micro-scale setting (24 deep-well). Data from the indicated analytical measurements are represented as a heat map with Rep2-values set to 100% (white) and the highest limit corresponding to 500% of Rep2 levels (red); n=2-3 biological replicates. Genomic titers ranged from 1×1E9 to 2×1E10 vg/mL, with values below 5×1E8 vg/mL classified as negative. Capsid titers ranged from 1×1E10 to 5×1E11 vp/mL.

FIG. 4B illustrates testing of selected Rep candidates in mini-scale (30 ml shake flasks; n=2-3 biological replicates). Assessed parameter: genomic titer. Genomic titers ranged from 6×1E9 to 2×1E11 vg/mL.

FIG. 4C illustrates testing of selected Rep candidates in mini-scale (30 ml shake flasks; n=2-3 biological replicates). Assessed parameter: capsid titer. Capsid titers ranged from 2×1E11 to 8×1E12 vp/mL.

FIG. 4D illustrates testing of selected Rep candidates in mini-scale (30 ml shake flasks; n=2-3 biological replicates). Assessed parameter: packaging rate.

FIG. 4E illustrates testing of selected Rep candidates in mini-scale (30 ml shake flasks; n=2-3 biological replicates). Assessed parameter: functionality of resulting AAV vectors (% of GFP positive cells) after transduction with crude cell lysates. Equal volumes were applied (1:10 dilution of stock).

FIG. 4F illustrates testing of selected Rep candidates in mini-scale (30 ml shake flasks; n=2-3 biological replicates). Assessed parameter: cell viability at day of harvest.

FIG. 4G illustrates overview of the clonal composition of selected rep hybrids (5′- to 3′-end). Color blocks represent underlying rep reference sequences, as indicated with the color code at the bottom.

FIGS. 5A, 5B, 5C, 5D, 5E & 5F illustrates functional validation of lead rep hybrids in the AMBR fermentation systems.

FIG. 5A illustrates production of rAAVp-GFP with the indicated Rep variants or Rep2 control in the AMBR15 system. Assessed parameter: genomic titer.

FIG. 5B illustrates production of rAAVp-GFP with the indicated Rep variants or Rep2 control in the AMBR15 system. Assessed parameter: capsid titer.

FIG. 5C illustrates production of rAAVp-GFP with the indicated Rep variants or Rep2 control in the AMBR15 system. Assessed parameter: packaging rate.

FIG. 5D illustrates western Blot analysis using the JESS Simple Western™ system. Cell lysates from the indicated rAAVp productions in the AMBR250 system were analyzed for capsid protein expression (VP1-VP3). A semi-quantitative analysis is provided in FIGS. 8B and 8C. TTG=therapeutic transgene.

FIG. 5E illustrates western Blot analysis using the JESS Simple Western™ system. Cell lysates from the indicated rAAVp productions in the AMBR250 system were analyzed for Replication protein (Rep) expression. A semi-quantitative analysis is provided in FIGS. 8B and 8C. TTG=therapeutic transgene.

FIG. 5F illustrates cryo-EM analysis of the indicated rAAVp productions in AMBR250 purified using small-scale AAVX-columns. Arrows indicate examples of full AAV capsids.

FIGS. 6A & 6B illustrates functional assessment of natural AAV Rep variants in adherent HEK293T cells.

FIG. 6A illustrates western Blot analysis using the JESS Simple Western™ system. Expi293FTM suspension cells were transfected with the indicated RepXCap2 plasmids (x=1-13), alongside the helper and an AAV-GFP reporter plasmid. Cells were harvested three days post-transfection, and lysates were analyzed for Rep protein expression (top panel) and GAPDH expression (bottom panel) as a loading control. The expected Rep protein variants are indicated.

FIG. 6B illustrates cell viability and transfection efficiency measured three days post-transfection. HEK293T cells were transfected with the indicated RepXCap2 plasmid, together with the Adenohelper and a GFP reporter transgene. Cells were harvested in PBS and viability and flow cytometry analyses were performed.

FIGS. 7A & 7B illustrates phylogenetic and distance analysis of Rep hybrid clones.

FIG. 7A illustrates phylogenetic analysis of the indicated rep variants (colored red). The evolutionary history was inferred in Geneious Prime by using the Neighbor-Joining algorithm based on the Tamura-Nei model. The percentage of replicate trees in which the associated taxa clustered together in the bootstrap test (500 replicates) were shown next to the branches. The scale bar represents the average number of nucleotide substitutions per site.

FIG. 7B illustrates nucleotide identity matrix. The percentage of nucleotide identity among the indicated nucleotide sequences.

FIGS. 8A, 8B, 8C & 8D illustrates composition and phylogenetic analysis of Rep 1.03.

FIG. 8A illustrates clone 1.03 from selection round 1 was aligned to the 13 parental rep references using MUSCLE (DNA alignment). Mismatches to the references at single nucleic acid positions were colored in black. Matching regions are shown in green and were ranked according to their length. A “maximum trace”-colored in dark green-reflects the highest probability of origin of a particular region. If several traces were identified, the longer stretch was colored in dark green and the others in light green. Blue bars reflect the percentage of each reference sequence in the clone composition. Above the alignment the approximate location of the underlying Rep domains is shown.

FIG. 8B illustrates phylogenetic analysis of the indicated Rep protein domains. OBD=origin binding domain (AA residues 1-225). Hybrid Rep variant 1.03 is colored red. The evolutionary history was inferred in Geneious Prime by using the Neighbor-Joining algorithm based on the Jukes-Cantor model. The percentage of replicate trees in which the associated taxa clustered together in the bootstrap test (500 replicates) were shown next to the branches. The scale bar represents the average number of nucleotide substitutions per site.

FIG. 8C illustrates phylogenetic analysis of the indicated Rep protein domains. Helicase (AA residues 225-490). Hybrid Rep variant 1.03 is colored red. The evolutionary history was inferred in Geneious Prime by using the Neighbor-Joining algorithm based on the Jukes-Cantor model. The percentage of replicate trees in which the associated taxa clustered together in the bootstrap test (500 replicates) were shown next to the branches. The scale bar represents the average number of nucleotide substitutions per site.

FIG. 8D illustrates phylogenetic analysis of the indicated Rep protein domains. ZF=Zinc finger domain (AA residues 531-625). Hybrid Rep variant 1.03 is colored red. The evolutionary history was inferred in Geneious Prime by using the Neighbor-Joining algorithm based on the Jukes-Cantor model. The percentage of replicate trees in which the associated taxa clustered together in the bootstrap test (500 replicates) were shown next to the branches. The scale bar represents the average number of nucleotide substitutions per site.

FIG. 9 illustrates genomic titers and genome intactness using Rep2 and Rep1.03. Absolute quantification of rAAV titers using dPCR. A total of 15 mL of the indicated rAAVps, produced in the AMBR 250 system, were purified using AAVX affinity resin. Singleplex and multiplex titer analyses were conducted using primer/probe sets targeting different regions of the recombinant genome: the 3′ end, 5′ end, and the middle.

### DETAILED DESCRIPTION OF EMBODIMENTS OF THE INVENTION

Herein is reported a nucleic acid encoding a functional adeno-associated virus Rep protein, characterized in that the nucleic acid comprises in 5′- to 3′-direction a first 5′-terminal part, a second 5′-terminal part, a central part, a second 3′-terminal part and a first 3′-terminal part, wherein the first 5′-terminal part is similar to a part of the rep gene of the AAV2 or AAV6 serotype and a second 5′-terminal part is similar to a part of the rep gene of an AAV1 serotype and the second 3′-terminal part is similar to a part of the rep gene of the AAV10 or AAV11 serotype and a first 3′-terminal part is similar to a part of the rep gene of an AAV13 serotype.

The current invention is based, at least in part, on the finding that the rAAVp production, especially the packaging efficiency, can be improved by genetic engineering of the replication and packaging protein of AAV (Rep), i.e. by combining elements of the Reps of different serotypes.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987). The content of which is incorporated herein by reference,

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless otherwise defined herein the term “comprising of”′ shall include the term “consisting of”.

The term “about” as used herein in connection with a specific value (e.g. temperature, concentration, time and others) shall refer to a variation of +/−1% of the specific value that the term “about” refers to.

The term “about” denotes a range of +/−20% of the following numerical value. In certain embodiments, the term about denotes a range of +/−10% of the thereafter following numerical value. In certain embodiments, the term about denotes a range of +/−5% of the thereafter following numerical value.

The term “similar” denotes that two sequences differ from each other at at most 10% of the residues, i.e. nucleic acid residues or amino acid residues.

The term “derived” denotes that a second sequence has been obtained or generated based on a first sequence. This includes that one or more changes have been introduced in the second sequence or that the second sequence is only a contiguous part, i.e. a fraction, of the first sequence or both of the before.

The terms “comprise(s),” “include(s),” “having,” “has,” “can,” “contain(s)” and variants thereof, as used herein, are intended to be open-ended transitional phrases, terms or words that do not preclude the possibility of additional acts or structures. The term “comprising” also encompasses the term “consisting of”. The present disclosure also contemplates other embodiments “comprising”, “consisting of” and “consisting essentially of” the embodiments or elements presented herein, whether explicitly set forth or not.

The terms “empty recombinant AAV particle” and “empty rAAVp”, which can be used interchangeably, denote a protein shell composed of adeno-associated capsid polypeptides without a therein encapsidated/packaged functional nucleic acid (rAAVp=recombinant adeno-associated virus particle). That is, an empty rAAVp either may be free of encapsidated nucleic acid or comprises a nucleic acid or part thereof that is not transcribed at all or not transcribed into a functional transcript. Accordingly, an empty rAAVp does not function to transfer a nucleic acid that encodes a functional protein or is transcribed into a functional transcript of interest into a target cell. In certain embodiments of all aspects and embodiments, the functional protein or the functional transcript of interest has a therapeutic effect.

The terms “full recombinant AAV particle” or “full rAAVp”, which can be used interchangeably, denote a non-covalent complex formed of a protein shell composed of adeno-associated capsid polypeptides and a therein encapsidated/packaged functional nucleic acid sequence. That is, a full rAAVp comprises a nucleic acid that is transcribed into a functional transcript. Accordingly, the full rAAVp functions to transfer a nucleic acid that encodes a protein or is transcribed into a transcript of interest into a target cell. In certain embodiments, a functional nucleic acid comprises at least one coding nucleic acid sequence interspaced between two adeno-associated viral inverted terminal repeats (ITRs).

The term “full to empty ratio” denotes the mathematical ratio of the number of full recombinant AAV particles (full rAA Vp) to the total number of recombinant AAV particles (sum of full rAAVp and empty rAAVp) in a sample or in a recombinant AAV particle preparation. As the number of full rAAVp can be at most the same as the total number of rAAVp, the ratio can be at most 1. Generally, the ratio is less than 1 and is expressed as a percentage. The number of full rAAVp can be determined by determining the number of nucleic acid sequences interspaced between two AAV ITRs in the sample or preparation. This can be done by PCR, especially digital droplet PCR (ddPCR) or quantitative PCR (qPCR). The total number of rAAVp can be determined by determining the number of protein shells formed of adeno-associated capsid polypeptides in the sample or preparation. This can be done by ELISA, especially by a capsid polypeptide specific ELISA.

The term “transgene” denotes a nucleic acid derived from a wild-type genome of an adeno-associated virus, wherein except for the ITR (adeno-associated virus Inverted Terminal Repeat) sequences all endogenous AAV nucleic acids are replaced by one or more exogenous nucleic acid(s). For example, such an exogenous nucleic acid can be a nucleic acid transcribed into a transcript of interest or that encodes a therapeutic protein or a therapeutic nucleic acid. Typically, for a transgene one or both ITR sequences of a wild-type AAV genome are retained. Thus, a transgene can be distinguished from a wild-type AAV genome, since all or at least a part of the viral genome has been replaced with a non-native (i.e. exogenous) nucleic acid with respect to the virus. Incorporation of a non-native nucleic acid therefore defines the transgene as a “recombinant”. It has to be pointed out that the serotype of the ITRs in the transgene does not need to be the same as the serotype of the adeno-associated capsid polypeptides forming the shell of the rAAVp comprising said transgene.

Thus, the term “transgene” also denotes the portion of a larger nucleic acid, e.g. of a recombinant plasmid, that is ultimately packaged or encapsulated or encapsidated either directly or in form of a single strand or in form of RNA into a protein shell composed of adeno-associated virus capsid polypeptides to form a rAAVp. In cases where recombinant plasmids are used to construct or manufacture rAAVps, the viral particle does not include the portion of the “plasmid” that does not correspond to the transgene part of the recombinant plasmid. For example, in case of a rAAVp the encapsidated nucleic acid comprises that part of the recombinant plasmid that is interspaced between two AAV ITRs. The non-transgene portion of the recombinant plasmid is referred to as the “plasmid backbone”. The plasmid backbone is important for cloning and amplification of the plasmid, a process that is needed for propagation and recombinant virus production, but is not itself packaged or encapsulated or encapsidated into the rAAVp. Thus, a “transgene” refers to the nucleic acid that is packaged or encapsulated or encapsidated by a protein shell composed of adeno-associated virus capsid polypeptides, i.e. in a rAAVp.

In principle, any non-AAV nucleic acid can be packaged into a shell composed of adeno-associated capsid polypeptides resulting in a rAAVp, e.g. for subsequent infection (transduction) of a cell, ex vivo, in vitro or in vivo.

The term “serotype” is used herein to denote the origin of the elements forming a rAAVp as well as the origin of elements used for the production of a rAAVp. Thus, the serotype can be used to denote the origin of the amino acid sequence of the polypeptides forming the protein shell (capsid) of the rAAVp as well as the origin of, e.g., the rep gene or its fragments used for the production/packaging of the rAAVp.

General Methods for Producing rAAvp

WO 1999/11764 reported methods for generating high titer helper-free preparations of recombinant AAV vectors. Not further defined AAV producer cells grown in suspension in bioreactors were infected with Adenovirus Type 5 (Ad5) at a multiplicity of infection (MOI) of 10 in low serum media at 1.5 L scale at different pH values. At a culture pH of 7.2, 4.7 E+12 total particles were obtained, at a culture pH of 7.4 1.95 E+13 total particles were obtained, at a culture pH of 7.6 1.84 E+13 total particles were obtained and at a culture pH of 8.0 1.63 E+13 total particles were obtained. The cultivation was performed in a 1.5 L bioreactor and, thus, the cultivation volume can be calculated (75% of the nominal value) to have been about 1.125 L. Therefore the total particle number correspond to 4.2 E+09 vp/mL (pH 7.2), 1.7 E+10 vp/mL (pH 7.4), 1.6 E+10 vp/mL (pH 7.6) and 1.5 E+10 vp/mL (pH 8), respectively.

WO 2000/14205 reported the production of AAV particles in a non-defined cell type denoted as JL-14 cells by co-infection with adenoviral helper virus, whereby at a pH value of 7.4 the highest number of AAV particles (sum of intracellular and secreted AAV particles), at a pH value of 8 AAV particles with the highest infectivity and at a pH of 7.6 the highest ratio of number of AAV particles to infectivity was obtained. The cultivation was performed in a volume of 1.5 L medium and, thus, the total particle number correspond to 3.0 E+09 vp/mL (pH 7.2), 1.3 E+10 vp/mL (pH 7.4), 1.2 E+10 vp/mL (pH 7.6), 3.3 E+09 vp/mL (pH 7.8) and 1.1 E+10 vp/mL (pH 8), respectively. Based on the provided infectivity data it can be assumed that the full/empty ratio of the thereby produced rAAVps is below 1%.

Piras, B. A., et al. (Mol. Ther. Meth. Clin. Dev. 3 (2016) 16015) compared distribution of AAV8 in cell culture media and lysates on days 3, 5, 6 and 7 post-transfection and found increasing viral production through day 6, with the proportion of viral particles in the media increasing from 76% at day 3 to 94% by day 7. Larger-scale productions showed that the ratio of full-to-empty AAV particles is similar in media and lysate, and that AAV harvested on day 6 post-transfection provides equivalent function in mice compared to AAV harvested on day 3. AAV-Piras et al. employed adherent HEK293T/17 cells cultured in Dulbecco's Modified Eagle's Medium with 10% fetal bovine serum supplemented with 2 mmol/l GlutaMAX (Life Technologies, Grand Island, NY). AAV was produced by two-plasmid transfection using PEIpro™ (Polyplus-transfection SA, Illkirch, France) 1 day after seeding cells at a density of 7.26×1 E+04 cells/cm2.

Powers, A. D., et al. (Hum. Gene Ther. Meth. 27 (2016) 112-121) reported the development and optimization of AAV hFIX particle production by transient transfection in an iCELLis® fixed-bed bioreactor. A yield to as high as 9 E+14 viral particles per square meter of fixed bed were obtained. On day 3 after inoculation with HEK293T/17 cells, the vessel was transfected with plasmid scAAV-LP1-hFIXco-helpv3 and plasmid CR21+LTAAV help 2-8 at a plasmid mass ratio of 3:1, respectively, using polyethylenimine (PEIpro™ Transfection Reagent Cat #115-375; Polyplus) in IMDM (Lonza) or DMEM supplemented with 10% FBS and 6 mM GlutaMAX™. The PEI and DNA solutions were combined at a 2:1 ratio.

Poulain, A., et al. (J. Biotechnol. 255 (2017) 16-27) reported rapid protein production from stable CHO cell pools using plasmid vector and the cumate gene-switch. Cells were transfected using linear polyethylenimine (PEIpro™) from Polyplus-Transfection (Illkirch, France). On the day of transfection, cells were suspended at a density of 2×1 E+06 cells/mL in CD DG44 medium (Life Technologies Inc., Burlington, ON, Canada), supplemented with 4 mM glutamine and 0.1% Kolliphor® P 188. The cell suspension was distributed in 6-well plates (1.8 mL/well). The DNA: PEIpro™ complexes were prepared at a ratio of 1:5 (w:w), with a total of 2 μg DNA per well to transfect in 100 μL of complete culture medium.

WO 2017/096039 reported scalable methods for producing recombinant AAV vectors in serum-free suspension cell culture systems suitable for clinical use. Production of rAAVp was performed in bioreactors with HEK293F cells using triple transfection at a cell density of 1 E+06 cells/mL (1.000.000 cells/mL) with a plasmid ratio of 1:1:1 and a PEI-based transfection reagent (PEI/DNA weight ratio of 2:1 with ½ of PEI as free PEI) at a temperature of 37° C. and a pH value of 7.2.

Nyamay'antu, A., et al. (Cell Gen. Ther. Ins. 4 (2018) 71-79) reported that PEI is widely used due to its affordability and high DNA delivery efficiency, in both adherent and suspension cells grown in serum-free medium. PEIpro™ is suited for small- to large-scale production of various viruses, notably AAV particles. In stirred-tank bioreactors using HEK293 or HEK293T cells titers in the range of 0.8-1.5 E+09-E+10 vg/mL can be obtained.

Koo, T., et al. (Nat. Commun. 9 (2018) 1855) reported that CRISPR-LbCpf1 prevents choroidal neovascularization in a mouse model of age-related macular degeneration. To produce AAV vectors, they were pseudotyped in AAV9 capsids. HEK293T cells (ATCC, CRL-3216) were transfected with pAAV-ITR-LbCpf1-crRNA, pAAV2/9 encoding for AAV2rep and AAV9cap, and helper plasmid. HEK293T cells were cultured in DMEM with 2% FBS. Recombinant pseudotyped AAV vector stocks were generated using PEI co-precipitation with PEIpro™ (Polyplus-transfection) and triple-transfection with plasmids at a molar ratio of 1:1:1 in HEK293T cells. After 72 h of incubation, cells were lysed and particles were purified by iodixanol step-gradient ultracentrifugation.

In the art the Rep proteins from AAV2 are commonly and nearly exclusively used in the production of rAAVps derived from the serotypes AAV1 to AAV13 (Daya, S., and Berns, K. I., Clin. Microbiol. Rev. 21 (2008) 583-593; Zincarelli, C., et al., Mol. Ther. 16 (2008) 1073-1080).

WO 2019/094253 reported means and methods for preparing viral vectors and uses thereof. Adherent HEK293 cells were cultivated in bioreactors at a pH value of 7.23 and triple transfected (plasmid ratio 1:1:1) with PEI/DNA at a PEI-plasmid ratio of about 1:1 by weight.

Collaud, F. et al. (Mol. Ther. Meth. Clin. Dev. 12 (2019) 157-174) reported titers for recombinant AAV8 particles of 6.0±1.89 E+04 vg/cell and 1.77±1.37 E+04 vg/cell for (single stranded) and (self-complementary) AAV, respectively, for adherent HEK293 cells. A fully scalable method based on triple transfection of HEK293 cells cultured in suspension was also reported. Triple transfection of HEK293 cells was performed with polyethylenimine (PEIpro™, Polyplus) directly in 10 L bioreactors. AAV vectors were recovered from both supernatant and cells by mild detergent lysis followed by AVB Sepharose affinity column purification. Purified vectors were then concentrated and tested for quality and potency. No information about the pH value and obtained titers are provided.

Nyamay'antu, A., et al. (Cell Gen. Ther. Ins. 6 (2020) 655-661) reported that the efficiency of the delivery process is essential to obtain a high number of producing cells. Of the existing transfection methods, the use of PEI-based transfection reagent is predominant in gene therapy as it combines affordability and compatibility for transfection of adherent and suspension cells. In comparison to the gold standard PEIpro™ used for viral vector manufacturing, FectoVIR™-AAV has been found to improve significantly recombinant AAV2 particle production yield of both viral genome production and packaging efficiency in suspension cells of an rAAV2-GFP of up to 10-fold compared to PEIMax™ and up to 2-fold compared to PEIpro™, respectively, when each transfection reagent is used under the recommended conditions. In more detail, suspension HEK293T cells were transfected using the respective transfection reagent under the recommended conditions. rAAV2p-GFP were harvested 72 hours post transfection. The obtained titer with FectoVIR™ is in the range of 1 E+04 to 4.5 E+04 vg/cell depending on the used volume of complexation (1%-10%) corresponding to 1 E+12 vg/mL. The respective functional titers are about 2-8 E+08 TU/mL. The results are almost independent of the employed cultivation medium.

In a blog article entitled “Optimization of AAV production for high-yielding and scalable GMP processes with Catalent” (www.polyplus-transfection.com) different transfection reagent to DNA ratios were tested with the two serotypes AAV9 (1:1 and 2:1) and AAV2 (3:1.5 and 5:2.5). The AAV2 vector yield was not affected as notably, with a 4-5-fold increase in the vector genome titer and a 3-6-fold increase in the viral particle titer with FectoVIR™-AAV as compared to PEIpro™. These results show that improvement in yield may vary with the AAV serotype. In a further study comparing additional AAV2 and AAV5 vectors (different from the previous AAV2 and AAV5 vectors) and using a DoE approach to optimization, experiments were conducted varying transfection reagent to DNA ratios (3:2, 3:1.5) and plasmid DNA molar ratios (1:1:1, 2:1:2, 1:2:1) were performed. A 3-5-fold increase for AAV2 and a 1.1-1.6-fold increase for AAV5 in the vector genome titer with FectoVIR™-AAV compared to PEIpro™ was observed. The viral particle titer increased 3.5-4.5-fold for AAV2 and 2.5-3.75-fold for AAV5. Reagent-to-DNA ratios of 2:1 and 1.5:1 and plasmid ratios of 1:1:1 to 2:1:2 to 1:2:1 were used. The obtained titer with FectoVIR™ was in the range of 4 E+11 to 1 E+12 vg/mL.

Rossi, A. and Peigné, C-M. (Cell Culture Dish Article May 17, 2021) outlined that, typically, AAV production titers are around 1 E+11 to 1 E+12 in vg/mL and 1 E+08 to 1 E+09 TU/mL.

Wosnitzka, K., et al. (Cell Gen. Ther. Ins. 7 (2021) 1-7) reported that analysis of physical titers revealed a 3-fold increase in both viral particles (VP) and viral genome (VG) per ml of cell culture when using FectoVIR™-AAV transfection reagent compared to PEIpro™.

Porte, M., et al. (poster entitled “Next-Generation Transfection Reagent for Large Scale AAV Manufacturing”, Polyplus, Illkirch, France) reported the transfection of suspension-HEK293T cells with the optimal conditions for the other PEI-based reagent (1.5 μg/million cells, ratio DNA:PEI of 1 μg: 4 μL) and FectoVIR™-AAV (1 μg/million cells, ratio DNA:reagent of 1 μg: 1 μL) following the recommended protocol for each reagent. A titer of about 5 E+11 vg/mL versus 1.5 E+11 vg/mL using FectoVIR™ and PEI-based transfection reagent, respectively, with a packaging efficiency of 20% vs. about 13.5%, respectively, was obtained.

Nakamura et al. (Eur. J. Haematol. 73 (2004) 285-294) reported about the development of packaging cell lines for generation of adeno-associated virus vectors by lentiviral gene transfer of trans-complementary components. It is outlined that adeno-associated virus (AAV) vector systems have several useful advantages with regard to in vitro and in vivo gene transfer. However, their usages have been limited by cumbersome and labor-intensive vector production in the traditional method. To overcome limitations in AAV production, Nakamura et al. explored the possibility of generating AAV packaging cell line, 293T R/C.VA.E2A.E4. cells, by using lentivirus-mediated transduction of Rep/Cap gene of AAV-2, VA RNA, E2A, and E4 genes of Ad5 into 293T cells. In packaging cell lines, it is important that supply of the AAV vector can be stably performed for long time. They showed that the 293T R/C.VA.E2A.E4. cells have stably maintained the transduced components after more than 10 passages and yielded high-titer AAV vectors, and the titer of AAV vectors did not decline even if culture of the packaging cells was continued for long time. The Rep/Cap and E4 gene products caused no remarkable cytotoxicity. The 293T R/C.VA.E2A.E4. cells might be able to tolerate the Rep/Cap and E4 gene products, or have less copy numbers of the Rep/Cap and E4 genes than the traditional method. Moreover, they showed that the AAV vectors derived from 293T R/C.VA.E2A.E4. cells infected the primary human CD34+ hematopoietic progenitor cells with high efficiency (50-70%). In the 293T R/C.VA.E2A.E4. cells, the AAV vectors can be generated by the transfection of one AAV vector plasmid, and large-scale AAV production can be easily achieved. It is important that cumbersome, variable, and costly transfection is avoided.

WO 2018/192983 reported an adeno-associated virus (AAV) producer cell comprising nucleic acid sequences encoding rep/cap gene; helper virus genes; and the DNA genome of the AAV vector particle, wherein said nucleic acid sequences are all integrated together at a single locus within the AAV producer cell genome. Reported are also nucleic acid vectors comprising a non-mammalian origin of replication and the ability to hold at least 25 kilo bases (kb) of DNA, characterized in that said nucleic acid vector comprises nucleic acid sequences encoding: rep/cap gene, and helper virus genes as well as uses and methods using said nucleic acid vector in order to produce stable AAV packaging and producer cell lines.

WO 2018/194438 reported a cell line for producing a non-replicating adenovirus, and a preparation method therefor and, more specifically, to: a cell line for producing a replication-deficient adenovirus by expressing any one or more selected from an E1 protein, and an ElA protein or an ElB protein of an adenovirus; and a method for preparing the same. In addition, it is reported the use of the cell line, for expressing any one or more selected from an E1 protein, and an E1A protein or an E1B protein of an adenovirus.

WO 2020/078953 reported adeno-associated virus (AAV) vector producer cell comprising nucleic acid sequences encoding AAV rep and cap genes, helper virus genes, and a DNA genome of the AAV vector; the AAV rep gene comprising an intron, the intron comprising a transcription termination sequence with a first recombination site located upstream and a second recombination site located downstream of the transcription termination sequence; and the nucleic acid sequences all integrated together at a single locus within the AAV vector producer cell genome.

WO 2020/132059 reported a mammalian cell line for producing adeno-associated virus (AAV), suitably including nucleic acids encoding helper genes and AAV genes, under the control of derepressible promoters. The disclosure also relates to isolated nucleic acid molecules that encode such genes, as well as methods of using the mammalian cells for producing AAVs. Especially is reported a mammalian cell for producing an adeno-associated virus (AAV), comprising (a) a nucleic acid molecule encoding a viral helper gene under control of a first derepressible promoter; (b) a nucleic acid molecule encoding an AAV gene under control of a second derepressible promoter; and (c) a nucleic acid molecule encoding a repressor element of the first and the second derepressible promoters.

EP 3 822 346 reported the use of an engineered mammalian packaging cell line for producing recombinant virus particles, wherein the cell line is engineered to lack cell surface expression of heparan sulfate. Further disclosed are a method for producing recombinant virus particles and a recombinant virus particle obtainable by the method. Further disclosed is a mammalian packaging cell line deposited under number DSM ACC3355 or DSM ACC3356.

WO 2022/112218 reported methods for the production of Adeno-associated vims (AAV), comprising steps of providing a stable AAV producer cell line in which at least some or all genes encoding the components necessary for the production of AAV are stably integrated into the cell genome, and culturing said cells in perfusion culture during the AAV production step (i.e., during the N step), wherein said perfusion culture encompasses continuous replacement of spent media with fresh media, and wherein said continuous replacement of spent media with fresh media continues after the induction of AAV production. In the cell at least (a) a gene encoding the AAV Rep protein Rep78 or Rep68, (b) a gene encoding the AAV Rep protein Rep52 or Rep40; (c) the genes encoding the adenoviral helper functions E4orf6 and E2A stably integrated into the host cell genome. Further at least the following genes are stably integrated into the host cell genome (a) the genes encoding the AAV Cap proteins VP1, VP2, VP3; (b) a gene encoding the AAV Rep protein Rep78 or Rep68; (c) a gene encoding the AAV Rep protein Rep52 or Rep40; (d) the genes encoding the adenoviral helper functions E4orf6 E2A; (e) the gene of interest flanked by AAV ITRs.

WO 2022/173944 reported methods for producing an adeno-associated virus (AAV) in an E1 complementary producer cell. Especially is reported a method of producing an adeno-associated virus (AAV) in an E1 complementary producer cell, comprising (a) transfecting the E1 complementary producer cell with one or more vectors comprising (1) an E1A adenovirus helper gene; (2) an adenovirus helper gene selected from E2A, E4, or both; (3) a viral-associated, non-coding RNA (VA RNA); and (4) an AA V gene selected from Rep, Cap, or both; (b) culturing the transfected E1 complementary producer cell under conditions suitable for producing the AAV; and (c) purifying the AAV from the cultured E1 complementary producer cell, thereby obtaining the AAV.

WO 2022/192261 reported compositions and methods for producing and characterizing stable viral vector producer cell lines that enable industrial scale production of viral vectors. Novel viral vector genome constructs, in which the constructs can be precisely mapped and viral vector genome constructs precisely quantified, are also disclosed for efficient production and characterization of viral vectors in mammalian cells.

WO 2023/077078 reported recombinant adeno-associated virus (rAAV) packaging and/or producer cell lines which have been engineered to reduce expression and/or activity of one or more genes and/or proteins to increase rAAV titers. Especially is reported a recombinant adeno-associated virus (rAAV) packaging and/or producer cell line comprising cells in which the expression of a gene selected from the group consisting of RIG-1 (DDX58), IFIT3, MDA5 (IFIH1), CGAS (cGAS), CHUK (IKK-a), DDX41, DHX58 (LGP2), IF16, IKBKB (IKK-α), IRF3, IRF7, MAVS, MYD88, NFKB1, NFKB2, TBK1, TRIP, and TRIM25, and any combination thereof is reduced compared to that in control parental cells.

WO 2023/102549 reported systems for increasing AAV particle production. These systems comprise producer cell lines adapted for the production of AAV particles, as well as methods of producing AAV particles using said producer cell lines. Also provided are AAV particles produced by said production systems, producer cell lines and methods. Especially it is reported a genetically engineered producer cell line in which the expression of at least one of TMED10, MON2, TMED2, HS2ST1, C3orfS8, SPPL3, SURF4, LSMS, ARFI, and PI4 KB is reduced as compared to a control cell line, and/or in which the expression of at least one of B4GALT7, B3GAT3, OAF, EXT2, COMMD3, SLC3SD1, B3GALT6, SDCI, NDSTI, RACI, CSK, GLCE, PDCL, FAM20B, TM4SFS, DGAT2, POMTI, YYI, and DPF2 is increased as compared to a control cell line.

WO 2023/114897 reported methods for the production of recombinant adeno-associated virus (rAAV) particles. These methods are particularly useful for the large-scale production of AAV particles. Especially it is reported A method for producing recombinant AAV (rAAV) particles, comprising (a) introducing into a mammalian cell a first polynucleotide comprising an rAAV genome, to generate an AAV producer cell; (b) culturing the AAV producer cell in a first culture medium at a first temperature for a first period of time; (c) culturing the AAV producer cell in a second culture medium at a second temperature for a second period of time, wherein the second temperature is about 38° C. to about 42° C., such that rAAV particles are produced by the AAV producer cell, wherein the rAAV particles comprise an rAAV genome comprising a transgene, and an AAV capsid comprising an AAV capsid protein.

WO 2023/166026 reported cell lines in which DNA fragmentation is inhibited, uses of cell lines in which DNA fragmentation is inhibited for the production of adeno-associated virus (AAV), related methods of producing AAV, and methods of producing AAV, comprising the step of exposing the cells in which AAV is produced to an inhibitor of DNA fragmentation during the AAV production phase.

WO 2023/171698 reported a producer cell for the production of an adeno-associated virus wherein cell damage is avoided or suppressed at the establishment of a cell line, a method for producing the producer cell, and a method for producing an AAV using the producer cell. In the producer cell a Cap gene under the control of a foreign promoter and a Rep gene under the control of a foreign promoter is integrated in the chromosome, that is free from a VA-RNA gene and/or an E4 gene, and that is a mammalian cell.

The content of all documents outlined in this section are expressly incorporated by reference herein.

### Recombinant Cell

Generally, for efficient as well as large-scale production of a rAAVp a cell expressing and, if possible, also secreting said rAAVp is used. Such a cell is termed “recombinant producer cell” or short “producer cell”.

For the generation of a recombinant producer cell a suitable mammalian cell is transfected with the nucleic acids required for producing said rAAVp, including the required AAV helper functions as well as a rep gene according to the current invention.

Generally, for expression of a coding sequence, i.e. of an open reading frame or a structural gene, beside the coding sequence additional regulatory elements, such as a promoter and a polyadenylation signal (sequence), are necessary. Thus, for functional transcription an open reading frame or structural gene has to be and is operably linked to said additional regulatory elements. This is achieved by combining these elements in operably-linked form in a so-called expression cassette. The minimal regulatory elements required for an expression cassette to be functional in a mammalian cell are a promoter functional in said mammalian cell, which is located upstream, i.e. 5′ to the open reading frame or structural gene, and a polyadenylation signal (sequence) functional in said mammalian cell, which is located downstream, i.e. 3′, to the open reading frame or structural gene. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case of an rAAVp, which is composed of different (monomeric) capsid polypeptides and a therein encapsidated single stranded DNA molecule and which in addition requires other viral helper functions for production and encapsidation, a multitude of expression cassettes differing in the contained open reading frames/coding sequences/structural genes are required. In this case, at least an expression cassette for each of the transgene, the rep gene, the polypeptides forming the capsid of the rAAVp, i.e. the cap gene, and the required viral helper functions are required. Thus, individual expression cassettes at least for each of the helper functions E1A, E1B, E2A, E4orf6, the rep gene and the cap gene are required. HEK293 cells express the E1A and EIB helper functions constitutively.

### Adeno-Associated Virus (AAV)

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV particles are described (see also WO 93/24641). Additional references describing AAV particles are West et al., Virol. 160 (1987) 38-47; Kotin, Hum. Gene Ther. 5 (1994) 793-801; and Muzyczka J. Clin. Invest. 94 (1994) 1351. Construction of recombinant AAV particles is described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An AAV is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV2 other integration sites have been found, such as, e.g., on chromosome 5 [(Chr) 5 (p13.3)], termed AAVS2, and on chromosome 3 [(Chr) 3 (p24.3)], termed AAVS3.

Naturally occurring, wild-type AAVs are categorized into different serotypes, mainly based on the properties of the capsid polypeptides. These have been allocated based on parameters, such as hemagglutination, tumorigenicity and DNA sequence homology. Up to now, more than 13 different serotypes and more than a hundred sequences corresponding to different clades of AAV have been identified.

The capsid protein type and symmetry determines the tissue tropism of the respective AAV. For example, AAV2, AAV4 and AAV5 are specific to retina, AAV2, AAV5, AAV8, AAV9 and AAV-rh. 10 are specific for brain, AAV1, AAV2, AAV6, AAV8 and AAV9 are specific for cardiac tissue, AAV1, AAV2, AAV5, AAV6, AAV7, AAV8, AAV9 and AAV10 are specific for liver, AAV1, AAV2, AAV5 and AAV9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding structural gene provides for four proteins of different size, which are termed Rep78, Rep68, Rep52 and Rep40. These are involved in replication, rescue and integration of the AAV. The Cap protein encoding structural gene provides four proteins, which are termed VP1, VP2, VP3 and AAP. VP1, VP2 and VP3 are part of the proteinaceous capsid of AAV particles. In the AAV genome the combined rep and cap genes are flanked at their 5′- and 3′-ends by so-called inverted terminal repeats (ITRs).

For replication, an AAV requires in addition to the Rep and Cap proteins the products of the genes E1A, E1B, E4orf6, E2A and VA of an adenovirus or corresponding factors of another helper virus.

In the case of a wild-type AAV of the serotype 2 (AAV2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The wild-type AAV genome harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products is required.

The rep gene comprises two internal promoters, termed P5 and P19. It comprises open reading frames for four proteins. Promoter P5 is operably linked to a nucleic acid sequence providing for a non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The overexpression of the Rep proteins results in inhibitory effects on cell growth (Li, J., et al., J. Virol. 71 (1997) 5236-5243).

The two larger Rep proteins, Rep78 and Rep68, are essential for AAV duplex DNA replication, whereas the smaller Rep proteins, Rep52 and Rep40, seem to be essential for progeny and single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is encapsidated. This may be either the “plus” or “minus” strand. AAV particles containing a DNA molecule are infectious. Inside the infected cell, the parental infecting single stranded DNA is converted into a double stranded DNA, which is subsequently amplified. The amplification results in a large pool of double stranded DNA molecules from which single strands are displaced and packaged into capsids.

Adeno-associated viral particles can transduce dividing cells as well as resting cells. It can be assumed that a transgene introduced using an AAV vector into a target cell will be expressed for a long period. One drawback of using an AAVp is the limitation of the size of the transgene that can be introduced into cells.

Parvovirus particles, including AAVps and variants thereof, provide a means for ex vivo, in vitro and in vivo delivery of nucleic acid, which encode proteins, into cells such that the infected cells express the encoded protein. AAVs are viruses useful as gene therapy vectors as they can penetrate cells and introduce nucleic acid/genetic material so that the nucleic acid/genetic material may be stably maintained in the infected cells. Because AAV are not associated with pathogenic disease in humans, AAVs are able to deliver heterologous polynucleotide sequences (e.g., therapeutic proteins and agents) to human patients without causing substantial AAV-related pathogenesis or disease.

AAVp used as vehicles for effective gene delivery possess a number of desirable features for such applications, including tropism for dividing and non-dividing cells.

Early clinical experience with these vectors also demonstrated no sustained toxicity.

AAV are known to infect a wide variety of cell types in vivo and in vitro by receptor-mediated endocytosis or by transcytosis. AAVs have been tested in humans targeting retinal epithelium, liver, skeletal muscle, airways, brain, joints and hematopoietic stem cells.

Recombinant AAV particles do not typically include viral genes associated with pathogenesis. Such particles typically comprise a genome, wherein one or more of the wild-type AAV genes have been deleted in whole or in part, for example, rep and/or cap genes, but retain at least one functional flanking ITR sequence, as necessary for the rescue, replication, and packaging of the recombinant vector into an rAAVp. Thus, a rAAVp includes sequences required in cis for replication and packaging (i.e. functional ITR sequences).

Recombinant AAV particles, as well as methods and uses thereof, can be based on any wild-type AAV genome or serotype or combination thereof. As a non-limiting example, a rAAVp can be based upon any wild-type AAV genome, i.e. comprise the respective ITR sequences, such as AAV1, -2, -3, -4, -5, -6, -7, -8, -9, -10, -11, -12, 218, rh. 74, rh. 10 or 7m8 for example. Such particles can be based on the same strain or serotype (or subgroup or variant), or be different from each other. As a non-limiting example, a rAAVp based upon one wild-type genome can be identical or different to one or more of the capsid proteins that package the genome or transgene. In addition, a recombinant AAV particle can be based upon an AAV (e.g., AAV2) wild-type serotype genome that is distinct from one or more of the AAV capsid proteins that package the genome/transgene. For example, the AAV genome/transgene can be based upon AAV2, i.e. comprises AAV2-derived ITRs, whereas at least one of the three capsid proteins could be an AAV1, AAV3, AAV4, AAV5, AAV6, AAV7, AAV8, AAV9, AAV10, AAV11, AAV12, AAV-218, AAV-rh.74, AAV-rh. 10 or AAV-7m8 or a variant thereof. AAV capsid variants include variants and chimeras of AAV1, AAV2, AAV3, AAV4, AAV5, AAV6, AAV7, AAV8, AAV9, AAV10, AAV11, AAV12, AAV-2i8, AAV-rh.74, AAV-rh. 10 and AAV-7m8 capsid polypeptides.

In certain embodiments of all aspects and embodiments of the invention, the rAAVp capsid is derived from a wild-type AAV particle selected from the group consisting of AAV1, AAV2, AAV3, AAV4, AAV5, AAV6, AAV7, AAV8, AAV9, AAV10, AAV11, AAV12, AAV-218, AAV rh.74, AAV rh. 10 and AAV-7m8, as well as variants (e.g., capsid variants with, e.g., amino acid insertions, additions, substitutions and deletions) thereof, for example, as set forth in WO 2013/158879, WO 2015/013313 and US 2013/0059732 (disclosing LK01, LK02, LK03, etc.).

In certain embodiments of all aspects and embodiments of the invention, the rAAVp comprises a capsid polypeptides with an amino acid sequence having 70% or more sequence identity to an wild-type AAV1, AAV2, AAV3, AAV4, AAV5, AAV6, AAV7, AAV8, AAV9, AAV10, AAV11, AAV12, AAV-218, AAV-rh.10, AAV rh.74, or AAV-7m8 capsid polypeptide sequence.

In certain embodiments of all aspects and embodiments of the invention, the rAAVp comprises one or two ITR sequence having 70% or more sequence identity to a wild-type AAV1, AAV2, AAV3, AAV4, AAV5, AAV6, AAV7, AAV8, AAV9, AAV10, AAV11 or AAV12 ITR sequence, in one preferred embodiment to a wild-type AAV2 ITR sequence.

Recombinant AAV particles can be incorporated into pharmaceutical compositions. Such pharmaceutical compositions are useful for, among other things, administration and delivery to a subject in vivo or ex vivo. In certain embodiments of all aspects and embodiments, the pharmaceutical composition contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

Recombinant Adeno-Associated Viral Particles (rAAvps)

Different methods are known in the art for generating recombinant AAV particles.

For example, transfection with a recombinant plasmid comprising a transgene, a plasmid comprising AAV rep and cap genes, and a helper function plasmid comprising the other adenoviral helper genes. Non-limiting methods for generating rAAVp are described, for example, in U.S. Pat. Nos. 6,001,650, 6,004,797, WO 2017/096039, and WO 2018/226887. Following rAAVp production, i.e. rAAVp generation in cell culture systems, the rAAVp are recovered from the host cells and/or cell culture supernatant and purified.

For the generation of recombinant AAV particles, expression of the Rep and Cap proteins, the helper proteins E1A, E1B, E2A and E4orf6 as well as optionally the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be operably linked to said genes for functional expression.

Generally, to produce rAAVp, different, complementing plasmids are co-transfected into a host cell. One of the plasmids comprises the transgene sandwiched between the two cis acting AAV

ITRs. The missing AAV elements required for replication and subsequent packaging of progeny recombinant genomes, i.e. the open reading frames for the Rep and Cap proteins, are contained in trans on a second plasmid. Additionally, a third plasmid comprising the genes of a helper virus, i.e. E1, E4orf6, E2A and VA from adenovirus, is required for rAAV production.

To reduce the number of required plasmids, rep, cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrids can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

In order to limit the transgene activity to specific tissues, i.e. to limit the site of action, the transgene can be operably linked to an inducible or tissue specific promoter (see, e.g., Yang, Y., et al. Hum. Gene. Ther. 6 (1995) 1203-1213).

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and EIB of human adenovirus serotype 5. Plasmid pSTK146 as reported in EP 1 230 354, as well as plasmids pGS119 and pGS122 as reported in WO 2007/056994, can also be used as a source for the E1A and E1B open reading frames.

E1A is the first viral helper gene that is expressed after adenoviral DNA enters the cell nucleus. The ELA gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will die (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of E1A-induced apoptosis of cells.

The E2 gene encodes different proteins. The E2A transcript codes for the single strand-binding protein (SSBP), which is essential for AAV replication

In addition, the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr.

Rel. 154 (2011) 285-289) from a type 2 polymerases III promoter. For recombinant AAV particle production, the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are consisting of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

### General Description of Recombinant Aav Particle Production

After entry into the host cell nucleus, AAV can follow either one of two distinct and interchangeable pathways of its life cycle: the lytic or the lysogenic cycle. The former develops in cells infected with a helper virus such as Ad or herpes simplex virus (HSV) or expressing the respective helper genes, whereas the latter is established in host cells in the absence of a helper virus or expression of helper genes.

When a latently infected cell is super-infected with a helper virus or the expression of the helper genes is initiated, the AAV gene expression program is activated leading to the AAV Rep-mediated rescue (i.e., excision) of the provirus DNA from the host cell chromosome followed by replication and packaging of the viral genome. Finally, upon cell lysis, the newly assembled virions (particles) are released. Thus, the lytic phase of the AAV life cycle is induced.

Therefore, in the presence of Ad helper functions, the rAAV transgene is subjected to the wild-type AAV lytic processes by being rescued from the plasmid backbone, replicated and packaged into preformed empty AAV particles as single-stranded molecules (Gonçalves, M. A. F. V., Virol. J., 2 (2005) 43).

Generation of a recombinant AAV particle involves replacing a majority of the AAV's wild-type genome with a desired transgene and providing the viral genes that are essential for virus packaging in-trans on a separate plasmid. Once all components are transfected together into a packaging cell line, recombinant AAV particles are assembled using the cell's cellular machineries. The process of viral assembly and encapsulation takes roughly two days, after which the cells are lysed to release the rAAV for further purification and concentration (https://old.abmgood.com/marketing/knowledge_base/Adeno_Associated_Virus_Production_and_Modification_of_AAV.php).

rAAVps are not released very efficiently from the cells, although major differences have been observed between serotypes (see, e.g., Strobel, B., et al., Hum. Gene Ther. Methods 26 (2015) 147-157). When harvesting the culture, a cell disruption method is usually applied to recover the particles entrapped in the cells.

Historically, manufacturing of rAAVps was performed by double transfection of a plasmid containing the rep and the cap ORFs and a plasmid with the gene of interest flanked by ITRs. Then, a helper virus, typically Adenovirus, was co-infected (see, e.g., Aponte-Ubillus, J. J., et al., Appl. Microbiol. Biotechnol. 102 (2018) 1045-1054; Muzyczka, N., Curr. Top. Microbiol. Immunol. 158 (1992) 97-129). In this setting, the separation of the helper virus from the final product was difficult, but a critical element to avoid induction of inflammatory responses after injection into patients (see, e.g., Schnell, M. A., et al., Mol. Ther. 3 (2001) 708-722). Therefore, production of rAAVps nowadays moved towards an adenovirus-free approach by utilizing triple transfection (see, e.g., Large, E. E., et al., Viruses 13 (2021) 1336). To this end, three components are needed: one plasmid encoding the genes for Rep and Cap without the ITRs, a second plasmid with the transgene of interest flanked by ITRs, and a helper plasmid to provide the helper genes of the helper virus (see, e.g., Aponte-Ubillus, J. J., et al., Appl. Microbiol. Biotechnol. 102 (2018) 1045-1054; Farris, K. D. and Pintel, D. J., Hum. Gene Ther. 19 (2008) 1421-1427; Grimm, D., et al.,
Hum. Gene Ther. 9 (1998) 2745-2760; Ferrari, F. K., et al., Nat. Med. 3 (1997) 1295-1297). For example, the helper plasmid comprises the minimal required adenoviral genes E2A, E4 and VA. It is important to note, that the Human Embryonic Kidney cells 293 (HEK293) constitutively express the adenoviral genes E1A and E1B, which are also required for production of rAAVps. Therefore, HEK293 cells are suitable producer cells for rAA Vps and for manufacturing. Other cell types require a supplementation of E1A/B genes.

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by an AAV genome or transgene, or, vice versa, when cells comprising an integrated AAV provirus are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

Producer cells contain the rep and cap gene sequences, as well as the transgene cassette flanked by ITR sequences on one or more plasmids that are retained, e.g., via drug selection. Production of rAAVp in these cell lines generally occurs only after infection with the required helper functions or the activation of their expression. Therefore, cells are infected either with a plasmid comprising the respective helper genes to supply helper virus proteins and initiate rAAVp production. A packaging cell line differs from a producer cell line as it only contains the rep and cap genes.

More generally, cells transfected or transduced with DNA for the recombinant production of AAV particles can be referred to as a “recombinant cell”. Such a cell can be any mammalian cell that has been used as recipient of a nucleic acid (plasmid) encoding packaging proteins, such as AAV packaging proteins, a nucleic acid (plasmid) encoding helper proteins, and a nucleic acid (plasmid) that encodes a protein or is transcribed into a transcript of interest, i.e. a transgene placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such media include serum free eukaryotic growth media, such as medium for sustaining viability or providing for the growth of mammalian (e.g., human) cells. Non-limiting examples include Ham's F12 or F12K medium (Sigma-Aldrich), Freestyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

For transiently producing rAAVp, three plasmids are co-transfected into a mammalian cell. The transgene plasmid encodes the expression cassette of the gene of interest, which is interspaced between the AAV ITRs. The rep and cap genes are provided in trans by co-transfecting a second, packaging plasmid (rep/cap plasmid) to ensure AAV replication and packaging. The third plasmid, also referred to as helper plasmid, contains the minimal helper virus factors, commonly adenoviral E1A and E1B (these only in case the mammalian cell is not a HEK cell), E2A, E4orf6 and VA genes, but lacking AAV ITRs.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection is used. Calcium phosphate and polyethylenimine are the most commonly used reagents for nucleic acid transfer in larger scales (see, e.g., Baldi et al., Biotechnol. Lett. 29 (2007) 677-684), whereof polyethylenimine is preferred.

The growth in serum-free suspension culture and improvement of efficiency and reproducibility of transfection conditions using PEI as a transfection reagent permits ready scale-up the AAV production using shake-flasks, wave, or stirred-tank bioreactors.

The composition may comprise further plasmids or/and cells. Such plasmids and cells may be in contact with free PEI.

In addition to PEI, valproic acid (VPA) can be used to improve transfection efficiency. VPA is a branched short-chain fatty acid and inhibits histone deacetylase activity. Due to this reason, it is commonly added to mammalian cell culture as an enhancer of recombinant protein production.

Encoded AAV packaging proteins include, in certain embodiments of all aspects and embodiments, AAV Cap proteins. Such AAV packaging proteins include, in certain embodiments of all aspects and embodiments, AAV cap proteins of any AAV serotype.

Encoded helper proteins include, in certain embodiments of all aspects and embodiments, adenovirus E1A and E1B (but only in case the cell is not a HEK cell), adenovirus E2 and/or E4, VA RNA, and/or non-AAV helper proteins.

In certain embodiments of all aspects and embodiments, the cultivation is performed using the same generally established conditions as for the cultivation of eukaryotic cells of about 37° C., 95% humidity and 8 vol.-% CO2. The cultivation can be performed in serum containing or serum free medium, in adherent culture or in suspension culture. In certain embodiments of all aspects and embodiments, the cultivation is performed in suspension culture in serum free medium. The suspension cultivation can be performed in any fermentation vessel, such as, e.g., in stirred tank reactors, wave reactors, rocking bioreactors, shaker vessels or spinner vessels or so called roller bottles. Transfection can be performed in high throughput format and screening, respectively, e.g. in a 96 or 384 well format.

The methods according to the current invention can be used to produce AAV particles of any serotype, or a variant thereof. In certain embodiments of all aspects and embodiments, a recombinant AAV particle produced with a method according to the current invention comprises a capsid of any of AAV serotypes 1-13, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises a capsid of an AAV serotype or of an AAV pseudotype, where the AAV pseudotype comprises an AAV capsid serotype different from an ITR serotype.

Expression control elements include constitutive or regulatable control elements, such as a tissue-specific expression control element or promoter.

In certain embodiments of all aspects and embodiments of the current invention, the rAAVp comprises ITRs of any of AAV2 or AAV6 or AAV8 or AAV9 serotypes, or a combination thereof. In certain embodiments of all aspects and embodiments of the current invention, the rAAVp comprises any VP1, VP2 and/or VP3 capsid protein having 75% or more sequence identity to any of AAV1, AAV2, AAV3, AAV4, AAV5, AAV6, AAV10, AAV11, AAV12, AAV 218, AAV rh. 10, AAV rh.74 or AAV 7m8 VP1, VP2 and/or VP3 capsid proteins, or comprises a modified or variant VP1, VP2 and/or VP3 capsid protein selected from any of AAV1, AAV2, AAV3, AAV4, AAV5, AAV6, AAV8, AAV9, AAV-218, AAV-rh. 10, AAV-rh.74 and AAV-7m8 AAV serotypes.

Following production of rAAVps, if desired, the rAAVps can be purified and/or isolated from host cells using a variety of conventional methods. Such methods include column chromatography, CsCl gradient, iodixanol gradient and the like.

For example, a plurality of column purification steps such as purification over an anion exchange column, an affinity column and/or a cation exchange column can be used (see, e.g., WO 02/12455 and US 2003/0207439). Alternatively, or in addition, an iodixanol or CsCl gradient steps can be used (see, e.g., US 2012/0135515; and US 2013/0072548). Thus, in certain embodiments of all aspects and embodiments according to the current invention, the rAAVps are purified by an anion exchange chromatography, an affinity chromatography and/or a cation exchange chromatography.

Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

An objective in the rAAV production and purification systems is to implement strategies to minimize/control the generation of production related impurities such as proteins, nucleic acids, and virus-related impurities, including wild-type/pseudo wild-type AAV species (wtAAV) and AAV-encapsulated residual DNA impurities.

Considering that the rAA Vps represent only a minor fraction of the biomass, rAAVps need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

In certain embodiments of all aspects and embodiments of the method according to the current invention, as an initial step, typically the cultivated cells that produce the rAAVps are harvested, optionally in combination with harvesting cell culture supernatant (medium) in which the cells (suspension or adherent) producing the recombinant AAV particles have been cultured. The harvested cells and optionally cell culture supernatant may be used as is, as appropriate, lysed or concentrated.

Further, if infection has been employed to express helper functions, residual helper virus can be inactivated. For example, adenovirus can be inactivated by heating to temperatures of approximately 60° C. for, e.g., 20 minutes or more, which inactivates only the helper virus since rAAVps are heat stable while the helper adenovirus is heat labile.

In certain embodiments of all aspects and embodiments, the cells in the harvested cultivation broth are lysed using methods now in the art, such as detergent lysis or freeze-thaw cycles, to release the rAAVps. Concurrently during cell lysis or subsequently after cell lysis, in certain embodiments a nuclease, such as benzonase, is added to degrade contaminating DNA. In certain embodiments, the resulting lysate is clarified to remove cell debris, e.g. by filtering or centrifuging, to render a clarified cell lysate. In a particular embodiment, the lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains recombinant AAV particles (comprising full as well as empty rAAVps) and production/process related impurities, such as soluble cellular components from the host cells that can include, inter alia, cellular proteins, lipids, and/or nucleic acids, and cell culture medium components. The optionally clarified lysate is in certain embodiments of all aspects and embodiments subjected to purification steps to purify the rAAVps (comprising the transgene) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify the rAAV.

The first chromatography step is in one preferred embodiment an affinity chromatography step using an AAV affinity chromatography ligand.

If the first chromatography step is affinity chromatography the second chromatography step can be anion exchange chromatography. Thus, in certain embodiments of all aspects and embodiments, rAAVp purification is via affinity chromatography, followed by anion exchange chromatography or/and cation exchange chromatography or/and size exclusion chromatography, in any order or sequence or combination.

The removal of empty rAAVps from full ones, for example, during downstream processing is based on their different isoelectric points (pI) in anion exchange chromatography. The average calculated pI across all serotypes is 5.9 for full capsids and 6.3 for empty capsids (Venkatakrishnan, B., et al., J. Virol. 87 (2013) 4974-4984).

Cation exchange chromatography functions to separate the rAAVps from cellular and other components present in the clarified lysate and/or column eluate from an affinity or size exclusion chromatography. Examples of strong cation exchange resins capable of binding rAAVps over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYST® families of resins available from Aldrich Chemical Company (Milwaukee, WI, USA). Weak cation exchange resins include, without limitation, any carboxylic acid based resin. Exemplary cation exchange resins include carboxymethyl (CM), phospho (based on the phosphate functional group), methyl sulfonate(S) and sulfopropyl (SP) resins.

Anion exchange chromatography functions to separate rAAVps from proteins, cellular and other components present in the clarified lysate and/or column eluate from an affinity or cation exchange or size exclusion chromatography. Anion exchange chromatography can also be used to reduce and thereby control the amount of empty rAAVps. For example, the anion exchange column having full and empty rAAVps bound thereto can be washed with a solution comprising NaCl at a modest concentration (e.g., about 100-125 mM, such as 110-115 mM) and a portion of the empty rAAVps can be eluted in the flow through without substantial elution of the full rAAVps. Subsequently, full rAAVps bound to the anion exchange column can be eluted using a solution comprising NaCl at a higher concentration (e.g., about 130-300 mM NaCl), thereby producing a column eluate with reduced or depleted amounts of empty rAAVps and proportionally increased amounts of full rAAVps comprising a transgene.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNOSPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

A commercial manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

Methods to determine infectious titer of rAAV particles containing a transgene are known in the art (see, e.g., Zhen et al., Hum. Gene Ther. 15 (2004) 709). Methods for assaying for empty rAAV and full rAAV with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAVps can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

### Rep ORFs and Proteins According to the Current Invention

By regulating AAV transcription, Rep proteins impact the balance between productive replication and maintenance of the latent state, both in the presence and absence of a helper virus infection [10]. Their interactions with viral DNA elements, including cis regulatory elements upstream of viral promoters and inverted terminal repeats (ITRs) [16], influence the processing of the AAV genome. These interactions are driven by the three main protein domains of the Rep protein:
(i) the DNA- or origin-binding domain (OBD), which drives Rep binding to dsDNA at the Rep-binding elements (RBE) located within the ITRs and the p5 promoter [17];
(ii) the helicase/ATPase domain, which is required for DNA replication and packaging of viral ssDNA into the AAV capsids [12, 13];
(iii) the zinc finger and protein kinase A (PKA) Inhibitor-like domain. The zinc finger motif is required for cell cycle arrest in the S-phase [18], while the PKA-inhibitor domain seems to interfere with the PKA-sensitive Adenovirus replication, preserving AAV2 replication fitness during a co-infection [19].

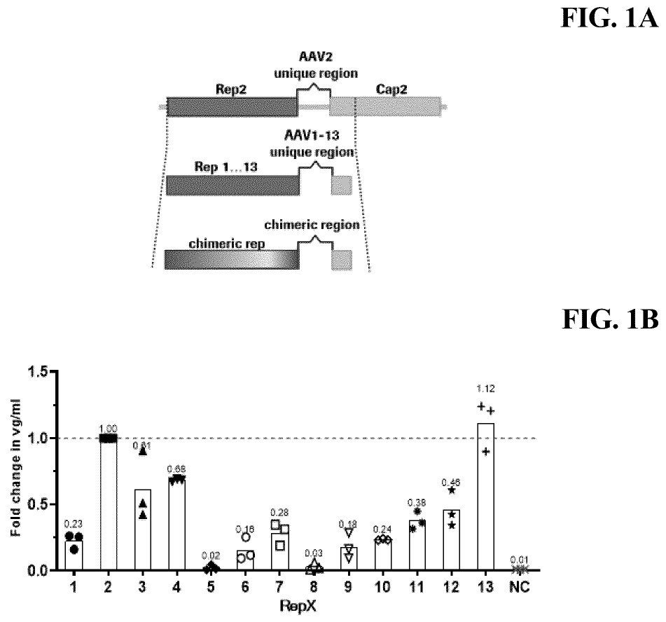

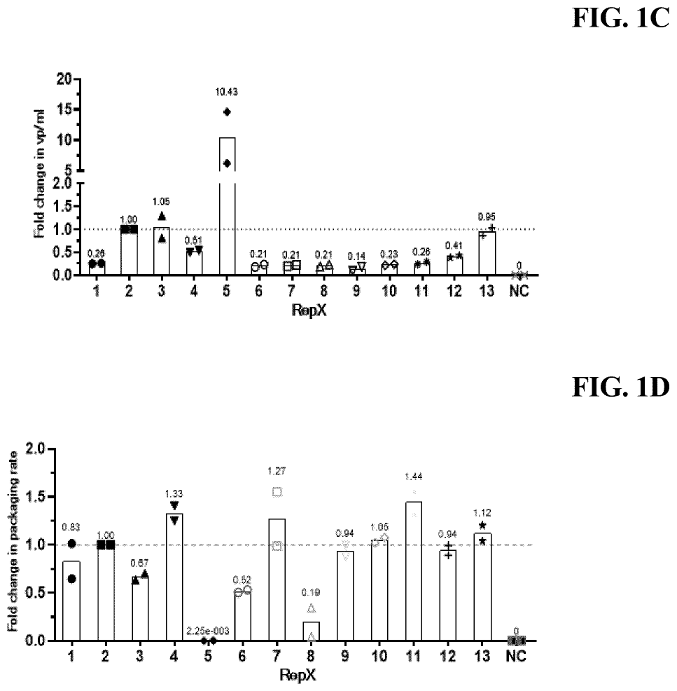

Currently more than 100 AAV serotypes are known. The basic serotypes account for 13 different AAV rep proteins. An alignment is shown in FIG. 1.

Each serotype has different properties, such as, e.g., different tissue or cell preferences, transduction rates or recombinant production capabilities.

So far, the Rep2 variant from the AAV2 serotype is commonly used in rAA Vp production systems, partly due to historical reasons, but also the reported superiority of Rep2 [26, 28].

### Cloning and Functional Assessment of 13 AAV Rep Proteins

Conventional rAAV production systems relying on the Rep2 protein from AAV2, have demonstrated efficacy in generating high viral titers across multiple AAV serotypes. However, the obtained viral titers remain notably lower than wild-type AAVs [33], and packaging efficiencies range from 5-50% of genome-containing capsids, depending on the production system used [33, 34, 35].

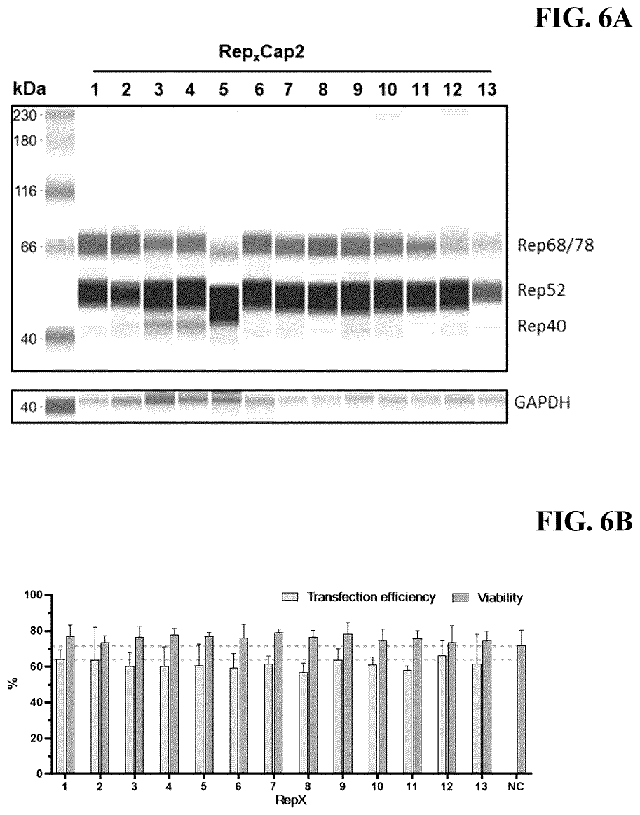

A side-by-side comparison of 13 naturally occurring Rep proteins in both adherent and suspension cells has been done in combination with Cap2 (cap gene of AAV2). The results are visualized in FIG. 1. To this end, the complete rep open reading frames (ORFs) were sourced from published wild-type AAV sequences (1-13). A 16-25 base pairs DNA stretch between rep and cap, varying among AAV isolates, was included in the final constructs (FIG. 1A). All rep ORFs were cloned upstream of the AAV2 cap gene and positioned behind a P5 mini promoter element derived from wild-type AAV2. Rep protein expression from the various plasmid backbones was confirmed through Western blot analysis of transfected cell lysates (FIG. 6A).

To assess the functionality of the different Rep proteins in an AAV2-based vector system, a GFP reporter transgene flanked by AAV2 ITRs was used. Adherent HEK293T cells were triple transfected with RepxCap (x=1 to 13) plasmid, GFP transgene plasmid, and an Adenovirus helper plasmid. GFP expression served to determine transfection efficiency, which consistently ranged between 59-66% (FIG. 6B). Similarly, comparable cell viabilities between 70-78% were observed across all conditions, including the non-transfection control (FIG. 6B). Cell lysates were utilized for various analytical measurements to determine viral vector production: (i) viral genomes per mL (vg/mL), (ii) total AAV particles per mL (vp/mL), and (iii) the calculated ratio of (i) to (ii) representing the percentage of full capsids and thereby packaging efficiency.

Differences were observed among the constructs in viral genomic titers, as determined via ddPCR analysis (see FIG. 1B). For instance, using Rep13, vg/mL titers improved to 112% compared to Rep2. Rep3 and Rep4 followed with 68% and 61%, respectively. Other Rep2 replacements resulted in a genomic titer reduction of >50%. Use of Rep5 and Rep8, reached 2-3% of the titers produced by Rep2.

To determine whether lower genomic titers resulted from low capsid expression or a defect in genome packaging, Enzyme-Linked Immunosorbent Assay (ELISA) was performed to measure total capsid formation. The data (FIG. 1C) depict the fold change of detected capsids normalized to Rep2Cap2. AAV2 capsid yields in Rep3 and Rep13 constructs were at the same level as Rep2Cap2. Slightly reduced capsid amounts were seen for Rep4 and Rep12 constructs with 51% and 41%, respectively. Surprisingly, Rep5 increased capsid production 10.43-fold relative to Rep2. All other constructs produced low levels of capsids at 14-26% of Rep2Cap2.

Based on the ELISA data packaging rates of all rAAVps with Rep1-13 compared to Rep2 were calculated (FIG. 1D). Rep1, Rep9, Rep10, Rep12, and Rep13 were able to produce a genome packaging level similar to Rep2, ranging from 83-112%. The best genome packaging efficiencies were obtained using Rep4, Rep7, and Rep11, showing 27-44% above reference conditions. In contrast, the lowest packaging rates were observed with Rep3 and Rep6. As expected, the Rep5 and Rep8 constructs with the very low genomic titers, could package even fewer or no genomes at all (FIG. 1C).

In more detail, FIG. 1A provides a schematic representation of the cloning strategy for natural rep serotypes 1 to 13, which is likewise applicable to the chimeric rep ORFs according to the current invention. The cloning procedure included a unique stretch between rep and cap in each AAV genome.

In FIG. 1B the fold change in viral genomes per mL cell culture lysate (vg/mL) is shown. Adherent HEK293T cells were transfected with rAAVps containing a GFP transgene as reporter, an Adenohelper plasmid containing the adenoviral helper genes E2A, E4 and VA (E1A and E1B are already produced by HEK cells) and one plasmid encoding the indicated Rep variant. AAV2 Cap was co-expressed from the RepX plasmids as shown in FIG. 1A. All genomic titers were normalized based on the averaged genomic titers obtained with wild-type Rep2 (n=3).

FIG. 1C shows the fold change in total capsids per mL cell culture lysate (vp/mL). HEK293T Cells were transfected as described above. All vp/mL titers were normalized based on the averaged titers obtained with wild-type Rep2 (n=2).

FIG. 1D shows the fold change in packaging rate calculated based on the data above. All rates were normalized based on the averaged rates, obtained with wild-typeRep2 (n=2).

As the aim for the production of rAAVp is to obtain the highest absolute number of full rAAVp, the sum of i) the normalized changes (normalized value of respective serotype-value for AAV2) of genomic titer (FIG. 1B) and ii) the normalized change of the packaging efficiency (FIG. 1D) shows which of the chimeric rep provides for the highest improvement relative to the reference system. The results including the sum are shown in the following Table 1.

TABLE 1
Normalized data.

| | | | | sum |
|---|---|---|---|---|
| | normalized | normalized | normalized | normalized |
| serotype | genomic titer | capsid titer | rate | change |
| 1 | 0.23 | 0.28 | 0.83 | −0.94 |
| 2 (basis | 1.00 | 1.00 | 1.00 | 0.00 |
| value) | | | | |
| 3 | 0.61 | 1.05 | 0.67 | −0.72 |
| 4 | 0.68 | 0.51 | 1.33 | 0.01 |
| 5 | 0.02 | 10.43 | 0.002 | −1.98 |
| 6 | 0.19 | 0.21 | 0.52 | −1.29 |
| 7 | 0.29 | 0.21 | 1.27 | −0.44 |
| 8 | 0.03 | 0.21 | 0.19 | −1.78 |
| 9 | 0.18 | 0.14 | 0.94 | −0.88 |
| 10 | 0.24 | 0.23 | 1.05 | −0.71 |
| 11 | 0.38 | 0.26 | 1.44 | −0.18 |
| 12 | 0.46 | 0.41 | 0.94 | −0.60 |
| 13 | 1.12 | 0.95 | 1.12 | 0.24 |

Western Blot analysis using the JESS Simple Western™ system of Rep protein expression in Expi293FTM suspension cells lysates three days post transfection is shown in FIG. 6A. Expected are the indicated Rep protein variants.

Cell viability and transfection efficiency measured three days post-transfection are shown in FIG. 6B. HEK293T cells were transfected with the indicated RepXCap2 plasmid, together with the Adenohelper and an AAV-GFP reporter. Cells were harvested in PBS and viability and flow cytometry analyses were performed.

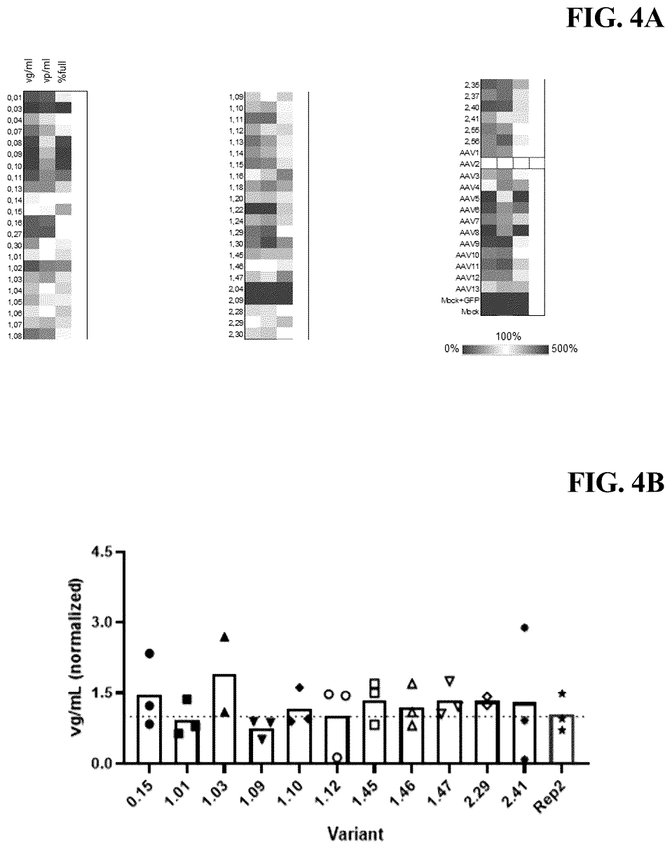

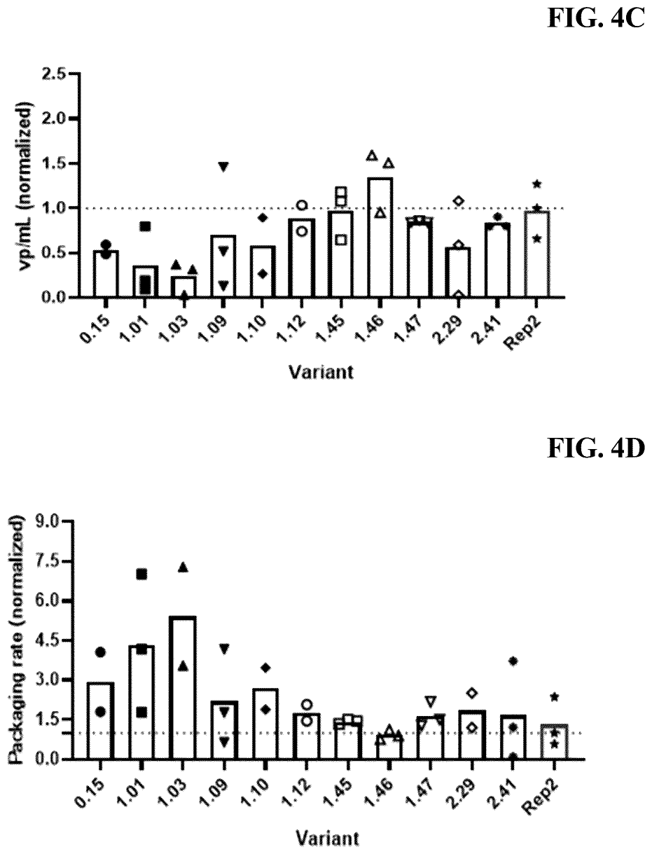

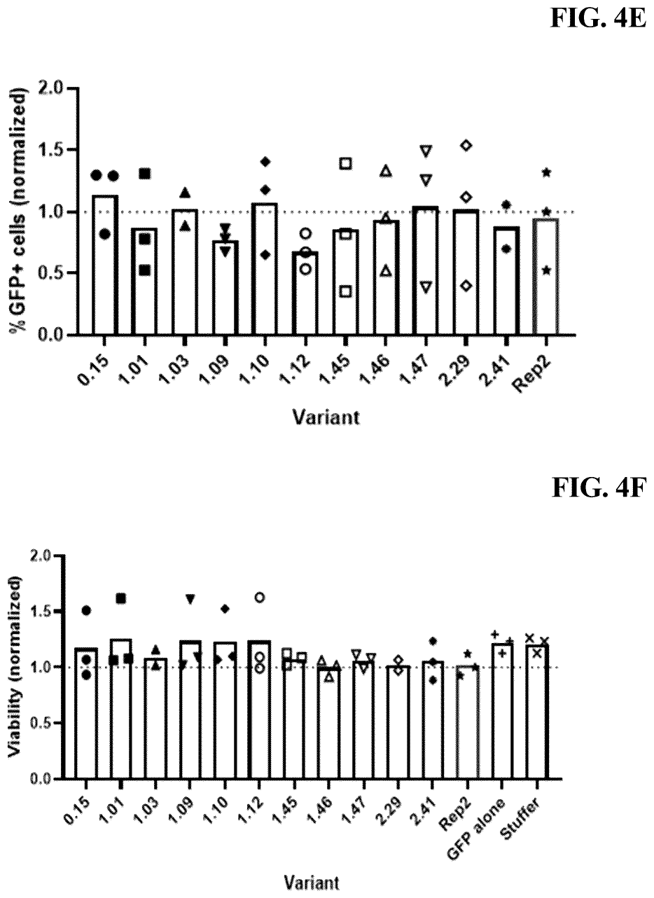

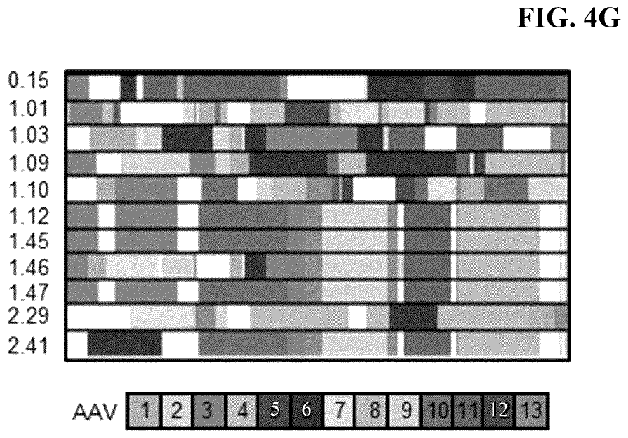

Overall, Rep3, Rep4 and Rep13 showed better genomic titers (vg/ml) than other Rep variants. On the other hand, Rep5 and Rep8 yielded the lowest genomic titers. Rep1, Rep3, Rep5, Rep6 and Rep8 capsid expression is low when using Rep proteins other than Rep2 (below 50% of Rep2 AAV2 capsid yield, see FIGS. 1C and 4A) [26,30]. It can be seen that overall Rep2 performs best.

Thus, it has been found that the Rep proteins can be roughly classified into (i) good viral genomic titers (Rep2, Rep4, Rep11, Rep13), (ii) good capsid titers (Rep2, Rep3, Rep5), and (iii) good packaging rates (Rep2, Rep4, Rep7, Rep10, Rep11, Rep13).

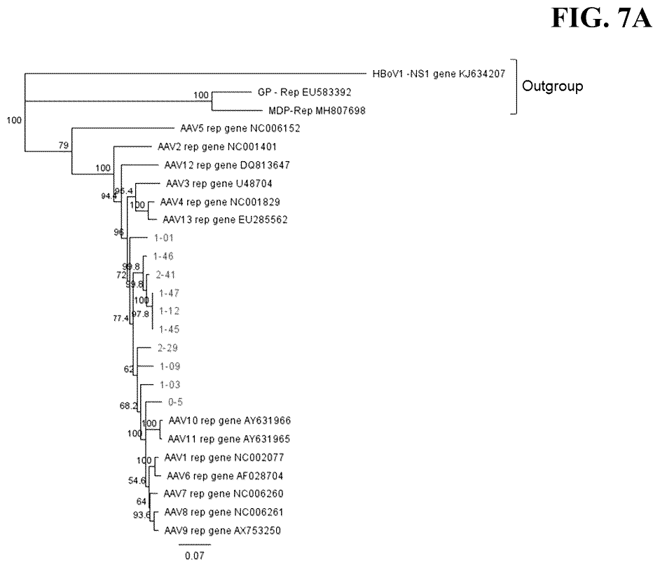

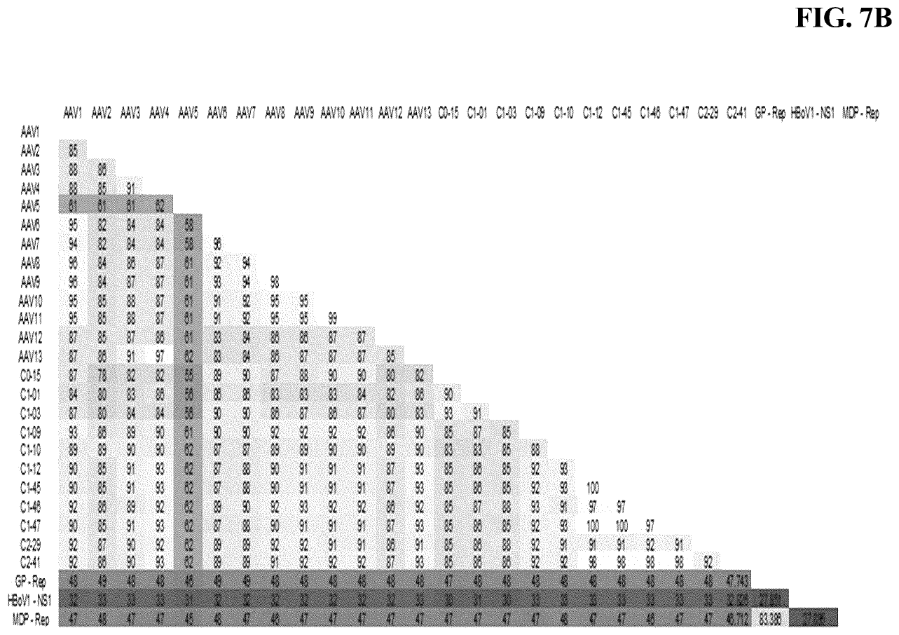

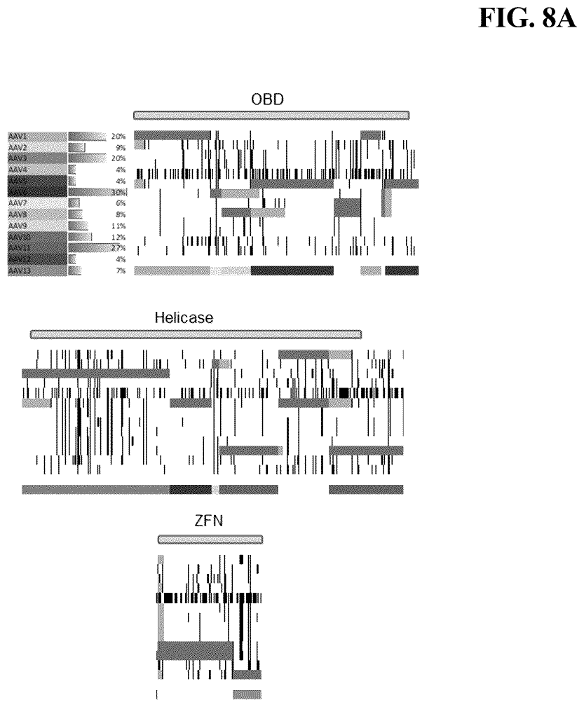

The rep ORFs show more than 80% sequence similarity (see FIGS. 7A and 7B).

The current invention is based, at least in part, on the finding that the rAAVp production, especially the packaging efficiency, can be improved by genetic engineering of the replication and packaging protein of AAV (Rep), i.e. by combining elements of the Reps of different serotypes. The encoding nucleic acid (rep), i.e. the structural gene, as well as the encoded protein (Rep) according to the current invention are chimeric sequences and chimeric proteins, respectively. A chimeric sequence is one which is different from its corresponding wild-type sequences and does not exist in nature. The term “rep” with small “r” in the beginning is denoting the encoding gene and the term “Rep” with capital “R” in the beginning is denoting the protein encoded by the respective rep.

The current invention is directed to hybrid Rep variants with distinct properties and an enrichment of combinatorial motifs. The Reps according to the current invention have advantageous properties, such as amongst others resulting in increasing rAAV capsid titer or enhancing packaging efficiency of the viral genome.

The invention is exemplified by replication and packaging of AAV2 ITR-based viral genomes. This is merely presented to exemplify the invention and shall not be construed as limitation. The scope of the current invention is set forth in the appended claims.

### DNA Family Shuffling (DFS) of AAV Rep ORFs 1-13 and Directed Evolution of Rep Proteins

The novel and advantageous Reps according to the current invention have been obtained by directed evolution of a complex Rep library generated by DNA family shuffling (DFS) of the Rep ORFs derived from the AAV serotypes 1-13 in an AAV producer cell line. After each round of selection, single clones were isolated and analyzed. Thereby it has been found that specific hybrid Rep domains were enriched. Comparative analysis of these enriched clones revealed considerable differences in their ability to package AAV2-based viral genomes. The Reps according to the current invention achieve an up to a 2.5-fold increase in packaging efficiency compared to their parental counterparts.

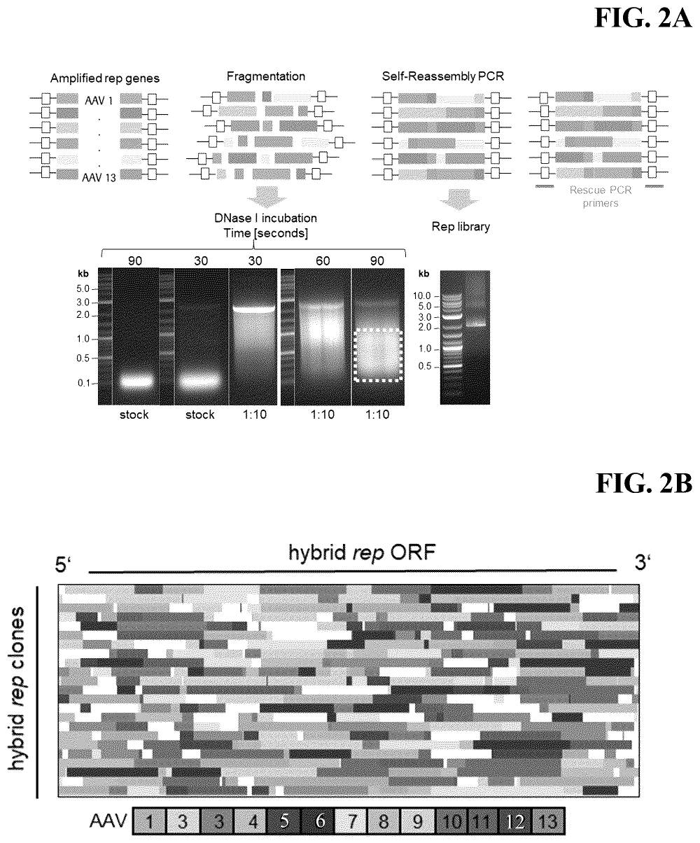

To generate a highly diverse Rep library, DFS was employed. In brief, rep ORFs were PCR amplified and subsequently digested using DNAseI (FIG. 2A). As previously described [36]. Subsequently, the digested fragments were extracted from the gel and reassembled in two consecutive PCRs to generate full-length rep hybrids, as demonstrated by the distinct 2 kb-sized band in agarose gel electrophoresis (FIG. 2A). The substantial homology exceeding 80% between the rep ORFs (except rep5) is required for the first primerless PCR (FIG. 7B). The clonal composition of the library, as depicted in FIG. 2B, evidences its high diversity, with representation from all parental serotypes and a various distribution of differently sized fragments. Rep5 is an exception, being underrepresented in the library and found in only one clone. Due to this high homology of the rep genes, some regions (white) were not clearly assignable to a single reference and therefore could not be annotated. The initial library displayed a total theoretical diversity of 3.3×1E6 clones, estimated from the number of colony-forming units per mL.

In more detail, first DNA family shuffling (DFS) was applied to diversify the rep ORF. This method requires a high sequence similarity of the used genes [36].

FIG. 2A shows the steps involved in DNA family shuffling. A pool of PCR-amplified rep ORFs was DNAseI digested under conditions indicated in the respective agarose gel electrophoresis image. This involved varying times of incubation (30, 60 or 90 seconds) and DNAseI concentration (Stock=1 U/μl or 1:10 dilution). Fragments in the indicated range (100-1000 bp) were extracted from the gel and served as input for a primerless, homology-based PCR. Finally, complete ORFs were amplified with a rescue PCR using primers binding outside of the shuffled region.

The rep ORFs have more than 80% sequence similarity except for rep5, which has less than 60% similarity (see FIG. 7B).

Thus, these genes were suitable substrates for random DFS, as exemplified with the diverse clonal composition (see FIG. 2B) and theoretical library diversity (>1E6 clones). As expected, rep5 was underrepresented. In more detail, a hybrid rep library was generated (denoted as library R0). Each line in FIG. 2B represents one clone from the 5′- to the 3′-end. Color blocks indicate the underlying parental rep sequences as shown by the color code at the bottom. “Maximum traces” were generated. Therefore, a phylogenetic analysis of the indicated rep variants (colored red in FIG. 7A) was carried out. The evolutionary history was inferred by using the Neighbor-Joining algorithm based on the Tamura-Nei model (FIG. 7A). The percentage of replicate trees in which the associated taxa clustered together in the bootstrap test (500 replicates) were shown next to the branches. The scale bar represents the average number of nucleotide substitutions per site. White annotated regions (FIG. 2B) could not be exclusively assigned to one rep reference sequence.

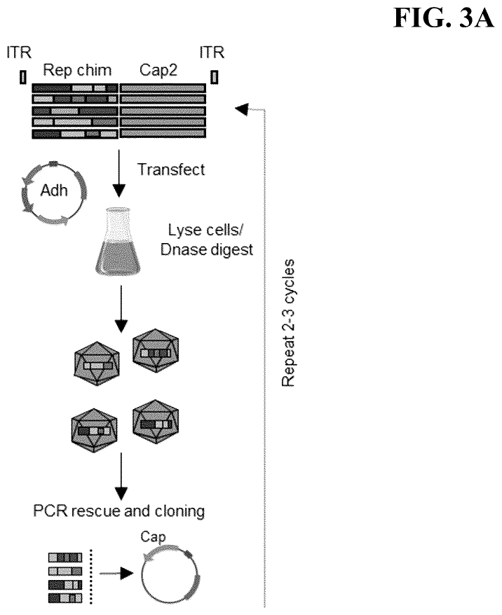

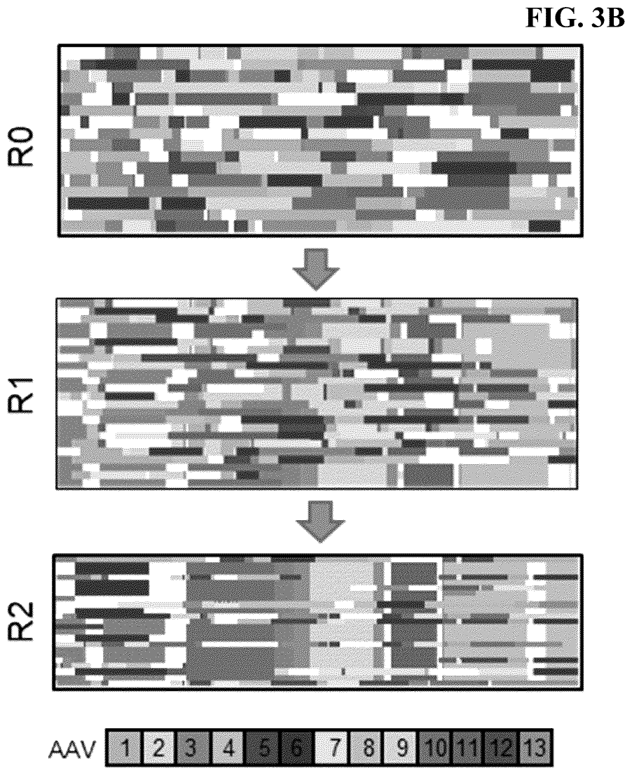

Directed evolution stands out as one of the most potent methodologies for engineering proteins and entire organisms [37-39]. A host was selected to undergo cycling with the engineered Rep library, and two screening rounds were conducted (FIG. 3A). In this process, the Rep plasmid library was transfected into suspension HEK293 cells alongside an adenovirus plasmid, facilitating wild-type AAV2 production. Unlike rAAVp production, rep and cap were packaged into the AAV2 capsid. To establish a direct link between phenotype and genotype, different DNA amounts were tested (see Table 2), ranging from 0.00025 ng to 300 ng (equivalent to 0.00347 to 41124 plasmids per cell). Subsequently, cells were harvested and lysed to release virus particles, followed by a DNAseI digest to ensure complete removal of residual plasmid DNA. Finally, packaged hybrid rep sequences were PCR amplified and cloned into the WT AAV2 acceptor plasmid for clonal assessment (see FIG. 3B) and initiation of the 2nd selection round (see FIG. 3A). Surprisingly, the genomic titer of the initial library was high enough to use the lowest plasmid amount tested (0.25 ng per 1×1E6 cells). This is surprising in view of the randomness of DFS and the estimated 50% frameshift rate in the library (as calculated from 25 sequenced clones; data not shown). After the second selection round, an increase in viral titers reflected an improved fitness of the library (compare 0.25 ng conditions in Table 2). Without being bound by this theory it is assumed that this is due to the enrichment of functional Rep hybrids.

Sequences from each selection round were aligned using MUSCLE, and clonal composition was assessed. Unexpectedly, a shift in the clonal composition of the library was observed already after the first selection round and further accumulated after the second selection round. The most prominent clone exhibited an enrichment of rep3-, rep10-, rep13-, rep9-, rep11-, and rep4-derived sequences from the 5′ to the 3′ end (5/25). In total 15 different rep fragments were present. After the 2nd round, a dominant clone composed of the aforementioned rep parental sequences, except rep3, emerged, featuring a novel sequence derived from rep6 at the 5′ end, not observed in sequenced clones from round 1. In total 14 different rep fragments were present in the dominant clone 2.41.

In contrast thereto, the most advantageous clones according to the current invention exhibited from the 5′ to the 3′ end (n/a=any AAV serotype)

Clone 0.15:
AAV3-n/a-AAV6-n/a-AAV11-AAV8-AAV11-AAV13-n/a-AAV6-
AAV12-AAV6-AAV11-AAV10;
Clone 1.01:
AAV7-AAV3-AAV4-AAV11-n/a-AAV9-AAV12-AAV2-AAV1-
AAV10-AAV4-n/a-AAV4-AAV12-AAV1-AAV7-AAV10-AAV4-
AAV9-AAV12-AAV3-AAV4-n/a-AAV4-AAV13-AAV7;
Clone 1.03:
n/a-AAV1-AAV7-AAV9-AAV6-AAV9-AAV1-n/a-AAV6-AAV3-
AAV6-AAV2-AAV11-n/a-AAV11-n/a-AAV13;
Clone 1.10:
n/a-AAV1-AAV3-n/a-AAV10-n/a-AAV2-AAV4-AAV13-AAV11-
n/a-AAV12-n/a-AAV12-AAV10-AAV7-AAV13-AAV1-AAV10-
AAV2;

derived sequences and comprised more than 13 different rep fragments. Thus, it can be seen that the combination of sequences from more than 16, preferably more than 19 or most preferably more than 25 different rep genes results in more advantageous rep genes.

In more detail, second, the library was subjected to a selection process by performing cycles of wild-type AAV2 production in suspension cells.

In FIG. 3A a schematic representation of the directed evolution approach is shown. Suspension HEK293 cells were transfected with the chimeric rep library and an Adenohelper plasmid for wild-type rAAVp of serotype 2 (rAAV2p) production. Cells were lysed to recover rAAV2p particles packaged with the chimeric rep ORFs. Primers binding outside of the shuffled region were used to PCR amplify the hybrid regions. Finally, chimeric rep ORFs were cloned into an acceptor plasmid with AAV2 cap for clonal assessment and initiation of 2nd selection round.

The employed chimeric Rep/Cap Plasmid amounts and corresponding genomic titers as fold changes are shown in the following Table 2. Titers obtained with the lowest DNA amount in round 1 (0.25 ng) were set to 1.

TABLE 2
Chimeric Rep/Cap Plasmid amounts and corresponding genomic
titers as fold changes. Titers obtained with the lowest
DNA amount in round 1 (0.25 ng) were set to 1.

| Round | Plasmid [ng] | Titer [vg/mL] |
|---|---|---|
| 1 | 300 | 9.4 |
| 1 | 25 | 6.6 |
| 1 | 2.5 | 1.9 |
| 1 | 0.25 | 1 |
| 2 | 0.25 | 2.4 |
| 2 | 0.025 | 1.9 |
| 2 | 0.0025 | 0.3 |
| 2 | 0.00025 | 0.15 |

FIG. 3B depicts the clonal composition of rep hybrids after each selection round. Each line corresponds to one clone from the 5′- to 3′-end. Color blocks represent underlying rep reference sequences, as indicated with the color code at the bottom. R0=primary, pre-selected library. R1/R2=selection rounds 1 and 2.

Unexpectedly and surprisingly, after only one selection round, there was an increase in specific domains' presence and an enrichment of clones, which became even more pronounced after the second selection round (see FIG. 3B). Further unexpectedly and surprisingly, none of the clones contained sequences from the superior rep2, and the domain composition remained highly chimeric, with the most dominant clone composed of sequences derived from seven parental rep ORFs.

Also noteworthy were the high titers of the unselected library, reflecting a high plasticity of the Rep proteins.

Without being bound by this theory, it is assumed that the observed enhancement in viral titers after each selection round indicates an increase in the fitness of the underlying clones, as described earlier for AAV capsid libraries [68].

### Small and Mid-Scale Assessment of Rep Hybrid Functionalities

To assess whether the enriched variant Rep producing clones provide for an advantage relative to wild-type Rep2, variants from all selection rounds were tested in a small-scale, high-throughput functional assessment in 24 deep well format.

That is, fifty variants were randomly selected from different selection rounds, with clone numbers starting at 0, 1, or 2 for the initial library or selection rounds 1/2, respectively (FIG. 4A). Parental Rep variants 1-13 served as controls. Assessed parameters included viral genomic titers in the harvest (vg/mL), capsid titer (vp/mL), packaging rate, and cell viability.

In line with observations in adherent HEK293 cells, Rep3, Rep4, and Rep13 provided for better results than other natural Rep variants, displaying vg/mL titers close to Rep2 (55.8%, 91.6%, and 75.8% of Rep2 titers, respectively). Furthermore, Rep4 and Rep13 demonstrated an enhanced packaging rate with 2.9-fold and 2.5-fold increases (median values).

The majority of clones from the initial library exhibited minimal to no viral genomic titers. Variants 0.04, 0.14, and 0.15 (3/14 clones) exhibited vg/mL titers above 50% of Rep2 titers. The vp/mL titer appeared less affected, with 10/14 variants from the initial library displaying either good (above 50%) or medium (above 30%) vp/mL titers compared to Rep2.

In contrast to the initial library, clones from the 1st and 2nd selection rounds were predominantly functional, yielding vg/mL titers ranging from 11.4% to 108% and vp/mL titers ranging from 6.3% to 120% of Rep2 levels. The packaging rate, calculated from the vg/mL and vp/mL ratio, identified several clones with a higher packaging rate than Rep2, i.e. clones 0.15, 1.03, 1.04, 1.16, 1.18, 1.20, 1.30, 1.45, 1.47, 2.29, 2.37, and 2.56. However, clones 1.30, 2.37, and 2.56, despite showing a favorable packaging rate, exhibited low viral vg/mL titers (below 50% of Rep2). Notably, cell viability remained unaffected, ranging from 96% to 108% of Rep2.

Based on the small-scale results, 11 variants were selected for further evaluation at a mid-scale level (30 mL shake flask; FIG. 4B-F). Most tested variants produced genomic titers similar to Rep2 (FIG. 4B). However some variants showed significant differences in vp/mL titers (FIG. 4C), i.e., the total viral capsids (e.g., variants 0.15, 1.01, and 1.03; FIG. 4C). This resulted in a shift towards a favorable packaging rate, which is the ratio between the two parameters (FIG. 4D). The functionality of the produced AAV2 capsids were assessed by using these Rep variants for packaging a GFP-encoding genome into the constructs and transducing adherent HEK293A cells with the resulting crude lysates at multiple dilutions. As shown in FIG. 4E, only small differences were observed. These small differences reflect the variations in the genomic titers, confirming the ability of all Rep hybrids to produce functional rAAVps. Likewise, no significant differences were observed in producer cell viability.

Sanger sequencing analysis of the clones was performed and the clonal composition was assessed (FIG. 4G). Thereby three identical sequences were found (1.12, 1.45, and 1.47) representing the leading clone after the first selection round. All three behaved nearly identically in the mid-scale experiments. Two clones, 1.46 and 2.41, were very similar to the leading clone, differing only in their 5′ sequence. All other clones showed a distinct, chimeric nature.

In more detail, functional validation of single clones confirmed the accumulation of viable rep ORFs, as the majority of the unselected clones were dysfunctional. In more detail, in FIG. 4A the results of a high-throughput screening of Rep variants in a micro-scale setting (24 deep-well) is shown. Data from the indicated analytical measurements are represented as a heat map with Rep2-values set to 100% (white) and the highest limit corresponding to 500% of Rep2 levels (red); n=2-3 biological replicates.

A number of clones were transferred for functional validation and further evaluation from micro- to mini-scale culture formats. Thereby a good transferability of the properties between the scales was observed.

The testing of selected Rep candidates was performed in mini-scale (30 ml shake flasks; n=2-3 biological replicates). The following parameters were assessed: (i) genomic titer (FIG. 4B), (ii) capsid titer (FIG. 4C), (iii) packaging rate (ratio of FIGS. 4B and 4C, FIG. 4D), (iv) functionality of resulting AAV vectors (% of GFP positive cells) after transduction with crude cell lysates (FIG. 4E) with equal volumes applied (1:10 dilution of stock), and (v) cell viability at day of harvest (FIG. 4F).

The data presented in FIGS. 4B to 4F for the different clones normalized to the reference system with Rep2 and a transgene with AAV2 ITRs is summarized in the following Table 3.

TABLE 3
Normalized data.

| | | | | FIG. 4E | |
|---|---|---|---|---|---|
| normalized | FIG. 4B | FIG. 4C | FIG. 4D | [% GFP | FIG. 4F |
| Clone | [vg/mL] | [vp/mL] | [% full] | positive] | viability |
| 0.15 | 1.4 | 0.5 | 3.0 | 1.15 | 1.15 |
| 1.01 | 0.9 | 0.3 | 4.3 | 0.85 | 1.2 |
| 1.03 | 1.9 | 0.2 | 5.2 | 1.0 | 1.1 |
| 1.09 | 0.7 | 0.7 | 2.0 | 0.75 | 1.2 |
| 1.10 | 1.1 | 0.6 | 2.6 | 1.1 | 1.18 |
| 1.12 | 1.0 | 0.8 | 1.7 | 0.6 | 1.2 |
| 1.45 | 1.4 | 0.9 | 1.4 | 0.8 | 1.1 |
| 1.46 | 1.2 | 1.3 | 0.9 | 0.9 | 0.95 |
| 1.47 | 1.4 | 0.8 | 1.6 | 1.05 | 1.1 |
| 2.29 | 1.3 | 0.6 | 1.7 | 1.0 | 1.0 |
| 2.41 | 1.3 | 0.8 | 1.6 | 0.8 | 1.05 |
| rep2 | 1.0 | 1.0 | 1.2 | 0.9 | 1.0 |

In order to visualize the difference between the reference system and the chimeric rep clones the normalized change relative to the reference system has been calculated and is shown in the following Table 4.

TABLE 4
Normalized change data.

| normalized | | | | FIG. 4E | |
|---|---|---|---|---|---|
| change | FIG. 4B | FIG. 4C | FIG. 4D | [% GFP | FIG. 4F |
| Clone | [vg/mL] | [vp/mL] | [% full] | positive] | viability |
| 0.15 | 0.4 | −0.5 | 1.8 | 0.25 | 0.15 |
| 1.01 | −0.1 | −0.7 | 3.1 | −0.05 | 0.2 |
| 1.03 | 0.9 | −0.8 | 4 | 0.1 | 0.1 |
| 1.09 | −0.3 | −0.3 | 0.8 | −0.15 | 0.2 |
| 1.10 | 0.1 | −0.4 | 1.4 | 0.2 | 0.18 |
| 1.12 | 0 | −0.2 | 0.5 | −0.3 | 0.2 |
| 1.45 | 0.4 | −0.1 | 0.2 | −0.1 | 0.1 |
| 1.46 | 0.2 | 0.3 | −0.3 | 0 | −0.05 |
| 1.47 | 0.4 | −0.2 | 0.4 | 0.15 | 0.1 |
| 2.29 | 0.3 | −0.4 | 0.5 | 0.1 | 0 |
| 2.41 | 0.3 | −0.2 | 0.4 | −0.1 | 0.05 |
| rep2 | 0 | 0 | 0 | 0 | 0 |

As the aim for the production of rAAVp is to obtain the highest absolute number of full rAAVp, the sum of i) the normalized changes of genomic titer (FIG. 4B) and ii) the packaging efficiency (FIG. 4D) shows which of the chimeric rep provides for the highest improvement relative to the reference system. It can be seen that clones 0.15, 1.01, 1.03 and 1.10 all provide for an increase relative to the reference system, whereas clones 1.09, 1.12, 1.45, 1.46, 1.47, 2.29 and 2.41 perform less than the reference system.

TABLE 5
Sum normalized change data.

| sum normalized | FIG. | FIG. | FIG. |
|---|---|---|---|
| change Clone | 4B + 4D | 4B + 4D + 4E | 4B + 4D + 4E + 4F |
| 0.15 | 2.20 | 2.45 | 2.60 |
| 1.01 | 3.00 | 2.95 | 3.15 |
| 1.03 | 4.90 | 5.00 | 5.10 |
| 1.09 | 0.50 | 0.35 | 0.55 |
| 1.10 | 1.50 | 1.70 | 1.88 |
| 1.12 | 0.50 | 0.20 | 0.40 |
| 1.45 | 0.60 | 0.50 | 0.60 |
| 1.46 | −0.10 | −0.10 | −0.15 |
| 1.47 | 0.80 | 0.95 | 1.05 |
| 2.29 | 0.80 | 0.90 | 0.90 |
| 2.41 | 0.70 | 0.60 | 0.65 |
| rep2 | 0.00 | 0.00 | 0.00 |

In FIG. 4G an overview of the clonal composition of selected rep hybrids (5′- to 3′-end) is shown. Color blocks represent the underlying rep reference sequences, as indicated with the color code at the bottom.

The composition of the respective clones are as follows:

Clone 0.15:
AAV3-n/a-AAV6-n/a-AAV11-AAV8-AAV11-AAV13-n/a-AAV6-
AAV12-AAV6-AAV11-AAV10;
Clone 1.01:
AAV7-AAV3-AAV4-AAV11-n/a-AAV9-AAV12-AAV2-AAV1-
AAV10-AAV4-n/a-AAV4-AAV12-AAV1-AAV7-AAV10-AAV4-
AAV9-AAV12-AAV3-AAV4-n/a-AAV4-AAV13-AAV7;
Clone 1.03:
n/a-AAV1-AAV7-AAV9-AAV6-AAV9-AAV1-n/a-AAV6-AAV3-
AAV6-AAV2-AAV11-n/a-AAV11-n/a-AAV13;
Clone 1.09:
AAV3-n/a-AAV9-AAV3-AAV2-AAV1-AAV6-AAV10-AAV8-AAV6-
AAV11-n/a-AAV12-AAV4-AAV13-n/a;
Clone 1.10:
n/a-AAV1-AAV3-n/a-AAV10-n/a-AAV2-AAV4-AAV13-AAV11-
n/a-AAV12-n/a-AAV12-AAV10-AAV7-AAV13-AAV1-AAV10-
AAV2;
Clone 1.12:
AAV3-n/a-AAV3-n/a-AAV10-AAV3-AAV13-AAV9-AAV13-n/a-
AAV11-n/a-AAV13-AAV4-n/a-AAV7-AAV4;
Clone 1.45:
AAV3-n/a-AAV3-n/a-AAV10-AAV3-AAV13-AAV9-AAV13-n/a-
AAV11-n/a-AAV13-AAV4-n/a-AAV7-AAV4;
Clone 1.46:
AAV3-AAV9-AAV1-AAV7-n/a-AAV9-AAV12-n/a-AAV1-n/a-
AAV6-AAV3-AAV13-AAV9-AAV13-AAV9-AAV11-n/a-AAV13-
AAV4-n/a-AAV7-AAV13;
Clone 1.47:
AAV3-n/a-AAV3-n/a-AAV10-AAV3-AAV13-AAV9-AAV13-n/a-
AAV11-n/a-AAV13-AAV4-n/a-AAV7-AAV13;
Clone 2.29:
n/a-AAV7-AAV13-AAV2-n/a-AAV4-n/a-AAV4-AAV6-AAV8-
AAV1-AAV13;
Clone 2.41:
n/a-AAV6-n/a-AAV10-AAV3-AAV13-AAV9-AAV13-n/a-
AAV11-n/a-AAV13-AAV4-n/a-AAV7-AAV13;

n/a—cannot be exclusively assigned to one rep reference sequence.

All selected clones, i.e. clones 0.15, 1.01, 1.03 and 1.10, performed comparable or better than Rep2 in packaging a GFP encoding reporter transgene flanked by wild-type AAV2 ITRs.

However, differences were observed in genome packaging efficiency, where several clones (0.15, 1.01, 1.03, 1.10) outperformed Rep2 while maintaining high viral genomic titers. It was further confirmed that none of the Rep hybrids affected the transduction ability of the resulting rAAVp. This is expected as Rep is not incorporated into the viral capsid.

### Functional Assessment of Clones 2.41 and 1.03 in the AMBR15 and 250 Fermentation Systems

Mini- and mid-scale high-throughput screening experiments in AMBR15 and AMBR250 systems were performed.

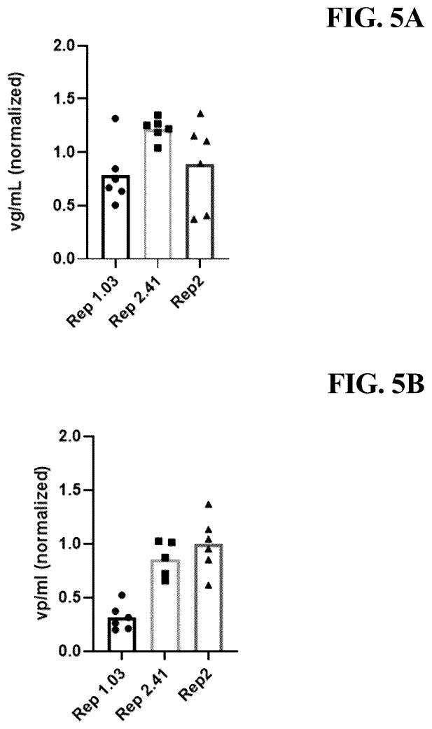

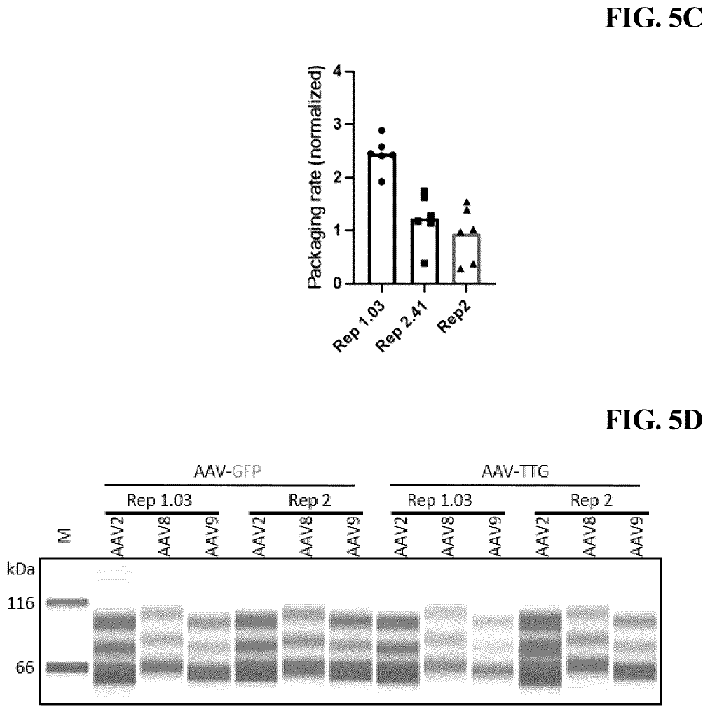

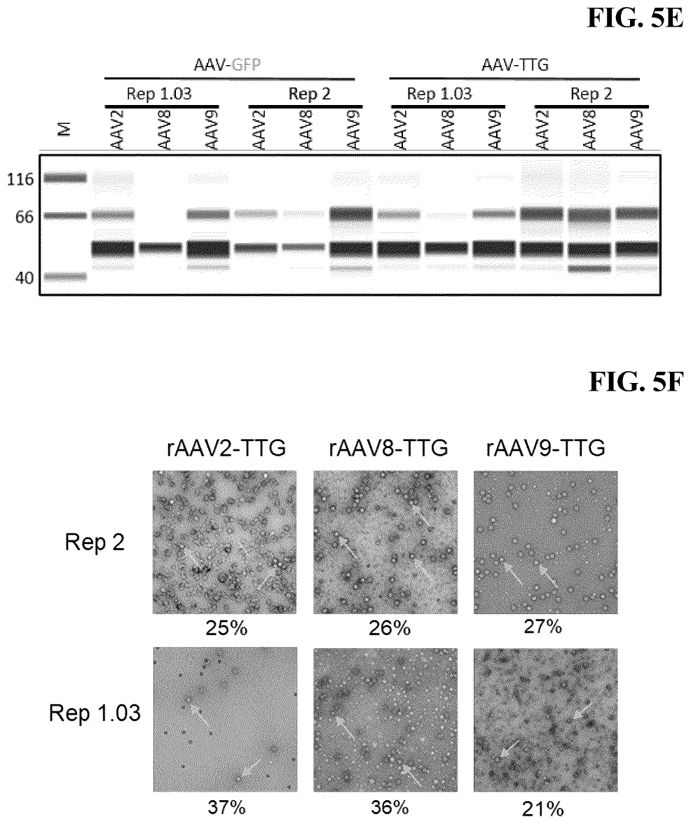

First a side-by-side comparison of Rep1.03, Rep2.41, and Rep2 was performed in the AMBR15 system, using GFP as transgene (FIG. 5A-C). Consistent with the before presented results, Rep2.41 showed slightly increased viral genomic titers (FIG. 5A) but did not affect the packaging rate (FIG. 5C). In contrast, Rep1.03 did not alter the viral genomic titers but increased the packaging rate by decreasing the total capsid amount by more than 50% (FIG. 5B).

Second, the Rep variant, 1.03, was combined with the capsid of two other AAV serotypes (AAV8 and AAV9). These constructs were tested for rAAV production in the AMBR250 system using two different transgenes, i.e. GFP and a therapeutic gene (TTG). Consistent with the before presented results in the mini- and midscale, Rep1.03 increased the amount of full capsids by 2-fold and 3.5-fold for GFP and TTG, respectively (Table 7). The packaging rate for AAV8 was also increased, with a 1.67- and 2.3-fold increase for GFP and TTG, respectively. Combination with AAV9 resulted in a general decrease in vp/mL but no increase in the packaging rate (see Table 7).

A western blot analysis of VP and Rep protein expression was performed (FIG. 5D). For AAV2, no decrease in the major capsid protein (VP3) expression using Rep1.03 compared to Rep2 (FIG. 5B and Table 8) was observed with both transgenes. Without being bound by this theory, it is assumed that the sharp decrease in capsid formation cannot be explained by a low availability of capsid protein, but the slight transgene-dependent decrease in AAV8 and AAV9 VP3 expression, might explain the noted decrease in capsid formation.

Rep protein expression (FIGS. 5D and Table 8) varied between the conditions, with a trend toward decreased large Rep68/78 expression in conditions containing Rep1.03. This is a preferred embodiment of the invention.

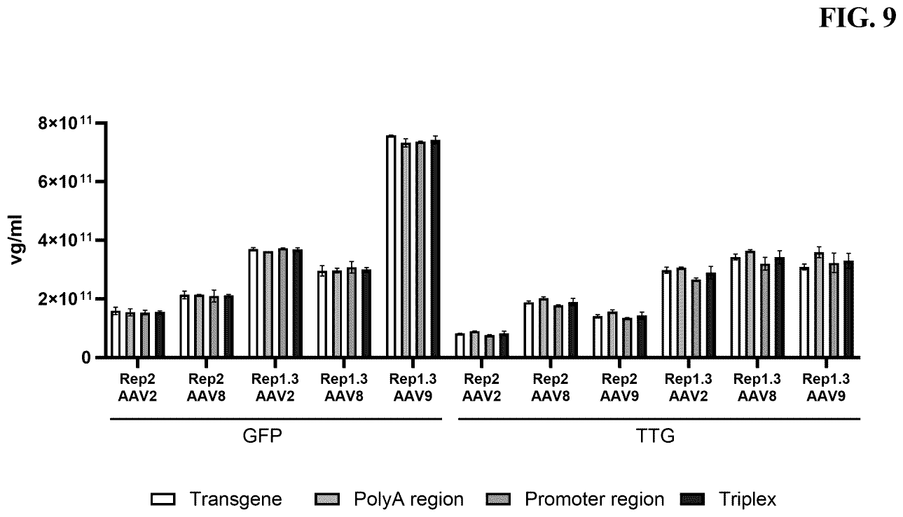

A small fraction of the AMBR250 lysates was purified using an AAVX affinity resin that binds both full and empty particles, thereby preserving their original ratio in the cell lysates. The viral genome titer (vg/mL) of these preparations was quantified using three distinct primer/probe sets targeting the 5′ end, 3′ end, and middle of the genome (see FIG. 9). Notably, highly similar titers were observed in the simplex and multiplex assays. Additionally, a multiple-occupancy analysis was performed to estimate the percentage of intact genomes. The analysis revealed that differences between Rep2 and Rep1.03 were either absent or small and depended on the transgene (Table 6).

TABLE 6
Estimation of viral genome intactness. The genome intactness
of the rAAV preparations shown in FIG. 9 was assessed using
the multiple-occupancy analysis feature of the QIAcuity Software
Suite (version 2.5.0.1). Percentages derived using Rep2 were
averaged and normalized to a reference value of 1.

| | | | Intact genomes |
|---|---|---|---|
| Transgene | Rep | capsid | (ratio) |
| GFP | 2 wild-type | AAV2 | 1 |
| GFP | clone 1.03 | AAV2 | 1.004 |
| GFP | 2 wild-type | AAV8 | 1 |
| GFP | clone 1.03 | AAV8 | 1.005 |
| TTG | 2 wild-type | AAV2 | 1 |
| TTG | clone 1.03 | AAV2 | 0.82 |
| TTG | 2 wild-type | AAV8 | 1 |
| TTG | clone 1.03 | AAV8 | 0.91 |
| TTG | 2 wild-type | AAV9 | 1 |
| TTG | clone 1.03 | AAV9 | 0.80 |

TTG = therapeutic transgene

Cryo-EM analysis of the purified lysates was performed, and the percentage of full particles was estimated using automated image analysis (FIG. 5F). It has been found that Rep1.03 increased the percentage of full capsids to 37% and 36% for AAV2 and AAV8, respectively (compared to 25% and 26%, respectively, using Rep2). The percentage of full capsids did not change for AAV9.

In more detail, two clones, clone 1.03 and the dominant clone 2.41, were tested in AMBR15 and AMBR250 fermentation systems, which are micro- and mini-bioreactors, respectively, that allow automated control of culturing conditions and offer scalability to larger bioreactor settings. The production of rAAVp comprising a GFP coding sequence as transgene with the indicated Rep variants or Rep2 control was performed and the following parameters were determined: (i) genomic titer (FIG. 5A), (ii) capsid titer (FIG. 5B), (iii) packaging rate (ratio of FIG. 5A to 5B, FIG. 5C).

The superior packaging rates for clone 1.03 (2.5-fold on average) were confirmed in these experiments.

Additionally, clone 1.03 was tested with two further AAV serotypes (AAV8 and AAV9) and two different transgenes (GFP and a therapeutic transgene; both of the same size). Consistently higher packaging rates for AAV2 and AAV8, but not AAV9, were observed.

Genomic and capsid titers were assessed using ddPCR and ECLIA, respectively. The results are shown in Table 7. The calculated ratio of both parameters represents the percentage of full capsids. Data were normalized to the Rep2-control (set to 1).

TABLE 7
Genomic and capsid titers were assessed using ddPCR and
ECLIA, respectively. The calculated ratio of both parameters
represents the percentage of full capsids. Data were
normalized to the Rep2-control (set to 1).

| | Parameter | | | | | |
|---|---|---|---|---|---|---|
| | [ng/mL] | | [vp/mL] | | [% full] | |
| Transgene | GFP | TTG | GFP | TTG | GFP | TTG |
| Rep1.03-AAV2 | 0.7 | 0.5 | 0.53 | 0.2 | 2.0 | 3.5 |
| Rep1.03-AAV8 | 1.1 | 1.25 | 0.61 | 0.5 | 1.67 | 2.3 |
| Rep1.03-AAV9 | n.d. | 0.65 | n.d. | 0.4 | n.d. | 1.3 |

n.d. = not determined

A western blot analysis using the JESS Simple Western™ system was performed. Cell lysates from the indicated rAAV productions in the AMBR250 system were semi-quantitative analyzed for capsid protein expression (VP1-VP3; FIG. 5D) or Replication protein (Rep) expression (FIG. 5E).

A semi-quantitative Western blot analysis was performed. The results are shown in Table 8. The area under the peak was calculated using the Compass for SW Version 6.3.0 and corrected to the total protein content. Data of each condition were normalized to the respective Rep2 control construct.

TABLE 8
Semi-quantitative Western blot analysis. The area under the
peak was calculated using the Compass for SW Version 6.3.0
and corrected to the total protein content. Data of each condition
were normalized to the respective Rep2 control construct

| | Parameter | | | | | |
|---|---|---|---|---|---|---|
| | VP1 | | VP2 | | VP3 | |
| Transgene | GFP | TTG | GFP | TTG | GFP | TTG |
| Rep1.03-AAV2 | 0.99 | 0.86 | 0.89 | 0.67 | 1.07 | 0.99 |
| Rep1.03-AAV8 | 0.77 | 0.67 | 0.75 | 0.65 | 0.81 | 0.65 |
| Rep1.03-AAV9 | 0.80 | 0.57 | 0.70 | 0.50 | 0.77 | 0.56 |

| | Condition | | | | | |
|---|---|---|---|---|---|---|
| | Rep1.03-AAV2 | | Rep1.03-AAV8 | | Rep1.03-AAV9 | |
| Transgene | GFP | TTG | GFP | TTG | GFP | TTG |
| Rep40 | 2.9 | 0.78 | 1.5 | 0.48 | 0.9 | 0.74 |
| Rep52 | 4.8 | 1.25 | 2.03 | 0.6 | 1.5 | 1.3 |
| Rep68/78 | 1.58 | 0.35 | 0.32 | 0.12 | 0.44 | 0.39 |

The rep of Clone 1.03 from selection round 1 was aligned to the 13 parental rep references using MUSCLE (DNA alignment) and is shown in FIG. 4SA.

Mismatches to the references at single nucleic acid positions are colored in black. Matching regions are shown in green and were ranked according to their length. A “maximum trace”-colored in dark green-reflects the highest probability of origin of a particular region. If several traces were identified, the longer stretch was colored in dark green and the others in light green. Blue bars reflect the percentage of each reference sequence in the clone composition. Above the alignment the approximate location of the underlying Rep domains is shown.

A phylogenetic analysis of the indicated Rep domains was performed. OBD=origin binding domain (AA residues 1-225); Helicase (AA residues 225-490); ZF=Zinc finger domain (AA residues 531-625). Hybrid Rep variant 1.03 is colored red. The evolutionary history was inferred by using the Neighbor-Joining algorithm based on the Jukes-Cantor model. The percentage of replicate trees in which the associated taxa clustered together in the bootstrap test (500 replicates) are shown next to the branches. The scale bar represents the average number of nucleotide substitutions per site. A visualization is presented in FIGS. 4SB, 4SC and 4SD (TTG=therapeutic transgene).

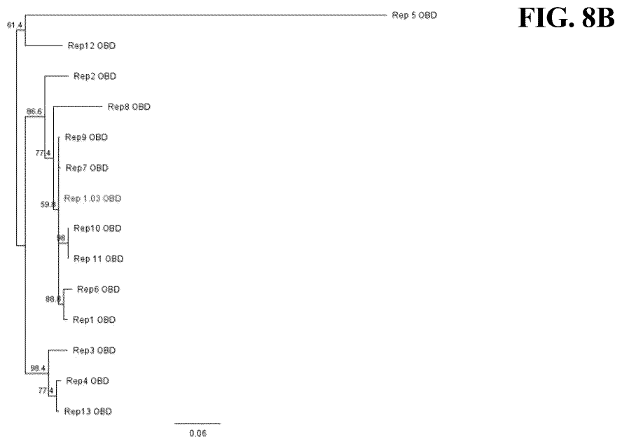

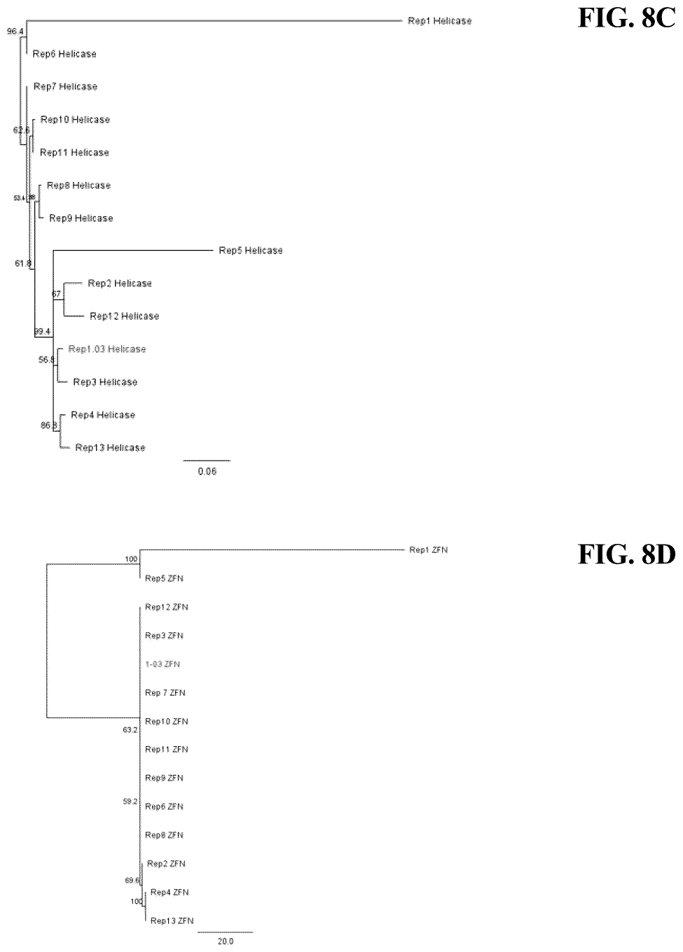

Interestingly, clone 1.03 outperformed the dominant clone 2.46 and displayed a very chimeric and distinct domain composition compared to the other clones (underlying serotypes 1, 2, 3, 6, 7, 9, 11 and 13; see FIG. 8A).

Clone 1.03 has a close phylogenetic relationship in the helicase domain to Rep3, Rep4, and Rep13 (see FIG. 8C). The other two protein domains-OBD and ZF—of clone 1.03 cluster with other Rep variants e.g. Rep7, but without a clear trend (see FIGS. 8B and 8D). This contrasts with clone 2.41, which contains more sequences from Rep9, 10 and 4 (see FIG. 4G).

Without being bound by this theory it is assumed that clone 1.03 did not show clonal selection despite its superior packaging efficiency due to the kinetics of vector amplification or the superiority in particle production with clone 2.41, which results in more viral capsids available for packaging viral genomes—a process known to occur post-assembly [71].

Clone 2.41 overall has no benefit over Rep2.

Cryo-EM analysis of the indicated rAAV productions in AMBR250 purified using small-scale AAVX-columns are shown in FIG. 5F. Arrows indicate examples of full AAV capsids.

In summary, aspects of the current invention are new Rep variants having superior packaging abilities for both AAV2 and AAV8 compared to Rep2.

The Rep2 clones 0.15, 1.01. 1.03, and 1.10 are all individual aspects of the current invention.

### Use of the Chimeric Rep/Rep According to the Current Invention

Recombinant AAV particle production involves the step of culturing cells, introducing into those cells the required genes that are desired to be packaged in and that are required for packaging of rAAVp and the harvest of the rAAVp. Thus, the cells are modified to package (or produce) rAAVp. Cells that package or produce rAAVp are denoted as “producer cells”. Genes that are introduced to generate a producer cell normally at least comprise rep, cap, helper genes (E1A, E1B, E4orf6) and a transgene comprising inverted terminal repeats (ITRs) which flank one or more genes of interest.

The rep gene encodes for four different Rep protein splice variants required for the AAV life cycle, i.e. Rep78, Rep68, Rep52 and Rep40.

The rep and Rep according to the current invention are chimeric genes and polypeptides, respectively, i.e. comprising base pairs of rep genes and amino acids of Rep proteins of different serotypes.

In certain embodiments of all aspects and embodiments, the chimeric Rep proteins according to the current invention as well as the chimeric rep genes according to the current invention are used in the production of rAAVp encapsidating one or more genes of interest flanked by ITR sequences.

Accordingly, one aspect according to the current invention is a composition comprising a nucleic acid comprising a rep gene, wherein the rep gene is a chimeric rep gene according to the current invention. In certain embodiments, the rep gene comprises a 5′-terminus and a 3′-terminus. In certain embodiments, the 5′-terminus comprises an N-terminal domain (n), a DNA binding domain (d), and a helicase domain (h). In certain embodiments, the 3′-terminus comprises a NLS/p40 promoter domain (y) and a Zinc finger domain (z).

In certain embodiments of all aspects and embodiments, the sequence of the 5′-terminus is similar to the sequence of an AAV3 serotype and the sequence of the 3′-terminus is similar to the sequence of an AAV10 serotype.

In certain embodiments of all aspects and embodiments, the sequence of the 5′-terminus is similar to the sequence of an AAV3 serotype and the sequence of the 3′-terminus is similar to the sequence of an AAV13 serotype.

In certain embodiments of all aspects and embodiments, the sequence of the 5′-terminus is similar to the sequence of an AAV1 serotype and the sequence of the 3′-terminus is similar to the sequence of an AAV13 serotype.

In certain embodiments of all aspects and embodiments, the sequence of the 5′-terminus is similar to the sequence of an AAV1 serotype and the sequence of the 3′-terminus is similar to the sequence of an AAV2 serotype.

In certain embodiments of all aspects and embodiments, the sequence of the 5′-terminus is similar to the sequence of an AAV3 serotype and the sequence of the 3′-terminus is similar to the sequence of an AAV4 serotype.

In certain embodiments of all aspects and embodiments, the sequence of the 5′-terminus is similar to the sequence of an AAV6 serotype and the sequence of the 3′-terminus is similar to the sequence of an AAV4 serotype.

In certain embodiments of all aspects and embodiments, the sequence of the 5′-terminus is similar to the sequence of any AAV serotype and the sequence of the 3′-terminus is similar to the sequence of an AAV2 serotype.

In certain embodiments of all aspects and embodiments, the sequence of the 5′-terminus is similar to the sequence of an AAV3 serotype and the sequence of the 3′-terminus is similar to the sequence of an AAV6 serotype.

In certain embodiments of all aspects and embodiments, the sequence of the 5′-terminus is similar to the sequence of an AAV6 serotype and the sequence of the 3′-terminus is similar to the sequence of an AAV1 serotype.

In one preferred embodiment of all aspects and embodiments, the sequence of the 5′-terminus is similar to the sequence of any AAV serotype and the sequence of the 3′-terminus is similar to the sequence of an AAV13 serotype. In certain embodiments, the sequence of the 5′-terminus is similar to the sequence of an AAV2 or AAV6 serotype.

In certain embodiments of all aspects and embodiments, the 5′-terminus comprises in 5′- to 3′-direction a first 5′-terminal sequence and a second 5′-terminal sequence and the 3′-terminus comprises in 5′- to 3′-direction a second 3′-terminal sequence and a first 3′-terminal sequence.

In certain embodiments of all aspects and embodiments, the first 5′-terminal sequence is similar to the sequence of an AAV3 serotype and a second 5′-terminal sequence is similar to the sequence of an AAV6 serotype and the second 3′-terminal sequence is similar to the sequence of an AAV11 serotype and a first 3′-terminal sequence is similar to the sequence of an AAV10 serotype.

In certain embodiments of all aspects and embodiments, the first 5′-terminal sequence is similar to the sequence of an AAV3 serotype and a second 5′-terminal sequence is similar to the sequence of an AAV4 serotype and the second 3′-terminal sequence is similar to the sequence of an AAV4 serotype and a first 3′-terminal sequence is similar to the sequence of an AAV13 serotype.

In certain embodiments of all aspects and embodiments, the first 5′-terminal sequence is similar to the sequence of an AAV3 serotype and a second 5′-terminal sequence is similar to the sequence of an AAV10 serotype and the second 3′-terminal sequence is similar to the sequence of an AAV11 serotype and a first 3′-terminal sequence is similar to the sequence of an AAV4 serotype.

In certain embodiments of all aspects and embodiments, the first 5′-terminal sequence is similar to the sequence of an AAV6 serotype and a second 5′-terminal sequence is similar to the sequence of an AAV10 serotype and the second 3′-terminal sequence is similar to the sequence of an AAV11 serotype and a first 3′-terminal sequence is similar to the sequence of an AAV4 serotype.

In certain embodiments of all aspects and embodiments, the first 5′-terminal sequence is similar to the sequence of any AAV serotype and a second 5′-terminal sequence is similar to the sequence of an AAV1 serotype and the second 3′-terminal sequence is similar to the sequence of an AAV10 serotype and a first 3′-terminal sequence is similar to the sequence of an AAV2 serotype.

In certain embodiments of all aspects and embodiments, the first 5′-terminal sequence is similar to the sequence of an AAV3 serotype and a second 5′-terminal sequence is similar to the sequence of an AAV1 serotype and the second 3′-terminal sequence is similar to the sequence of a part of the rep gene of any AAV serotype and a first 3′-terminal sequence is similar to the sequence of an AAV13 serotype.

In certain embodiments of all aspects and embodiments, the first 5′-terminal sequence is similar to the sequence of an AAV3 serotype and a second 5′-terminal sequence is similar to the sequence of an AAV1 serotype and the second 3′-terminal sequence is similar to the sequence of a part of a rep gene of any AAV serotype and a first 3′-terminal sequence is similar to the sequence of an AAV6 serotype.

In one preferred embodiment of all aspects and embodiments, the first 5′-terminal sequence is similar to the sequence of any AAV serotype and a second 5′-terminal sequence is similar to the sequence of an AAV1 serotype and the second 3′-terminal sequence is similar to the sequence of any AAV serotype and a first 3′-terminal sequence is similar to the sequence of an AAV13 serotype. In certain embodiments the first 5′-terminal sequence is similar to an AAV2 or AAV6 serotype and the second 3′-terminal sequence is similar to an AAV10 or AAV11 serotype.

Accordingly, one aspect according to the current invention is the rep gene clone 0.15.

In certain embodiments of all aspects and embodiments, the chimeric rep gene clone 0.15 comprises in 5′ to 3′-direction fragments of the following wild-type rep genes

AAV3-AAVx-AAV6-AAVx-AAV11-AAV8-AAV11-AAV13-AAVX-
AAV6-AAV12-AAV6-AAV11-AAV10

with AAVx denoting any wild-type rep gene.

In certain embodiments of all aspects and embodiments, the chimeric rep gene clone 0.15 comprises the nucleic acid sequence of

(SEQ ID NO: 37)
ATGCCGGGGTTCTACGAGATTGTCCTGAAGGTCCCGAGTGACCTGGACGAGCACCTGCCGG
GCATTTCTAACTCGTTTGTTAACTGGGTGGCCGAGAAGGAATGGGAGCTGCCCCCGGATTC
TGACATGGATCGGAATCTGATCGAGCAGGCACCCCTGACCGTGGCCGAGAAGCTGCAGCGC
GACTTCCTGGTCCAGTGGCGCCGCGTGAGTAAGGCCCCGGAGGCCCTCTTCTTTGTTCAGT
TCGAGAAGGGCGAGAGCTACTTTCACCTGCACGTTCTGGTCGAGACCACGGGGGTCAAGTC
CATGGTCCTGGGCCGCTTCCTGAGTCAGATCAGAGACAGGCTGGTGCAGACCATCTACCGC
GGGGTCGAGCCCACGCTGCCCAACTGGTTCGCGGTGACCAAAGACGCGGTAATGGCGCCGG
CGGGGGGGAACAAGGTGGTGGACGAGTGCTACATCCCCAACTACCTCCTGCCCAAGACCCA
GCCCGAGCTGCAGTGGGCGTGGACTAACATGGAGGAGTATATAAGCGCGTGTCTAAACCTC
GCGGAGCGTAAACGGCTCGTGGCGCAGCACCTGACCCACGTCAGCCAGACGCAGGAGCAGA
ACAAGGAGAATCTGAACCCGAATTCTGACGCGCCCGTGATCAGGTCAAAAACCTCCGCGCG
CTACATGGAGCTGGTCGGGTGGCTGGTGGACCGGGGCATCACCTCCGAGAAGCAGTGGATC
CAGGAGGACCAGGCCTCGTACATCTCCTTCAACGCCGCCTCCAACTCGCGGTCACAAATCA
AGGCCGCACTGGACAATGCCGGCAAGATCATGGCGCTGACCAAATCCGCGCCCGACTACCT
GGTAGGCCCGTCCTTACCCGCGGACATTAAGGCCAACCGCATCTACCGCATCCTGGAGCTC
AACGGCTACGACCCCGCCTACGCGGCCTCCGTCTTCCTGGGCTGGGCGCAAAAGAAGTTCG
GGAAGAGGAACACCATCTGGCTCTTTGGGCCGGCCACGACGGGTAAAACCAACATCGCGGA
AGCCATCGCCCACGCCGTGCCCTTCTACGGCTGCGTCAACTGGACCAATGAGAACTTTCCG
TTCAACGACTGTGTCGACAAGATGGTGATCTGGTGGGAGGAGGGCAAGATGACGGCCAAGG
TCGTGGAGTCCGCCAAGGCCATTCTCGGCGGCAGCAAGGTGCGCGTGGACCAAAAGTGCAA
GTCGTCCGCCCAGATCGATCCCACCCCCGTGATCGTCACCTCCAACACCAACATGTGCGCC
GTGATTGACGGGAACAGCACCACCTTCGAGCACCAGCAGCCCCTGCAGGACCGGATGTTCA
AGTTTGAACTCACCCGCCGCCTCGACCACGACTTTGGCAAGGTCACCAAGCAGGAAGTCAA
GGACTTTTTCCGGTGGGCGCAGGATCACGTGACCGAGGTGGCGCATGAGTTCTACGTCAGA
AAGGGTGGAGCCAACAAGAGACCCGCCCCCAGTGACGCGGATATAAGCGAGCCCAAGCGGG
CCTGCCCCTCAGTTCCGGAGCCATCGACGTCAGACGCGGAAGCACCGGTGGACTTTGCGGA
CAGGTACCAAAACAAATGTTCTCGTCACGCGGGCATGCTTCAGATGCTGTTTCCCTGCAAG
ACATGCGAGAGAATGAATCAGAATTTCAACGTCTGCTTCACGCACGGGGTCAGAGACTGCT
CAGAGTGCTTCCCCGGCGCGTCAGAATCTCAACCTGTCGTCAGAAAAAAGACGTATCAGAA
ACTGTGCGCGATTCATCATCTGCTGGGGGGGGCACCCGAGATTGCGTGTTCGGCCTGCGAT
CTCGTCAACGTGGACTTGGATGACTGKGTTTCTGAACAATAA.

Accordingly, one aspect according to the current invention is the rep gene clone 1.01.

In certain embodiments of all aspects and embodiments, the chimeric rep gene clone 1.01 comprises in 5′ to 3′-direction fragments of the following wild-type rep genes

AAV7-AAV3-AAV4-AAV11-AAVx-AAV9-AAV12-AAV2-AAV1-
AAV10-AAV4-AAVx-AAV4-AAV12-AAV1-AAV7-AAV10-AAV4-
AAV9-AAV12-AAV3-AAV4-AAVx-AAV4-AAV13-AAV7

with AAVx denoting any wild-type rep gene.

In certain embodiments of all aspects and embodiments, the chimeric rep gene clone 1.01 comprises the nucleic acid sequence of

(SEQ ID NO: 38)
ATGCCGGGCTTCTACGAGATTGTCCTGAAGGTCCCGAGTGACCTGGACGAGCACCTGCCGG
GCATTTCTAACTCGTTTGTTAACTGGGTGGCCGAGAAGGAATGGGAGCTGCCGCCGGATTC
TGACATGGACTTGAATCTGATTGAGCAGGCACCCCTGACCGTGGCCGAAAAGCTGCAGCGC
GACTTCCTGGTCCACTGGCGCCGCGTGAGTAAGGCCCCGGAGGCCCTCTTCTTTGTTCAGT
TCGAGAAGGGCGAGTCCTACTTCCACCTCCATATTCTGGTGGAGACCACGGGGGTCAAATC
CATGGTGCTGGGCCGCTTCCTGAGTCAGATTAGGGACAAGCTGGTGCAGACCATCTACCGC
GGGATCGAGCCGACCCTGCCCAACTGGTTCGCGGTGACCAAGACGCGTAATGGCGCCGGCG
GGGGGAACAAGGTGGTGGACGAGTGCTACATCCCCAACTACCTGCTCCCCAAGACCCAGCC
CGAGCTGCAGTGGGCGTGGACTAACATGGAGGAGTATATAAGCGCCTGTTTGAACCTCGCG
GAGCGTAAACGGCTCGTGGCGCAGCATCTGACGCACGTGTCGCAGACGCAGGAGCAGAACA
AGGAGAATCTGAACCCGAATTCTGACGCGCCCGTGATCAGGTCAAAAACCTCCGCGCGCTA
CATGGAGCTGGTCGGGTGGCTGGTGGACCGCGGGATCACGTCAGAAAAGCAATGGATCCAG
GAGGACCAGGCGTCCTACATCTCCTTCAACGCCGCCTCCAACTCGCGGTCACAAATCAAGG
CCGCGCTGGACAATGCCTCCAAAATCATGAGCCTCACCAAAACGGCTCCGGACTATCTCAT
CGGGCAGCAGCCCGTGGGGGACATTACCACCAACCGGATCTACAAAATCCTGGAACTGAAC
GGGTACGACCCCCAGTACGCCGCCTCCGTCTTTCTCGGCTGGGCCCAGAAAAGGTTCGGGA
AGCGCAACACCATCTGGCTGTTTGGGCCGGCCACCACCGGCAAGACCAACATTGCGGAAGC
CATCGCCCACGCCGTGCCCTTCTACGGCTGCGTCAACTGGACCAATGAGAACTTTCCCTTC
AACGATTGCGTCGACAAGATGGTGATCTGGTGGGAGGAGGGCAAGATGACCGCCAAGGTCG
TAGAGAGCGCCAAGGCCATCCTGGGCGGAAGCAAGGTGCGCGTGGACCAAAAGTGCAAGTC
GTCCGCCCAGATCGACCCCACTCCCGTGATCGTCACCTCCAACACCAACATGTGCGCCGTG
ATTGACGGGAACAGCACCACCTTCGAGCACCAGCAGCCCCTGCAGGACCGGATGTTCAAAT
TTGAACTTACCCGCCGTTTGGACCATGACTTTGGCAAGGTCACCAAGCAGGAAGTCAAAGA
CTTTTTCCGGTGGGCGTCAGATCACGTGACCGAGGTGACTCACGAGTTTTACGTCAGAAAG
GGCGGAGCCAGCAAAAGACCCGCCCCCGATGACGCGGATAAAAGCGAGCCCAAGCGGGCCT
GTCCGTCAGTTGCGCAGCCATCGACGTCAGACGCGGAAGCTCCGGTGGACTACGCGGACAG
GTACCAAAACAAATGTTCTCGTCACGTGGGTATGAATCTGATGCTTTTTCCCTGCCGGCAA
TGCGAGAGAATGAATCAGAATGTGGACATTTGCTTCACGCACGGGGTCATGGACTGTGCCG
AGTGCTTCCCCGTGTCAGAATCTCAACCCGTGTCTGTCGTCAGAAAGCGGACATATCAGAA
ACTGTGTTTGATTCATCACATCATGGGGAGGGCGCCCGAGGTGGCTTGTTCGGCCTGCGAA
CTGGCCAATGTGGACTTGGATGACTGTGACATGGAACAATAA.

Accordingly, one preferred aspect according to the current invention is the rep gene clone 1.03.

In certain embodiments of all aspects and embodiments, the chimeric rep gene clone 1.03 comprises in 5′ to 3′-direction fragments of the following wild-type rep genes

AAVx-AAV1-AAV7-AAV9-AAV6-AAV9-AAV1-AAVx-AAV6-AAV3-
AAV6-AAV2-AAV11-AAVx-AAV11-AAVx-AAV13

with AAVx denoting any wild-type rep gene.

In certain embodiments of all aspects and embodiments, the chimeric rep gene clone 1.03 comprises the nucleic acid sequence of

(SEQ ID NO: 39)
ATGCCGGGGTTTTACGAGATTGTGATTAAGGTCCCCAGCGACCTTGACGAGCATCTGCCCG
GCATTTCTGACAGCTTTGTGAACTGGGTGGCCGAGAAGGAATGGGAGCTGCCCCCGGATTC
TGACATGGATCTGAATCTGATTGAGCAGGCACCCCTGACCGTGGCCGAGAAGCTGCAGCGC
GACTTCCTGGTCCAATGGCGCCGCGTGAGTAAGGCCCCGGAGGCCCTCTTCTTTGTTCAGT
TCGAGAAGGGCGAGAGCTACTTCCACCTTCACGTTCTGGTGGAGACCACGGGGGTCAAGTC
CATGGTGCTAGGCCGCTTCCTGAGTCAGATTCGGGAGAAGCTGGTCCAGACCATCTACCGC
GGGATCGAGCCGACCCTGCCCAACTGGTTCGCGGTGACCAAGACGCGTAATGGCGCCGGAG
GGGGGAACAAGGTGGTGGACGAGTGCTACATCCCCAACTACCTCCTGCCCAAGACTCAGCC
CGAGCTGCAGTGGGCGTGGACTAACATGGAGGAGTATATAAGCGCGTGCTTGAACCTGGCC
GAGCGCAAACGGCTCGTGGCGCAGCACCTGACCCACGTCAGCCAGACCCAGGAGCAGAACA
AGGAGAATCTGAACCCCAATTCTGACGCGCCCGTGATCAGGTCAAAAACCTCCGCACGCTA
CATGGAGCTGGTCGGGTGGCTGGTGGACCGGGGCATCACCTCCGAGAAGCAGTGGATCCAG
GAGGACCAGGCCTCGTACATCTCCTTCAACGCCGCCTCCAACTCGCGGTCCCAGATCAAGG
CCGCGCTGGACAATGCCTCCAAGATCATGAGCCTGACAAAGACGGCTCCGGACTACCTGGT
GGGCAGCAACCCGCCGGAGGACATTACCAAAAATCGGATCTACCAAATCCTGGAGCTGAAC
GGGTACGATCCGCAGTACGCGGCCTCCGTCTTCCTGGGCTGGGCGCAAAAGAAGTTCGGGA
AGAGGAACACCATCTGGCTCTTTGGGCCGGCCACGACGGGTAAAACCAACATCGCGGAAGC
CATCGCCCACGCCGTGCCCTTCTACGGCTGCGTCAACTGGACCAATGAGAACTTTCCCTTC
AACGATTGCGTCGACAAGATGGTGATCTGGTGGGAGGAGGGCAAGATGACGGCCAAGGTCG
TGGAGTCGGCCAAAGCCATTCTCGGAGGAAGCAAGGTGCGCGTGGACCAAAAGTGCAAGTC
CTCGGCCCAGATCGACCCCACGCCCGTGATCGTCACCTCCAACACCAACATGTGCGCCGTG
ATCGACGGGAACAGCACCACCTTCGAGCACCAGCAGCCGCTGCAGGACCGGATGTTCAAAT
TTGAACTCACCCGCCGTCTGGAGCATGACTTTGGCAAGGTGACAAAGCAGGAAGTCAAAGA
GTTCTTCCGCTGGGCGCAGGATCACGTGACCGAGGTGGCGCATGAGTTCTACGTCAGAAAG
GGCGGAGCCACCAAAAGACCCGCCCCCAGTGACGCGGATATAAGCGAGCCCAAGCGGGCCT
GCCCCTCAGTTCCGGAGCCATCGACGTCAGACGCGGAAGCGCCGGTGGACTTTGCGGACAG
GTACCAAAACAAATGTTCTCGTCACGCGGGCATGCTTCAGATGCTGTTTCCCTGCAAGACA
TGCGAGAGAATGAATCAGAATTTCAACGTCTGCTTCACGCACGGGGTCAGAGACTGCTCAG
AGTGCTTCCCCGGCGTGTCAGAATCTCAACCCGTGTCTGTCGTCAGAAAGCGGACATATCA
GAAACTGTGTCCGATTCATCACATCATGGGGAGGGCGCCCGAGATTGCTTGCTCGGCCTGC
GATCTGGTCAACGTGGACCTGGATGACTGTGTTTCTGAGCAATAA.

Accordingly, one aspect according to the current invention is the rep gene clone 1.10.

In certain embodiments of all aspects and embodiments, the chimeric rep gene clone 1.10 comprises in 5′ to 3′-direction fragments of the following wild-type rep genes

AAVx-AAV1-AAV3-AAVx-AAV10-AAVx-AAV2-AAV4-AAV13-
AAV11-AAVx-AAV12-AAVx-AAV12-AAV10-AAV7-AAV13-
AAVI-AAV10-AAV2

with AAVx denoting any wild-type rep gene.

In certain embodiments of all aspects and embodiments, the chimeric rep gene clone 1.10 comprises the nucleic acid sequence of

(SEQ ID NO: 41)
ATGCCGGGGTTTTACGAGATTGTGATTAAGGTCCCCAGCGACCTTGACGAGCATCTGCCCG
GCATTTCTGACAGCTTTGTGAACTGGGTGGCCGAGAAGGAATGGGAGCTGCCCCCGGATTC
TGACATGGATCTGAATCTGATTGAGCAGGCACCCCTGACCGTGGCCGAGAAGCTGCAGCGC
GAGTTCCTGGTGGAGTGGCGCCGCGTGAGTAAGGCCCCGGAGGCCCTCTTTTTTGTCCAGT
TCGAAAAGGGGGAGACCTACTTCCACCTGCACGTGCTGATTGAGACCATCGGGGTCAAATC
CATGGTGGTCGGCCGCTACGTGAGCCAGATTAAAGAGAAGCTGGTGACCCGCATCTACCGC
GGGGTCGAGCCGCAGCTTCCGAACTGGTTCGCGGTGACCAAGACGCGTAATGGCGCCGGAG
GCGGGAACAAGGTGGTGGACGACTGCTACATCCCCAACTACCTGCTCCCCAAGACCCAGCC
CGAGCTGCAGTGGGCGTGGACTAACATGGAGGAGTATATAAGCGCGTGTCTGAACCTCGCG
GAGCGTAAACGGCTCGTGGCGCAGCACCTGACCCACGTCAGCCAGACGCAGGAGCAGAACA
AGGAGAATCTGAACCCCAATTCTGACGCGCCCGTGATCAGGTCAAAAACCTCCGCGCGCTA
CATGGAGCTGGTCGGGTGGCTCGTGGACAAGGGGATTACCTCGGAGAAGCAGTGGATCCAG
GAGGACCAGGCCTCGTACATCTCCTTCAACGCCGCCTCCAACTCGCGGTCACAAATCAAGG
CCGCGCTGGACAATGCCTCCAAAATCATGAGCCTGACAAAGACGGCTCCGGACTACCTGGT
GGGCCAGAACCCGCCGGAGGACATTACCAGCAACCGGATCTACAAAATCCTCGAGATGAAC
GGGTACGATCCGCAGTACGCGGCCTCCGTCTTCCTGGGCTGGGCGCAAAAGAAGTTCGGTA
AACGCAACACCATCTGGCTGTTTGGGCCTGCAACTACCGGCAAGACCAACATCGCGGAAGC
CATCGCCCACGCGGTCCCCTTCTACGGCTGCGTCAACTGGACCAATGAGAACTTTCCCTTC
AATGATTGCGTCGACAAGATGGTGATCTGGTGGGAGGAGGGCAAGATGACGGCCAAGGTCG
TGGAGTCCGCCAAGGCCATTCTCGGCGGCAGCAAGGTGCGCGTGGACCAAAAATGCAAGGC
CTCTGCGCAGATCGACCCCACCCCCGTGATCGTCACCTCCAACACCAACATGTGCGCCGTG
ATCGACGGGAACAGCACCACCTTCGAGCACCAGCAGCCCCTGCAGGACCGCATGTTCAAAT
TTGAACTCACCCGCCGTCTGGAGCACGACTTTGGCAAGGTGACGAAGCAGGAAGTCAAAGA
GTTCTTCCGCTGGGCCAGTGATCACGTGACTGAGGTGTCTCACGAGTTTTACGTCAGAAAG
GGTGGAGCCAACAAAAGACCCGCCCCCGATGACGCGGATAAAAGCGAGCCCAAGCGGGCCT
GCCCCTCAGTTGCGGAGCCATCGACGTCAGACGCGGAAGCACCGGTGGACTTTGCGGACAG
GTACCAAAACAAATGTTCTCGTCACGCGGGCATGCTTCAGATGCTGTTTCCCTGCAGACAA
TGCGAGAGAATGAATCAGAATTCAAATATCTGCTTCACTCACGGACAGAAAGACTGTTTAG
AGTGCTTTCCCGTGTCAGAATCTCAACCCGTTTCTGTCGTCAAAAAGGCGTATCAGAAACT
GTGCTACATTCATCATATCATGGGAAAGGTGCCAGACGCTTGCACTGCCTGCGATCTGGTC
AATGTGGATTTGGATGACTGCATCTTTGAACAATAA.

The term similar denotes that two sequences differ from each other at at most 10% of the residues, i.e. nucleic acid residues or amino acid residues.

In certain embodiments of all aspects and embodiments, the rep gene according to the current invention has a start codon of sequence ATG.

In certain embodiments of all aspects and embodiments, any one of the compositions according to the current invention further comprises a nucleic acid comprising a cap gene. The cap gene may be of any serotype (e.g., 1, 2, 3, 4, 5, 6, 7, 8, 9, 10, 11, 12, or 13).

In certain embodiments of all aspects and embodiments, the nucleic acid comprising the rep gene according to the current invention and the nucleic acid comprising the cap gene are contained in a plasmid.

Accordingly, one aspect according to the current invention is a method of packaging a recombinant adeno-associated virus particle comprising contacting a cell that expresses a rep gene according to the current invention with a recombinant nucleic acid that comprises a gene of interest to be packaged interspaced between a pair of inverted terminal repeats (ITRs) of one serotype. In certain embodiments, the rep gene according to the current invention is expressed by transfecting or infecting the cell with a nucleic acid comprising the rep gene.

In certain embodiments of all aspects and embodiments, the one serotype of the ITRs is serotype 2.

In certain embodiments of all aspects and embodiments, the cell is also contacted with a recombinant nucleic acid that comprises a cap gene.

In certain embodiments of all aspects according to the current invention, a cell that comprises and expresses a rep gene according to the current invention is further contacted with a recombinant nucleic acid that comprises a gene of interest to be packaged into a rAAVp interspaced between a pair of inverted terminal repeats (ITRs) of a second serotype also expresses a cap gene.

The difference (positions with at least one difference marked with an * above the sequence) between the preferred rep genes according to the current invention is shown in the following alignment.

| | | |
|---|---|---|
| | * * * | 60 |
| Clone 0-15 | MPGFYEIVLK VPSDLDEHLP GISNSFVNWV AEKEWELPPD SDMDRNLIEQ APLTVAEKLQ | 60 |
| Clone 1-01 | MPGFYEIVLK VPSDLDEHLP GISNSFVNWV AEKEWELPPD SDMDLNLIEQ APLTVAEKLQ | 60 |
| Clone 1-03 | MPGFYEIVIK VPSDLDEHLP GISDSFVNWV AEKEWELPPD SDMDLNLIEQ APLTVAEKLQ | 60 |
| Clone 1-10 | MPGFYEIVIK VPSDLDEHLP GISDSFVNWV AEKEWELPPD SDMDLNLIEQ APLTVAEKLQ | 60 |
| | * * * * * * * ** *** ** | 120 |
| Clone 0-15 | RDFLVQWRRV SKAPEALFFV QFEKGESYFH LHVLVETTGV KSMVLGRFLS QIRDRLVQTI | 120 |
| Clone 1-01 | RDFLVHWRRV SKAPEALFFV QFEKGESYFH LHILVETTGV KSMVLGRFLS QIRDKLVQTI | 120 |
| Clone 1-03 | RDFLVQWRRV SKAPEALFFV QFEKGESYFH LHVLVETTGV KSMVLGRFLS QIREKLVQTI | 120 |
| Clone 1-10 | REFLVEWRRV SKAPEALFFV QFEKGETYFH LHVLIETIGV KSMVVGRYVS QIKEKLVTRI | 120 |
| | * * **** *** * | 180 |
| Clone 0-15 | YRGVEPTLPN WFAVTKDAVM APAGGNKVVD ECYIPNYLLP KTQPELQWAW TNMEEYISAC | 180 |
| Clone 1-01 | YRGIEPTLPN WFAVTKTR.N GAGGGNKVVD ECYIPNYLLP KTQPELQWAW TNMEEYISAC | 179 |
| Clone 1-03 | YRGIEPTLPN WFAVTKTR.N GAGGGNKVVD ECYIPNYLLP KTQPELQWAW TNMEEYISAC | 179 |
| Clone 1-10 | YRGVEPQLPN WFAVTKTR.N GAGGGNKVVD DCYIPNYLLP KTQPELQWAW TNMEEYISAC | 179 |
| | * | 240 |
| Clone 0-15 | LNLAERKRLV AQHLTHVSQT QEQNKENLNP NSDAPVIRSK TSARYMELVG WLVDRGITSE | 240 |
| Clone 1-01 | LNLAERKRLV AQHLTHVSQT QEQNKENLNP NSDAPVIRSK TSARYMELVG WLVDRGITSE | 239 |
| Clone 1-03 | LNLAERKRLV AQHLTHVSQT QEQNKENLNP NSDAPVIRSK TSARYMELVG WLVDRGITSE | 239 |
| Clone 1-10 | LNLAERKRLV AQHLTHVSQT QEQNKENLNP NSDAPVIRSK TSARYMELVG WLVDKGITSE | 239 |
| | * * * * *** ** ** | 300 |
| Clone 0-15 | KQWIQEDQAS YISFNAASNS RSQIKAALDN AGKIMALTKS APDYLVGPSL PADIKANRIY | 300 |
| Clone 1-01 | KQWIQEDQAS YISFNAASNS RSQIKAALDN ASKIMSLIKT APDYLIGQQP VGDITTNRIY | 299 |
| Clone 1-03 | KQWIQEDQAS YISFNAASNS RSQIKAALDN ASKIMSLIKT APDYLVGSNP PEDITKNRIY | 299 |
| Clone 1-10 | KQWIQEDQAS YISFNAASNS RSQIKAALDN ASKIMSLIKT APDYLVGQNP PEDITSNRIY | 299 |
| | * * * * | 360 |
| Clone 0-15 | RILELNGYDP AYAASVFLGW AQKKFGKRNT IWLFGPATTG KTNIAEAIAH AVPFYGCVNW | 360 |
| Clone 1-01 | KILELNGYDP QYAASVFLGW AQKRFGKRNT IWLFGPATTG KTNIAEAIAH AVPFYGCVNW | 359 |
| Clone 1-03 | QILELNGYDP QYAASVFLGW AQKKFGKRNT IWLFGPATTG KTNIAEAIAH AVPFYGCVNW | 359 |
| Clone 1-10 | KILEMNGYDP QYAASVFLGW AQKKFGKRNT IWLFGPATTG KTNIAEAIAH AVPFYGCVNW | 359 |
| | * | 420 |
| Clone 0-15 | TNENFPFNDC VDKMVIWWEE GKMTAKVVES AKAILGGSKV RVDQKCKSSA QIDPTPVIVT | 420 |
| Clone 1-01 | TNENFPFNDC VDKMVIWWEE GKMTAKVVES AKAILGGSKV RVDQKCKSSA QIDPTPVIVT | 419 |
| Clone 1-03 | TNENFPFNDC VDKMVIWWEE GKMTAKVVES AKAILGGSKV RVDQKCKSSA QIDPTPVIVT | 419 |
| Clone 1-10 | TNENFPFNDC VDKMVIWWEE GKMTAKVVES AKAILGGSKV RVDQKCKASA QIDPTPVIVT | 419 |
| | * * * | 480 |
| Clone 0-15 | SNTNMCAVID GNSTTFEHQQ PLQDRMFKFE LTRRLDHDFG KVTKQEVKDF FRWAQDHVTE | 480 |
| Clone 1-01 | SNTNMCAVID GNSTTFEHQQ PLQDRMEKFE LTRRLDHDFG KVTKQEVKDF FRWASDHVTE | 479 |
| Clone 1-03 | SNTNMCAVID GNSTTFEHQQ PLQDRMFKFE LTRRLEHDFG KVTKQEVKEF FRWAQDHVTE | 479 |
| Clone 1-10 | SNTNMCAVID GNSTTFEHQQ PLQDRMFKFE LTRRLEHDFG KVTKQEVKEF FRWASDHVTE | 479 |
| | * * * * ** * * | 540 |
| Clone 0-15 | VAHEFYVRKG GANKRPAPSD ADISEPKRAC PSVPEPSTSD AEAPVDFADR YQNKCSRHAG | 540 |
| Clone 1-01 | VTHEFYVRKG GASKRPAPDD ADKSEPKRAC PSVAQPSTSD AEAPVDYADR YQNKCSRHVG | 539 |
| Clone 1-03 | VAHEFYVRKG GATKRPAPSD ADISEPKRAC PSVPEPSTSD AEAPVDFADR YQNKCSRHAG | 539 |
| Clone 1-10 | VSHEFYVRKG GANKRPAPDD ADKSEPKRAC PSVAEPSTSD AEAPVDFADR YQNKCSRHAG | 539 |
| | ** ** *** ** * ** ** * ** * ** | 600 |
| Clone 0-15 | MLQMLFPCKT CERMNQNFNV CFTHGVRDCS ECFPGASESQ P..VVRKKTY QKLCAIHHLL | 598 |
| Clone 1-01 | MNLMLFPCRQ CERMNQNVDI CFTHGVMDCA ECFP.VSESQ PVSVVRKRTY QKLCLIHHIM | 598 |
| Clone 1-03 | MLQMLFPCKT CERMNQNFNV CFTHGVRDCS ECFPGVSESQ PVSVVRKRTY QKLCPIHHIM | 599 |
| Clone 1-10 | MLQMLFPCRQ CERMNQNSNI CFTHGQKDCL ECFP.VSESQ PVSVV.KKAY QKLCYIHHIM | 597 |
| | ** *** * * * *** | 625 |
| Clone 0-15 | GRAPEIACSA CDLVNVDLDD XVSEQ | 622 |
| Clone 1-01 | GRAPEVACSA CELANVDLDD CDMEQ | 622 |
| Clone 1-03 | GRAPEIACSA CDLVNVDLDD CVSEQ | 624 |
| Clone 1-10 | GKVPDA.CTA CDLVNVDLDD CIFEQ | 621 |

All publications, patents, and patent applications cited herein are hereby incorporated by reference herein in their entirety for all purposes to the same extent as if each individual publication, patent, and patent application were specifically and individually indicated to be so incorporated by reference. In the event that one or more of the incorporated literature and similar materials differs from or contradicts this application, including but not limited to defined terms, term usage, described techniques, or the like, this application controls.

The following examples and figures as well as the sequences are provided to aid the understanding of the present invention, the true scope of which is set forth in the appended claims. It is understood that modifications can be made in the procedures set forth without departing from the spirit of the invention.

That is, although the disclosed teachings have been described with reference to various applications, methods, and compositions, it will be appreciated that various changes and modifications can be made without departing from the teachings herein and the claimed invention below. The examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### REFERENCES

1. Lyle, A., et al., Biotechnol. Bioeng. (2023) 1-14.
2. Williams, C., https://desciappliedresearch.com/2024/01/at-cost-aav/(2024).
3. McIntosh, N. L., et al., Sci. Rep. 11, 3012 (2021).
4. https://sciex.com/content/dam/SCIEX/pdf/tech-notes/all/AAV-Full-Partial-Empty.pdf. Srivastava, A., et al., J. Virol. 45, 555-564 (1983).
6. Sonntag, F., et al., J. Virol. 85, 12686-12697 (2011).
7. Ogden, P. J., et al., Science 366, 1139-1143 (2019).
8. Stutika, C., et al., J. Virol. 90, 1278-1289 (2016).
9. Tratschin, J. D., et al., J. Virol. 51, 611-619 (1984).
10. Pereira, D. J., et al., J. Virol. 71, 1079-1088 (1997).
11. Dyall, J., et al., Proc. Natl. Acad. Sci. 96, 12849-12854 (1999).
12. Im, D.-S. and Muzyczka, N., Cell 61, 447-457 (1990).
13. King, J. A., et al., EMBO J. 20, 3282-3291 (2001).
14. Timpe, J., et al., Curr. Gene Ther. 5, 273-284 (2005).
15. McCarty, D. M., et al., J. Virol. 65, 2936-2945 (1991).
16. Im, D. S. and Muzyczka, N., J. Virol. 63, 3095-3104 (1989).
17. Smith, R. H. and Kotin, R. M., J. Virol. 74, 3122-3129 (2000).
18. Saudan, P., et al., EMBO J. 19, 4351-4361 (2000).
19. Pasquale, G. D. and Chiorini, J. A., EMBO J. 22, 1716-1724 (2003).
20. Tejero, M., et al., Genes 14, 1866 (2023).
21. Maurer, A. C. and Weitzman, M. D., Hum. Gene Ther. 31, 499-511 (2020).
22 Vincent, K. A., et al., J. Virol. 71, 1897-1905 (1997).
23. Li, J., et al., J. Virol. 71, 5236-5243 (1997).
24. Mizukami, H., et al., Mol. Biotechnol. 27, 7-14 (2004).
25. Rabinowitz, J. E., et al., J. Virol. 76, 791-801 (2002).
26. Grimm, D., et al., Mol. Ther. 7, 839-850 (2003).
27. Beck, S. E., et al., J. Virol. 73, 9446-9455 (1999).
28. Halbert, C. L., et al., J. Virol. 75, 6615-6624 (2001).
29. Chiorini, J. A., et al., J. Virol. 71, 6823-6833 (1997).
30. Mietzsch, M. et al., J. Virol. 95, e00773-21 (2021).
31. Ling, C., et al., Mol. Ther. Meth. Clin. Dev. 3, 16029 (2016).
32. Jain, N. K., et al., eLife 12: RP87730 (2024).
33. Zeltner, N., et al., Gene Ther. 17, 872-879 (2010). Li, X., et al., Front. Bioeng. Biotechnol. 11, 1327433 (2023). 34
35. Nass, S. A., et al., Mol. Ther. Meth. Clin. Dev. 9, 33-46 (2018).
36. Herrmann, A.-K., et al., ACS Synth. Biol. 8, 194-206 (2019).
37 Koerber, J. T., et al., Jang, J.-H. & Schaffer, D. V. DNA Shuffling of Adeno-associated Virus Yields Functionally Diverse Viral Progeny. Mol. Ther. 16, 1703-1709 (2008).
38. Grimm, D. et al., J. Virol. 82, 5887-5911 (2008).
39. Kikuchi, M., et al., Gene 236, 159-167 (1999).
40. Landegger, L. D., et al., Nat. Biotechnol. 35, 280-284 (2017).
41. Fakhiri, J., et al., Methods Mol. Biol. 1961, 111-126 (2019).
42. WO 2017/096039.
43. Dobnik, D., et al., Front. Microbiol. 10, 1570 (2019).
44. Tang, G., et al., J. Struct. Biol. 157, 38-46 (2007).
45. Shukla, V., et al., Hum. Gene Ther. Clin. Dev. 30, 102-113 (2019).
46. Schnödt, M., et al., Mol. Ther. Nucl. Acids 5, e355 (2016).
47. Williams, J. A. and Paez, P. A., Mol. Ther. Nucl. Acids 32, 494-503 (2023).
48. Barreira, M., et al., Gene Ther. 30, 122-131 (2023).
49. Karbowniczek, K., et al., Cell Gene Ther. Ins. 3, 731-738 (2017).
50. Guan, J.-S., et al., Front. Chem. Eng. 4, (2022).
51. Grieger, J. C., et al., Mol. Ther. 24, 287-297 (2016).
52. Lee, Z., et al., ACS Synth. Biol. 11, 3285-3295 (2022).
53. Cytiva. ELEVECTA™ producer cell line. https://www.cytivalifesciences.com/en/us/shop/cell-culture-and-fermentation/cell-lines/elevecta-producer-cell-line-p-42420 (2024).
54. Su, W., et al., Nat. Commun. 13, 1182 (2022).
55. Hakim, C. H., et al., Mol. Ther.—Methods Clin. Dev. 18, 664-678 (2020).
56. Kurasawa, J. H., et al., Mol. Ther. Meth. Clin. Dev. 19, 330-340 (2020).
57. Mietzsch, M., et al., Hum. Gene Ther. 25, 212-222 (2014).
58. Becker, J., et al., Pathogens 11, 756 (2022).
59. Andari, J. E., et al., Sci. Adv. 8, eabn4704 (2022).
60. Grosse, S., et al., J. Virol. 91, (2017).
61. Galibert, L., et al., Sci. Rep. 11, 21698 (2021).
62. Herrmann, A.-K., et al., Hum. Gene Ther. 30, 21-35 (2019).
63. Schieferecke, A. J., et al., Mol. Ther. 32, 340-351 (2024).
64. Chiorini, J. A., et al., J. Virol. 73, 1309-1319 (1999).
65. Grimm, D., et al., Hum. Gene Ther. 9, 2745-2760 (1998).
66. Crameri, A., et al., Nat. Biotechnol. 14, 315-319 (1996).
67. O'Maille, P. E., et al., J. Mol. Biol. 321, 677-691 (2002).
68. Perabo, L., et al., Mol. Ther. 8, 151-157 (2003).
69. Warr, S. R. C., Mol. Biol. 2095, 43-67 (2019).
70. Manahan, M., et al., Biotechnol. Prog. 35, e2870 (2019).
71. Bennett, A., et al., Futur. Virol. 12, 283-297 (2017).
72. Chen, Y., et al., Microorganisms 12, 310 (2024).
73. Earley, L. F., et al., Hum. Gene Ther. 31, 151-162 (2020).
74. Pan, X., et al., Gene Ther. 29, 333-345 (2022).

Sequences

| SEQ ID NO- | |
|---|---|
| description | SEQUENCE |
| 1-exemplary | TTGCCCACTCCCTCTCTGCGCGCTCGCTCGCTCGGTGGGGCCTG |
| sequence of wild-type | CGGACCAAAGGTCCGCAGACGGCAGAGCTCTGCTCTGCCGGCCC |
| AAV1 ITR | CACCGAGCGAGCGAGCGCGCAGAGAGGGAGTGGGCAACTCCATC |
| | ACTAGGGGTAATCGC |
| 2-exemplary | AGGAACCCCTAGTGATGGAGTTGGCCACTCCCTCTCTGCGCGCT |
| sequence of wild-type | CGCTCGCTCACTGAGGCCGGGCGACCAAAGGTCGCCCGACGCCC |
| AAV2 ITR | GGGCTTTGCCCGGGCGGCCTCAGTGAGCGAGCGAGCGCGCAGAG |
| | AGGGAGTGGCCAA |
| 3-exemplary | TGGCCACTCCCTCTATGCGCACTCGCTCGCTCGGTGGGGCCTGG |
| sequence of wild-type | CGACCAAAGGTCGCCAGACGGACGTGCTTTGCACGTCCGGCCCC |
| AAV3 ITR | ACCGAGCGAGCGAGTGCGCATAGAGGGAGTGGCCAACTCCATCA |
| | CTAGAGGTATGGCA |
| 4-exemplary | GGGCAAACCTAGATGATGGAGTTGGCCACTCCCTCTATGCGCGC |
| sequence of wild-type | TCGCTCACTCACTCGGCCCTGCCGGCCAGAGGCCGGCAGTCTGG |
| AAV4 ITR | AGACCTTTGGTCTCCAGGGCCGAGTGAGTGAGCGAGCGCGCATA |
| | GAGGGAGTGGCCAA |
| 5-exemplary | CTCTCCCCCCTGTCGCGTTCGCTCGCTCGCTGGCTCGTTTGGGG |
| sequence of wild-type | GGGTGGCAGCTCAAAGAGCTGCCAGACGACGGCCCTCTGGCCGT |
| AAV5 ITR | CGCCCCCCCAAACGAGCCAGCGAGCGAGCGAACGCGACAGGGGG |
| | GAGAGTGCCACACTCTCAAGCAAGGGGGTTTTGTAAGCAGTGAT |
| 6-exemplary | ATACCCCTAGTGATGGAGTTGCCCACTCCCTCTATGCGCGCTCG |
| sequence of wild-type | CTCGCTCGGTGGGGCCTGCGGACCAAAGGTCCGCAGACGGCAGA |
| AAV6 ITR | GCTCTGCTCTGCCGGCCCCACCGAGCGAGCGAGCGCGCATAGAG |
| | GGAGTGGGCAA |
| 7-exemplary | TTGGCCACTCCCTCTATGCGCGCTCGCTCGCTCGGTGGGGCCTG |
| sequence of wild-type | CGGACCAAAGGTCCGCAGACGGCAGAGCTCTGCTCTGCCGGCCC |
| AAV7 ITR | CACCGAGCGAGCGAGCGCGCATAGAGGGAGTGGCCAACTCCATC |
| | ACTAGGGGTACCGC |
| 11-exemplary wild- | ATGCCGGGCTTCTACGAGATCGTGATCAAGGTGCCGAGCGACCT |
| type AAV1 rep | GGACGAGCACCTGCCGGGCATTTCTGACTCGTTTGTGAGCTGGG |
| (derived from NCBI | TGGCCGAGAAGGAATGGGAGCTGCCCCCGGATTCTGACATGGAT |
| GenBank entry | CTGAATCTGATTGAGCAGGCACCCCTGACCGTGGCCGAGAAGCT |
| NC002077) | GCAGCGCGACTTCCTGGTCCAATGGCGCCGCGTGAGTAAGGCCC |
| | CGGAGGCCCTCTTCTTTGTTCAGTTCGAGAAGGGCGAGTCCTAC |
| | TTCCACCTCCATATTCTGGTGGAGACCACGGGGGTCAAATCCAT |
| | GGTGCTGGGCCGCTTCCTGAGTCAGATTAGGGACAAGCTGGTGC |
| | AGACCATCTACCGCGGGATCGAGCCGACCCTGCCCAACTGGTTC |
| | GCGGTGACCAAGACGCGTAATGGCGCCGGAGGGGGGAACAAGGT |
| | GGTGGACGAGTGCTACATCCCCAACTACCTCCTGCCCAAGACTC |
| | AGCCCGAGCTGCAGTGGGCGTGGACTAACATGGAGGAGTATATA |
| | AGCGCCTGTTTGAACCTGGCCGAGCGCAAACGGCTCGTGGCGCA |
| | GCACCTGACCCACGTCAGCCAGACCCAGGAGCAGAACAAGGAGA |
| | ATCTGAACCCCAATTCTGACGCGCCTGTCATCCGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCTTCCAACTCGCGGTCCCAGATCAAG |
| | GCCGCTCTGGACAATGCCGGCAAGATCATGGCGCTGACCAAATC |
| | CGCGCCCGACTACCTGGTAGGCCCCGCTCCGCCCGCGGACATTA |
| | AAACCAACCGCATCTACCGCATCCTGGAGCTGAACGGCTACGAA |
| | CCTGCCTACGCCGGCTCCGTCTTTCTCGGCTGGGCCCAGAAAAG |
| | GTTCGGGAAGCGCAACACCATCTGGCTGTTTGGGCCGGCCACCA |
| | CGGGCAAGACCAACATCGCGGAAGCCATCGCCCACGCCGTGCCC |
| | TTCTACGGCTGCGTCAACTGGACCAATGAGAACTTTCCCTTCAA |
| | TGATTGCGTCGACAAGATGGTGATCTGGTGGGAGGAGGGCAAGA |
| | TGACGGCCAAGGTCGTGGAGTCCGCCAAGGCCATTCTCGGCGGC |
| | AGCAAGGTGCGCGTGGACCAAAAGTGCAAGTCGTCCGCCCAGAT |
| | CGACCCCACCCCCGTGATCGTCACCTCCAACACCAACATGTGCG |
| | CCGTGATTGACGGGAACAGCACCACCTTCGAGCACCAGCAGCCG |
| | TTGCAGGACCGGATGTTCAAATTTGAACTCACCCGCCGTCTGGA |
| | GCATGACTTTGGCAAGGTGACAAAGCAGGAAGTCAAAGAGTTCT |
| | TCCGCTGGGCGCAGGATCACGTGACCGAGGTGGCGCATGAGTTC |
| | TACGTCAGAAAGGGTGGAGCCAACAAAAGACCCGCCCCCGATGA |
| | CGCGGATAAAAGCGAGCCCAAGCGGGCCTGCCCCTCAGTCGCGG |
| | ATCCATCGACGTCAGACGCGGAAGGAGCTCCGGTGGACTTTGCC |
| | GACAGGTACCAAAACAAATGTTCTCGTCACGCGGGCATGCTTCA |
| | GATGCTGTTTCCCTGCAAGACATGCGAGAGAATGAATCAGAATT |
| | TCAACATTTGCTTCACGCACGGGACGAGAGACTGTTCAGAGTGC |
| | TTCCCCGGCGTGTCAGAATCTCAACCGGTCGTCAGAAAGAGGAC |
| | GTATCGGAAACTCTGTGCCATTCATCATCTGCTGGGGGGGGCTC |
| | CCGAGATTGCTTGCTCGGCCTGCGATCTGGTCAACGTGGACCTG |
| | GATGACTGTGTTTCTGAGCAATAA |
| 12-exemplary wild- | ATGCCGGGGTTTTACGAGATTGTGATTAAGGTCCCCAGCGACCT |
| type AAV2 rep | TGACGAGCATCTGCCCGGCATTTCTGACAGCTTTGTGAACTGGG |
| (derived from NCBI | TGGCCGAGAAGGAATGGGAGTTGCCGCCAGATTCTGACATGGAT |
| GenBank entry | CTGAATCTGATTGAGCAGGCACCCCTGACCGTGGCCGAGAAGCT |
| NC001401) | GCAGCGCGACTTTCTGACGGAATGGCGCCGTGTGAGTAAGGCCC |
| | CGGAGGCCCTTTTCTTTGTGCAATTTGAGAAGGGAGAGAGCTAC |
| | TTCCACATGCACGTGCTCGTGGAAACCACCGGGGTGAAATCCAT |
| | GGTTTTGGGACGTTTCCTGAGTCAGATTCGCGAAAAACTGATTC |
| | AGAGAATTTACCGCGGGATCGAGCCGACTTTGCCAAACTGGTTC |
| | GCGGTCACAAAGACCAGAAATGGCGCCGGAGGCGGGAACAAGGT |
| | GGTGGATGAGTGCTACATCCCCAATTACTTGCTCCCCAAAACCC |
| | AGCCTGAGCTCCAGTGGGCGTGGACTAATATGGAACAGTATTTA |
| | AGCGCCTGTTTGAATCTCACGGAGCGTAAACGGTTGGTGGCGCA |
| | GCATCTGACGCACGTGTCGCAGACGCAGGAGCAGAACAAAGAGA |
| | ATCAGAATCCCAATTCTGATGCGCCGGTGATCAGATCAAAAACT |
| | TCAGCCAGGTACATGGAGCTGGTCGGGTGGCTCGTGGACAAGGG |
| | GATTACCTCGGAGAAGCAGTGGATCCAGGAGGACCAGGCCTCAT |
| | ACATCTCCTTCAATGCGGCCTCCAACTCGCGGTCCCAAATCAAG |
| | GCTGCCTTGGACAATGCGGGAAAGATTATGAGCCTGACTAAAAC |
| | CGCCCCCGACTACCTGGTGGGCCAGCAGCCCGTGGAGGACATTT |
| | CCAGCAATCGGATTTATAAAATTTTGGAACTAAACGGGTACGAT |
| | CCCCAATATGCGGCTTCCGTCTTTCTGGGATGGGCCACGAAAAA |
| | GTTCGGCAAGAGGAACACCATCTGGCTGTTTGGGCCTGCAACTA |
| | CCGGGAAGACCAACATCGCGGAGGCCATAGCCCACACTGTGCCC |
| | TTCTACGGGTGCGTAAACTGGACCAATGAGAACTTTCCCTTCAA |
| | CGACTGTGTCGACAAGATGGTGATCTGGTGGGAGGAGGGGAAGA |
| | TGACCGCCAAGGTCGTGGAGTCGGCCAAAGCCATTCTCGGAGGA |
| | AGCAAGGTGCGCGTGGACCAGAAATGCAAGTCCTCGGCCCAGAT |
| | AGACCCGACTCCCGTGATCGTCACCTCCAACACCAACATGTGCG |
| | CCGTGATTGACGGGAACTCAACGACCTTCGAACACCAGCAGCCG |
| | TTGCAAGACCGGATGTTCAAATTTGAACTCACCCGCCGTCTGGA |
| | TCATGACTTTGGGAAGGTCACCAAGCAGGAAGTCAAAGACTTTT |
| | TCCGGTGGGCAAAGGATCACGTGGTTGAGGTGGAGCATGAATTC |
| | TACGTCAAAAAGGGTGGAGCCAAGAAAAGACCCGCCCCCAGTGA |
| | CGCAGATATAAGTGAGCCCAAACGGGTGCGCGAGTCAGTTGCGC |
| | AGCCATCGACGTCAGACGCGGAAGCTTCGATCAACTACGCAGAC |
| | AGGTACCAAAACAAATGTTCTCGTCACGTGGGCATGAATCTGAT |
| | GCTGTTTCCCTGCAGACAATGCGAGAGAATGAATCAGAATTCAA |
| | ATATCTGCTTCACTCACGGACAGAAAGACTGTTTAGAGTGCTTT |
| | CCCGTGTCAGAATCTCAACCCGTTTCTGTCGTCAAAAAGGCGTA |
| | TCAGAAACTGTGCTACATTCATCATATCATGGGAAAGGTGCCAG |
| | ACGCTTGCACTGCCTGCGATCTGGTCAATGTGGATTTGGATGAC |
| | TGCATCTTTGAACAATAA |
| 13-exemplary wild- | ATGCCGGGGTTCTACGAGATTGTCCTGAAGGTCCCGAGTGACCT |
| type AAV3 rep | GGACGAGCACCTGCCGGGCATTTCTAACTCGTTTGTTAACTGGG |
| (derived from NCBI | TGGCCGAGAAGGAATGGGAGCTGCCGCCGGATTCTGACATGGAT |
| GenBank entry | CCGAATCTGATTGAGCAGGCACCCCTGACCGTGGCCGAAAAGCT |
| U48704) | TCAGCGCGAGTTCCTGGTGGAGTGGCGCCGCGTGAGTAAGGCCC |
| | CGGAGGCCCTCTTTTTTGTCCAGTTCGAAAAGGGGGAGACCTAC |
| | TTCCACCTGCACGTGCTGATTGAGACCATCGGGGTCAAATCCAT |
| | GGTGGTCGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAAACGCGAAATGGCGCCGGGGGCGGGAACAAGGT |
| | GGTGGACGACTGCTACATCCCCAACTACCTGCTCCCCAAGACCC |
| | AGCCCGAGCTCCAGTGGGCGTGGACTAACATGGACCAGTATTTA |
| | AGCGCCTGTTTGAATCTCGCGGAGCGTAAACGGCTGGTGGCGCA |
| | GCATCTGACGCACGTGTCGCAGACGCAGGAGCAGAACAAAGAGA |
| | ATCAGAACCCCAATTCTGACGCGCCGGTCATCAGGTCAAAAACC |
| | TCAGCCAGGTACATGGAGCTGGTCGGGTGGCTGGTGGACCGCGG |
| | GATCACGTCAGAAAAGCAATGGATTCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTA |
| | CCAAAAATCGGATCTACCAAATCCTGGAGCTGAACGGGTACGAT |
| | CCGCAGTACGCGGCCTCCGTCTTCCTGGGCTGGGCGCAAAAGAA |
| | GTTCGGGAAGAGGAACACCATCTGGCTCTTTGGGCCGGCCACGA |
| | CGGGTAAAACCAACATCGCGGAAGCCATCGCCCACGCCGTGCCC |
| | TTCTACGGCTGCGTAAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGAGCGCCAAGGCCATTCTGGGCGGA |
| | AGCAAGGTGCGCGTGGACCAAAAGTGCAAGTCATCGGCCCAGAT |
| | CGAACCCACTCCCGTGATCGTCACCTCCAACACCAACATGTGCG |
| | CCGTGATTGACGGGAACAGCACCACCTTCGAGCATCAGCAGCCG |
| | CTGCAGGACCGGATGTTTAAATTTGAACTTACCCGCCGTTTGGA |
| | CCATGACTTTGGGAAGGTCACCAAACAGGAAGTAAAGGACTTTT |
| | TCCGGTGGGCTTCCGATCACGTGACTGACGTGGCTCATGAGTTC |
| | TACGTCAGAAAGGGTGGAGCTAAGAAACGCCCCGCCTCCAATGA |
| | CGCGGATGTAAGCGAGCCAAAACGGCAGTGCACGTCACTTGCGC |
| | AGCCGACAACGTCAGACGCGGAAGCACCGGCGGACTACGCGGAC |
| | AGGTACCAAAACAAATGTTCTCGTCACGTGGGCATGAATCTGAT |
| | GCTTTTTCCCTGTAAAACATGCGAGAGAATGAATCAAATTTCCA |
| | ATGTCTGTTTTACGCATGGTCAAAGAGACTGTGGGGAATGCTTC |
| | CCTGGAATGTCAGAATCTCAACCCGTTTCTGTCGTCAAAAAGAA |
| | GACTTATCAGAAACTGTGTCCAATTCATCATATCCTGGGAAGGG |
| | CACCCGAGATTGCCTGTTCGGCCTGCGATTTGGCCAATGTGGAC |
| | TTGGATGACTGTGTTTCTGAGCAATAA |
| 14-exemplary wild- | ATGCCGGGGTTCTACGAGATCGTGCTGAAGGTGCCCAGCGACCT |
| type AAV4 rep | GGACGAGCACCTGCCCGGCATTTCTGACTCTTTTGTGAGCTGGG |
| (derived from NCBI | TGGCCGAGAAGGAATGGGAGCTGCCGCCGGATTCTGACATGGAC |
| GenBank entry | TTGAATCTGATTGAGCAGGCACCCCTGACCGTGGCCGAAAAGCT |
| NC001829) | GCAACGCGAGTTCCTGGTCGAGTGGCGCCGCGTGAGTAAGGCCC |
| | CGGAGGCCCTCTTCTTTGTCCAGTTCGAGAAGGGGGACAGCTAC |
| | TTCCACCTGCACATCCTGGTGGAGACCGTGGGCGTCAAATCCAT |
| | GGTGGTGGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTCCAGTGGGCGTGGACTAACATGGACCAGTATATA |
| | AGCGCCTGTTTGAATCTCGCGGAGCGTAAACGGCTGGTGGCGCA |
| | GCATCTGACGCACGTGTCGCAGACGCAGGAGCAGAACAAGGAAA |
| | ACCAGAACCCCAATTCTGACGCGCCGGTCATCAGGTCAAAAACC |
| | TCCGCCAGGTACATGGAGCTGGTCGGGTGGCTGGTGGACCGCGG |
| | GATCACGTCAGAAAAGCAATGGATCCAGGAGGACCAGGCGTCCT |
| | ACATCTCCTTCAACGCCGCCTCCAACTCGCGGTCACAAATCAAG |
| | GCCGCGCTGGACAATGCCTCCAAAATCATGAGCCTGACAAAGAC |
| | GGCTCCGGACTACCTGGTGGGCCAGAACCCGCCGGAGGACATTT |
| | CCAGCAACCGCATCTACCGAATCCTCGAGATGAACGGGTACGAT |
| | CCGCAGTACGCGGCCTCCGTCTTCCTGGGCTGGGCGCAAAAGAA |
| | GTTCGGGAAGAGGAACACCATCTGGCTCTTTGGGCCGGCCACGA |
| | CGGGTAAAACCAACATCGCGGAAGCCATCGCCCACGCCGTGCCC |
| | TTCTACGGCTGCGTGAACTGGACCAATGAGAACTTTCCGTTCAA |
| | CGATTGCGTCGACAAGATGGTGATCTGGTGGGAGGAGGGCAAGA |
| | TGACGGCCAAGGTCGTAGAGAGCGCCAAGGCCATCCTGGGCGGA |
| | AGCAAGGTGCGCGTGGACCAAAAGTGCAAGTCATCGGCCCAGAT |
| | CGACCCAACTCCCGTGATCGTCACCTCCAACACCAACATGTGCG |
| | CGGTCATCGACGGAAACTCGACCACCTTCGAGCACCAACAACCA |
| | CTCCAGGACCGGATGTTCAAGTTCGAGCTCACCAAGCGCCTGGA |
| | GCACGACTTTGGCAAGGTCACCAAGCAGGAAGTCAAAGACTTTT |
| | TCCGGTGGGCGTCAGATCACGTGACCGAGGTGACTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACGCACGGGGTCATGGACTGTGCCGAGTGCTTC |
| | CCCGTGTCAGAATCTCAACCCGTGTCTGTCGTCAGAAAGCGGAC |
| | GTATCAGAAACTGTGTCCGATTCATCACATCATGGGGAGGGCGC |
| | CCGAGGTGGCCTGCTCGGCCTGCGAACTGGCCAATGTGGACTTG |
| | GATGACTGTGACATGGAACAATAA |
| 15-exemplary wild- | ATGGCTACCTTCTATGAAGTCATTGTTCGCGTCCCATTTGACGT |
| type AAV5 rep | GGAGGAACATCTGCCTGGAATTTCTGACAGCTTTGTGGACTGGG |
| (derived from NCBI | TAACTGGTCAAATTTGGGAGCTGCCTCCAGAGTCAGATTTAAAT |
| GenBank entry | TTGACTCTGGTTGAACAGCCTCAGTTGACGGTGGCTGATAGAAT |
| NC006152) | TCGCCGCGTGTTCCTGTACGAGTGGAACAAATTTTCCAAGCAGG |
| | AGTCCAAATTCTTTGTGCAGTTTGAAAAGGGATCTGAATATTTT |
| | CATCTGCACACGCTTGTGGAGACCTCCGGCATCTCTTCCATGGT |
| | CCTCGGCCGCTACGTGAGTCAGATTCGCGCCCAGCTGGTGAAAG |
| | TGGTCTTCCAGGGAATTGAACCCCAGATCAACGACTGGGTCGCC |
| | ATCACCAAGGTAAAGAAGGGCGGAGCCAATAAGGTGGTGGATTC |
| | TGGGTATATTCCCGCCTACCTGCTGCCGAAGGTCCAACCGGAGC |
| | TTCAGTGGGCGTGGACAAACCTGGACGAGTATAAATTGGCCGCC |
| | CTGAATCTGGAGGAGCGCAAACGGCTCGTCGCGCAGTTTCTGGC |
| | AGAATCCTCGCAGCGCTCGCAGGAGGCGGCTTCGCAGCGTGAGT |
| | TCTCGGCTGACCCGGTCATCAAAAGCAAGACTTCCCAGAAATAC |
| | ATGGCGCTCGTCAACTGGCTCGTGGAGCACGGCATCACTTCCGA |
| | GAAGCAGTGGATCCAGGAAAATCAGGAGAGCTACCTCTCCTTCA |
| | ACTCCACCGGCAACTCTCGGAGCCAGATCAAGGCCGCGCTCGAC |
| | AACGCGACCAAAATTATGAGTCTGACAAAAAGCGCGGTGGACTA |
| | CCTCGTGGGGAGCTCCGTTCCCGAGGACATTTCAAAAAACAGAA |
| | TCTGGCAAATTTTTGAGATGAATGGCTACGACCCGGCCTACGCG |
| | GGATCCATCCTCTACGGCTGGTGTCAGCGCTCCTTCAACAAGAG |
| | GAACACCGTCTGGCTCTACGGACCCGCCACGACCGGCAAGACCA |
| | ACATCGCGGAGGCCATCGCCCACACTGTGCCCTTTTACGGCTGC |
| | GTGAACTGGACCAATGAAAACTTTCCCTTTAATGACTGTGTGGA |
| | CAAAATGCTCATTTGGTGGGAGGAGGGAAAGATGACCAACAAGG |
| | TGGTTGAATCCGCCAAGGCCATCCTGGGGGGCTCAAAGGTGCGG |
| | GTCGATCAGAAATGTAAATCCTCTGTTCAAATTGATTCTACCCC |
| | TGTCATTGTAACTTCCAATACAAACATGTGTGTGGTGGTGGATG |
| | GGAATTCCACGACCTTTGAACACCAGCAGCCGCTGGAGGACCGC |
| | ATGTTCAAATTTGAACTGACTAAGCGGCTCCCGCCAGATTTTGG |
| | CAAGATTACTAAGCAGGAAGTCAAGGACTTTTTTGCTTGGGCAA |
| | AGGTCAATCAGGTGCCGGTGACTCACGAGTTTAAAGTTCCCAGG |
| | GAATTGGCGGGAACTAAAGGGGCGGAGAAATCTCTAAAACGCCC |
| | ACTGGGTGACGTCACCAATACTAGCTATAAAAGTCTGGAGAAGC |
| | GGGCCAGGCTCTCATTTGTTCCCGAGACGCCTCGCAGTTCAGAC |
| | GTGACTGTTGATCCCGCTCCTCTGCGACCGCTCAATTGGAATTC |
| | AAGGTATGATTGCAAATGTGACTATCATGCTCAATTTGACAACA |
| | TTTCTAACAAATGTGATGAATGTGAATATTTGAATCGGGGCAAA |
| | AATGGATGTATCTGTCACAATGTAACTCACTGTCAAATTTGTCA |
| | TGGGATTCCCCCCTGGGAAAAGGAAAACTTGTCAGATTTTGGGG |
| | ATTTTGACGATGCCAATAAAGAACAGTAA |
| 16-exemplary wild- | ATGCCGGGGTTTTACGAGATTGTGATTAAGGTCCCCAGCGACCT |
| type AAV6 rep | TGACGAGCATCTGCCCGGCATTTCTGACAGCTTTGTGAACTGGG |
| (derived from NCBI | TGGCCGAGAAGGAATGGGAGTTGCCGCCAGATTCTGACATGGAT |
| GenBank entry | CTGAATCTGATTGAGCAGGCACCCCTGACCGTGGCCGAGAAGCT |
| AF028704) | GCAGCGCGACTTCCTGGTCCAGTGGCGCCGCGTGAGTAAGGCCC |
| | CGGAGGCCCTCTTCTTTGTTCAGTTCGAGAAGGGCGAGTCCTAC |
| | TTCCACCTCCATATTCTGGTGGAGACCACGGGGGTCAAATCCAT |
| | GGTGCTGGGCCGCTTCCTGAGTCAGATTAGGGACAAGCTGGTGC |
| | AGACCATCTACCGCGGGATCGAGCCGACCCTGCCCAACTGGTTC |
| | GCGGTGACCAAGACGCGTAATGGCGCCGGAGGGGGGAACAAGGT |
| | GGTGGACGAGTGCTACATCCCCAACTACCTCCTGCCCAAGACTC |
| | AGCCCGAGCTGCAGTGGGCGTGGACTAACATGGAGGAGTATATA |
| | AGCGCGTGTTTAAACCTGGCCGAGCGCAAACGGCTCGTGGCGCA |
| | CGACCTGACCCACGTCAGCCAGACCCAGGAGCAGAACAAGGAGA |
| | ATCTGAACCCCAATTCTGACGCGCCTGTCATCCGGTCAAAAACC |
| | TCCGCACGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCTCTGGACAATGCCGGCAAGATCATGGCGCTGACCAAATC |
| | CGCGCCCGACTACCTGGTAGGCCCCGCTCCGCCCGCCGACATTA |
| | AAACCAACCGCATTTACCGCATCCTGGAGCTGAACGGCTACGAC |
| | CCTGCCTACGCCGGCTCCGTCTTTCTCGGCTGGGCCCAGAAAAG |
| | GTTCGGAAAACGCAACACCATCTGGCTGTTTGGGCCGGCCACCA |
| | CGGGCAAGACCAACATCGCGGAAGCCATCGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTCGGCGGC |
| | AGCAAGGTGCGCGTGGACCAAAAGTGCAAGTCGTCCGCCCAGAT |
| | CGATCCCACCCCCGTGATCGTCACCTCCAACACCAACATGTGCG |
| | CCGTGATTGACGGGAACAGCACCACCTTCGAGCACCAGCAGCCG |
| | TTGCAGGACCGGATGTTCAAATTTGAACTCACCCGCCGTCTGGA |
| | GCATGACTTTGGCAAGGTGACAAAGCAGGAAGTCAAAGAGTTCT |
| | TCCGCTGGGCGCAGGATCACGTGACCGAGGTGGCGCATGAGTTC |
| | TACGTCAGAAAGGGTGGAGCCAACAAGAGACCCGCCCCCGATGA |
| | CGCGGATAAAAGCGAGCCCAAGCGGGCCTGCCCCTCAGTCGCGG |
| | ATCCATCGACGTCAGACGCGGAAGGAGCTCCGGTGGACTTTGCC |
| | GACAGGTACCAAAACAAATGTTCTCGTCACGCGGGCATGCTTCA |
| | GATGCTGTTTCCCTGCAAAACATGCGAGAGAATGAATCAGAATT |
| | TCAACATTTGCTTCACGCACGGGACCAGAGACTGTTCAGAATGT |
| | TTCCCCGGCGTGTCAGAATCTCAACCGGTCGTCAGAAAGAGGAC |
| | GTATCGGAAACTCTGTGCCATTCATCATCTGCTGGGGGGGGCTC |
| | CCGAGATTGCTTGCTCGGCCTGCGATCTGGTCAACGTGGATCTG |
| | GATGACTGTGTTTCTGAGCAATAA |
| 17-exemplary wild- | ATGCCGGGTTTCTACGAGATCGTGATCAAGGTGCCGAGCGACCT |
| type AAV7 rep | GGACGAGCACCTGCCGGGCATTTCTGACTCGTTTGTGAACTGGG |
| (derived from NCBI | TGGCCGAGAAGGAATGGGAGCTGCCCCCGGATTCTGACATGGAT |
| GenBank entry | CTGAATCTGATCGAGCAGGCACCCCTGACCGTGGCCGAGAAGCT |
| NC006260) | GCAGCGCGACTTCCTGGTCCAATGGCGCCGCGTGAGTAAGGCCC |
| | CGGAGGCCCTGTTCTTTGTTCAGTTCGAGAAGGGCGAGAGCTAC |
| | TTCCACCTTCACGTTCTGGTGGAGACCACGGGGGTCAAGTCCAT |
| | GGTGCTAGGCCGCTTCCTGAGTCAGATTCGGGAGAAGCTGGTCC |
| | AGACCATCTACCGCGGGGTCGAGCCCACGCTGCCCAACTGGTTC |
| | GCGGTGACCAAGACGCGTAATGGCGCCGGCGGGGGGAACAAGGT |
| | GGTGGACGAGTGCTACATCCCCAACTACCTCCTGCCCAAGACCC |
| | AGCCCGAGCTGCAGTGGGCGTGGACTAACATGGAGGAGTATATA |
| | AGCGCGTGTTTGAACCTGGCCGAACGCAAACGGCTCGTGGCGCA |
| | GCACCTGACCCACGTCAGCCAGACGCAGGAGCAGAACAAGGAGA |
| | ATCTGAACCCCAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCGGCAAGATCATGGCGCTGACCAAATC |
| | CGCGCCCGACTACCTGGTGGGGCCCTCGCTGCCCGCGGACATTA |
| | AAACCAACCGCATCTACCGCATCCTGGAGCTGAACGGGTACGAT |
| | CCTGCCTACGCCGGCTCCGTCTTTCTCGGCTGGGCCCAGAAAAA |
| | GTTCGGGAAGCGCAACACCATCTGGCTGTTTGGGCCCGCCACCA |
| | CCGGCAAGACCAACATTGCGGAAGCCATCGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTCGGCGGC |
| | AGCAAGGTGCGCGTGGACCAAAAGTGCAAGTCGTCCGCCCAGAT |
| | CGACCCCACCCCCGTGATCGTCACCTCCAACACCAACATGTGCG |
| | CCGTGATTGACGGGAACAGCACCACCTTCGAGCACCAGCAGCCG |
| | TTGCAGGACCGGATGTTCAAATTTGAACTCACCCGCCGTCTGGA |
| | GCACGACTTTGGCAAGGTGACGAAGCAGGAAGTCAAAGAGTTCT |
| | TCCGCTGGGCCAGTGATCACGTGACCGAGGTGGCGCATGAGTTC |
| | TACGTCAGAAAGGGCGGAGCCAGCAAAAGACCCGCCCCCGATGA |
| | CGCGGATATAAGCGAGCCCAAGCGGGCCTGCCCCTCAGTCGCGG |
| | ATCCATCGACGTCAGACGCGGAAGGAGCTCCGGTGGACTTTGCC |
| | GACAGGTACCAAAACAAATGTTCTCGTCACGCGGGCATGATTCA |
| | GATGCTGTTTCCCTGCAAAACGTGCGAGAGAATGAATCAGAATT |
| | TCAACATTTGCTTCACACACGGGGTCAGAGACTGTTTAGAGTGT |
| | TTCCCCGGCGTGTCAGAATCTCAACCGGTCGTCAGAAAAAAGAC |
| | GTATCGGAAACTCTGCGCGATTCATCATCTGCTGGGGGGGGCGC |
| | CCGAGATTGCTTGCTCGGCCTGCGACCTGGTCAACGTGGACCTG |
| | GACGACTGCGTTTCTGAGCAATAA |
| 18-exemplary wild- | ATGCCGGGCTTCTACGAGATCGTGATCAAGGTGCCGAGCGACCT |
| type AAV8 rep | GGACGAGCACCTGCCGGGCATTTCTGACTCGTTTGTGAACTGGG |
| (derived from NCBI | TGGCCGAGAAGGAATGGGAGCTGCCCCCGGATTCTGACATGGAT |
| GenBank entry | CGGAATCTGATCGAGCAGGCACCCCTGACCGTGGCCGAGAAGCT |
| NC006261) | GCAGCGCGACTTCCTGGTCCAATGGCGCCGCGTGAGTAAGGCCC |
| | CGGAGGCCCTCTTCTTTGTTCAGTTCGAGAAGGGCGAGAGCTAC |
| | TTTCACCTGCACGTTCTGGTCGAGACCACGGGGGTCAAGTCCAT |
| | GGTGCTAGGCCGCTTCCTGAGTCAGATTCGGGAAAAGCTTGGTC |
| | CAGACCATCTACCCGCGGGGTCGAGCCCCACCTTGCCCAACTGG |
| | TTCGCGGTGACCAAAGACGCGGTAATGGCGCCGGCGGGGGGGAA |
| | CAAGGTGGTGGACGAGTGCTACATCCCCAACTACCTCCTGCCCA |
| | AGACTCAGCCCGAGCTGCAGTGGGCGTGGACTAACATGGAGGAG |
| | TATATAAGCGCGTGCTTGAACCTGGCCGAGCGCAAACGGCTCGT |
| | GGCGCAGCACCTGACCCACGTCAGCCAGACGCAGGAGCAGAACA |
| | AGGAGAATCTGAACCCCAATTCTGACGCGCCCGTGATCAGGTCA |
| | AAAACCTCCGCGCGCTATATGGAGCTGGTCGGGTGGCTGGTGGA |
| | CCGGGGCATCACCTCCGAGAAGCAGTGGATCCAGGAGGACCAGG |
| | CCTCGTACATCTCCTTCAACGCCGCCTCCAACTCGCGGTCCCAG |
| | ATCAAGGCCGCGCTGGACAATGCCGGCAAGATCATGGCGCTGAC |
| | CAAATCCGCGCCCGACTACCTGGTGGGGCCCTCGCTGCCCGCGG |
| | ACATTACCCAGAACCGCATCTACCGCATCCTCGCTCTCAACGGC |
| | TACGACCCTGCCTACGCCGGCTCCGTCTTTCTCGGCTGGGCTCA |
| | GAAAAAGTTCGGGAAACGCAACACCATCTGGCTGTTTGGACCCG |
| | CCACCACCGGCAAGACCAACATTGCGGAAGCCATCGCCCACGCC |
| | GTGCCCTTCTACGGCTGCGTCAACTGGACCAATGAGAACTTTCC |
| | CTTCAATGATTGCGTCGACAAGATGGTGATCTGGTGGGAGGAGG |
| | GCAAGATGACGGCCAAGGTCGTGGAGTCCGCCAAGGCCATTCTC |
| | GGCGGCAGCAAGGTGCGCGTGGACCAAAAGTGCAAGTCGTCCGC |
| | CCAGATCGACCCCACCCCCGTGATCGTCACCTCCAACACCAACA |
| | TGTGCGCCGTGATTGACGGGAACAGCACCACCTTCGAGCACCAG |
| | CAGCCTCTCCAGGACCGGATGTTTAAGTTCGAACTCACCCGCCG |
| | TCTGGAGCACGACTTTGGCAAGGTGACAAAGCAGGAAGTCAAAG |
| | AGTTCTTCCGCTGGGCCAGTGATCACGTGACCGAGGTGGCGCAT |
| | GAGTTTTACGTCAGAAAGGGCGGAGCCAGCAAAAGACCCGCCCC |
| | CGATGACGCGGATAAAAGCGAGCCCAAGCGGGCCTGCCCCTCAG |
| | TCGCGGATCCATCGACGTCAGACGCGGAAGGAGCTCCGGTGGAC |
| | TTTGCCGACAGGTACCAAAACAAATGTTCTCGTCACGCGGGCAT |
| | GCTTCAGATGCTGTTTCCCTGCAAAACGTGCGAGAGAATGAATC |
| | AGAATTTCAACATTTGCTTCACACACGGGGTCAGAGACTGCTCA |
| | GAGTGTTTCCCCGGCGTGTCAGAATCTCAACCGGTCGTCAGAAA |
| | GAGGACGTATCGGAAACTCTGTGCGATTCATCATCTGCTGGGGC |
| | GGGCTCCCGAGATTGCTTGCTCGGCCTGCGATCTGGTCAACGTG |
| | GACCTGGATGACTGTGTTTCTGAGCAATAA |
| 19-exemplary wild- | ATGCCGGGCTTCTACGAGATTGTGATCAAGGTGCCGAGCGACCT |
| type AAV9 rep | GGACGAGCACCTGCCGGGCATTTCTGACTCTTTTGTGAACTGGG |
| (derived from NCBI | TGGCCGAGAAGGAATGGGAGCTGCCCCCGGATTCTGACATGGAT |
| GenBank entry | CGGAATCTGATCGAGCAGGCACCCCTGACCGTGGCCGAGAAGCT |
| AX753250) | GCAGCGCGACTTCCTGGTCCAATGGCGCCGCGTGAGTAAGGCCC |
| | CGGAGGCCCTCTTCTTTGTTCAGTTCGAGAAGGGCGAGAGCTAC |
| | TTTCACCTGCACGTTCTGGTCGAGACCACGGGGGTCAAGTCCAT |
| | GGTGCTAGGCCGCTTCCTGAGTCAGATTCGGGAGAAGCTGGTCC |
| | AGACCATCTACCGCGGGATCGAGCCGACCCTGCCCAACTGGTTC |
| | GCGGTGACCAAGACGCGTAATGGCGCCGGCGGGGGGAACAAGGT |
| | GGTGGACGAGTGCTACATCCCCAACTACCTCCTGCCCAAGACTC |
| | AGCCCGAGCTGCAGTGGGCGTGGACTAACATGGAGGAGTATATA |
| | AGCGCGTGCTTGAACCTGGCCGAGCGCAAACGGCTCGTGGCGCA |
| | GCACCTGACCCACGTCAGCCAGACGCAGGAGCAGAACAAGGAGA |
| | ATCTGAACCCCAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCGGCAAGATCATGGCGCTGACCAAATC |
| | CGCGCCCGACTACCTGGTAGGCCCTTCACTTCCGGTGGACATTA |
| | CGCAGAACCGCATCTACCGCATCCTGCAGCTCAACGGCTACGAC |
| | CCTGCCTACGCCGGCTCCGTCTTTCTCGGCTGGGCACAAAAGAA |
| | GTTCGGGAAACGCAACACCATCTGGCTGTTTGGGCCGGCCACCA |
| | CGGGAAAGACCAACATCGCAGAAGCCATTGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTCGGCGGC |
| | AGCAAGGTGCGCGTGGACCAAAAGTGCAAGTCGTCCGCCCAGAT |
| | CGACCCCACTCCCGTGATCGTCACCTCCAACACCAACATGTGCG |
| | CCGTGATTGACGGGAACAGCACCACCTTCGAGCACCAGCAGCCT |
| | CTCCAGGACCGGATGTTTAAGTTCGAACTCACCCGCCGTCTGGA |
| | GCACGACTTTGGCAAGGTGACAAAGCAGGAAGTCAAAGAGTTCT |
| | TCCGCTGGGCCAGTGATCACGTGACCGAGGTGGCGCATGAGTTT |
| | TACGTCAGAAAGGGCGGAGCCAGCAAAAGACCCGCCCCCGATGA |
| | CGCGGATAAAAGCGAGCCCAAGCGGGCCTGCCCCTCAGTCGCGG |
| | ATCCATCGACGTCAGACGCGGAAGGAGCTCCGGTGGACTTTGCC |
| | GACAGGTACCAAAACAAATGTTCTCGTCACGCGGGCATGCTTCA |
| | GATGCTGCTTCCCTGCAAAACGTGCGAGAGAATGAATCAGAATT |
| | TCAACATTTGCTTCACACACGGGGTCAGAGACTGCTCAGAGTGT |
| | TTCCCCGGCGTGTCAGAATCTCAACCGGTCGTCAGAAAGAGGAC |
| | GTATCGGAAACTCTGTGCGATTCATCATCTGCTGGGGGGGGCTC |
| | CCGAGATTGCTTGCTCGGCCTGCGATCTGGTCAACGTGGACCTG |
| | GATGACTGTGTTTCTGAGCAATAA |
| 20-exemplary wild- | ATGCCGGGCTTCTACGAGATCGTGATCAAGGTGCCGAGCGACCT |
| type AAV10 rep | GGACGAGCACCTGCCGGGCATTTCTGACTCGTTTGTGAACTGGG |
| (derived from NCBI | TGGCCGAGAAGGAATGGGAGCTGCCCCCGGATTCTGACATGGAT |
| GenBank entry | CGGAATCTGATCGAGCAGGCACCCCTGACCGTGGCCGAGAAGCT |
| AY631966) | GCAGCGCGACTTCCTGGTCCACTGGCGCCGCGTGAGTAAGGCCC |
| | CGGAGGCCCTCTTCTTTGTTCAGTTCGAGAAGGGCGAGTCCTAC |
| | TTTCACCTGCACGTTCTGGTCGAGACCACGGGGGTCAAGTCCAT |
| | GGTCCTGGGCCGCTTCCTGAGTCAGATCAGAGACAGGCTGGTGC |
| | AGACCATCTACCGCGGGGTAGAGCCCACGCTGCCCAACTGGTTC |
| | GCGGTGACCAAGACGCGAAATGGCGCCGGCGGGGGGAACAAGGT |
| | GGTGGACGAGTGCTACATCCCCAACTACCTCCTGCCCAAGACGC |
| | AGCCCGAGCTGCAGTGGGCGTGGACTAACATGGAGGAGTATATA |
| | AGCGCGTGTCTGAACCTCGCGGAGCGTAAACGGCTCGTGGCGCA |
| | GCACCTGACCCACGTCAGCCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCGGAAAGATCATGGCGCTGACCAAATC |
| | CGCGCCCGACTACCTGGTAGGCCCGTCCTTACCCGCGGACATTA |
| | AGGCCAACCGCATCTACCGCATCCTGGAGCTCAACGGCTACGAC |
| | CCCGCCTACGCCGGCTCCGTCTTCCTGGGCTGGGCGCAGAAAAA |
| | GTTCGGTAAAAGGAATACAATTTGGCTGTTCGGGCCCGCCACCA |
| | CCGGCAAGACCAACATCGCGGAAGCCATCGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACCGCCAAGGTCGTGGAGTCCGCCAAGGCCATTCTGGGCGGA |
| | AGCAAGGTGCGCGTCGACCAAAAGTGCAAGTCCTCGGCCCAGAT |
| | CGACCCCACGCCCGTGATCGTCACCTCCAACACCAACATGTGCG |
| | CCGTGATCGACGGGAACAGCACCACCTTCGAGCACCAGCAGCCC |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCTCAGGATCACGTGACTGAGGTGACGCATGAGTTC |
| | TACGTCAGAAAGGGCGGAGCCACCAAAAGACCCGCCCCCAGTGA |
| | CGCGGATATAAGCGAGCCCAAGCGGGCCTGCCCCTCAGTTGCGG |
| | AGCCATCGACGTCAGACGCGGAAGCACCGGTGGACTTTGCGGAC |
| | AGGTACCAAAACAAATGTTCTCGTCACGCGGGCATGCTTCAGAT |
| | GCTGTTTCCCTGCAAGACATGCGAGAGAATGAATCAGAATTTCA |
| | ACGTCTGCTTCACGCACGGGGTCAGAGACTGCTCAGAGTGCTTC |
| | CCCGGCGCGTCAGAATCTCAACCTGTCGTCAGAAAAAAGACGTA |
| | TCAGAAACTGTGCGCGATTCATCATCTGCTGGGGGGGGCACCCG |
| | AGATTGCGTGTTCGGCCTGCGATCTCGTCAACGTGGACTTGGAT |
| | GACTGTGTTTCTGAGCAATAA |
| 21-exemplary wild- | ATGCCGGGCTTCTACGAGATCGTGATCAAGGTGCCGAGCGACCT |
| type AAV11 rep | GGACGAGCACCTGCCGGGCATTTCTGACTCGTTTGTGAACTGGG |
| (derived from NCBI | TGGCCGAGAAGGAATGGGAGCTGCCCCCGGATTCTGACATGGAT |
| GenBank entry | CGGAATCTGATCGAGCAGGCACCCCTGACCGTGGCCGAGAAGCT |
| AY631965) | GCAGCGCGACTTCCTGGTCCACTGGCGCCGCGTGAGTAAGGCCC |
| | CGGAGGCCCTCTTCTTTGTTCAGTTCGAGAAGGGCGAGTCCTAC |
| | TTCCACCTCCACGTTCTCGTCGAGACCACGGGGGTCAAGTCCAT |
| | GGTCCTGGGCCGCTTCCTGAGTCAGATCAGAGACAGGCTGGTGC |
| | AGACCATCTACCGCGGGGTCGAGCCCACGCTGCCCAACTGGTTC |
| | GCGGTGACCAAGACGCGAAATGGCGCCGGCGGGGGGAACAAGGT |
| | GGTGGACGAGTGCTACATCCCCAACTACCTCCTGCCCAAGACCC |
| | AGCCCGAGCTGCAGTGGGCGTGGACTAACATGGAGGAGTATATA |
| | AGCGCGTGTCTAAACCTCGCGGAGCGTAAACGGCTCGTGGCGCA |
| | GCACCTGACCCACGTCAGCCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCGGAAAGATCATGGCGCTGACCAAATC |
| | CGCGCCCGACTACCTGGTAGGCCCGTCCTTACCCGCGGACATTA |
| | AGGCCAACCGCATCTACCGCATCCTGGAGCTCAACGGCTACGAC |
| | CCCGCCTACGCCGGCTCCGTCTTCCTGGGCTGGGCGCAGAAAAA |
| | GTTCGGTAAACGCAACACCATCTGGCTGTTTGGGCCCGCCACCA |
| | CCGGCAAGACCAACATCGCGGAAGCCATAGCCCACGCCGTGCCC |
| | TTCTACGGCTGCGTGAACTGGACCAATGAGAACTTTCCCTTCAA |
| | CGATTGCGTCGACAAGATGGTGATCTGGTGGGAGGAGGGCAAGA |
| | TGACCGCCAAGGTCGTGGAGTCCGCCAAGGCCATTCTGGGCGGA |
| | AGCAAGGTGCGCGTGGACCAAAAGTGCAAGTCCTCGGCCCAGAT |
| | CGACCCCACGCCCGTGATCGTCACCTCCAACACCAACATGTGCG |
| | CCGTGATCGACGGGAACAGCACCACCTTCGAGCACCAGCAGCCG |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCTCAGGATCACGTGACTGAGGTGGCGCATGAGTTC |
| | TACGTCAGAAAGGGCGGAGCCACCAAAAGACCCGCCCCCAGTGA |
| | CGCGGATATAAGCGAGCCCAAGCGGGCCTGCCCCTCAGTTCCGG |
| | AGCCATCGACGTCAGACGCGGAAGCACCGGTGGACTTTGCGGAC |
| | AGGTACCAAAACAAATGTTCTCGTCACGCGGGCATGCTTCAGAT |
| | GCTGTTTCCCTGCAAGACATGCGAGAGAATGAATCAGAATTTCA |
| | ACGTCTGCTTCACGCACGGGGTCAGAGACTGCTCAGAGTGCTTC |
| | CCCGGCGCGTCAGAATCTCAACCCGTCGTCAGAAAAAAGACGTA |
| | TCAGAAACTGTGCGCGATTCATCATCTGCTGGGGGGGGCACCCG |
| | AGATTGCGTGTTCGGCCTGCGATCTCGTCAACGTGGACTTGGAT |
| | GACTGTGTTTCTGAGCAATAA |
| 22-exemplary wild- | ATGCCGGGGTTCTACGAGGTGGTGATCAAGGTGCCCAGCGACCT |
| type AAV12 rep | GGACGAGCACCTGCCCGGCATTTCTGACTCCTTTGTGAACTGGG |
| (derived from NCBI | TGGCCGAGAAGGAATGGGAGTTGCCCCCGGATTCTGACATGGAT |
| GenBank entry | CAGAATCTGATTGAGCAGGCACCCCTGACCGTGGCCGAGAAGCT |
| DQ813647) | GCAGCGCGAGTTCCTGGTGGAATGGCGCCGAGTGAGTAAATTTC |
| | TGGAGGCCAAGTTTTTTGTGCAGTTTGAAAAGGGGGACTCGTAC |
| | TTTCATTTGCATATTCTGATTGAAATTACCGGCGTGAAATCCAT |
| | GGTGGTGGGCCGCTACGTGAGTCAGATTAGGGATAAACTGATCC |
| | AGCGCATCTACCGCGGGGTCGAGCCCCAGCTGCCCAACTGGTTC |
| | GCGGTCACAAAGACCCGAAATGGCGCCGGAGGCGGGAACAAGGT |
| | GGTGGACGAGTGCTACATCCCCAACTACCTGCTCCCCAAGGTCC |
| | AGCCCGAGCTTCAGTGGGCGTGGACTAACATGGAGGAGTATATA |
| | AGCGCCTGTTTGAACCTCGCGGAGCGTAAACGGCTCGTGGCGCA |
| | GCACCTGACGCACGTCTCCCAGACCCAGGAGGGCGACAAGGAGA |
| | ATCTGAACCCGAATTCTGACGCGCCGGTGATCCGGTCAAAAACC |
| | TCCGCCAGGTACATGGAGCTGGTCGGGTGGCTGGTGGACAAGGG |
| | CATCACGTCCGAGAAGCAGTGGATCCAGGAGGACCAGGCCTCGT |
| | ACATCTCCTTCAACGCGGCCTCCAACTCCCGGTCGCAGATCAAG |
| | GCGGCCCTGGACAATGCCTCCAAAATCATGAGCCTCACCAAAAC |
| | GGCTCCGGACTATCTCATCGGGCAGCAGCCCGTGGGGGACATTA |
| | CCACCAACCGGATCTACAAAATCCTGGAACTGAACGGGTACGAC |
| | CCCCAGTACGCCGCCTCCGTCTTTCTCGGCTGGGCCCAGAAAAA |
| | GTTTGGAAAGCGCAACACCATCTGGCTGTTTGGGCCCGCCACCA |
| | CCGGCAAGACCAACATCGCGGAAGCCATCGCCCACGCGGTCCCC |
| | TTCTACGGCTGCGTCAACTGGACCAATGAGAACTTTCCCTTCAA |
| | CGACTGCGTCGACAAAATGGTGATTTGGTGGGAGGAGGGCAAGA |
| | TGACCGCCAAGGTCGTAGAGTCCGCCAAGGCCATTCTGGGCGGC |
| | AGCAAGGTGCGCGTGGACCAAAAATGCAAGGCCTCTGCGCAGAT |
| | CGACCCCACCCCCGTGATCGTCACCTCCAACACCAACATGTGCG |
| | CCGTGATTGACGGGAACAGCACCACCTTCGAGCACCAGCAGCCC |
| | CTGCAGGACCGGATGTTCAAGTTTGAACTCACCCGCCGCCTCGA |
| | CCACGACTTTGGCAAGGTCACCAAGCAGGAAGTCAAGGACTTTT |
| | TCCGGTGGGCGGCTGATCACGTGACTGACGTGGCTCATGAGTTT |
| | TACGTCACAAAGGGTGGAGCTAAGAAAAGGCCCGCCCCCTCTGA |
| | CGAGGATATAAGCGAGCCCAAGCGGCCGCGCGTGTCATTTGCGC |
| | AGCCGGAGACGTCAGACGCGGAAGCTCCCGGAGACTTCGCCGAC |
| | AGGTACCAAAACAAATGTTCTCGTCACGCGGGTATGCTGCAGAT |
| | GCTCTTTCCCTGCAAGACGTGCGAGAGAATGAATCAGAATTCCA |
| | ACGTCTGCTTCACGCACGGTCAGAAAGATTGCGGGGAGTGCTTT |
| | CCCGGGTCAGAATCTCAACCGGTTTCTGTCGTCAGAAAAACGTA |
| | TCAGAAACTGTGCATCCTTCATCAGCTCCGGGGGGCACCCGAGA |
| | TCGCCTGCTCTGCTTGCGACCAACTCAACCCCGATTTGGACGAT |
| | TGCCAATTTGAGCAATAA |
| 23-exemplary wild- | ATGCCGGGATTCTACGAGATTGTCCTGAAGGTGCCCAGCGACCT |
| type AAV13 rep | GGACGAGCACCTGCCTGGCATTTCTGACTCTTTTGTAAACTGGG |
| (derived from NCBI | TGGCGGAGAAGGAATGGGAGCTGCCGCCGGATTCTGACATGGAT |
| GenBank entry | CTGAATCTGATTGAGCAGGCACCCCTAACCGTGGCCGAAAAGCT |
| EU285562) | GCAACGCGAATTCCTGGTCGAGTGGCGCCGCGTGAGTAAGGCCC |
| | CGGAGGCCCTCTTCTTTGTTCAGTTCGAGAAGGGGGACAGCTAC |
| | TTCCACCTACACATTCTGGTGGAGACCGTGGGCGTGAAATCCAT |
| | GGTGGTGGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTCCAGTGGGCGTGGACTAATATGGACCAGTATTTA |
| | AGCGCCTGTTTGAATCTCGCGGAGCGTAAACGGCTGGTGGCGCA |
| | GCATCTGACGCACGTGTCGCAGACGCAGGAGCAGAACAAAGAGA |
| | ACCAGAATCCCAATTCTGACGCGCCGGTGATCAGATCAAAAACC |
| | TCCGCGAGGTACATGGAGCTGGTCGGGTGGCTGGTGGACCGCGG |
| | GATCACGTCAGAAAAGCAATGGATCCAGGAGGACCAGGCCTCTT |
| | ACATCTCCTTCAACGCCGCCTCCAACTCGCGGTCACAAATCAAG |
| | GCCGCACTGGACAATGCCTCCAAATTTATGAGCCTGACAAAAAC |
| | GGCTCCGGACTACCTGGTGGGAAACAACCCGCCGGAGGACATTA |
| | CCAGCAACCGGATCTACAAAATCCTCGAGATGAACGGGTACGAT |
| | CCGCAGTACGCGGCCTCCGTCTTCCTGGGCTGGGCGCAAAAGAA |
| | GTTCGGGAAGAGGAACACCATCTGGCTCTTTGGGCCGGCCACGA |
| | CGGGTAAAACCAACATCGCTGAAGCTATCGCCCACGCCGTGCCC |
| | TTTTACGGCTGCGTGAACTGGACCAATGAGAACTTTCCGTTCAA |
| | CGATTGCGTCGACAAGATGGTGATCTGGTGGGAGGAGGGCAAGA |
| | TGACGGCCAAGGTCGTGGAGTCCGCCAAGGCCATTCTGGGCGGA |
| | AGCAAGGTGCGCGTGGACCAAAAGTGCAAGTCATCGGCCCAGAT |
| | CGACCCAACTCCCGTCATCGTCACCTCCAACACCAACATGTGCG |
| | CGGTCATCGACGGAAATTCCACCACCTTCGAGCACCAACAACCA |
| | CTCCAAGACCGGATGTTCAAGTTCGAGCTCACCAAGCGCCTGGA |
| | GCACGACTTTGGCAAGGTCACCAAGCAGGAAGTCAAGGACTTTT |
| | TCCGGTGGGCGTCAGATCACGTGACTGAGGTGTCTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGCATGAATCTGAT |
| | GCTTTTTCCCTGCCGGCAATGCGAGAGAATGAATCAGAATGTGG |
| | ACATTTGCTTCACGCACGGGGTCATGGACTGTGCCGAGTGCTTC |
| | CCCGTGTCAGAATCTCAACCCGTGTCTGTCGTCAGAAAGCGGAC |
| | ATATCAGAAACTGTGTCCGATTCATCACATCATGGGGAGGGCGC |
| | CCGAGGTGGCTTGTTCGGCCTGCGATCTGGCCAATGTGGACTTG |
| | GATGACTGTGACATGGAGCAATAA |
| 24-exemplary wild- | MPGFYEIVIKVPSDLDEHLPGISDSFVSWVAEKEWELPPDSDMD |
| type AAV1 Rep78 | LNLIEQAPLTVAEKLQRDFLVQWRRVSKAPEALFFVQFEKGESY |
| | FHLHILVETTGVKSMVLGRFLSQIRDKLVQTIYRGIEPTLPNWF |
| | AVTKTRNGAGGGNKVVDECYIPNYLLPKTQPELQWAWTNMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNAGKIMALTKSAPDYLVGPAPPADIKTNRIYRILELNGYE |
| | PAYAGSVFLGWAQKRFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWAQDHVTEVAHEF |
| | YVRKGGANKRPAPDDADKSEPKRACPSVADPSTSDAEGAPVDFA |
| | DRYQNKCSRHAGMLQMLFPCKTCERMNQNFNICFTHGTRDCSEC |
| | FPGVSESQPVVRKRTYRKLCAIHHLLGRAPEIACSACDLVNVDL |
| | DDCVSEQ |
| 25-exemplary wild- | MAADGYLPDWLEDTLSEGIRQWWKLKPGPPPPKPAERHKDDSRG |
| type AAV2 Rep78 | LVLPGYKYLGPFNGLDKGEPVNEADAAALEHDKAYDRQLDSGDN |
| | PYLKYNHADAEFQERLKEDTSFGGNLGRAVFQAKKRVLEPLGLV |
| | EEPVKTAPGKKRPVEHSPVEPDSSSGTGKAGQQPARKRLNFGQT |
| | GDADSVPDPQPLGQPPAAPSGLGTNTMATGSGAPMADNNEGADG |
| | VGNSSGNWHCDSTWMGDRVITTSTRTWALPTYNNHLYKQISSQS |
| | GASNDNHYFGYSTPWGYFDFNRFHCHFSPRDWQRLINNNWGFRP |
| | KRLNFKLFNIQVKEVTQNDGTTTIANNLTSTVQVFTDSEYQLPY |
| | VLGSAHQGCLPPFPADVFMVPQYGYLTLNNGSQAVGRSSFYCLE |
| | YFPSQMLRTGNNFTFSYTFEDVPFHSSYAHSQSLDRLMNPLIDQ |
| | YLYYLSRTNTPSGTTTQSRLQFSQAGASDIRDQSRNWLPGPCYR |
| | QQRVSKTSADNNNSEYSWTGATKYHLNGRDSLVNPGPAMASHKD |
| | DEEKFFPQSGVLIFGKQGSEKTNVDIEKVMITDEEEIRTTNPVA |
| | TEQYGSVSTNLQRGNRQAATADVNTQGVLPGMVWQDRDVYLQGP |
| | IWAKIPHTDGHFHPSPLMGGFGLKHPPPQILIKNTPVPANPSTT |
| | FSAAKFASFITQYSTGQVSVEIEWELQKENSKRWNPEIQYTSNY |
| | NKSVNVDFTVDTNGVYSEPRPIGTRYLTRNL |
| 26-exemplary wild- | MPGFYEIVLKVPSDLDEHLPGISNSFVNWVAEKEWELPPDSDMD |
| type AAV3 Rep78 | PNLIEQAPLTVAEKLQREFLVEWRRVSKAPEALFFVQFEKGETY |
| | FHLHVLIETIGVKSMVVGRYVSQIKEKLVTRIYRGVEPQLPNWF |
| | AVTKTRNGAGGGNKVVDDCYIPNYLLPKTQPELQWAWTNMDQYL |
| | SACLNLAERKRLVAQHLTHVSQTQEQNKENQNPNSDAPVIRSKT |
| | SARYMELVGWLVDRGITSEKQWIQEDQASYISFNAASNSRSQIK |
| | AALDNASKIMSLTKTAPDYLVGSNPPEDITKNRIYQILELNGYD |
| | PQYAASVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIEPTPVIVTSNTNMCAVIDGNSTTFEHQQP |
| | LQDRMFKFELTRRLDHDFGKVTKQEVKDFFRWASDHVTDVAHEF |
| | YVRKGGAKKRPASNDADVSEPKRQCTSLAQPTTSDAEAPADYAD |
| | RYQNKCSRHVGMNLMLFPCKTCERMNQISNVCFTHGQRDCGECF |
| | PGMSESQPVSVVKKKTYQKLCPIHHILGRAPEIACSACDLANVD |
| | LDDCVSEQ |
| 27-exemplary wild- | MPGFYEIVLKVPSDLDEHLPGISDSFVSWVAEKEWELPPDSDMD |
| type AAV4 Rep78 | LNLIEQAPLTVAEKLQREFLVEWRRVSKAPEALFFVQFEKGDSY |
| | FHLHILVETVGVKSMVVGRYVSQIKEKLVTRIYRGVEPQLPNWF |
| | AVTKTRNGAGGGNKVVDDCYIPNYLLPKTQPELQWAWTNMDQYI |
| | SACLNLAERKRLVAQHLTHVSQTQEQNKENQNPNSDAPVIRSKT |
| | SARYMELVGWLVDRGITSEKQWIQEDQASYISFNAASNSRSQIK |
| | AALDNASKIMSLTKTAPDYLVGQNPPEDISSNRIYRILEMNGYD |
| | PQYAASVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTKRLEHDFGKVTKQEVKDFFRWASDHVTEVTHEF |
| | YVRKGGARKRPAPNDADISEPKRACPSVAQPSTSDAEAPVDYAD |
| | RYQNKCSRHVGMNLMLFPCRQCERMNQNVDICFTHGVMDCAECF |
| | PVSESQPVSVVRKRTYQKLCPIHHIMGRAPEVACSACELANVDL |
| | DDCDMEQ |
| 28-exemplary wild- | MATFYEVIVRVPFDVEEHLPGISDSFVDWVTGQIWELPPESDLN |
| type AAV5 Rep78 | LTLVEQPQLTVADRIRRVFLYEWNKFSKQESKFFVQFEKGSEYF |
| | HLHTLVETSGISSMVLGRYVSQIRAQLVKVVFQGIEPQINDWVA |
| | ITKVKKGGANKVVDSGYIPAYLLPKVQPELQWAWTNLDEYKLAA |
| | LNLEERKRLVAQFLAESSQRSQEAASQREFSADPVIKSKTSQKY |
| | MALVNWLVEHGITSEKQWIQENQESYLSFNSTGNSRSQIKAALD |
| | NATKIMSLTKSAVDYLVGSSVPEDISKNRIWQIFEMNGYDPAYA |
| | GSILYGWCQRSFNKRNTVWLYGPATTGKTNIAEAIAHTVPFYGC |
| | VNWTNENFPFNDCVDKMLIWWEEGKMTNKVVESAKAILGGSKVR |
| | VDQKCKSSVQIDSTPVIVTSNTNMCVVVDGNSTTFEHQQPLEDR |
| | MFKFELTKRLPPDFGKITKQEVKDFFAWAKVNQVPVTHEFKVPR |
| | ELAGTKGAEKSLKRPLGDVTNTSYKSLEKRARLSFVPETPRSSD |
| | VTVDPAPLRPLNWNSRYDCKCDYHAQFDNISNKCDECEYLNRGK |
| | NGCICHNVTHCQICHGIPPWEKENLSDFGDFDDANKEQ |
| 29-exemplary wild- | MPGFYEIVIKVPSDLDEHLPGISDSFVNWVAEKEWELPPDSDMD |
| type AAV6 Rep78 | LNLIEQAPLTVAEKLQRDFLVQWRRVSKAPEALFFVQFEKGESY |
| | FHLHILVETTGVKSMVLGRFLSQIRDKLVQTIYRGIEPTLPNWF |
| | AVTKTRNGAGGGNKVVDECYIPNYLLPKTQPELQWAWTNMEEYI |
| | SACLNLAERKRLVAHDLTHVSQTQEQNKENLNPNSDAPVIRSKT |
| | SARYMELVGWLVDRGITSEKQWIQEDQASYISFNAASNSRSQIK |
| | AALDNAGKIMALTKSAPDYLVGPAPPADIKTNRIYRILELNGYD |
| | PAYAGSVFLGWAQKRFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWAQDHVTEVAHEF |
| | YVRKGGANKRPAPDDADKSEPKRACPSVADPSTSDAEGAPVDFA |
| | DRYQNKCSRHAGMLQMLFPCKTCERMNQNFNICFTHGTRDCSEC |
| | FPGVSESQPVVRKRTYRKLCAIHHLLGRAPEIACSACDLVNVDL |
| | DDCVSEQ |
| 30-exemplary wild- | MPGFYEIVIKVPSDLDEHLPGISDSFVNWVAEKEWELPPDSDMD |
| type AAV7 Rep78 | LNLIEQAPLTVAEKLQRDFLVQWRRVSKAPEALFFVQFEKGESY |
| | FHLHVLVETTGVKSMVLGRFLSQIREKLVQTIYRGVEPTLPNWF |
| | AVTKTRNGAGGGNKVVDECYIPNYLLPKTQPELQWAWTNMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNAGKIMALTKSAPDYLVGPSLPADIKTNRIYRILELNGYD |
| | PAYAGSVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWASDHVTEVAHEF |
| | YVRKGGASKRPAPDDADISEPKRACPSVADPSTSDAEGAPVDFA |
| | DRYQNKCSRHAGMIQMLFPCKTCERMNQNENICFTHGVRDCLEC |
| | FPGVSESQPVVRKKTYRKLCAIHHLLGRAPEIACSACDLVNVDL |
| | DDCVSEQ |
| 31-exemplary wild- | MPGFYEIVIKVPSDLDEHLPGISDSFVNWVAEKEWELPPDSDMD |
| type AAV8 Rep78 | RNLIEQAPLTVAEKLQRDFLVQWRRVSKAPEALFFVQFEKGESY |
| | FHLHVLVETTGVKSMVLGRFLSQIREKLGPDHLPAGSSPTLPNW |
| | FAVTKDAVMAPAGGNKVVDECYIPNYLLPKTQPELQWAWTNMEE |
| | YISACLNLAERKRLVAQHLTHVSQTQEQNKENLNPNSDAPVIRS |
| | KTSARYMELVGWLVDRGITSEKQWIQEDQASYISFNAASNSRSQ |
| | IKAALDNAGKIMALTKSAPDYLVGPSLPADITQNRIYRILALNG |
| | YDPAYAGSVFLGWAQKKFGKRNTIWLFGPATTGKTNIAEAIAHA |
| | VPFYGCVNWTNENFPFNDCVDKMVIWWEEGKMTAKVVESAKAIL |
| | GGSKVRVDQKCKSSAQIDPTPVIVTSNTNMCAVIDGNSTTFEHQ |
| | QPLQDRMFKFELTRRLEHDFGKVTKQEVKEFFRWASDHVTEVAH |
| | EFYVRKGGASKRPAPDDADKSEPKRACPSVADPSTSDAEGAPVD |
| | FADRYQNKCSRHAGMLQMLFPCKTCERMNQNENICFTHGVRDCS |
| | ECFPGVSESQPVVRKRTYRKLCAIHHLLGRAPEIACSACDLVNV |
| | DLDDCVSEQ |
| 32-exemplary wild- | MPGFYEIVIKVPSDLDEHLPGISDSFVNWVAEKEWELPPDSDMD |
| type AAV9 Rep78 | RNLIEQAPLTVAEKLQRDFLVQWRRVSKAPEALFFVQFEKGESY |
| | FHLHVLVETTGVKSMVLGRFLSQIREKLVQTIYRGIEPTLPNWF |
| | AVTKTRNGAGGGNKVVDECYIPNYLLPKTQPELQWAWTNMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNAGKIMALTKSAPDYLVGPSLPVDITQNRIYRILQLNGYD |
| | PAYAGSVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWASDHVTEVAHEF |
| | YVRKGGASKRPAPDDADKSEPKRACPSVADPSTSDAEGAPVDFA |
| | DRYQNKCSRHAGMLQMLLPCKTCERMNQNENICFTHGVRDCSEC |
| | FPGVSESQPVVRKRTYRKLCAIHHLLGRAPEIACSACDLVNVDL |
| | DDCVSEQ |
| 33-exemplary wild- | MPGFYEIVIKVPSDLDEHLPGISDSFVNWVAEKEWELPPDSDMD |
| type AAV10 Rep78 | RNLIEQAPLTVAEKLQRDFLVHWRRVSKAPEALFFVQFEKGESY |
| | FHLHVLVETTGVKSMVLGRFLSQIRDRLVQTIYRGVEPTLPNWF |
| | AVTKTRNGAGGGNKVVDECYIPNYLLPKTQPELQWAWTNMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNAGKIMALTKSAPDYLVGPSLPADIKANRIYRILELNGYD |
| | PAYAGSVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWAQDHVTEVTHEF |
| | YVRKGGATKRPAPSDADISEPKRACPSVAEPSTSDAEAPVDEAD |
| | RYQNKCSRHAGMLQMLFPCKTCERMNQNFNVCFTHGVRDCSECF |
| | PGASESQPVVRKKTYQKLCAIHHLLGRAPEIACSACDLVNVDLD |
| | DCVSEQ |
| 34-exemplary wild- | MPGFYEIVIKVPSDLDEHLPGISDSFVNWVAEKEWELPPDSDMD |
| type AAV11 Rep78 | RNLIEQAPLTVAEKLQRDFLVHWRRVSKAPEALFFVQFEKGESY |
| | FHLHVLVETTGVKSMVLGRFLSQIRDRLVQTIYRGVEPTLPNWF |
| | AVTKTRNGAGGGNKVVDECYIPNYLLPKTQPELQWAWTNMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNAGKIMALTKSAPDYLVGPSLPADIKANRIYRILELNGYD |
| | PAYAGSVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWAQDHVTEVAHEF |
| | YVRKGGATKRPAPSDADISEPKRACPSVPEPSTSDAEAPVDFAD |
| | RYQNKCSRHAGMLQMLFPCKTCERMNQNFNVCFTHGVRDCSECF |
| | PGASESQPVVRKKTYQKLCAIHHLLGRAPEIACSACDLVNVDLD |
| | DCVSEQ |
| 35-exemplary wild- | MPGFYEVVIKVPSDLDEHLPGISDSFVNWVAEKEWELPPDSDMD |
| type AAV12 Rep78 | QNLIEQAPLTVAEKLQREFLVEWRRVSKFLEAKFFVQFEKGDSY |
| | FHLHILIEITGVKSMVVGRYVSQIRDKLIQRIYRGVEPQLPNWF |
| | AVTKTRNGAGGGNKVVDECYIPNYLLPKVQPELQWAWTNMEEYI |
| | SACLNLAERKRLVAQHLTHVSQTQEGDKENLNPNSDAPVIRSKT |
| | SARYMELVGWLVDKGITSEKQWIQEDQASYISFNAASNSRSQIK |
| | AALDNASKIMSLTKTAPDYLIGQQPVGDITTNRIYKILELNGYD |
| | PQYAASVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKASAQIDPTPVIVTSNTNMCAVIDGNSTTFEHQQP |
| | LQDRMFKFELTRRLDHDFGKVTKQEVKDFFRWAADHVTDVAHEF |
| | YVTKGGAKKRPAPSDEDISEPKRPRVSFAQPETSDAEAPGDFAD |
| | RYQNKCSRHAGMLQMLFPCKTCERMNQNSNVCFTHGQKDCGECF |
| | PGSESQPVSVVRKTYQKLCILHQLRGAPEIACSACDQLNPDLDD |
| | CQFEQ |
| 36-exemplary wild- | MPGFYEIVLKVPSDLDEHLPGISDSFVNWVAEKEWELPPDSDMD |
| type AAV13 Rep78 | LNLIEQAPLTVAEKLQREFLVEWRRVSKAPEALFFVQFEKGDSY |
| | FHLHILVETVGVKSMVVGRYVSQIKEKLVTRIYRGVEPQLPNWF |
| | AVTKTRNGAGGGNKVVDDCYIPNYLLPKTQPELQWAWTNMDQYL |
| | SACLNLAERKRLVAQHLTHVSQTQEQNKENQNPNSDAPVIRSKT |
| | SARYMELVGWLVDRGITSEKQWIQEDQASYISFNAASNSRSQIK |
| | AALDNASKFMSLTKTAPDYLVGNNPPEDITSNRIYKILEMNGYD |
| | PQYAASVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTKRLEHDFGKVTKQEVKDFFRWASDHVTEVSHEF |
| | YVRKGGARKRPAPNDADISEPKRACPSVAQPSTSDAEAPVDYAD |
| | RYQNKCSRHVGMNLMLFPCRQCERMNQNVDICFTHGVMDCAECF |
| | PVSESQPVSVVRKRTYQKLCPIHHIMGRAPEVACSACDLANVDL |
| | DDCDMEQ |
| 37-clone 0.15 | ATGCCGGGGTTCTACGAGATTGTCCTGAAGGTCCCGAGTGACCT |
| | GGACGAGCACCTGCCGGGCATTTCTAACTCGTTTGTTAACTGGG |
| | TGGCCGAGAAGGAATGGGAGCTGCCCCCGGATTCTGACATGGAT |
| | CGGAATCTGATCGAGCAGGCACCCCTGACCGTGGCCGAGAAGCT |
| | GCAGCGCGACTTCCTGGTCCAGTGGCGCCGCGTGAGTAAGGCCC |
| | CGGAGGCCCTCTTCTTTGTTCAGTTCGAGAAGGGCGAGAGCTAC |
| | TTTCACCTGCACGTTCTGGTCGAGACCACGGGGGTCAAGTCCAT |
| | GGTCCTGGGCCGCTTCCTGAGTCAGATCAGAGACAGGCTGGTGC |
| | AGACCATCTACCGCGGGGTCGAGCCCACGCTGCCCAACTGGTTC |
| | GCGGTGACCAAAGACGCGGTAATGGCGCCGGCGGGGGGGAACAA |
| | GGTGGTGGACGAGTGCTACATCCCCAACTACCTCCTGCCCAAGA |
| | CCCAGCCCGAGCTGCAGTGGGCGTGGACTAACATGGAGGAGTAT |
| | ATAAGCGCGTGTCTAAACCTCGCGGAGCGTAAACGGCTCGTGGC |
| | GCAGCACCTGACCCACGTCAGCCAGACGCAGGAGCAGAACAAGG |
| | AGAATCTGAACCCGAATTCTGACGCGCCCGTGATCAGGTCAAAA |
| | ACCTCCGCGCGCTACATGGAGCTGGTCGGGTGGCTGGTGGACCG |
| | GGGCATCACCTCCGAGAAGCAGTGGATCCAGGAGGACCAGGCCT |
| | CGTACATCTCCTTCAACGCCGCCTCCAACTCGCGGTCACAAATC |
| | AAGGCCGCACTGGACAATGCCGGCAAGATCATGGCGCTGACCAA |
| | ATCCGCGCCCGACTACCTGGTAGGCCCGTCCTTACCCGCGGACA |
| | TTAAGGCCAACCGCATCTACCGCATCCTGGAGCTCAACGGCTAC |
| | GACCCCGCCTACGCGGCCTCCGTCTTCCTGGGCTGGGCGCAAAA |
| | GAAGTTCGGGAAGAGGAACACCATCTGGCTCTTTGGGCCGGCCA |
| | CGACGGGTAAAACCAACATCGCGGAAGCCATCGCCCACGCCGTG |
| | CCCTTCTACGGCTGCGTCAACTGGACCAATGAGAACTTTCCGTT |
| | CAACGACTGTGTCGACAAGATGGTGATCTGGTGGGAGGAGGGCA |
| | AGATGACGGCCAAGGTCGTGGAGTCCGCCAAGGCCATTCTCGGC |
| | GGCAGCAAGGTGCGCGTGGACCAAAAGTGCAAGTCGTCCGCCCA |
| | GATCGATCCCACCCCCGTGATCGTCACCTCCAACACCAACATGT |
| | GCGCCGTGATTGACGGGAACAGCACCACCTTCGAGCACCAGCAG |
| | CCCCTGCAGGACCGGATGTTCAAGTTTGAACTCACCCGCCGCCT |
| | CGACCACGACTTTGGCAAGGTCACCAAGCAGGAAGTCAAGGACT |
| | TTTTCCGGTGGGCGCAGGATCACGTGACCGAGGTGGCGCATGAG |
| | TTCTACGTCAGAAAGGGTGGAGCCAACAAGAGACCCGCCCCCAG |
| | TGACGCGGATATAAGCGAGCCCAAGCGGGCCTGCCCCTCAGTTC |
| | CGGAGCCATCGACGTCAGACGCGGAAGCACCGGTGGACTTTGCG |
| | GACAGGTACCAAAACAAATGTTCTCGTCACGCGGGCATGCTTCA |
| | GATGCTGTTTCCCTGCAAGACATGCGAGAGAATGAATCAGAATT |
| | TCAACGTCTGCTTCACGCACGGGGTCAGAGACTGCTCAGAGTGC |
| | TTCCCCGGCGCGTCAGAATCTCAACCTGTCGTCAGAAAAAAGAC |
| | GTATCAGAAACTGTGCGCGATTCATCATCTGCTGGGGGGGGCAC |
| | CCGAGATTGCGTGTTCGGCCTGCGATCTCGTCAACGTGGACTTG |
| | GATGACTGKGTTTCTGAACAATAA |
| 38-clone 1.01 | ATGCCGGGCTTCTACGAGATTGTCCTGAAGGTCCCGAGTGACCT |
| | GGACGAGCACCTGCCGGGCATTTCTAACTCGTTTGTTAACTGGG |
| | TGGCCGAGAAGGAATGGGAGCTGCCGCCGGATTCTGACATGGAC |
| | TTGAATCTGATTGAGCAGGCACCCCTGACCGTGGCCGAAAAGCT |
| | GCAGCGCGACTTCCTGGTCCACTGGCGCCGCGTGAGTAAGGCCC |
| | CGGAGGCCCTCTTCTTTGTTCAGTTCGAGAAGGGCGAGTCCTAC |
| | TTCCACCTCCATATTCTGGTGGAGACCACGGGGGTCAAATCCAT |
| | GGTGCTGGGCCGCTTCCTGAGTCAGATTAGGGACAAGCTGGTGC |
| | AGACCATCTACCGCGGGATCGAGCCGACCCTGCCCAACTGGTTC |
| | GCGGTGACCAAGACGCGTAATGGCGCCGGCGGGGGGAACAAGGT |
| | GGTGGACGAGTGCTACATCCCCAACTACCTGCTCCCCAAGACCC |
| | AGCCCGAGCTGCAGTGGGCGTGGACTAACATGGAGGAGTATATA |
| | AGCGCCTGTTTGAACCTCGCGGAGCGTAAACGGCTCGTGGCGCA |
| | GCATCTGACGCACGTGTCGCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGCGG |
| | GATCACGTCAGAAAAGCAATGGATCCAGGAGGACCAGGCGTCCT |
| | ACATCTCCTTCAACGCCGCCTCCAACTCGCGGTCACAAATCAAG |
| | GCCGCGCTGGACAATGCCTCCAAAATCATGAGCCTCACCAAAAC |
| | GGCTCCGGACTATCTCATCGGGCAGCAGCCCGTGGGGGACATTA |
| | CCACCAACCGGATCTACAAAATCCTGGAACTGAACGGGTACGAC |
| | CCCCAGTACGCCGCCTCCGTCTTTCTCGGCTGGGCCCAGAAAAG |
| | GTTCGGGAAGCGCAACACCATCTGGCTGTTTGGGCCGGCCACCA |
| | CCGGCAAGACCAACATTGCGGAAGCCATCGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACCGCCAAGGTCGTAGAGAGCGCCAAGGCCATCCTGGGCGGA |
| | AGCAAGGTGCGCGTGGACCAAAAGTGCAAGTCGTCCGCCCAGAT |
| | CGACCCCACTCCCGTGATCGTCACCTCCAACACCAACATGTGCG |
| | CCGTGATTGACGGGAACAGCACCACCTTCGAGCACCAGCAGCCC |
| | CTGCAGGACCGGATGTTCAAATTTGAACTTACCCGCCGTTTGGA |
| | CCATGACTTTGGCAAGGTCACCAAGCAGGAAGTCAAAGACTTTT |
| | TCCGGTGGGCGTCAGATCACGTGACCGAGGTGACTCACGAGTTT |
| | TACGTCAGAAAGGGCGGAGCCAGCAAAAGACCCGCCCCCGATGA |
| | CGCGGATAAAAGCGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACGCACGGGGTCATGGACTGTGCCGAGTGCTTC |
| | CCCGTGTCAGAATCTCAACCCGTGTCTGTCGTCAGAAAGCGGAC |
| | ATATCAGAAACTGTGTTTGATTCATCACATCATGGGGAGGGCGC |
| | CCGAGGTGGCTTGTTCGGCCTGCGAACTGGCCAATGTGGACTTG |
| | GATGACTGTGACATGGAACAATAA |
| 39-clone 1.03 | ATGCCGGGGTTTTACGAGATTGTGATTAAGGTCCCCAGCGACCT |
| | TGACGAGCATCTGCCCGGCATTTCTGACAGCTTTGTGAACTGGG |
| | TGGCCGAGAAGGAATGGGAGCTGCCCCCGGATTCTGACATGGAT |
| | CTGAATCTGATTGAGCAGGCACCCCTGACCGTGGCCGAGAAGCT |
| | GCAGCGCGACTTCCTGGTCCAATGGCGCCGCGTGAGTAAGGCCC |
| | CGGAGGCCCTCTTCTTTGTTCAGTTCGAGAAGGGCGAGAGCTAC |
| | TTCCACCTTCACGTTCTGGTGGAGACCACGGGGGTCAAGTCCAT |
| | GGTGCTAGGCCGCTTCCTGAGTCAGATTCGGGAGAAGCTGGTCC |
| | AGACCATCTACCGCGGGATCGAGCCGACCCTGCCCAACTGGTTC |
| | GCGGTGACCAAGACGCGTAATGGCGCCGGAGGGGGGAACAAGGT |
| | GGTGGACGAGTGCTACATCCCCAACTACCTCCTGCCCAAGACTC |
| | AGCCCGAGCTGCAGTGGGCGTGGACTAACATGGAGGAGTATATA |
| | AGCGCGTGCTTGAACCTGGCCGAGCGCAAACGGCTCGTGGCGCA |
| | GCACCTGACCCACGTCAGCCAGACCCAGGAGCAGAACAAGGAGA |
| | ATCTGAACCCCAATTCTGACGCGCCCGTGATCAGGTCAAAAACC |
| | TCCGCACGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTA |
| | CCAAAAATCGGATCTACCAAATCCTGGAGCTGAACGGGTACGAT |
| | CCGCAGTACGCGGCCTCCGTCTTCCTGGGCTGGGCGCAAAAGAA |
| | GTTCGGGAAGAGGAACACCATCTGGCTCTTTGGGCCGGCCACGA |
| | CGGGTAAAACCAACATCGCGGAAGCCATCGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCGGCCAAAGCCATTCTCGGAGGA |
| | AGCAAGGTGCGCGTGGACCAAAAGTGCAAGTCCTCGGCCCAGAT |
| | CGACCCCACGCCCGTGATCGTCACCTCCAACACCAACATGTGCG |
| | CCGTGATCGACGGGAACAGCACCACCTTCGAGCACCAGCAGCCG |
| | CTGCAGGACCGGATGTTCAAATTTGAACTCACCCGCCGTCTGGA |
| | GCATGACTTTGGCAAGGTGACAAAGCAGGAAGTCAAAGAGTTCT |
| | TCCGCTGGGCGCAGGATCACGTGACCGAGGTGGCGCATGAGTTC |
| | TACGTCAGAAAGGGCGGAGCCACCAAAAGACCCGCCCCCAGTGA |
| | CGCGGATATAAGCGAGCCCAAGCGGGCCTGCCCCTCAGTTCCGG |
| | AGCCATCGACGTCAGACGCGGAAGCGCCGGTGGACTTTGCGGAC |
| | AGGTACCAAAACAAATGTTCTCGTCACGCGGGCATGCTTCAGAT |
| | GCTGTTTCCCTGCAAGACATGCGAGAGAATGAATCAGAATTTCA |
| | ACGTCTGCTTCACGCACGGGGTCAGAGACTGCTCAGAGTGCTTC |
| | CCCGGCGTGTCAGAATCTCAACCCGTGTCTGTCGTCAGAAAGCG |
| | GACATATCAGAAACTGTGTCCGATTCATCACATCATGGGGAGGG |
| | CGCCCGAGATTGCTTGCTCGGCCTGCGATCTGGTCAACGTGGAC |
| | CTGGATGACTGTGTTTCTGAGCAATAA |
| 40-clone 1.09 | ATGCCGGGGTTCTACGAGATTGTCCTGAAGGTCCCGAGTGACCT |
| | GGACGAGCACCTGCCGGGCATTTCTAACTCGTTTGTTAACTGGG |
| | TGGCCGAGAAGGAATGGGAGTTGCCCCCGGATTCTGACATGGAT |
| | CGGAATCTGATCGAGCAGGCACCCCTGACCGTGGCCGAGAAGCT |
| | GCAGCGCGACTTCCTGGTCCACTGGCGCCGCGTGAGTAAGGCCC |
| | CGGAGGCCCTCTTCTTTGTTCAGTTCGAGAAGGGCGAGAGCTAC |
| | TTTCACCTGCACGTTCTGGTCGAGACCACGGGGGTCAAGTCCAT |
| | GGTGCTAGGCCGCTTCCTGAGTCAGATTCGGGAGAAGCTGGTCC |
| | AGACCATCTACCGCGGGATCGAGCCGACCCTGCCCAACTGGTTC |
| | GCGGTGACCAAGACGCGTAATGGCGCCGGCGGGGGGAACAAGGT |
| | GGTGGACGACTGCTACATCCCCAACTACCTGCTCCCCAAGACCC |
| | AGCCCGAGCTCCAGTGGGCGTGGACTAACATGGACCAGTATTTA |
| | AGCGCCTGTTTGAATCTCACGGAGCGTAAACGGTTGGTGGCGCA |
| | GCATCTGACGCACGTGTCGCAGACGCAGGAGCAGAACAAGGAGA |
| | ATCTGAACCCCAATTCTGACGCGCCTGTCATCCGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCTCTGGACAATGCCGGCAAGATCATGGCGCTGACCAAATC |
| | CGCGCCCGACTACCTGGTAGGCCCCGCTCCGCCCGCCGACATTA |
| | AAACCAACCGCATTTACCGCATCCTGGAGCTGAACGGCTACGAC |
| | CCTGCCTACGCCGGCTCCGTCTTTCTCGGCTGGGCTCAGAAAAA |
| | GTTCGGTAAAAGGAATACAATTTGGCTGTTCGGGCCCGCCACCA |
| | CCGGCAAGACCAACATTGCGGAAGCCATCGCCCACGCCGTGCCC |
| | TTCTACGGCTGCGTCAACTGGACCAATGAGAACTTTCCCTTCAA |
| | TGATTGCGTCGACAAGATGGTGATCTGGTGGGAGGAGGGCAAGA |
| | TGACGGCCAAGGTCGTGGAGTCCGCCAAGGCCATTCTCGGCGGC |
| | AGCAAGGTGCGCGTGGACCAAAAGTGCAAGTCGTCCGCCCAGAT |
| | CGATCCCACCCCCGTGATCGTCACCTCCAACACCAACATGTGCG |
| | CCGTGATTGACGGGAACAGCACCACCTTCGAGCACCAGCAGCCG |
| | TTGCAGGACCGGATGTTCAAATTTGAACTCACCCGCCGTCTGGA |
| | GCATGACTTTGGCAAGGTGACAAAGCAGGAAGTCAAAGAGTTCT |
| | TCCGCTGGGCGCAGGATCACGTGACTGAGGTGGCGCATGAGTTC |
| | TACGTCAGAAAGGGTGGAGCCAAGAAAAGGCCCGCCCCCTCTGA |
| | CGAGGATATAAGCGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACGCACGGGGTCATGGACTGTGCCGAGTGCTTC |
| | CCCGTGTCAGAATCTCAACCCGTGTCTGTCGTCAGAAAGCGGAC |
| | ATATCAGAAACTGTGTTTGATTCATCACATCATGGGGAGGGCGC |
| | CCGAGGTGGCTTGTTCGGCCTGCGAACTGGCCAATGTGGACTTG |
| | GATGACTGTGACATGGAACAATAA |
| 41-clone 1.10 | ATGCCGGGGTTTTACGAGATTGTGATTAAGGTCCCCAGCGACCT |
| | TGACGAGCATCTGCCCGGCATTTCTGACAGCTTTGTGAACTGGG |
| | TGGCCGAGAAGGAATGGGAGCTGCCCCCGGATTCTGACATGGAT |
| | CTGAATCTGATTGAGCAGGCACCCCTGACCGTGGCCGAGAAGCT |
| | GCAGCGCGAGTTCCTGGTGGAGTGGCGCCGCGTGAGTAAGGCCC |
| | CGGAGGCCCTCTTTTTTGTCCAGTTCGAAAAGGGGGAGACCTAC |
| | TTCCACCTGCACGTGCTGATTGAGACCATCGGGGTCAAATCCAT |
| | GGTGGTCGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTGCAGTGGGCGTGGACTAACATGGAGGAGTATATA |
| | AGCGCGTGTCTGAACCTCGCGGAGCGTAAACGGCTCGTGGCGCA |
| | GCACCTGACCCACGTCAGCCAGACGCAGGAGCAGAACAAGGAGA |
| | ATCTGAACCCCAATTCTGACGCGCCCGTGATCAGGTCAAAAACC |
| | TCCGCGCGCTACATGGAGCTGGTCGGGTGGCTCGTGGACAAGGG |
| | GATTACCTCGGAGAAGCAGTGGATCCAGGAGGACCAGGCCTCGT |
| | ACATCTCCTTCAACGCCGCCTCCAACTCGCGGTCACAAATCAAG |
| | GCCGCGCTGGACAATGCCTCCAAAATCATGAGCCTGACAAAGAC |
| | GGCTCCGGACTACCTGGTGGGCCAGAACCCGCCGGAGGACATTA |
| | CCAGCAACCGGATCTACAAAATCCTCGAGATGAACGGGTACGAT |
| | CCGCAGTACGCGGCCTCCGTCTTCCTGGGCTGGGCGCAAAAGAA |
| | GTTCGGTAAACGCAACACCATCTGGCTGTTTGGGCCTGCAACTA |
| | CCGGCAAGACCAACATCGCGGAAGCCATCGCCCACGCGGTCCCC |
| | TTCTACGGCTGCGTCAACTGGACCAATGAGAACTTTCCCTTCAA |
| | TGATTGCGTCGACAAGATGGTGATCTGGTGGGAGGAGGGCAAGA |
| | TGACGGCCAAGGTCGTGGAGTCCGCCAAGGCCATTCTCGGCGGC |
| | AGCAAGGTGCGCGTGGACCAAAAATGCAAGGCCTCTGCGCAGAT |
| | CGACCCCACCCCCGTGATCGTCACCTCCAACACCAACATGTGCG |
| | CCGTGATCGACGGGAACAGCACCACCTTCGAGCACCAGCAGCCC |
| | CTGCAGGACCGCATGTTCAAATTTGAACTCACCCGCCGTCTGGA |
| | GCACGACTTTGGCAAGGTGACGAAGCAGGAAGTCAAAGAGTTCT |
| | TCCGCTGGGCCAGTGATCACGTGACTGAGGTGTCTCACGAGTTT |
| | TACGTCAGAAAGGGTGGAGCCAACAAAAGACCCGCCCCCGATGA |
| | CGCGGATAAAAGCGAGCCCAAGCGGGCCTGCCCCTCAGTTGCGG |
| | AGCCATCGACGTCAGACGCGGAAGCACCGGTGGACTTTGCGGAC |
| | AGGTACCAAAACAAATGTTCTCGTCACGCGGGCATGCTTCAGAT |
| | GCTGTTTCCCTGCAGACAATGCGAGAGAATGAATCAGAATTCAA |
| | ATATCTGCTTCACTCACGGACAGAAAGACTGTTTAGAGTGCTTT |
| | CCCGTGTCAGAATCTCAACCCGTTTCTGTCGTCAAAAAGGCGTA |
| | TCAGAAACTGTGCTACATTCATCATATCATGGGAAAGGTGCCAG |
| | ACGCTTGCACTGCCTGCGATCTGGTCAATGTGGATTTGGATGAC |
| | TGCATCTTTGAACAATAA |
| 42-clone 1.12 | ATGCCGGGGTTCTACGAGATTGTCCTGAAGGTCCCGAGTGACCT |
| | GGACGAGCACCTGCCGGGCATTTCTAACTCGTTTGTTAACTGGG |
| | TGGCCGAGAAGGAATGGGAGCTGCCGCCAGATTCTGACATGGAT |
| | CGGAATCTGATCGAGCAGGCACCCCTGACCGTGGCCGAGAAGCT |
| | GCAGCGCGAGTTCCTGGTGGAGTGGCGCCGCGTGAGTAAGGCCC |
| | CGGAGGCCCTCTTTTTTGTCCAGTTCGAAAAGGGGGAGACCTAC |
| | TTCCACCTGCACGTGCTGATTGAGACCATCGGGGTCAAATCCAT |
| | GGTGGTCGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTGCAGTGGGCGTGGACTAACATGGAGGAGTATATA |
| | AGCGCGTGTCTGAACCTCGCGGAGCGTAAACGGCTCGTGGCGCA |
| | GCACCTGACCCACGTCAGCCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTT |
| | CCAGCAACCGGATCTACAAAATCCTCGAGATGAACGGGTACGAT |
| | CCGCAGTACGCGGCCTCCGTCTTTCTCGGCTGGGCACAAAAGAA |
| | GTTCGGGAAACGCAACACCATCTGGCTGTTTGGGCCGGCCACCA |
| | CGGGAAAGACCAACATCGCAGAAGCCATTGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTGGGCGGA |
| | AGCAAGGTGCGCGTGGACCAAAAGTGCAAGTCGTCCGCCCAGAT |
| | CGACCCCACCCCCGTGATCGTCACCTCCAACACCAACATGTGCG |
| | CCGTGATCGACGGGAACAGCACCACCTTCGAGCACCAGCAGCCG |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCGCAGGATCACGTGACCGAGGTGTCTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACACACGGGGTCAGAGACTGCTCAGAGTGTTTC |
| | CCCGGCGTGTCAGAATCTCAACCGGTCGTCAGAAAAAAGACGTA |
| | TCAGAAACTGTGTCCGATTCATCACATCATGGGGAGGGCGCCCG |
| | AGGTGGCCTGCTCGGCCTGCGAACTGGCCAATGTGGACTTGGAT |
| | GACTGTGACATGGAACAATAA |
| 43-clone 1.45 | ATGCCGGGGTTCTACGAGATTGTCCTGAAGGTCCCGAGTGACCT |
| | GGACGAGCACCTGCCGGGCATTTCTAACTCGTTTGTTAACTGGG |
| | TGGCCGAGAAGGAATGGGAGCTGCCGCCAGATTCTGACATGGAT |
| | CGGAATCTGATCGAGCAGGCACCCCTGACCGTGGCCGAGAAGCT |
| | GCAGCGCGAGTTCCTGGTGGAGTGGCGCCGCGTGAGTAAGGCCC |
| | CGGAGGCCCTCTTTTTTGTCCAGTTCGAAAAGGGGGAGACCTAC |
| | TTCCACCTGCACGTGCTGATTGAGACCATCGGGGTCAAATCCAT |
| | GGTGGTCGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTGCAGTGGGCGTGGACTAACATGGAGGAGTATATA |
| | AGCGCGTGTCTGAACCTCGCGGAGCGTAAACGGCTCGTGGCGCA |
| | GCACCTGACCCACGTCAGCCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTT |
| | CCAGCAACCGGATCTACAAAATCCTCGAGATGAACGGGTACGAT |
| | CCGCAGTACGCGGCCTCCGTCTTTCTCGGCTGGGCACAAAAGAA |
| | GTTCGGGAAACGCAACACCATCTGGCTGTTTGGGCCGGCCACCA |
| | CGGGAAAGACCAACATCGCAGAAGCCATTGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTGGGCGGA |
| | AGCAAGGTGCGCGTGGACCAAAAGTGCAAGTCGTCCGCCCAGAT |
| | CGACCCCACCCCCGTGATCGTCACCTCCAACACCAACATGTGCG |
| | CCGTGATCGACGGGAACAGCACCACCTTCGAGCACCAGCAGCCG |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCGCAGGATCACGTGACCGAGGTGTCTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACACACGGGGTCAGAGACTGCTCAGAGTGTTTC |
| | CCCGGCGTGTCAGAATCTCAACCGGTCGTCAGAAAAAAGACGTA |
| | TCAGAAACTGTGTCCGATTCATCACATCATGGGGAGGGCGCCCG |
| | AGGTGGCCTGCTCGGCCTGCGAACTGGCCAATGTGGACTTGGAT |
| | GACTGTGACATGGAACAATAA |
| 44-clone 1.46 | ATGCCGGGGTTCTACGAGATTGTCCTGAAGGTCCCGAGTGACCT |
| | GGACGAGCACCTGCCGGGCATTTCTAACTCGTTTGTGAACTGGG |
| | TGGCCGAGAAGGAATGGGAGCTGCCCCCGGATTCTGACATGGAT |
| | CTGAATCTGATTGAGCAGGCACCCCTGACCGTGGCCGAGAAGCT |
| | GCAGCGCGACTTCCTGGTCCAATGGCGCCGCGTGAGTAAGGCCC |
| | CGGAGGCCCTGTTCTTTGTTCAGTTCGAGAAGGGCGAGAGCTAC |
| | TTCCACCTTCACGTTCTGGTGGAGACCACGGGGGTCAAGTCCAT |
| | GGTGCTAGGCCGCTTCCTGAGTCAGATTCGGGACAAGCTGGTGC |
| | AGACCATCTACCGCGGGATCGAGCCGACCCTGCCCAACTGGTTC |
| | GCGGTGACCAAGACGCGTAATGGCGCCGGCGGGGGGAACAAGGT |
| | GGTGGACGAGTGCTACATCCCCAACTACCTGCTCCCCAAGACCC |
| | AGCCCGAGCTGCAGTGGGCGTGGACTAACATGGAGGAGTATATA |
| | AGCGCGTGCTTGAACCTGGCCGAGCGCAAACGGCTCGTGGCGCA |
| | GCACCTGACCCACGTCAGCCAGACCCAGGAGCAGAACAAGGAGA |
| | ATCTGAACCCCAATTCTGACGCGCCCGTGATCAGGTCAAAAACC |
| | TCCGCACGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTT |
| | CCAGCAACCGGATCTACAAAATCCTCGAGATGAACGGGTACGAT |
| | CCGCAGTACGCGGCCTCCGTCTTTCTCGGCTGGGCACAAAAGAA |
| | GTTCGGGAAACGCAACACCATCTGGCTGTTTGGGCCGGCCACCA |
| | CGGGAAAGACCAACATCGCAGAAGCCATTGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTGGGCGGA |
| | AGCAAGGTGCGCGTGGACCAAAAGTGCAAGTCGTCCGCCCAGAT |
| | CGACCCCACTCCCGTGATCGTCACCTCCAACACCAACATGTGCG |
| | CCGTGATCGACGGGAACAGCACCACCTTCGAGCACCAGCAGCCG |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCGCAGGATCACGTGACCGAGGTGTCTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACACACGGGGTCAGAGACTGCTCAGAGTGTTTC |
| | CCCGGCGTGTCAGAATCTCAACCGGTCGTCAGAAAAAAGACGTA |
| | TCAGAAACTGTGTCCGATTCATCACATCATGGGGAGGGCGCCCG |
| | AGGTGGCCTGCTCGGCCTGCGAACTGGCCAATGTGGACTTGGAT |
| | GACTGTGACATGGAACAATAA |
| 45-clone 1.47 | ATGCCGGGGTTCTACGAGATTGTCCTGAAGGTCCCGAGTGACCT |
| | GGACGAGCACCTGCCGGGCATTTCTAACTCGTTTGTTAACTGGG |
| | TGGCCGAGAAGGAATGGGAGCTGCCGCCAGATTCTGACATGGAT |
| | CGGAATCTGATCGAGCAGGCACCCCTGACCGTGGCCGAGAAGCT |
| | GCAGCGCGAGTTCCTGGTGGAGTGGCGCCGCGTGAGTAAGGCCC |
| | CGGAGGCCCTCTTTTTTGTCCAGTTCGAAAAGGGGGAGACCTAC |
| | TTCCACCTGCACGTGCTGATTGAGACCATCGGGGTCAAATCCAT |
| | GGTGGTCGGCCGCTACGTGAGCCAGATTAAAGAGAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTGCAGTGGGCGTGGACTAACATGGAGGAGTATATA |
| | AGCGCGTGTCTGAACCTCGCGGAGCGTAAACGGCTCGTGGCGCA |
| | GCACCTGACCCACGTCAGCCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTT |
| | CCAGCAACCGGATCTACAAAATCCTCGAGATGAACGGGTACGAT |
| | CCGCAGTACGCGGCCTCCGTCTTTCTCGGCTGGGCACAAAAGAA |
| | GTTCGGGAAACGCAACACCATCTGGCTGTTTGGGCCGGCCACCA |
| | CGGGAAAGACCAACATCGCAGAAGCCATTGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTGGGCGGA |
| | AGCAAGGTGCGCGTGGACCAAAAGTGCAAGTCGTCCGCCCAGAT |
| | CGACCCCACCCCCGTGATCGTCACCTCCAACACCAACATGTGCG |
| | CCGTGATCGACGGGAACAGCACCACCTTCGAGCACCAGCAGCCG |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCGCAGGATCACGTGACCGAGGTGTCTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACACACGGGGTCAGAGACTGCTCAGAGTGTTTC |
| | CCCGGCGTGTCAGAATCTCAACCGGTCGTCAGAAAAAAGACGTA |
| | TCAGAAACTGTGTCCGATTCATCACATCATGGGGAGGGCGCCCG |
| | AGGTGGCCTGCTCGGCCTGCGAACTGGCCAATGTGGACTTGGAT |
| | GACTGTGACATGGAACAATAA |
| 46-clone 2.29 | ATGCCGGGGTTTTACGAGATTGTGATTAAGGTCCCCAGCGACCT |
| | TGACGAGCATCTGCCCGGCATTTCTGACAGCTTTGTGAACTGGG |
| | TGGCCGAGAAGGAATGGGAGCTGCCCCCGGATTCTGACATGGAT |
| | CGGAATCTGATCGAGCAGGCACCCCTGACCGTGGCCGAGAAGCT |
| | GCAGCGCGACTTCCTGGTCCACTGGCGCCGCGTGAGTAAGGCCC |
| | CGGAGGCCCTCTTCTTTGTTCAGTTCGAGAAGGGCGAGAGCTAC |
| | TTCCACCTTCACGTTCTGGTGGAGACCACGGGGGTCAAGTCCAT |
| | GGTGCTAGGCCGCTTCCTGAGTCAGATTCGGGAGAAGCTGGTCC |
| | AGACCATCTACCGCGGGGTCGAGCCCACGCTGCCCAACTGGTTC |
| | GCGGTGACCAAGACGCGTAATGGCGCCGGCGGGGGGAACAAGGT |
| | GGTGGACGAGTGCTACATCCCCAACTACCTGCTCCCCAAGACCC |
| | AGCCCGAGCTCCAGTGGGCGTGGACTAATATGGACCAGTATTTA |
| | AGCGCCTGTTTGAATCTCACGGAGCGTAAACGGTTGGTGGCGCA |
| | GCATCTGACGCACGTGTCGCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGCGG |
| | GATCACGTCAGAAAAGCAATGGATCCAGGAGGACCAGGCGTCCT |
| | ACATCTCCTTCAACGCCGCCTCCAACTCGCGGTCACAAATCAAG |
| | GCCGCGCTGGACAATGCCTCCAAAATCATGAGCCTGACAAAGAC |
| | GGCTCCGGACTACCTGGTGGGCCAGAACCCGCCGGAGGACATTT |
| | CCAGCAACCGCATCTACCGAATCCTCGAGATGAACGGGTACGAT |
| | CCGCAGTACGCGGCCTCCGTCTTCCTGGGCTGGGCGCAAAAGAA |
| | GTTCGGGAAGAGGAACACCATCTGGCTCTTTGGGCCGGCCACGA |
| | CGGGTAAAACCAACATCGCGGAGGCCATCGCCCACGCCGTGCCC |
| | TTCTACGGCTGCGTCAACTGGACCAATGAGAACTTTCCCTTCAA |
| | CGACTGCGTCGACAAGATGGTGATCTGGTGGGAGGAGGGCAAGA |
| | TGACGGCCAAGGTCGTAGAGAGCGCCAAGGCCATCCTGGGCGGA |
| | AGCAAGGTGCGCGTGGACCAAAAGTGCAAGTCGTCCGCCCAGAT |
| | CGATCCCACCCCCGTGATCGTCACCTCCAACACCAACATGTGCG |
| | CCGTGATTGACGGGAACAGCACCACCTTCGAGCACCAGCAGCCG |
| | TTGCAGGACCGGATGTTCAAATTTGAACTCACCCGCCGTCTGGA |
| | GCATGACTTTGGCAAGGTGACAAAGCAGGAAGTCAAAGAGTTCT |
| | TCCGCTGGGCCAGTGATCACGTGACCGAGGTGGCGCATGAGTTT |
| | TACGTCAGAAAGGGCGGAGCCAGCAAAAGACCCGCCCCCGATGA |
| | CGCGGATAAAAGCGAGCCCAAGCGGGCCTGCCCCTCAGTCGCGG |
| | ATCCATCGACGTCAGACGCGGAAGGAGCTCCGGTGGACTTTGCC |
| | GACAGGTACCAAAACAAATGTTCTCGTCACGCGGGCATGCTTCA |
| | GATGCTGTTTCCCTGCAAGACATGCGAGAGAATGAATCAGAATT |
| | TCAACATTTGCTTCACGCACGGGACGAGAGACTGTTCAGAGTGC |
| | TTCCCCGGCGTGTCAGAATCTCAACCCGTGTCTGTCGTCAGAAA |
| | GCGGACATATCAGAAACTGTGTCCGATTCATCACATCATGGGGA |
| | GGGCGCCCGAGGTGGCTTGTTCGGCCTGCGATCTGGCCAATGTG |
| | GACTTGGATGACTGTGACATGGAGCAATAA |
| 47-clone 2.41 | ATGCCGGGCTTCTACGAGATCGTGATCAAGGTGCCGAGCGACCT |
| | GGACGAGCACCTGCCGGGCATTTCTGACTCGTTTGTGAACTGGG |
| | TGGCCGAGAAGGAATGGGAGTTGCCGCCAGATTCTGACATGGAT |
| | CTGAATCTGATTGAGCAGGCACCCCTGACCGTGGCCGAGAAGCT |
| | GCAGCGCGACTTCCTGGTCCAGTGGCGCCGCGTGAGTAAGGCCC |
| | CGGAGGCCCTCTTCTTTGTTCAGTTCGAGAAGGGCGAGTCCTAC |
| | TTCCACCTCCATATTCTGGTGGAGACCACGGGGGTCAAATCCAT |
| | GGTGCTGGGCCGCTTCCTGAGTCAGATTAGGGACAAGCTGGTGA |
| | CCCGCATCTACCGCGGGGTCGAGCCGCAGCTTCCGAACTGGTTC |
| | GCGGTGACCAAGACGCGTAATGGCGCCGGAGGCGGGAACAAGGT |
| | GGTGGACGACTGCTACATCCCCAACTACCTGCTCCCCAAGACCC |
| | AGCCCGAGCTGCAGTGGGCGTGGACTAACATGGAGGAGTATATA |
| | AGCGCGTGTCTGAACCTCGCGGAGCGTAAACGGCTCGTGGCGCA |
| | GCACCTGACCCACGTCAGCCAGACGCAGGAGCAGAACAAGGAGA |
| | ATCTGAACCCGAATTCTGACGCGCCCGTGATCAGGTCAAAAACC |
| | TCCGCGCGCTACATGGAGCTGGTCGGGTGGCTGGTGGACCGGGG |
| | CATCACCTCCGAGAAGCAGTGGATCCAGGAGGACCAGGCCTCGT |
| | ACATCTCCTTCAACGCCGCCTCCAACTCGCGGTCCCAGATCAAG |
| | GCCGCGCTGGACAATGCCTCCAAGATCATGAGCCTGACAAAGAC |
| | GGCTCCGGACTACCTGGTGGGCAGCAACCCGCCGGAGGACATTT |
| | CCAGCAACCGGATCTACAAAATCCTCGAGATGAACGGGTACGAT |
| | CCGCAGTACGCGGCCTCCGTCTTTCTCGGCTGGGCACAAAAGAA |
| | GTTCGGGAAACGCAACACCATCTGGCTGTTTGGGCCGGCCACCA |
| | CGGGAAAGACCAACATCGCAGAAGCCATTGCCCACGCCGTGCCC |
| | TTCTACGGCTGCGTCAACTGGACCAATGAGAACTTTCCCTTCAA |
| | CGATTGCGTCGACAAGATGGTGATCTGGTGGGAGGAGGGCAAGA |
| | TGACGGCCAAGGTCGTGGAGTCCGCCAAGGCCATTCTGGGCGGA |
| | AGCAAGGTGCGCGTGGACCAAAAGTGCAAGTCGTCCGCCCAGAT |
| | CGACCCCACCCCCGTGATCGTCACCTCCAACACCAACATGTGCG |
| | CCGTGATCGACGGGAACAGCACCACCTTCGAGCACCAGCAGCCG |
| | CTGCAGGACCGCATGTTCAAGTTCGAGCTCACCCGCCGTCTGGA |
| | GCACGACTTTGGCAAGGTGACCAAGCAGGAAGTCAAAGAGTTCT |
| | TCCGCTGGGCGCAGGATCACGTGACCGAGGTGTCTCACGAGTTT |
| | TACGTCAGAAAGGGTGGAGCTAGAAAGAGGCCCGCCCCCAATGA |
| | CGCAGATATAAGTGAGCCCAAGCGGGCCTGTCCGTCAGTTGCGC |
| | AGCCATCGACGTCAGACGCGGAAGCTCCGGTGGACTACGCGGAC |
| | AGGTACCAAAACAAATGTTCTCGTCACGTGGGTATGAATCTGAT |
| | GCTTTTTCCCTGCCGGCAATGCGAGAGAATGAATCAGAATGTGG |
| | ACATTTGCTTCACACACGGGGTCAGAGACTGCTCAGAGTGTTTC |
| | CCCGGCGTGTCAGAATCTCAACCGGTCGTCAGAAAAAAGACGTA |
| | TCAGAAACTGTGTCCGATTCATCACATCATGGGGAGGGCGCCCG |
| | AGGTGGCCTGCTCGGCCTGCGAACTGGCCAATGTGGACTTGGAT |
| | GACTGTGACATGGAACAATAA |
| 8-Primer_fwd | AGCGCATTGCGTAGAATAC |
| 9-Primer_rev | CTGCGTGGACACTCACTT |
| 10-Primer_fw_ | TGAAGCGGCGCGCCGAATGCGGGAGGTTTGAACGCGC |
| AscI_BsmI | |
| 48-Primer_rev_ | TTAGTATTAATTAAGAATGCGAGCCAATCTGGAAGATAACC |
| PacI_BsmI | |
| 49-clone 0.15 AA | MPGFYEIVLKVPSDLDEHLPGISNSFVNWVAEKEWELPPDSDMD |
| | RNLIEQAPLTVAEKLQRDFLVQWRRVSKAPEALFFVQFEKGESY |
| | FHLHVLVETTGVKSMVLGRFLSQIRDRLVQTIYRGVEPTLPNWF |
| | AVTKDAVMAPAGGNKVVDECYIPNYLLPKTQPELQWAWTNMEEY |
| | ISACLNLAERKRLVAQHLTHVSQTQEQNKENLNPNSDAPVIRSK |
| | TSARYMELVGWLVDRGITSEKQWIQEDQASYISFNAASNSRSQI |
| | KAALDNAGKIMALTKSAPDYLVGPSLPADIKANRIYRILELNGY |
| | DPAYAASVFLGWAQKKFGKRNTIWLFGPATTGKTNIAEAIAHAV |
| | PFYGCVNWTNENFPFNDCVDKMVIWWEEGKMTAKVVESAKAILG |
| | GSKVRVDQKCKSSAQIDPTPVIVTSNTNMCAVIDGNSTTFEHQQ |
| | PLQDRMFKFELTRRLDHDFGKVTKQEVKDFFRWAQDHVTEVAHE |
| | FYVRKGGANKRPAPSDADISEPKRACPSVPEPSTSDAEAPVDFA |
| | DRYQNKCSRHAGMLQMLFPCKTCERMNQNFNVCFTHGVRDCSEC |
| | FPGASESQPVVRKKTYQKLCAIHHLLGRAPEIACSACDLVNVDL |
| | DDXVSEQ |
| 50-clone 1.01 AA | MPGFYEIVLKVPSDLDEHLPGISNSFVNWVAEKEWELPPDSDMD |
| | LNLIEQAPLTVAEKLQRDFLVHWRRVSKAPEALFFVQFEKGESY |
| | FHLHILVETTGVKSMVLGRFLSQIRDKLVQTIYRGIEPTLPNWF |
| | AVTKTRNGAGGGNKVVDECYIPNYLLPKTQPELQWAWINMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNASKIMSLTKTAPDYLIGQQPVGDITTNRIYKILELNGYD |
| | PQYAASVFLGWAQKRFGKRNTIWLFGPATTGKTNIAEAIAHAVP |
| | FYGCVNWTNENFPFNDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLDHDFGKVTKQEVKDFFRWASDHVTEVTHEF |
| | YVRKGGASKRPAPDDADKSEPKRACPSVAQPSTSDAEAPVDYAD |
| | RYQNKCSRHVGMNLMLFPCRQCERMNQNVDICFTHGVMDCAECF |
| | PVSESQPVSVVRKRTYQKLCLIHHIMGRAPEVACSACELANVDL |
| | DDCDMEQ |
| 51-clone 1.03 AA | MPGFYEIVIKVPSDLDEHLPGISDSFVNWVAEKEWELPPDSDMD |
| | LNLIEQAPLTVAEKLQRDFLVQWRRVSKAPEALFFVQFEKGESY |
| | FHLHVLVETTGVKSMVLGRFLSQIREKLVQTIYRGIEPTLPNWF |
| | AVTKTRNGAGGGNKVVDECYIPNYLLPKTQPELQWAWTNMEEYI |
| | SACLNLAERKRLVAQHLTHVSQTQEQNKENLNPNSDAPVIRSKT |
| | SARYMELVGWLVDRGITSEKQWIQEDQASYISFNAASNSRSQIK |
| | AALDNASKIMSLTKTAPDYLVGSNPPEDITKNRIYQILELNGYD |
| | PQYAASVFLGWAQKKFGKRNTIWLFGPATTGKTNIAEAIAHAVP |
| | FYGCVNWTNENFPENDCVDKMVIWWEEGKMTAKVVESAKAILGG |
| | SKVRVDQKCKSSAQIDPTPVIVTSNTNMCAVIDGNSTTFEHQQP |
| | LQDRMFKFELTRRLEHDFGKVTKQEVKEFFRWAQDHVTEVAHEF |
| | YVRKGGATKRPAPSDADISEPKRACPSVPEPSTSDAEAPVDFAD |
| | RYQNKCSRHAGMLQMLFPCKTCERMNQNENVCFTHGVRDCSECF |
| | PGVSESQPVSVVRKRTYQKLCPIHHIMGRAPEIACSACDLVNVD |
| | LDDCVSEQ |
| 52-clone 1.10 AA | MPGFYEIVIKVPSDLDEHLPGISDSFVNWVAEKEWELPPDSDMD |
| | LNLIEQAPLTVAEKLQREFLVEWRRVSKAPEALFFVQFEKGETY |
| | FHLHVLIETIGVKSMVVGRYVSQIKEKLVTRIYRGVEPQLPNWF |
| | AVTKTRNGAGGGNKVVDDCYIPNYLLPKTQPELQWAWINMEEYI |
| | SACLNLAERKRLVAQHLTHVSQTQEQNKENLNPNSDAPVIRSKT |
| | SARYMELVGWLVDKGITSEKQWIQEDQASYISFNAASNSRSQIK |
| | AALDNASKIMSLTKTAPDYLVGQNPPEDITSNRIYKILEMNGYD |
| | PQYAASVFLGWAQKKFGKRNTIWLFGPATTGKTNIAEAIAHAVP |
| | FYGCVNWTNENFPFNDCVDKMVIWWEEGKMTAKVVESAKAILGG |
| | SKVRVDQKCKASAQIDPTPVIVTSNTNMCAVIDGNSTTFEHQQP |
| | LQDRMFKFELTRRLEHDFGKVTKQEVKEFFRWASDHVTEVSHEF |
| | YVRKGGANKRPAPDDADKSEPKRACPSVAEPSTSDAEAPVDFAD |
| | RYQNKCSRHAGMLQMLFPCRQCERMNQNSNICFTHGQKDCLECF |
| | PVSESQPVSVVKKAYQKLCYIHHIMGKVPDACTACDLVNVDLDD |
| | CIFEQ |

The rep sequences comprise an N-terminal domain (n), a DNA binding domain (d), and a helicase domain (h), a NLS/p40 promoter domain (y) and a Zinc finger domain (z).

| | n domain | d domain | h domain | y domain | z domain |
|---|---|---|---|---|---|
| AAV | nt | nt | nt | nt | nt |
| serotype | AA | AA | AA | AA | AA |
| 1 | 1-306 | 307-726 | 727-1107 | 1108-1590 | 1591-1872 |
| | 1-102 | 103-242 | 243-369 | 370-530 | 531-623 |
| 2 | 1-306 | 307-726 | 727-1107 | 1108-1587 | 1588-1866 |
| | 1-102 | 103-242 | 243-369 | 370-529 | 530-621 |
| 3 | 1-306 | 307-726 | 727-1107 | 1108-1587 | 1588-1875 |
| | 1-102 | 103-242 | 243-369 | 370-529 | 530-624 |
| 4 | 1-306 | 307-726 | 727-1107 | 1108-1587 | 1588-1872 |
| | 1-102 | 103-242 | 243-369 | 370-529 | 530-623 |
| 5 | 1-306 | 307-714 | 715-1095 | 1096-1632 | 1633-1833 |
| | 1-101 | 102-238 | 239-365 | 366-544 | 545-610 |
| 6 | 1-306 | 307-726 | 727-1107 | 1108-1590 | 1591-1872 |
| | 1-102 | 103-242 | 243-369 | 370-530 | 531-623 |
| 7 | 1-306 | 307-726 | 727-1107 | 1108-1590 | 1591-1872 |
| | 1-102 | 103-242 | 243-369 | 370-530 | 531-623 |
| 8 | 1-306 | 307-732 | 733-1113 | 1114-1596 | 1597-1878 |
| | 1-102 | 103-242 | 243-369 | 370-532 | 533-623 |
| 9 | 1-306 | 307-726 | 727-1107 | 1108-1590 | 1591-1872 |
| | 1-102 | 103-242 | 243-369 | 370-530 | 531-623 |
| 10 | 1-306 | 307-726 | 727-1107 | 1108-1590 | 1591-1869 |
| | 1-102 | 103-242 | 243-369 | 370-530 | 531-622 |
| 11 | 1-306 | 307-726 | 727-1107 | 1108-1590 | 1591-1869 |
| | 1-102 | 103-242 | 243-369 | 370-530 | 531-622 |
| 12 | 1-306 | 307-726 | 727-1107 | 1108-1590 | 1591-1866 |
| | 1-102 | 103-242 | 243-369 | 370-530 | 531-621 |
| 13 | 1-306 | 307-726 | 727-1107 | 1108-1590 | 1591-1872 |
| | 1-102 | 103-242 | 243-369 | 370-530 | 531-623 |

### EXAMPLES

### Example 1

### Cloning of Replication-Competent and -Incompetent Acceptor Plasmids for Rep ORF Cloning

The pWTAAV2_2×BsmI_Cap2 acceptor plasmid contained two AAV2 ITRs flanking a ccdb gene and the AAV2 cap ORF. The ccdb gene (see, e.g., Bernard, P., Biotechniques 21 (1996) 320-323) is of bacterial origin and part of a type II toxin-antitoxin system and has been used to increase cloning efficiency. The ccdb gene was flanked by inverted BsmI sites to allow the seamless cloning of rep ORFs. A gene block containing the whole sequence was ordered from GeneWiz, NJ, USA. The replication-incompetent pAAV2_2×BsmICap2, i.e. lacking AAV2 ITRs, was generated from pWTAAV2_2×BsmI_Cap2 by PCR amplifying the complete region between AAV2 ITRs and cloning (SnabI/EcoRV) into an acceptor plasmid that contains a constitutive minimal p5 promoter proximal to rep and a distal full-length constitutive p5 promoter at the end of the cap ORF (distal to rep) (see, e.g., U.S. Pat. No. 5,622,856).

### Example 2

### Cloning of Rep 1-13 ORFs and Hybrid Rep Library

Gene blocks of the rep genes originating from AAV serotypes 1-13 were ordered at GeneArt (Thermo Fisher Scientific, Waltham, MA, USA). The rep gene sequences were derived from the NCBI GenBank entries: rep1: NC002077 (SEQ ID NO: 11); rep2: NC001401 (SEQ ID NO: 12); rep3: U48704 (SEQ ID NO: 13); rep4: NC001829 (SEQ ID NO: 14); rep5: NC006152 (SEQ ID NO: 15); rep6: AF028704 (SEQ ID NO: 16); rep7: NC006260 (SEQ ID NO: 17); rep8: NC006261 (SEQ ID NO: 18); rep9: AX753250 (SEQ ID NO: 19); rep10: AY631966 (SEQ ID NO: 20); rep11: AY631965 (SEQ ID NO: 21); rep12: DQ813647 (SEQ ID NO: 22); rep13: EU285562 (SEQ ID NO: 23). To allow the cloning of Rep3 using BsmI, a silent mutation A>G was introduced at nucleotide position 1827. Rep gene blocks were flanked with sequences from the AAV2 genome to enable the use of common primers for amplification (Primer_fwd: AGC GCA TTG CGT AGA ATA C (SEQ ID NO: 8), Primer_rev: CTG CGT GGA CAC TCA CTT (SEQ ID NO: 9)). The resulting PCR amplicons were separated using agarose gel electrophoresis according to standard procedures and purified with the QIAquick PCR Purification Kit (Qiagen, Hilden, Germany) according to manufacturers' instructions. The PCR amplicons served as input for both individual rep ORF amplification and DNA family shuffling. Two other sets of primers were used to introduce restriction enzyme recognition sites into the rep amplicons (for both individual rep 1-13 and hybrid rep ORFs): Primer_fw_AscI_BsmI: TGA AGC GGC GCG CCG AAT GCG GGA GGT TTG AAC GCG C SEQ ID NO: 10); Primer_rev_PacI_BsmI: TTA GTA TTA ATT AAG AAT GCG AGC CAA TCT GGA AGA TAA CC (SEQ ID NO: 48)). AcsI/PacI double digest were performed and the digested PCR amplicons were cloned into a plasmid backbone containing only an ampicillin resistance gene and a multiple cloning site. From this plasmid subcloning of the individual rep ORFs or amplified rep library was performed using BsmI into the final pAAV2_2×BsmICap2 or pWTAAV2_2×BsmI_Cap2 plasmid, respectively.

### Example 3

### DNA Family Shuffling of Rep ORFs

DNA family shuffling was performed according to the art (see, e.g., [36]). In brief, PCR-amplified rep gene blocks (see Example 2) were pooled (total 4 μg) and subjected to a DNAseI digest using the conditions shown in FIG. 2A. Fragments between 100 bp and 1000 bp were extracted from the agarose gel and purified with the NucleoSpin Gel and PCR Clean-up kit according to manufacturer's instructions (Macherey-Nagel, Düren, Germany). From the digest, 500 ng were used as template in a primerless PCR (98° C. for 30 sec; 40 cycles of 98° C. for 10 sec, 42° C. for 30 sec, and 72° C. for 45 sec; and final elongation at 72° C. for 10 min), followed by an amplification of the final approx. 1900 bp hybrid rep ORFs using the above-mentioned primers Primer_fw_AscI_BsmI (SEQ ID NO: 10)/Primer_rev_PacI_BsmI (SEQ ID NO: 48) that bind to common regions outside of the hybrid rep ORFs (98° C. for 30 sec; 40 cycles of 98° C. for 10 sec, 64° C. for 30 sec, and 72° C. for 60 sec; and final elongation at 72° C. for 5 min). The final amplicons were cloned as described in Example 2.

### Example 4

### Cell Culture

Expi293FTM suspension cells were grown in Expi293TM Expression Medium (Thermo Fisher Scientific) and maintained at 37° C. with 8% CO₂ according to supplier's instructions. HEK293T and HEK293A cells were grown in Dulbecco's modified Eagle's medium (DMEM) with high glucose (Thermo Fisher Scientific) supplemented with fetal bovine serum (Merck, Darmstadt, Germany), sodium pyruvate and GlutaMAX (both Thermo Fisher Scientific). Cells were grown at 37° C. with 5% CO₂.

### Example 5

### Recombinant AAV Particle Production

Small Scale Production of rAAVps in adherent HEK293T cells was performed as previously described using a standard triple transfection protocol including a pRepxCap plasmid (x=1-13 or hybrid Rep variant), the Adenohelper (Takara Bio, Kusatsu, Japan) and a transgene flanked by AAV2 ITRs (either GFP or a therapeutic gene).

For rAAVp production in suspension cells, Expi293FTM cells were grown at a density of 1.8×1E6 cells per mL in the following formats and volumes: Microscale in 4 mL cultures, 24-well plate; Miniscale in 30 mL shake flasks; AMBR15 in 15 mL bioreactor, AMBR250 in 250 mL bioreactor. Transfection was performed using the same plasmids listed above for rAAV production in adherent cells and according to a previously-described protocol [42].

When using adherent cells, all assays were performed with crude cell lysates. The lysates were generated by centrifuging cells (at 800×g, 10 min.) and resuspending the pellet in PBS. The cell suspensions were transferred each to individual thin-walled PCR-tubes and placed at 4° C. in a bath sonicator (Q700MPXC Microplate Horn System, QSonica, CT, Newtown, USA). Samples were sonicated for 3 min. with 30 sec on-off-increments at 25% amplitude (intensity).

When using suspension cultures, the assays were performed from crude lysates. The lysates were generated by chemical lysis (CG110, 1%). For transduction assays, however, crude lysates were also generated by sonication as the lysis conditions interfered with the assay.

### Example 6

### Recombinant AAV Particle Purification

Crude cell lysates were centrifuged for 5 min. at 1000×g at room temperature and subsequently purified on columns containing 80 μL of AAVX resin per column (Biotage, Uppsala, Sweden; PTR-91-80-33). Purification was performed according to the manufacturer's instructions using buffers recommended in the manufacturer's protocol. To increase particle concentration for Cryo-EM analysis, final elution fractions and individual columns were re-used for 3 runs of purification. PBS was used as a negative control during the purification process.

### Example 7

### Transduction Assay

For transduction of HEK293A cell monolayers with rAAVps, cells were seeded in 96-well plates one day prior to transduction at a density of 2.0×1E4 cells per well. Next, 10 μL of crude cell lysates (stock or diluted 1:10 in PBS) were added individually to each well. After 72 h, the medium was removed and the cells subjected to flow cytometry analysis.

### Example 8

### Droplet Digital Polymerase Chain Reaction

To determine the amount of encapsidated viral genomes per mL (vg/mL), droplet digital (dd) PCR was performed. Therefore, 10 μL of crude cell lysates (generated by sonication or chemical lysis) were subjected to a pre-treatment with DNAseI (RQ1 RNase-free #M610A, Promega, WI, Madison, USA) to remove plasmid DNA, followed by Proteinase K digest (New England Biolabs, Ipswich, USA) to open the rAA Vp (capsids) as previously described [43]. Droplets were generated in the Droplet Digital PCR System according to manufacturer's instructions (QX200 AutoDG, Bio-Rad Laboratories, CA, Hercules, USA). PCR was performed in a Thermal Cycler (C1000, Bio-Rad Laboratories) and final quantification of viral genomes in the Droplet Reader (Bio-Rad Laboratories).

Data presented in Table 6 was generated using the QIAcuity digital (dPCR) system following the manufacturer's instructions (Qiagen). The same pre-treatment steps as described above (DNase I digestion, Proteinase K digestion) were applied to remove contaminating plasmid and genomic DNA. Control singleplex dPCR reactions were con-ducted to evaluate the performance of individual primer/probe sets. Subsequently, all three primer/probe sets-targeting the 5′ end, 3′ end, and mid-region of the genome-were combined in a triplex reaction.

The QIAcuity Software Suite (version 2.5.0.1) provides a downloadable multi-ple-occupancy CSV file. This file incorporates mathematical approximations based on Poisson distribution statistics to estimate the percentage of intact viral genomes. For both viral titers and genome integrity analyses, the average value from four different dilutions was calculated for each replicate. Final reported values represent the mean of two independent replicates. Primer sets used to quantify the rAAV-GFP genome are shown Table 9.

TABLE 9
Primer/probe sets used for singleplex and multiplex dPCR analysis of rAAV titer.

| target | Primer/probe name | Sequence (5′→3′) | SEQ ID NO |
|---|---|---|---|
| 5′ end | 5′GFP_CMVenh5_F | TTGACGTCAATGGGTGGAGT | 53 |
| | 5′GFP_CMVenh5_R | CGGGCCATTTACCGTCATTG | 54 |
| | 5′GFP_CMV3nh5_P | ACATCAAGTGTATCATATGCCAAGTACGC | 55 |
| 3′ end | 3′GFP_BGHpolA_F | [CY5]CATTGTCTGAGTAGGTGTCATTC[BHQ2] | 56 |
| | 3′GFP_BGHpolA_R | TGCCTGCTATTGTCTTCCCA | 57 |
| | 3′GFP_BGHpolA_P | [HEX]CCTCCCCCTTGCTGTCCTGC[BHQ1] | 58 |
| middle | eGFP_mid_F | TAGCCGCTACCCTGATCATA | 59 |
| | eGFP_mid_R | TAAAGAAGATGGTCCGCTCC | 60 |
| | eGFP_mid_P | [FAM]TCTTTAAGTCCGCTATGCCAGAAGG[BHQ1] | 61 |

### Example 9

### Flow Cytometry Analysis of Transduction and Transfection

Efficiencies of transfection and transduction of cells were evaluated via flow cytometry (FACSCanto II, BD Biosciences, NJ, Franklin Lakes, USA) using green fluorescent protein (GFP) as marker. Therefore, transduced HEK293A cells were detached using 25 μL of 0.25% Trypsin/EDTA solution. Thereafter the cells were resuspended in 175 μL PBS supplemented with 1% bovine serum albumin (BSA). Next, 100 μL of the cell suspension was measured by flow cytometry. Expi293TM suspension growing cells that were used for recombinant or wild-type AAV production were also analyzed for their transfection efficiency by including a control transfection with a GFP expression plasmid. Flow cytometry measurements were performed72 h after transfection or transduction. Therefore, 100 μL of the cell suspension was directly pipetted into a 96 well plate and used for the flow cytometry measurement.

### Example 10

### Western Blot Analysis

A Western Blot Analysis was performed to detect AAV VP1-3 or Rep protein expression during rAAVp production.

Capillary electrophoresis (Jess) was used for separation of proteins for high sensitivity. Therefore, suspension cells were centrifuged for 3 min. at 300×g, washed with PBS and lysed using 100 μL RIPA buffer, supplemented with 1× Pierce™ Protease Inhibitor Mini tablets, EDTA-Free (Thermo Fisher Scientific). Lysates were incubated on ice for 20 min., sonicated for 3 min. and thereafter centrifuged at 20,000×g for 15 min. Supernatants were collected, and protein concentrations were determined using the BCA protein assay kit according to the manufacturer's instructions (Thermo Fisher Scientific). Prior to electrophoresis, protein samples were prepared according to manufacturer's instructions (ProteinSimple, part of Bio-Techne, San Jose, CA, USA). Final concentration was set to 0.5 mg/mL. The 12-230 kDa Separation Module was used for the detection of Rep proteins and the 66-440 kDa Separation Module was used for the detection of Cap proteins.

A monoclonal mouse anti-AAV2 Replicase antibody (clone 303.9, lyophilized, purified; PROGEN) was used as primary antibody for detection of Rep proteins and a polyclonal rabbit anti-AAV VP1/VP2/VP3 antibody (VP51, serum; PROGEN) was used as primary antibody for detection of Cap proteins. Both antibodies were used at a 1:100 dilution in Antibody Diluent 2 (ProteinSimple, part of Bio-Techne). For the detection of the housekeeping protein Glycerinalde-hyd-3-phosphat-Dehydrogenase (GAPDH), a monoclonal mouse antibody (GAPDH (D4C6R) Mouse mAb #97166; Cell Signaling) was used at a 1:25 dilution in Antibody Diluent 2 (ProteinSimple, part of Bio-Techne).

The Anti-Mouse Detection Module (ProteinSimple, part of Bio-Techne) was used for the detection of Rep proteins and the Anti-Rabbit Detection Module (ProteinSimple, part of Bio-Techne) was used for the detection of Cap proteins. The total protein detection kit (ProteinSimple, part of Bio-Techne) was used for normalization of protein concentrations and prepared according to manufacturers' instructions.

The RePlex kit (ProteinSimple, part of Bio-Techne) was used to allow binding of the total protein detection antibody and was prepared according to manufacturer's instructions (ProteinSimple, part of Bio-Techne).

For the separation of Rep and Cap proteins the Jess system was programmed according to the following protocol: Separation time, 28 min.: Separation Voltage, 375 volts; RePlex Purge Time, 30 min.; Biotin Labeling Time, 30 min-; Antibody Diluent Time, 5 min.; Primary Antibody Time, 60 min.; Secondary Antibody Time, 30 min. Total Protein HRP Time, 30 min. Chemiluminescent signals were detected and quantified using the Compass software (ProteinSimple, part of Bio-Techne). Protein bands were identified based on the molecular weight ladder and peak intensities were normalized using the total protein detection kit.

For the separation of Rep and GAPDH the Jess system was programmed according to the following protocol: Separation time, 30 min: Separation Voltage, 375 volts; Biotin Labeling Time, 30 min; Antibody Diluent Time; 5 min; Primary Antibody Time, 60 min; Secondary Antibody Time, 30 min.

The results are shown in FIG. 6A.

### Example 11

### Enzyme Linked Immunosorbent Assay and Electrochemiluminescence Immunoassay

Enzyme linked Immunosorbent Assay (ELISA) was performed to detect total AAV capsids in cell lysates. Therefore, the CaptureSelect™ anti-AAV affinity reagents were used according to the manufacturer's instructions. In brief, streptavidin-coated 96-well plates (Pierce, Thermo Fisher Scientific) were incubated with 100 μL/well anti-AAVX antibody conjugated to biotin diluted 1:4000 in 1×PBS (#7103522100, Thermo Fisher Scientific) for 2 hours at room temperature. rAAVp containing lysates and a standard control (AAV2-CMV-GFP, Virovek, CA, Hayward, USA) were diluted using 1×PBST. Samples were measured in duplicates and at different dilutions to fit in the limited linear range of the standard curve. For detection, 100 μL anti-AAVX antibody conjugated to HRP diluted 1:10,000 in 1×PBST (#7303522100, Thermo Fisher Scientific) was applied to each well and incubated for 1 hour at room temperature. The color development reagent (Ultra TMB ELISA substrate, Thermo Fisher Scientific) was used at a volume of 100 μL/well (incubation 15 min.). The reaction was stopped by addition of 100 μL of 1 M HCl and the OD was measured at a wavelength of 450 nm using a microtiter plate reader (Infinite 200 PRO, Tecan, Maennedorf, Switzerland).

The Electrochemiluminescence Immunoassay (ECLIA) assay was used for increased sensitivity in the small-scale experiments (with expected lower titers) and for testing of Rep1.3 for the production of different AAV isolates. Therefore a heterogeneous sandwich assay based on AAVX (for AAV2 and AAV8 production) or anti-AAV9 antibody for AAV9 production (both Thermo Fisher Scientific) was used.

### Example 12

### Electron Microscopy

For negative staining electron microscopy (EM), electron microscopy grids (T600H-Cu 698 l/inch Hex. mesh Thin Bar; EMS) were coated with an approx. 2 nm carbon film generated by floating the carbon on H2O and letting the water level drop till the carbon covered the grids. After at least 2 days of drying, the grids were used. Three μL of sample was incubated on a glow-discharged carbon coated grid for 30 seconds, followed by 2 steps of washing with H2O, one step of washing with uranyl acetate (UAc) 2% and 30 seconds incubation in UAc 2% staining solution. After the final blotting step, the sample was left to dry at the air.

For cryo-EM, 3 μL of sample was incubated on a glow-discharged carbon coated Quantifoil grid for 60 seconds (Quantifoil−R1.2/1.3, 300, Cu+2 nm, Germany), before blotting of 2.5 or 3 seconds. Subsequently the grid was plunged into liquid Ethan at −180° C. using a Leica EM GP automated plunging device (Leica Microsystems, Vianna, Austria).

Grids were loaded into a Jeol JEM-1400 Plus transmission electron microscope operating a Lab6 electron source at 120 kV. Electron micrographs were recorded on TVIPS XF416 4000 by 4000 pixel charge-coupled device camera (Tietz Video and Image Processing System, Gauting, Germany). The negative staining images were used to check the presence of whole particles, ascertain that the particle concentration is suitable for cryo-EM and check for aggregations and impurities, prior to freezing the samples. Cryo-EM datasets were recorded at a nominal magnification of 30,000× yielding pictures with a pixel size corresponding to 0.3914 nm at the specimen level. Cryo-EM grids were imaged using low dose mode.

Forty to eighty cryo-EM images per sample were imported into the EMAN2 software package where picking and sorting of viral particles was done manually to determine the ratio between empty and full capsids. Some intermediately filled particles were observed and counted as empty, since they lack the complete genome.

1. A nucleic acid encoding a functional adeno-associated virus Rep protein, characterized in that the nucleic acid comprises at least 14 different fragments derived from naturally occurring rep genes of at least 8 different serotypes.

2. The nucleic acid of claim 1, characterized in comprising at least 17 different part fragments derived from naturally occurring rep genes of at least 8 different serotypes.

3. A nucleic acid encoding a functional adeno-associated virus Rep protein, characterized in that the nucleic acid comprises a 5′-terminal part, a central part and a 3′-terminal part, wherein the 5′-terminal part, the central part and the 3′-terminal part are independently of each other derived from naturally occurring rep genes of different serotypes.

4. The nucleic acid of claim 3, wherein
a) the 5′-terminal part is similar to a part of the rep gene of the AAV3 serotype and the 3′-terminal part is similar to a part of the rep gene of the AAV10 serotype;
b) the 5′-terminal part is similar to a part of the rep gene of the AAV3 serotype and the 3′-terminal part is similar to a part of the rep gene of the AAV13 serotype;
c) the 5′-terminal part is similar to a part of the rep gene of the AAV1 serotype and the 3′-terminal part is similar to a part of the rep gene of the AAV13 serotype;
d) the 5′-terminal part is similar to a part of the rep gene of the AAV1 serotype and the 3′-terminal part is similar to a part of the rep gene of the AAV2 serotype;
e) the 5′-terminal part is similar to a part of the rep gene of the AAV3 serotype and the 3′-terminal part is similar to a part of the rep gene of the AAV4 serotype;
f) the 5′-terminal part is similar to a part of the rep gene of the AAV6 serotype and the 3′-terminal part is similar to a part of the rep gene of the AAV4 serotype;
g) the 5′-terminal part is similar to a part of the rep gene of any AAV serotype and the 3′-terminal part is similar to a part of the rep gene of the AAV2 serotype; or
h) the 5′-terminal part is similar to a part of the rep gene of any AAV serotype and the 3′-terminal part is similar to a part of the rep gene of the AAV13 serotype; or
i) the 5′-terminal part is similar to a part of the rep gene of the AAV3 serotype and the 3′-terminal part is similar to a part of the rep gene of the AAV6 serotype; or
j) the 5′-terminal part is similar to a part of the rep gene of the AAV1 serotype and the 3′-terminal part is similar to a part of the rep gene of the AAV6 serotype.

5. The nucleic acid of claim 3, wherein the 5′-terminal part is similar to a part of the rep gene of the AAV2 or AAV6 serotype and the 3′-terminal part is similar to a part of the rep gene of the AAV13 serotype.

6. The nucleic acid of claim 3, characterized in that the 5′-terminal part comprises in 5′- to 3′-direction a first 5′-terminal part and a second 5′-terminal part and the 3′-terminal part comprises in 5′- to 3′-direction a second 3′-terminal part and a first 3′-terminal part.

7. The nucleic acid of claim 3, wherein
a) the first 5′-terminal part is similar to a part of the rep gene of the AAV3 serotype and the second 5′-terminal part is similar to a part of the rep gene of the AAV6 serotype and the second 3′-terminal part is similar to a part of the rep gene of the AAV11 serotype and the first 3′-terminal part is similar to a part of the rep gene of the AAV10 serotype;
b) the first 5′-terminal part is similar to a part of the rep gene of the AAV3 serotype and the second 5′-terminal part is similar to a part of the rep gene of the AAV4 serotype and the second 3′-terminal part is similar to a part of the rep gene of the AAV4 serotype and the first 3′-terminal part is similar to a part of the rep gene of an AAV13 serotype;
c) the first 5′-terminal part is similar to a part of the rep gene of the AAV3 serotype and the second 5′-terminal part is similar to a part of the rep gene of the AAV10 serotype and the second 3′-terminal part is similar to a part of the rep gene of the AAV11 serotype and the first 3′-terminal part is similar to a part of the rep gene of the AAV4 serotype;
d) the first 5′-terminal part is similar to a part of the rep gene of the AAV6 serotype and the second 5′-terminal part is similar to a part of the rep gene of the AAV10 serotype and the second 3′-terminal part is similar to a part of the rep gene of the AAV11 serotype and the first 3′-terminal part is similar to a part of the rep gene of an AAV4 serotype;
e) the first 5′-terminal part is similar to a part of the rep gene of any AAV serotype and the second 5′-terminal part is similar to a part of the rep gene of the AAV1 serotype and the second 3′-terminal part is similar to a part of the rep gene of the AAV10 serotype and the first 3′-terminal part is similar to a part of the rep gene of the AAV2 serotype; or
f) the first 5′-terminal part is similar to a part of the rep gene of any AAV serotype and the second 5′-terminal part is similar to a part of the rep gene of the AAV1 serotype and the second 3′-terminal part is similar to a part of the rep gene of any AAV serotype and the first 3′-terminal part is similar to a part of the rep gene of an AAV13 serotype; or
g) the first 5′-terminal part is similar to a part of the rep gene of the AAV3 serotype and the second 5′-terminal part is similar to a part of the rep gene of the AAV1 serotype and the second 3′-terminal part is similar to a part of the rep gene of any AAV serotype and the first 3′-terminal part is similar to a part of the rep gene of an AAV13 serotype; or
h) the first 5′-terminal part is similar to a part of the rep gene of the AAV3 serotype and the second 5′-terminal part is similar to a part of the rep gene of the AAV1 serotype and the second 3′-terminal part is similar to a part of the rep gene of any AAV serotype and the first 3′-terminal part is similar to a part of the rep gene of an AAV6 serotype; or.

8. The nucleic acid of claim 3, wherein the first 5′-terminal part is similar to a part of the rep gene of the AAV2 or AAV6 serotype and a second 5′-terminal part is similar to a part of the rep gene of an AAV1 serotype and the second 3′-terminal part is similar to a part of the rep gene of the AAV10 or AAV11 serotype and a first 3′-terminal part is similar to a part of the rep gene of an AAV13 serotype.

9. The nucleic acid of claim 3, wherein the central part comprises in 5′ to 3′ direction a first part that is similar to a part of the rep gene of the AAV10 serotype, a second part that is similar to a part of the rep gene of the AAV3 serotype, a third part that is similar to a part of the rep gene of the AAV13 serotype. a fourth part that is similar to a part of the rep gene of the AAV9 serotype, a fifth part that is similar to a part of the rep gene of the AAV13 serotype and a sixth part that is similar to a part of the rep gene of the AAV11 serotype.

10. The nucleic acid according to claim 9, wherein the 5′-terminal part is similar to a part of the rep gene of the AAV1 or AAV6 serotype and the 3′-terminal part is similar to a part of the rep gene of the AAV4 serotype.

11.-13. (canceled)

14. An adeno-associated virus Rep protein with the amino acid sequence of
(SEQ ID NO: 49)
MPGFYEIVLK VPSDLDEHLP GISNSFVNWV AEKEWELPPD
SDMDRNLIEQ APLTVAEKLQ RDFLVQWRRV SKAPEALFFV
QFEKGESYFH LHVLVETTGV KSMVLGRFLS QIRDRLVQTI
YRGVEPTLPN WFAVTKDAVM APAGGNKVVD ECYIPNYLLP
KTQPELQWAW TNMEEYISAC LNLAERKRLV AQHLTHVSQT
QEQNKENLNP NSDAPVIRSK TSARYMELVG WLVDRGITSE
KQWIQEDQAS YISFNAASNS RSQIKAALDN AGKIMALTKS
APDYLVGPSL PADIKANRIY RILELNGYDP AYAASVFLGW
AQKKFGKRNT IWLFGPATTG KTNIAEAIAH AVPFYGCVNW
TNENFPFNDC VDKMVIWWEE GKMTAKVVES AKAILGGSKV
RVDQKCKSSA QIDPTPVIVT SNTNMCAVID GNSTTFEHQQ
PLQDRMFKFE LTRRLDHDFG KVTKQEVKDF FRWAQDHVTE
VAHEFYVRKG GANKRPAPSD ADISEPKRAC PSVPEPSTSD
AEAPVDFADR YQNKCSRHAG MLQMLFPCKT CERMNQNFNV
CFTHGVRDCS ECFPGASESQ PVVRKKTYQK LCAIHHLLGR
APEIACSACD LVNVDLDDXV SEQ.

15.-103. (canceled)


SEQUENCE LISTING (61 sequences)

SEQ ID NO: 1 - DNA, 147 bp, Adeno-associated dependoparvovirus A
  source 1..147: mol_type = genomic DNA
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaatcgc

SEQ ID NO: 2 - DNA, 145 bp, Adeno-associated dependoparvovirus A
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 3 - DNA, 146 bp, Adeno-associated dependoparvovirus A
  source 1..146: mol_type = genomic DNA
tggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggca

SEQ ID NO: 4 - DNA, 146 bp, Adeno-associated dependoparvovirus A
  source 1..146: mol_type = genomic DNA
gggcaaacctagatgatggagttggccactccctctatgcgcgctcgctcactcactcggccctgccggccagaggccggcagtctggagacctttggtctccagggccgagtgagtgagcgagcgcgcatagagggagtggccaa

SEQ ID NO: 5 - DNA, 176 bp, Adeno-associated dependoparvovirus B
  source 1..176: mol_type = genomic DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgtaagcagtgat

SEQ ID NO: 6 - DNA, 143 bp, Adeno-associated dependoparvovirus A
  source 1..143: mol_type = genomic DNA
atacccctagtgatggagttgcccactccctctatgcgcgctcgctcgctcggtggggcctgcggaccaaaggtccgcagacggcagagctctgctctgccggccccaccgagcgagcgagcgcgcatagagggagtgggcaa

SEQ ID NO: 7 - DNA, 146 bp, Adeno-associated dependoparvovirus A
  source 1..146: mol_type = genomic DNA
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccgc

SEQ ID NO: 8 - DNA, 19 bp, synthetic construct
  source 1..19: mol_type = other DNA
agcgcattgcgtagaatac

SEQ ID NO: 9 - DNA, 18 bp, synthetic construct
  source 1..18: mol_type = other DNA
ctgcgtggacactcactt

SEQ ID NO: 10 - DNA, 37 bp, synthetic construct
  source 1..37: mol_type = other DNA
tgaagcggcgcgccgaatgcgggaggtttgaacgcgc

SEQ ID NO: 11 - DNA, 1872 bp, Adeno-associated dependoparvovirus A
  source 1..1872: mol_type = genomic DNA
atgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataa

SEQ ID NO: 12 - DNA, 1866 bp, Adeno-associated dependoparvovirus A
  source 1..1866: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 13 - DNA, 1875 bp, Adeno-associated dependoparvovirus A
  source 1..1875: mol_type = genomic DNA
atgccggggttctacgagattgtcctgaaggtcccgagtgacctggacgagcacctgccgggcatttctaactcgtttgttaactgggtggccgagaaggaatgggagctgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttaaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacggcagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataa

SEQ ID NO: 14 - DNA, 1872 bp, Adeno-associated dependoparvovirus A
  source 1..1872: mol_type = genomic DNA
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataa

SEQ ID NO: 15 - DNA, 1833 bp, Adeno-associated dependoparvovirus B
  source 1..1833: mol_type = genomic DNA
atggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 16 - DNA, 1872 bp, Adeno-associated dependoparvovirus A
  source 1..1872: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataa

SEQ ID NO: 17 - DNA, 1872 bp, Adeno-associated dependoparvovirus A
  source 1..1872: mol_type = genomic DNA
atgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataa

SEQ ID NO: 18 - DNA, 1878 bp, Adeno-associated dependoparvovirus A
  source 1..1878: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataa

SEQ ID NO: 19 - DNA, 1872 bp, Adeno-associated dependoparvovirus A
  source 1..1872: mol_type = genomic DNA
atgccgggcttctacgagattgtgatcaaggtgccgagcgacctggacgagcacctgccgggcatttctgactcttttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggagaagctggtccagaccatctaccgcgggatcgagccgaccctgcccaactggttcgcggtgaccaagacgcgtaatggcgccggcggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtaggcccttcacttccggtggacattacgcagaaccgcatctaccgcatcctgcagctcaacggctacgaccctgcctacgccggctccgtctttctcggctgggcacaaaagaagttcgggaaacgcaacaccatctggctgtttgggccggccaccacgggaaagaccaacatcgcagaagccattgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccactcccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgcttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataa

SEQ ID NO: 20 - DNA, 1869 bp, Adeno-associated dependoparvovirus A
  source 1..1869: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctactttcacctgcacgttctggtcgagaccacgggggtcaagtccatggtcctgggccgcttcctgagtcagatcagagacaggctggtgcagaccatctaccgcggggtagagcccacgctgcccaactggttcgcggtgaccaagacgcgaaatggcgccggcggggggaacaaggtggtggacgagtgctacatccccaactacctcctgcccaagacgcagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaaaggaatacaatttggctgttcgggcccgccaccaccggcaagaccaacatcgcggaagccatcgcccacgccgtgcccttctacggctgcgtcaactggaccaatgagaactttcccttcaacgattgcgtcgacaagatggtgatctggtgggaggagggcaagatgaccgccaaggtcgtggagtccgccaaggccattctgggcggaagcaaggtgcgcgtcgaccaaaagtgcaagtcctcggcccagatcgaccccacgcccgtgatcgtcacctccaacaccaacatgtgcgccgtgatcgacgggaacagcaccaccttcgagcaccagcagcccctgcaggaccgcatgttcaagttcgagctcacccgccgtctggagcacgactttggcaaggtgaccaagcaggaagtcaaagagttcttccgctgggctcaggatcacgtgactgaggtgacgcatgagttctacgtcagaaagggcggagccaccaaaagacccgcccccagtgacgcggatataagcgagcccaagcgggcctgcccctcagttgcggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacctgtcgtcagaaaaaagacgtatcagaaactgtgcgcgattcatcatctgctggggcgggcacccgagattgcgtgttcggcctgcgatctcgtcaacgtggacttggatgactgtgtttctgagcaataa

SEQ ID NO: 21 - DNA, 1869 bp, Adeno-associated dependoparvovirus A
  source 1..1869: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctacttccacctccacgttctcgtcgagaccacgggggtcaagtccatggtcctgggccgcttcctgagtcagatcagagacaggctggtgcagaccatctaccgcggggtcgagcccacgctgcccaactggttcgcggtgaccaagacgcgaaatggcgccggcggggggaacaaggtggtggacgagtgctacatccccaactacctcctgcccaagacccagcccgagctgcagtgggcgtggactaacatggaggagtatataagcgcgtgtctaaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaacgcaacaccatctggctgtttgggcccgccaccaccggcaagaccaacatcgcggaagccatagcccacgccgtgcccttctacggctgcgtgaactggaccaatgagaactttcccttcaacgattgcgtcgacaagatggtgatctggtgggaggagggcaagatgaccgccaaggtcgtggagtccgccaaggccattctgggcggaagcaaggtgcgcgtggaccaaaagtgcaagtcctcggcccagatcgaccccacgcccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggctcaggatcacgtgactgaggtggcgcatgagttctacgtcagaaagggcggagccaccaaaagacccgcccccagtgacgcggatataagcgagcccaagcgggcctgcccctcagttccggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacccgtcgtcagaaaaaagacgtatcagaaactgtgcgcgattcatcatctgctggggcgggcacccgagattgcgtgttcggcctgcgatctcgtcaacgtggacttggatgactgtgtttctgagcaataa

SEQ ID NO: 22 - DNA, 1866 bp, Adeno-associated dependoparvovirus A
  source 1..1866: mol_type = genomic DNA
atgccggggttctacgaggtggtgatcaaggtgcccagcgacctggacgagcacctgcccggcatttctgactcctttgtgaactgggtggccgagaaggaatgggagttgcccccggattctgacatggatcagaatctgattgagcaggcacccctgaccgtggccgagaagctgcagcgcgagttcctggtggaatggcgccgagtgagtaaatttctggaggccaagttttttgtgcagtttgaaaagggggactcgtactttcatttgcatattctgattgaaattaccggcgtgaaatccatggtggtgggccgctacgtgagtcagattagggataaactgatccagcgcatctaccgcggggtcgagccccagctgcccaactggttcgcggtcacaaagacccgaaatggcgccggaggcgggaacaaggtggtggacgagtgctacatccccaactacctgctccccaaggtccagcccgagcttcagtgggcgtggactaacatggaggagtatataagcgcctgtttgaacctcgcggagcgtaaacggctcgtggcgcagcacctgacgcacgtctcccagacccaggagggcgacaaggagaatctgaacccgaattctgacgcgccggtgatccggtcaaaaacctccgccaggtacatggagctggtcgggtggctggtggacaagggcatcacgtccgagaagcagtggatccaggaggaccaggcctcgtacatctccttcaacgcggcctccaactcccggtcgcagatcaaggcggccctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaagtttggaaagcgcaacaccatctggctgtttgggcccgccaccaccggcaagaccaacatcgcggaagccatcgcccacgcggtccccttctacggctgcgtcaactggaccaatgagaactttcccttcaacgactgcgtcgacaaaatggtgatttggtgggaggagggcaagatgaccgccaaggtcgtagagtccgccaaggccattctgggcggcagcaaggtgcgcgtggaccaaaaatgcaaggcctctgcgcagatcgaccccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcggctgatcacgtgactgacgtggctcatgagttttacgtcacaaagggtggagctaagaaaaggcccgccccctctgacgaggatataagcgagcccaagcggccgcgcgtgtcatttgcgcagccggagacgtcagacgcggaagctcccggagacttcgccgacaggtaccaaaacaaatgttctcgtcacgcgggtatgctgcagatgctctttccctgcaagacgtgcgagagaatgaatcagaattccaacgtctgcttcacgcacggtcagaaagattgcggggagtgctttcccgggtcagaatctcaaccggtttctgtcgtcagaaaaacgtatcagaaactgtgcatccttcatcagctccggggggcacccgagatcgcctgctctgcttgcgaccaactcaaccccgatttggacgattgccaatttgagcaataa

SEQ ID NO: 23 - DNA, 1872 bp, Adeno-associated dependoparvovirus A
  source 1..1872: mol_type = genomic DNA
atgccgggattctacgagattgtcctgaaggtgcccagcgacctggacgagcacctgcctggcatttctgactcttttgtaaactgggtggcggagaaggaatgggagctgccgccggattctgacatggatctgaatctgattgagcaggcacccctaaccgtggccgaaaagctgcaacgcgaattcctggtcgagtggcgccgcgtgagtaaggccccggaggccctcttctttgttcagttcgagaagggggacagctacttccacctacacattctggtggagaccgtgggcgtgaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaatatggaccagtatttaagcgcctgtttgaatctcgcggagcgtaaacggctggtggcgcagcatctgacgcacgtgtcgcagacgcaggagcagaacaaagagaaccagaatcccaattctgacgcgccggtgatcagatcaaaaacctccgcgaggtacatggagctggtcgggtggctggtggaccgcgggatcacgtcagaaaagcaatggatccaggaggaccaggcctcttacatctccttcaacgccgcctccaactcgcggtcacaaatcaaggccgcactggacaatgcctccaaatttatgagcctgacaaaaacggctccggactacctggtgggaaacaacccgccggaggacattaccagcaaccggatctacaaaatcctcgagatgaacgggtacgatccgcagtacgcggcctccgtcttcctgggctgggcgcaaaagaagttcgggaagaggaacaccatctggctctttgggccggccacgacgggtaaaaccaacatcgctgaagctatcgcccacgccgtgcccttttacggctgcgtgaactggaccaatgagaactttccgttcaacgattgcgtcgacaagatggtgatctggtgggaggagggcaagatgacggccaaggtcgtggagtccgccaaggccattctgggcggaagcaaggtgcgcgtggaccaaaagtgcaagtcatcggcccagatcgacccaactcccgtcatcgtcacctccaacaccaacatgtgcgcggtcatcgacggaaattccaccaccttcgagcaccaacaaccactccaagaccggatgttcaagttcgagctcaccaagcgcctggagcacgactttggcaaggtcaccaagcaggaagtcaaggactttttccggtgggcgtcagatcacgtgactgaggtgtctcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggcatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacatatcagaaactgtgtccgattcatcacatcatggggagggcgcccgaggtggcttgttcggcctgcgatctggccaatgtggacttggatgactgtgacatggagcaataa

SEQ ID NO: 24 - protein, 623 aa, Adeno-associated dependoparvovirus A
MPGFYEIVIKVPSDLDEHLPGISDSFVSWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYEPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 25 - protein, 735 aa, Adeno-associated dependoparvovirus A [sequence omitted]

SEQ ID NO: 26 - protein, 624 aa, Adeno-associated dependoparvovirus A
MPGFYEIVLKVPSDLDEHLPGISNSFVNWVAEKEWELPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFKFELTRRLDHDFGKVTKQEVKDFFRWASDHVTDVAHEFYVRKGGAKKRPASNDADVSEPKRQCTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 27 - protein, 623 aa, Adeno-associated dependoparvovirus A
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 28 - protein, 610 aa, Adeno-associated dependoparvovirus B
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 29 - protein, 623 aa, Adeno-associated dependoparvovirus A
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 30 - protein, 623 aa, Adeno-associated dependoparvovirus A
MPGFYEIVIKVPSDLDEHLPGISDSFVNWVAEKEWELPPDSDMDLNLIEQAPLTVAEKLQRDFLVQWRRVSKAPEALFFVQFEKGESYFHLHVLVETTGVKSMVLGRFLSQIREKLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKTNRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADISEPKRACPSVADPSTSDAEGAPVDFADRYQNKCSRHAGMIQMLFPCKTCERMNQNFNICFTHGVRDCLECFPGVSESQPVVRKKTYRKLCAIHHLLGRAPEIACSACDLVNVDLDDCVSEQ

SEQ ID NO: 31 - protein, 625 aa, Adeno-associated dependoparvovirus A
MPGFYEIVIKVPSDLDEHLPGISDSFVNWVAEKEWELPPDSDMDRNLIEQAPLTVAEKLQRDFLVQWRRVSKAPEALFFVQFEKGESYFHLHVLVETTGVKSMVLGRFLSQIREKLGPDHLPAGSSPTLPNWFAVTKDAVMAPAGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADITQNRIYRILALNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADKSEPKRACPSVADPSTSDAEGAPVDFADRYQNKCSRHAGMLQMLFPCKTCERMNQNFNICFTHGVRDCSECFPGVSESQPVVRKRTYRKLCAIHHLLGRAPEIACSACDLVNVDLDDCVSEQ

SEQ ID NO: 32 - protein, 623 aa, Adeno-associated dependoparvovirus A
MPGFYEIVIKVPSDLDEHLPGISDSFVNWVAEKEWELPPDSDMDRNLIEQAPLTVAEKLQRDFLVQWRRVSKAPEALFFVQFEKGESYFHLHVLVETTGVKSMVLGRFLSQIREKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPVDITQNRIYRILQLNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADKSEPKRACPSVADPSTSDAEGAPVDFADRYQNKCSRHAGMLQMLLPCKTCERMNQNFNICFTHGVRDCSECFPGVSESQPVVRKRTYRKLCAIHHLLGRAPEIACSACDLVNVDLDDCVSEQ

SEQ ID NO: 33 - protein, 622 aa, Adeno-associated dependoparvovirus A
MPGFYEIVIKVPSDLDEHLPGISDSFVNWVAEKEWELPPDSDMDRNLIEQAPLTVAEKLQRDFLVHWRRVSKAPEALFFVQFEKGESYFHLHVLVETTGVKSMVLGRFLSQIRDRLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVTHEFYVRKGGATKRPAPSDADISEPKRACPSVAEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDCVSEQ

SEQ ID NO: 34 - protein, 622 aa, Adeno-associated dependoparvovirus A
MPGFYEIVIKVPSDLDEHLPGISDSFVNWVAEKEWELPPDSDMDRNLIEQAPLTVAEKLQRDFLVHWRRVSKAPEALFFVQFEKGESYFHLHVLVETTGVKSMVLGRFLSQIRDRLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGATKRPAPSDADISEPKRACPSVPEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDCVSEQ

SEQ ID NO: 35 - protein, 621 aa, Adeno-associated dependoparvovirus A
MPGFYEVVIKVPSDLDEHLPGISDSFVNWVAEKEWELPPDSDMDQNLIEQAPLTVAEKLQREFLVEWRRVSKFLEAKFFVQFEKGDSYFHLHILIEITGVKSMVVGRYVSQIRDKLIQRIYRGVEPQLPNWFAVTKTRNGAGGGNKVVDECYIPNYLLPKVQPELQWAWTNMEEYISACLNLAERKRLVAQHLTHVSQTQEGDKENLNPNSDAPVIRSKTSARYMELVGWLVDKGITSEKQWIQEDQASYISFNAASNSRSQIKAALDNASKIMSLTKTAPDYLIGQQPVGDITTNRIYKILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKASAQIDPTPVIVTSNTNMCAVIDGNSTTFEHQQPLQDRMFKFELTRRLDHDFGKVTKQEVKDFFRWAADHVTDVAHEFYVTKGGAKKRPAPSDEDISEPKRPRVSFAQPETSDAEAPGDFADRYQNKCSRHAGMLQMLFPCKTCERMNQNSNVCFTHGQKDCGECFPGSESQPVSVVRKTYQKLCILHQLRGAPEIACSACDQLNPDLDDCQFEQ

SEQ ID NO: 36 - protein, 623 aa, Adeno-associated dependoparvovirus A
MPGFYEIVLKVPSDLDEHLPGISDSFVNWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKFMSLTKTAPDYLVGNNPPEDITSNRIYKILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVSHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACDLANVDLDDCDMEQ

SEQ ID NO: 37 - DNA, 1872 bp, synthetic construct
  source 1..1872: mol_type = other DNA
atgccggggttctacgagattgtcctgaaggtcccgagtgacctggacgagcacctgccgggcatttctaactcgtttgttaactgggtggccgagaaggaatgggagctgcccccggattctgacatggatcggaatctgatcgagcaggcacccctgaccgtggccgagaagctgcagcgcgacttcctggtccagtggcgccgcgtgagtaaggccccggaggccctcttctttgttcagttcgagaagggcgagagctactttcacctgcacgttctggtcgagaccacgggggtcaagtccatggtcctgggccgcttcctgagtcagatcagagacaggctggtgcagaccatctaccgcggggtcgagcccacgctgcccaactggttcgcggtgaccaaagacgcggtaatggcgccggcgggggggaacaaggtggtggacgagtgctacatccccaactacctcctgcccaagacccagcccgagctgcagtgggcgtggactaacatggaggagtatataagcgcgtgtctaaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcacaaatcaaggccgcactggacaatgccggcaagatcatggcgctgaccaaatccgcgcccgactacctggtaggcccgtccttacccgcggacattaaggccaaccgcatctaccgcatcctggagctcaacggctacgaccccgcctacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtcaactggaccaatgagaactttccgttcaacgactgtgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcgcaggatcacgtgaccgaggtggcgcatgagttctacgtcagaaagggtggagccaacaagagacccgcccccagtgacgcggatataagcgagcccaagcgggcctgcccctcagttccggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacctgtcgtcagaaaaaagacgtatcagaaactgtgcgcgattcatcatctgctggggcgggcacccgagattgcgtgttcggcctgcgatctcgtcaacgtggacttggatgactgkgtttctgaacaataa

SEQ ID NO: 38 - DNA, 1872 bp, synthetic construct
  source 1..1872: mol_type = other DNA
atgccgggcttctacgagattgtcctgaaggtcccgagtgacctggacgagcacctgccgggcatttctaactcgtttgttaactgggtggccgagaaggaatgggagctgccgccggattctgacatggacttgaatctgattgagcaggcacccctgaccgtggccgaaaagctgcagcgcgacttcctggtccactggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggcggggggaacaaggtggtggacgagtgctacatccccaactacctgctccccaagacccagcccgagctgcagtgggcgtggactaacatggaggagtatataagcgcctgtttgaacctcgcggagcgtaaacggctcgtggcgcagcatctgacgcacgtgtcgcagacgcaggagcagaacaaggagaatctgaacccgaattctgacgcgcccgtgatcaggtcaaaaacctccgcgcgctacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaggttcgggaagcgcaacaccatctggctgtttgggccggccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgaccgccaaggtcgtagagagcgccaaggccatcctgggcggaagcaaggtgcgcgtggaccaaaagtgcaagtcgtccgcccagatcgaccccactcccgtgatcgtcacctccaacaccaacatgtgcgccgtgattgacgggaacagcaccaccttcgagcaccagcagcccctgcaggaccggatgttcaaatttgaacttacccgccgtttggaccatgactttggcaaggtcaccaagcaggaagtcaaagactttttccggtgggcgtcagatcacgtgaccgaggtgactcacgagttttacgtcagaaagggcggagccagcaaaagacccgcccccgatgacgcggataaaagcgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacatatcagaaactgtgtttgattcatcacatcatggggagggcgcccgaggtggcttgttcggcctgcgaactggccaatgtggacttggatgactgtgacatggaacaataa

SEQ ID NO: 39 - DNA, 1875 bp, synthetic construct
  source 1..1875: mol_type = other DNA
atgccggggttttacgagattgtgattaaggtccccagcgaccttgacgagcatctgcccggcatttctgacagctttgtgaactgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagagctacttccaccttcacgttctggtggagaccacgggggtcaagtccatggtgctaggccgcttcctgagtcagattcgggagaagctggtccagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacccaggagcagaacaaggagaatctgaaccccaattctgacgcgcccgtgatcaggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtcggccaaagccattctcggaggaagcaaggtgcgcgtggaccaaaagtgcaagtcctcggcccagatcgaccccacgcccgtgatcgtcacctccaacaccaacatgtgcgccgtgatcgacgggaacagcaccaccttcgagcaccagcagccgctgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggcggagccaccaaaagacccgcccccagtgacgcggatataagcgagcccaagcgggcctgcccctcagttccggagccatcgacgtcagacgcggaagcgccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgtgtcagaatctcaacccgtgtctgtcgtcagaaagcggacatatcagaaactgtgtccgattcatcacatcatggggagggcgcccgagattgcttgctcggcctgcgatctggtcaacgtggacctggatgactgtgtttctgagcaataa

SEQ ID NO: 40 - DNA, 1872 bp, synthetic construct
  source 1..1872: mol_type = other DNA
atgccggggttctacgagattgtcctgaaggtcccgagtgacctggacgagcacctgccgggcatttctaactcgtttgttaactgggtggccgagaaggaatgggagttgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggagaagctggtccagaccatctaccgcgggatcgagccgaccctgcccaactggttcgcggtgaccaagacgcgtaatggcgccggcggggggaacaaggtggtggacgactgctacatccccaactacctgctccccaagacccagcccgagctccagtgggcgtggactaacatggaccagtatttaagcgcctgtttgaatctcacggagcgtaaacggttggtggcgcagcatctgacgcacgtgtcgcagacgcaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggctcagaaaaagttcggtaaaaggaatacaatttggctgttcgggcccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgactgaggtggcgcatgagttctacgtcagaaagggtggagccaagaaaaggcccgccccctctgacgaggatataagcgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacatatcagaaactgtgtttgattcatcacatcatggggagggcgcccgaggtggcttgttcggcctgcgaactggccaatgtggacttggatgactgtgacatggaacaataa

SEQ ID NO: 41 - DNA, 1866 bp, synthetic construct
  source 1..1866: mol_type = other DNA
atgccggggttttacgagattgtgattaaggtccccagcgaccttgacgagcatctgcccggcatttctgacagctttgtgaactgggtggccgagaaggaatgggagctgcccccggattctgacatggatctgaatctgattgagcaggcacccctgaccgtggccgagaagctgcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctcgtggacaaggggattacctcggagaagcagtggatccaggaggaccaggcctcgtacatctccttcaacgccgcctccaactcgcggtcacaaatcaaggccgcgctggacaatgcctccaaaatcatgagcctgacaaagacggctccggactacctggtgggccagaacccgccggaggacattaccagcaaccggatctacaaaatcctcgagatgaacgggtacgatccgcagtacgcggcctccgtcttcctgggctgggcgcaaaagaagttcggtaaacgcaacaccatctggctgtttgggcctgcaactaccggcaagaccaacatcgcggaagccatcgcccacgcggtccccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaaatgcaaggcctctgcgcagatcgaccccacccccgtgatcgtcacctccaacaccaacatgtgcgccgtgatcgacgggaacagcaccaccttcgagcaccagcagcccctgcaggaccgcatgttcaaatttgaactcacccgccgtctggagcacgactttggcaaggtgacgaagcaggaagtcaaagagttcttccgctgggccagtgatcacgtgactgaggtgtctcacgagttttacgtcagaaagggtggagccaacaaaagacccgcccccgatgacgcggataaaagcgagcccaagcgggcctgcccctcagttgcggagccatcgacgtcagacgcggaagcaccggtggactttgcggacaggtaccaaaacaaatgttctcgtcacgcgggcatgcttcagatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 42 - DNA, 1869 bp, synthetic construct
  source 1..1869: mol_type = other DNA
atgccggggttctacgagattgtcctgaaggtcccgagtgacctggacgagcacctgccgggcatttctaactcgtttgttaactgggtggccgagaaggaatgggagctgccgccagattctgacatggatcggaatctgatcgagcaggcacccctgaccgtggccgagaagctgcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacatttccagcaaccggatctacaaaatcctcgagatgaacgggtacgatccgcagtacgcggcctccgtctttctcggctgggcacaaaagaagttcgggaaacgcaacaccatctggctgtttgggccggccaccacgggaaagaccaacatcgcagaagccattgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctgggcggaagcaaggtgcgcgtggaccaaaagtgcaagtcgtccgcccagatcgaccccacccccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggcgcaggatcacgtgaccgaggtgtctcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacacacggggtcagagactgctcagagtgtttccccggcgtgtcagaatctcaaccggtcgtcagaaaaaagacgtatcagaaactgtgtccgattcatcacatcatggggagggcgcccgaggtggcctgctcggcctgcgaactggccaatgtggacttggatgactgtgacatggaacaataa

SEQ ID NO: 43 - DNA, 1869 bp, synthetic construct; identical to SEQ ID NO: 42
  source 1..1869: mol_type = other DNA

SEQ ID NO: 44 - DNA, 1869 bp, synthetic construct
  source 1..1869: mol_type = other DNA
atgccggggttctacgagattgtcctgaaggtcccgagtgacctggacgagcacctgccgggcatttctaactcgtttgtgaactgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggacaagctggtgcagaccatctaccgcgggatcgagccgaccctgcccaactggttcgcggtgaccaagacgcgtaatggcgccggcggggggaacaaggtggtggacgagtgctacatccccaactacctgctccccaagacccagcccgagctgcagtgggcgtggactaacatggaggagtatataagcgcgtgcttgaacctggccgagcgcaaacggctcgtggcgcagcacctgacccacgtcagccagacccaggagcagaacaaggagaatctgaaccccaattctgacgcgcccgtgatcaggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacatttccagcaaccggatctacaaaatcctcgagatgaacgggtacgatccgcagtacgcggcctccgtctttctcggctgggcacaaaagaagttcgggaaacgcaacaccatctggctgtttgggccggccaccacgggaaagaccaacatcgcagaagccattgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctgggcggaagcaaggtgcgcgtggaccaaaagtgcaagtcgtccgcccagatcgaccccactcccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggcgcaggatcacgtgaccgaggtgtctcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacacacggggtcagagactgctcagagtgtttccccggcgtgtcagaatctcaaccggtcgtcagaaaaaagacgtatcagaaactgtgtccgattcatcacatcatggggagggcgcccgaggtggcctgctcggcctgcgaactggccaatgtggacttggatgactgtgacatggaacaataa

SEQ ID NO: 45 - DNA, 1869 bp, synthetic construct; identical to SEQ ID NO: 42
  source 1..1869: mol_type = other DNA

SEQ ID NO: 46 - DNA, 1878 bp, synthetic construct
  source 1..1878: mol_type = other DNA
atgccggggttttacgagattgtgattaaggtccccagcgaccttgacgagcatctgcccggcatttctgacagctttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctgctccccaagacccagcccgagctccagtgggcgtggactaatatggaccagtatttaagcgcctgtttgaatctcacggagcgtaaacggttggtggcgcagcatctgacgcacgtgtcgcagacgcaggagcagaacaaggagaatctgaacccgaattctgacgcgcccgtgatcaggtcaaaaacctccgcgcgctacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaggccatcgcccacgccgtgcccttctacggctgcgtcaactggaccaatgagaactttcccttcaacgactgcgtcgacaagatggtgatctggtgggaggagggcaagatgacggccaaggtcgtagagagcgccaaggccatcctgggcggaagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaacccgtgtctgtcgtcagaaagcggacatatcagaaactgtgtccgattcatcacatcatggggagggcgcccgaggtggcttgttcggcctgcgatctggccaatgtggacttggatgactgtgacatggagcaataa

SEQ ID NO: 47 - DNA, 1869 bp, synthetic construct
  source 1..1869: mol_type = other DNA
atgccgggcttctacgagatcgtgatcaaggtgccgagcgacctggacgagcacctgccgggcatttctgactcgtttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacatttccagcaaccggatctacaaaatcctcgagatgaacgggtacgatccgcagtacgcggcctccgtctttctcggctgggcacaaaagaagttcgggaaacgcaacaccatctggctgtttgggccggccaccacgggaaagaccaacatcgcagaagccattgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctgggcggaagcaaggtgcgcgtggaccaaaagtgcaagtcgtccgcccagatcgaccccacccccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggcgcaggatcacgtgaccgaggtgtctcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacacacggggtcagagactgctcagagtgtttccccggcgtgtcagaatctcaaccggtcgtcagaaaaaagacgtatcagaaactgtgtccgattcatcacatcatggggagggcgcccgaggtggcctgctcggcctgcgaactggccaatgtggacttggatgactgtgacatggaacaataa

SEQ ID NO: 48 - DNA, 41 bp, synthetic construct
  source 1..41: mol_type = other DNA
ttagtattaattaagaatgcgagccaatctggaagataacc

SEQ ID NO: 49 - protein, 623 aa, synthetic construct
MPGFYEIVLKVPSDLDEHLPGISNSFVNWVAEKEWELPPDSDMDRNLIEQAPLTVAEKLQRDFLVQWRRVSKAPEALFFVQFEKGESYFHLHVLVETTGVKSMVLGRFLSQIRDRLVQTIYRGVEPTLPNWFAVTKDAVMAPAGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAQDHVTEVAHEFYVRKGGANKRPAPSDADISEPKRACPSVPEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDXVSEQ

SEQ ID NO: 50 - protein, 623 aa, synthetic construct
MPGFYEIVLKVPSDLDEHLPGISNSFVNWVAEKEWELPPDSDMDLNLIEQAPLTVAEKLQRDFLVHWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNASKIMSLTKTAPDYLIGQQPVGDITTNRIYKILELNGYDPQYAASVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWASDHVTEVTHEFYVRKGGASKRPAPDDADKSEPKRACPSVAQPSTSDAEAPVDYADRYQNKCSRHVGMNLMLFPCRQCERMNQNVDICFTHGVMDCAECFPVSESQPVSVVRKRTYQKLCLIHHIMGRAPEVACSACELANVDLDDCDMEQ

SEQ ID NO: 51 - protein, 624 aa, synthetic construct
MPGFYEIVIKVPSDLDEHLPGISDSFVNWVAEKEWELPPDSDMDLNLIEQAPLTVAEKLQRDFLVQWRRVSKAPEALFFVQFEKGESYFHLHVLVETTGVKSMVLGRFLSQIREKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGATKRPAPSDADISEPKRACPSVPEPSTSDAEAPVDFADRYQNKCSRHAGMLQMLFPCKTCERMNQNFNVCFTHGVRDCSECFPGVSESQPVSVVRKRTYQKLCPIHHIMGRAPEIACSACDLVNVDLDDCVSEQ

SEQ ID NO: 52 - protein, 621 aa, synthetic construct
MPGFYEIVIKVPSDLDEHLPGISDSFVNWVAEKEWELPPDSDMDLNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMEEYISACLNLAERKRLVAQHLTHVSQTQEQNKENLNPNSDAPVIRSKTSARYMELVGWLVDKGITSEKQWIQEDQASYISFNAASNSRSQIKAALDNASKIMSLTKTAPDYLVGQNPPEDITSNRIYKILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKASAQIDPTPVIVTSNTNMCAVIDGNSTTFEHQQPLQDRMFKFELTRRLEHDFGKVTKQEVKEFFRWASDHVTEVSHEFYVRKGGANKRPAPDDADKSEPKRACPSVAEPSTSDAEAPVDFADRYQNKCSRHAGMLQMLFPCRQCERMNQNSNICFTHGQKDCLECFPVSESQPVSVVKKAYQKLCYIHHIMGKVPDACTACDLVNVDLDDCIFEQ

SEQ ID NO: 53 - DNA, 20 bp, synthetic construct
  source 1..20: mol_type = other DNA
ttgacgtcaatgggtggagt

SEQ ID NO: 54 - DNA, 20 bp, synthetic construct
  source 1..20: mol_type = other DNA
cgggccatttaccgtcattg

SEQ ID NO: 55 - DNA, 29 bp, synthetic construct
  source 1..29: mol_type = other DNA
acatcaagtgtatcatatgccaagtacgc

SEQ ID NO: 56 - DNA, 23 bp, synthetic construct
  source 1..23: mol_type = other DNA
  modified_base 1: mod_base = OTHER; note = CY5
  modified_base 23: mod_base = OTHER; note = BHQ2
cattgtctgagtaggtgtcattc

SEQ ID NO: 57 - DNA, 20 bp, synthetic construct
  source 1..20: mol_type = other DNA
tgcctgctattgtcttccca

SEQ ID NO: 58 - DNA, 20 bp, synthetic construct
  source 1..20: mol_type = other DNA
  modified_base 1: mod_base = OTHER; note = HEX
  modified_base 20: mod_base = OTHER; note = BHQ1
cctcccccttgctgtcctgc

SEQ ID NO: 59 - DNA, 20 bp, synthetic construct
  source 1..20: mol_type = other DNA
tagccgctaccctgatcata

SEQ ID NO: 60 - DNA, 20 bp, synthetic construct
  source 1..20: mol_type = other DNA
taaagaagatggtccgctcc

SEQ ID NO: 61 - DNA, 25 bp, synthetic construct
  source 1..25: mol_type = other DNA
  modified_base 1: mod_base = OTHER; note = FAM
  modified_base 25: mod_base = OTHER; note = BHQ1
tctttaagtccgctatgccagaagg